(19) United States
(12) Patent Application Publication
(10) Pub. No.: US 2023/0135868 A1
(43) Pub. Date: May 4, 2023

(54) ORGANIC COMPOUND, ORGANIC LIGHT-EMITTING ELEMENT, DISPLAY APPARATUS, PHOTOELECTRIC CONVERSION APPARATUS, ELECTRONIC EQUIPMENT, LIGHTING APPARATUS, MOVING BODY, AND EXPOSURE LIGHT SOURCE

(71) Applicant: CANON KABUSHIKI KAISHA, Tokyo (JP)

(72) Inventors: Hirokazu Miyashita, Kanagawa (JP); Jun Kamatani, Tokyo (JP); Naoki Yamada, Tokyo (JP); Yosuke Nishide, Kanagawa (JP); Hironobu Iwawaki, Kanagawa (JP); Hiroki Ohrui, Tokyo (JP)

(21) Appl. No.: 17/932,148

(22) Filed: Sep. 14, 2022

(30) Foreign Application Priority Data
Sep. 16, 2021 (JP) 2021-151163

(51) Int. Cl. C07F 15/00; H01L 51/00; H01L 51/50; H01L 51/44

(52) U.S. Cl. CPC C07F 15/0033; H01L 51/0085; H01L 51/009; H01L 51/0094; H01L 51/0067; H01L 51/0072; H01L 51/0052; H01L 51/0071; H01L 51/0054; H01L 51/0056; H01L 51/0073; H01L 51/0074; H01L 51/5012; H01L 51/44; C07B 2200/05

(57) ABSTRACT

An organic compound represented by the following general formula [1] is provided. In the following general formula [1] IrLₘL′ₙ, where L and L′ denote different bidentate ligands, the partial structure IrL denotes a partial structure represented by the general formula [A-1] or [A-2], and the partial structure IrL′ denotes a partial structure represented by the general formula [B-1] or [B-2].

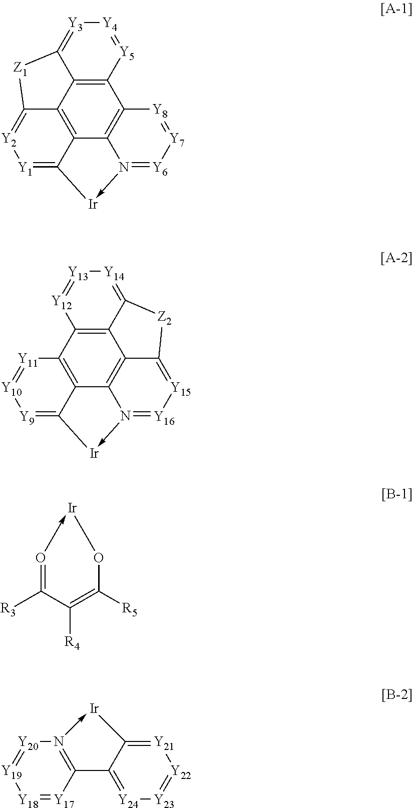

### PRIORITY AND INCORPORATION BY REFERENCE

This application claims the benefit of Japanese Patent Application No. 2021-151163 filed Sep. 16, 2021, which is hereby incorporated by reference herein in its entirety.

### BACKGROUND OF THE INVENTION

### Field of the Invention

The present disclosure relates to an organic compound, an organic light-emitting element, a display apparatus, a photoelectric conversion apparatus, electronic equipment, a lighting apparatus, a moving body, and an exposure light source.

### Description of the Related Art

An organic light-emitting element (hereinafter sometimes referred to as an “organic electroluminescent element” or an “organic EL element”) is an electronic element that includes a pair of electrodes and an organic compound layer between the electrodes. Electrons and holes are injected from the pair of electrodes to generate an exciton of a light-emitting organic compound in the organic compound layer. When the exciton returns to its ground state, the organic light-emitting element emits light.

With recent significant advances in organic light-emitting elements, it is characteristically possible to realize low drive voltage, various emission wavelengths, high-speed responsivity, and thin and light light-emitting devices.

Light-emitting organic compounds have been actively developed. This is because the development of compounds with good emission properties is important for high-performance organic light-emitting elements.

U.S. Patent Application Publication No. 2010/0327736 (PTL 1) discloses the following compound 1-a as a compound developed so far.

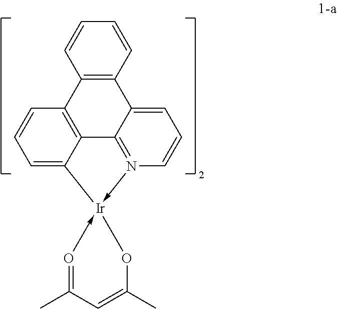

It has been found that the compound 1-a described in PTL 1 has room for improvement in emission properties. An organic light-emitting element with higher luminescence efficiency is desired.

### SUMMARY OF THE INVENTION

In view of such a situation, the present disclosure provides an organic compound with good emission properties. The present disclosure also provides an organic light-emitting element with good emission properties.

An organic compound according to one aspect of the present disclosure is represented by the following general formula [1]:

Ir Lₘ L′ₙ [1]

wherein Ir denotes iridium. L and L′ denote different bidentate ligands. m denotes an integer in the range of 1 to 3, n is 2 when m is 1, n is 1 when m is 2, and n is 0 when m is 3. The partial structure IrL denotes a partial structure represented by the following general formula [A-1] or [A-2], and the partial structure IrL′ denotes a partial structure represented by the following general formula [B-1] or [B-2]. When m is 2 or more, the Ls may be the same or different. When n is 2, the L′s may be the same or different.

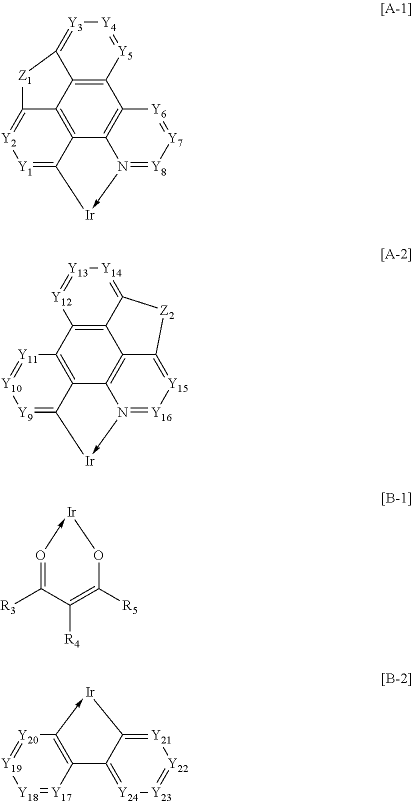

Y₁ to Y₂₄ in the general formulae [A-1], [A-2], and [B-2] are independently selected from a carbon atom and a nitrogen atom. When Y₁ to Y₂₄ denote a carbon atom, the carbon atom has a hydrogen atom, a deuterium atom, or a substituent R. When two or more of Y₁ to Y₂₄ denote a carbon atom with the substituent R, the substituents R may have the same or different structures.

The substituent R denotes a substituent independently selected from a halogen atom, a substituted or unsubstituted alkyl group, a substituted or unsubstituted alkoxy group, a substituted or unsubstituted amino group, a substituted or unsubstituted aryloxy group, a substituted or unsubstituted silyl group, a cyano group, a substituted or unsubstituted aromatic hydrocarbon group, and a substituted or unsubstituted heterocyclic group.

When any adjacent two of Y₁ to Y₂₄ in the general formulae [A-1], [A-2], and [B-2] simultaneously denote a carbon atom and have the substituent R, the substituents R may be bonded together and form a ring. The ring structure is a benzene ring, a naphthalene ring, an azine ring, a thiophene ring, or a furan ring.

Z₁ and Z₂ in the general formulae [A-1] and [A-2] are independently selected from an oxygen atom, a sulfur atom, SiR₁R₂, CR₁R₂, GeR₁R₂, NR₁, and CR₁═CR₂. R₁ and R₂ may be bonded together and form a ring.

R₁ to R₅ in the general formulae [A-1], [A-2], and [B-1] are independently selected from a halogen atom, a substituted or unsubstituted alkyl group, a cyano group, a substituted or unsubstituted aromatic hydrocarbon group, and a substituted or unsubstituted heterocyclic group.

Further features of the present disclosure will become apparent from the following description of exemplary embodiments with reference to the attached drawings.

### BRIEF DESCRIPTION OF THE DRAWINGS

FIG. 1A is a schematic cross-sectional view of an example of a pixel of a display apparatus according to an embodiment of the present disclosure.

FIG. 1B is a schematic cross-sectional view of an example of a display apparatus including an organic light-emitting element according to an embodiment of the present disclosure.

FIG. 2 is a schematic view of an example of a display apparatus according to an embodiment of the present disclosure.

FIG. 3A is a schematic view of an example of an imaging apparatus according to an embodiment of the present disclosure.

FIG. 3B is a schematic view of an example of a mobile device according to an embodiment of the present disclosure.

FIG. 4A is a schematic view of an example of a display apparatus according to an embodiment of the present disclosure.

FIG. 4B is a schematic view of an example of a foldable display apparatus according to an embodiment of the present disclosure.

FIG. 5A is a schematic view of an example of a lighting apparatus according to an embodiment of the present disclosure.

FIG. 5B is a schematic view of an automobile of an example of a moving body according to an embodiment of the present disclosure.

FIG. 6A is a schematic view of an example of a wearable device according to an embodiment of the present disclosure.

FIG. 6B is a schematic view of an example of a wearable device according to an embodiment of the present disclosure with an imaging apparatus.

FIG. 7 is a schematic view of an example of an image-forming apparatus according to an embodiment of the present disclosure.

FIG. 8A is a schematic view of an example of an exposure light source of an image-forming apparatus according to an embodiment of the present disclosure.

FIG. 8B is a schematic view of an example of an exposure light source of an image-forming apparatus according to an embodiment of the present disclosure.

FIG. 9 is a schematic view of the structures of exemplary compounds and a comparative compound and the symmetries of ligands.

FIG. 10 is a schematic view of the structures of an exemplary compound and a comparative compound and the symmetries of ligands.

FIG. 11 a schematic view of the structures of an exemplary compound and a comparative compound and the three-dimensional structures of ligands.

### DESCRIPTION OF THE EMBODIMENTS

### Organic Compound

First, an organic compound according to the present embodiment is described below.

The organic compound according to the present embodiment is an organic compound represented by the following general formula [1]. The organic compound may also be referred to as an organometallic complex because organic ligands coordinate to a metal.

IrLₘ L′ₙ [1]

In the general formula [1], Ir denotes iridium. L and L′ denote different bidentate ligands. m denotes an integer in the range of 1 to 3, n is 2 when m is 1, n is 1 when m is 2, and n is 0 when m is 3. The partial structure IrL denotes a partial structure represented by the following general formula [A-1] or [A-2], and the partial structure IrL′ denotes a partial structure represented by the following general formula [B-1] or [B-2]. When m is 2 or more, the Ls may be the same or different. When n is 2, the L′s may be the same or different.

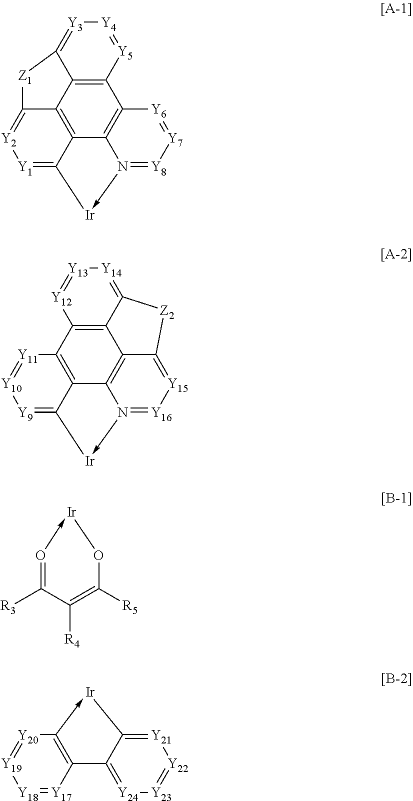

Y₁ to Y₂₄ in the general formulae [A-1], [A-2], and [B-2] are independently selected from a carbon atom and a nitrogen atom. When Y₁ to Y₂₄ denote a carbon atom, the carbon atom has a hydrogen atom, a deuterium atom, or a substituent R. When two or more of Y₁ to Y₂₄ denote a carbon atom with the substituent R, the substituents R may have the same or different structures.

The substituent R denotes a substituent independently selected from a halogen atom, a substituted or unsubstituted alkyl group, a substituted or unsubstituted alkoxy group, a substituted or unsubstituted amino group, a substituted or unsubstituted aryloxy group, a substituted or unsubstituted silyl group, a cyano group, a substituted or unsubstituted aromatic hydrocarbon group, and a substituted or unsubstituted heterocyclic group.

When any adjacent two of Y₁ to Y₂₄ in the general formulae [A-1], [A-2], and [B-2] simultaneously denote a carbon atom and have the substituent R, the substituents R may be bonded together and form a ring. The ring structure is a benzene ring, a naphthalene ring, an azine ring, a thiophene ring, or a furan ring.

Z₁ and Z₂ in the general formulae [A-1] and [A-2] are independently selected from an oxygen atom, a sulfur atom, SiR₁R₂, CR₁R₂, GeR₁R₂, NR₁, and CR₁═CR₂. R₁ and R₂ may be bonded together and form a ring.

R₁ to R₅ in the general formulae [A-1], [A-2], and [B-1] are independently selected from a halogen atom, a substituted or unsubstituted alkyl group, a cyano group, a substituted or unsubstituted aromatic hydrocarbon group, and a substituted or unsubstituted heterocyclic group.

In the organic compound according to the present embodiment, the partial structure IrL in the general formula [1] can be a partial structure represented by one of the following general formulae [A-11] to [A-14] and [A-21] to [A-24].

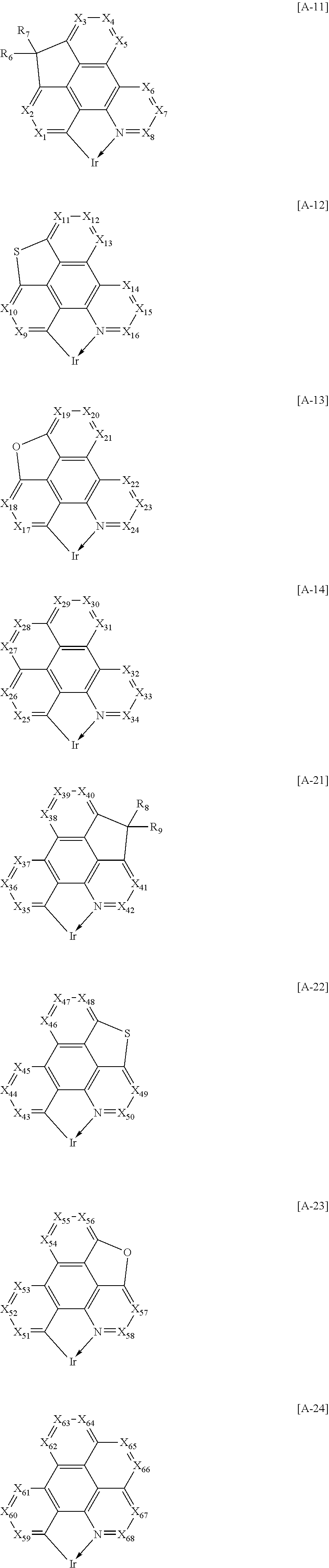

X₁ to X₆₈ in the general formulae [A-11] to [A-14] and [A-21] to [A-24] are independently selected from a carbon atom and a nitrogen atom. When X₁ to X₆₈ denote a carbon atom, the carbon atom has a hydrogen atom, a deuterium atom, or a substituent R. When two or more of X₁ to X₆₈ denote a carbon atom with the substituent R, the substituents R may have the same or different structures.

The substituent R denotes a substituent independently selected from a halogen atom, a substituted or unsubstituted alkyl group, a substituted or unsubstituted alkoxy group, a substituted or unsubstituted amino group, a substituted or unsubstituted aryloxy group, a substituted or unsubstituted silyl group, a cyano group, a substituted or unsubstituted aromatic hydrocarbon group, and a substituted or unsubstituted heterocyclic group.

When any adjacent two of X₁ to X₆₈ in the general formulae [A-11] to [A-14] and [A-1] to [A-24] simultaneously denote a carbon atom and have the substituent R, the substituents R may be bonded together and form a ring. The ring structure is a benzene ring, a naphthalene ring, an azine ring, a thiophene ring, or a furan ring.

R₆ to R₉ in the general formulae [A-11] and [A-21] are independently selected from a halogen atom, a substituted or unsubstituted alkyl group, a cyano group, a substituted or unsubstituted aromatic hydrocarbon group, and a substituted or unsubstituted heterocyclic group.

The optional substituent R of the carbon atom when Y₁ to Y₂₄ denote a carbon atom and the optional substituent R of the carbon atom when X₁ to X₆₈ denote a carbon atom can denote a substituent independently selected from a halogen atom, a substituted or unsubstituted alkyl group having 1 to 10 carbon atoms, a substituted or unsubstituted alkoxy group having 1 to 10 carbon atoms, a substituted or unsubstituted amino group having 1 to 6 carbon atoms, a substituted or unsubstituted aryloxy group, a substituted or unsubstituted silyl group, a cyano group, a substituted or unsubstituted aromatic hydrocarbon group having 6 to 30 carbon atoms, and a substituted or unsubstituted heterocyclic group having 3 to 27 carbon atoms.

R₁ to R₅ in the general formulae [A-1], [A-2], and [B-1] can be independently selected from a halogen atom, a substituted or unsubstituted alkyl group having 1 to 10 carbon atoms, a cyano group, a substituted or unsubstituted aromatic hydrocarbon group, and a substituted or unsubstituted heterocyclic group.

R₆ to R₉ in the general formulae [A-11] and [A-21] can be independently selected from a halogen atom, a substituted or unsubstituted alkyl group having 1 to 10 carbon atoms, a cyano group, a substituted or unsubstituted aromatic hydrocarbon group, and a substituted or unsubstituted heterocyclic group.

The optional halogen atom as the optional substituent R of the carbon atom when Y₁ to Y₂₄ denote a carbon atom and as the optional substituent R of the carbon atom when X₁ to X₆₈ denote a carbon atom and the halogen atom of R₁ to R₅ may be, but are not limited to, fluorine, chlorine, bromine, or iodine.

The optional alkyl group as the optional substituent R of the carbon atom when Y₁ to Y₂₄ denote a carbon atom and as the optional substituent R of the carbon atom when X₁ to X₆₈ denote a carbon atom and the alkyl group of R₁ to R₅ may be, but are not limited to, a methyl group, an ethyl group, a n-propyl group, an isopropyl group, a n-butyl group, a t-butyl group, a sec-butyl group, an octyl group, a cyclohexyl group, a 1-adamantyl group, or a 2-adamantyl group.

The optional alkoxy group as the optional substituent R of the carbon atom when Y₁ to Y₂₄ denote a carbon atom and as the optional substituent R of the carbon atom when X₁ to X₆₈ denote a carbon atom may be, but are not limited to, a methoxy group, an ethoxy group, a propoxy group, a 2-ethyl-octyloxy group, or a benzyloxy group.

The optional amino group as the optional substituent R of the carbon atom when Y₁ to Y₂₄ denote a carbon atom and as the optional substituent R of the carbon atom when X₁ to X₆₈ denote a carbon atom may be, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-t-butylphenyl)amino group, an N-phenyl-N-(4-trifluoromethylphenyl)amino group, or an N-piperidyl group.

The optional aryloxy group and heteroaryloxy group as the optional substituent R of the carbon atom when Y₁ to Y₂₄ denote a carbon atom and as the optional substituent R of the carbon atom when X₁ to X₆₈ denote a carbon atom may be, but are not limited to, a phenoxy group or a thienyloxy group.

The optional silyl group as the optional substituent R of the carbon atom when Y₁ to Y₂₄ denote a carbon atom and as the optional substituent R of the carbon atom when X₁ to X₆₈ denote a carbon atom may be, but are not limited to, a trimethylsilyl group or a triphenylsilyl group.

The optional aromatic hydrocarbon group as the optional substituent R of the carbon atom when Y₁ to Y₂₄ denote a carbon atom and as the optional substituent R of the carbon atom when X₁ to X₆₈ denote a carbon atom and the aromatic hydrocarbon group of R₁ to R₅ may be, but are not limited to, a phenyl group, a naphthyl group, an indenyl group, a biphenyl group, a terphenyl group, a fluorenyl group, a phenanthryl group, a fluoranthenyl group, or a triphenylenyl group.

The optional heterocyclic group as the optional substituent R of the carbon atom when Y₁ to Y₂₄ denote a carbon atom and as the optional substituent R of the carbon atom when X₁ to X₆₈ denote a carbon atom and the heterocyclic group of R₁ to R₅ may be, but are not limited to, a pyridyl group, an oxazolyl group, an oxadiazolyl group, a thiazolyl group, a thiadiazolyl group, a carbazolyl group, an acridinyl group, a phenanthrolyl group, a dibenzofuranyl group, or a dibenzothiophenyl group.

The additional optional substituent of the alkyl group, alkoxy group, amino group, aryloxy group, silyl group, aromatic hydrocarbon group, and heterocyclic group may be, but are not limited to, a halogen atom, such as fluorine, chlorine, bromine, or iodine; an alkyl group, such as a methyl group, an ethyl group, a n-propyl group, an isopropyl group, a n-butyl group, or a t-butyl group; an alkoxy group, such as a methoxy group, an ethoxy group, or a propoxy group; an amino group, such as a dimethylamino group, a diethylamino group, a dibenzylamino group, a diphenylamino group, or a ditolylamino group; an aryloxy group, such as a phenoxy group; an aromatic hydrocarbon group, such as a phenyl group or a biphenyl group; a heterocyclic group, such as a pyridyl group or a pyrrolyl group; or a cyano group.

Method for Synthesizing Organic Compound

Next, a method for synthesizing the organic compound according to the present embodiment is described. For example, the organic compound according to the present embodiment is synthesized in accordance with the following reaction scheme.

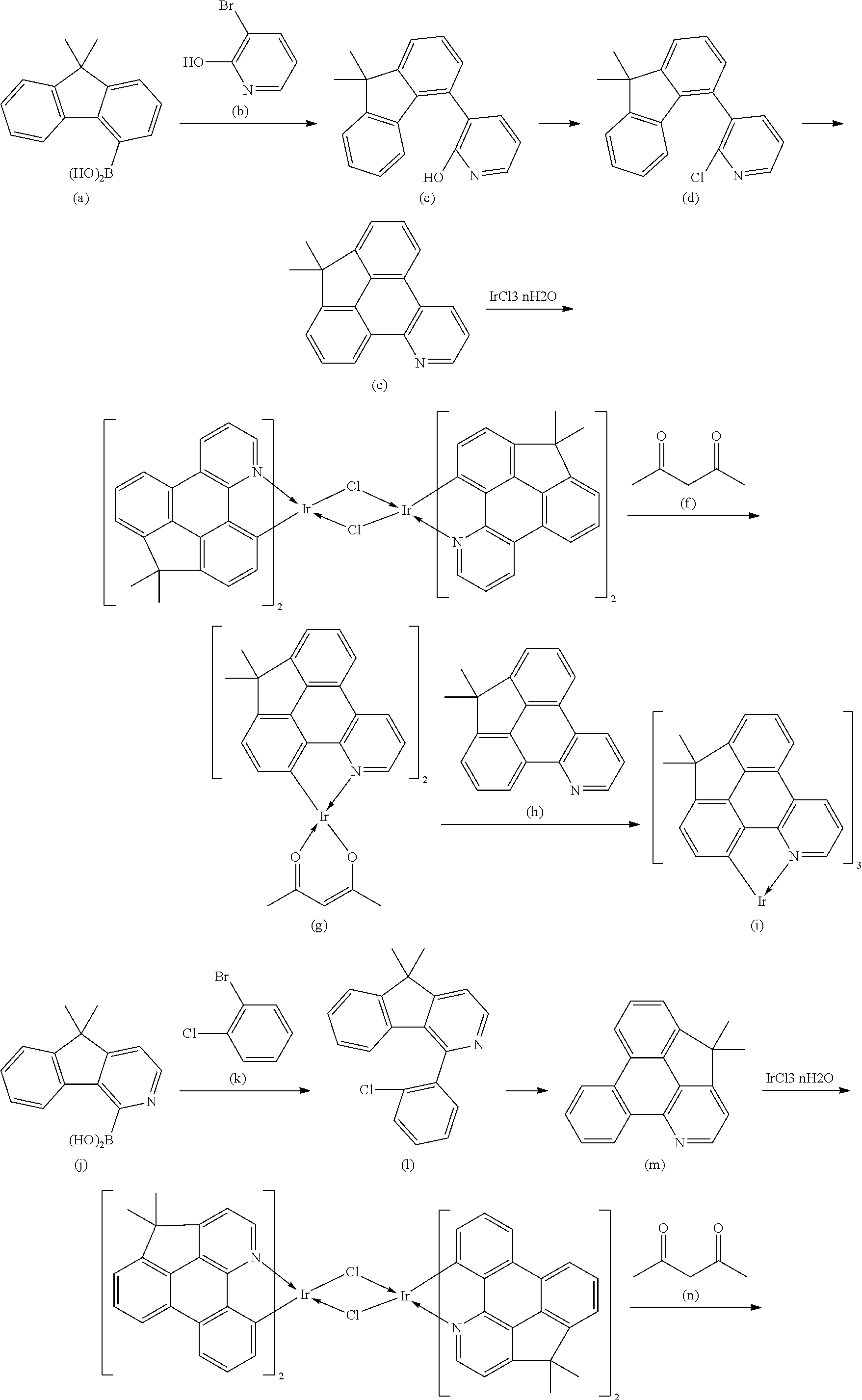

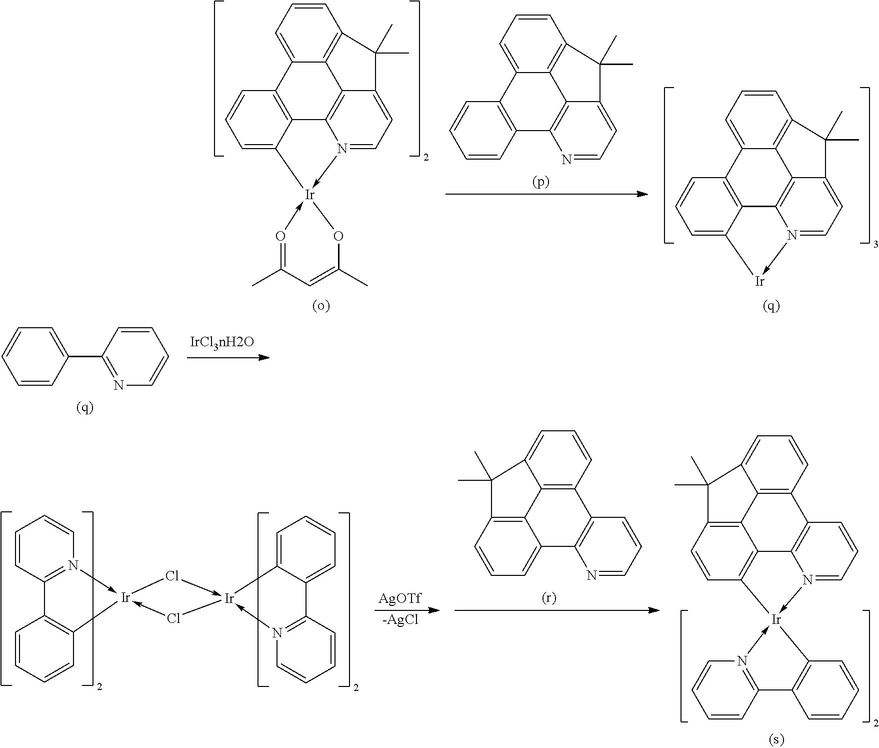

Various compounds can be produced by appropriately changing the compounds represented by (a), (b), (f), (h), (j), (k), (n), (p), (q), and (r). The present disclosure is not limited to the synthesis scheme and the compounds synthesized by the synthesis scheme, and various synthesis schemes and reagents may be used. The synthesis method is described in detail in exemplary embodiments.

Characteristics of Organic Compounds According to the Present Embodiment

Next, characteristics of the organic compound according to the present embodiment are described. In the organic compound according to the present embodiment, the partial structure IrL is a partial structure represented by the general formula [A-1] or [A-2]. Thus, it can also be said that the ligand L has a dibenzo[f,h]quinoline skeleton.

The organic compound according to the present embodiment has the following characteristics and characteristically has a high quantum yield. The organic compound according to the present embodiment is also highly sublimable. Furthermore, the organic compound can be used to provide an organic light-emitting element with high luminescence efficiency. Furthermore, the organic compound can be used to provide an organic light-emitting element with high durability.

(1) High quantum yield because the ligand has a ring structure with the dibenzo[f,h]quinoline skeleton bridged by Z₁ or Z₂.

(2) Lower symmetry of the ligand and high sublimability because the ligand has a ring structure with the dibenzo[f,h]quinoline skeleton bridged by Z₁ or Z₂.

These characteristics are described below with reference to a comparative compound 1-b as a comparison target. The comparative compound 1-b is a compound in which an ancillary ligand of the compound 1-a described in PTL 1 is changed from acetylacetone to phenylpyridine.

(1) High quantum yield because the ligand has a ring structure with the dibenzo[f,h]quinoline skeleton bridged by Z₁ or Z₂.

The present inventors have focused on the structure of a ligand of an organic compound in the development of an organic compound according to the present disclosure. More specifically, in an Ir complex having a ligand with the dibenzo[f,h]quinoline skeleton, the dibenzo[f,h]quinoline skeleton of the ligand is bridged with Z₁ or Z₂ to form a ring structure and improve the quantum yield.

Table 1 shows the comparison results of the emission properties of an exemplary compound A21, which is an organic compound according to the present embodiment, and the comparative compound 1-b. The emission wavelength was measured with F-4500 manufactured by Hitachi, Ltd. in photoluminescence (PL) measurement of a diluted toluene solution at room temperature at an excitation wavelength of 350 nm. The quantum yield was determined by measuring the absolute quantum yield of a diluted toluene solution with an absolute PL quantum yield measurement system (C9920-02) manufactured by Hamamatsu Photonics K. K. The quantum yield is expressed by a value relative to the quantum yield of the exemplary compound A21, which is set to 1.0.

TABLE 1

| | | λmax | Quantum |
|---|---|---|---|
| Compound | Structure | [nm] | yield |
| Exemplary compound A21 | 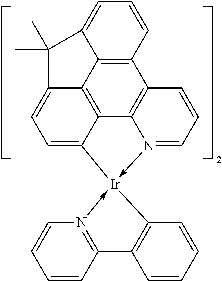 | 507 | 1.0 |
| Comparative compound 1-b | 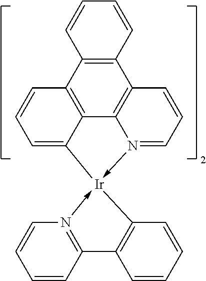 | 513 | 0.9 |

Table 1 shows that the exemplary compound A21 has a higher quantum yield and better emission properties than the comparative compound 1-b. The present inventors have considered this as described below.

The structural difference between the two compounds is whether or not the dibenzol[f,h]quinoline structure in the ligand forms a bridged ring structure. More specifically, in the comparative compound 1-b, the ligand does not form a ring structure having two bridged carbon atoms in the dibenzo[f,h]quinoline skeleton. In contrast, the exemplary compound A21 has a ring structure having two carbon atoms bridged by a dimethylmethylene group in the dibenzo[f,h]quinoline skeleton in the ligand.

As expressed by the following formula, the photoluminescence quantum yield (PLQY) is determined from the rate constants of radiative transition (light emission) and non-radiative transition (no light emission) from the excited state to the ground state. In the following formula, kr denotes the rate constant of radiative transition (radiative decay rate), and knr denotes the rate constant of non-radiative transition (non-radiative decay rate). The radiative decay rate (kr) is proportional to the square of the transition dipole moment (TDM) as expressed by the following formula (see Phys. Chem. Chem. Phys. 16, 1719-1758 (2014)).

$$PLQY = \frac{kr}{kr + knr}$$

$$kr = \frac{64 {π}^{4} Δ {E}^{3}}{3 {h}^{4} {c}^{3}} {\vert TDM \vert}^{2}$$

$$Δ E : Energy difference between {T}_{1} and {S}_{0}$$

$$h : Planck constant$$

$$c : Speed of light$$

This formula shows that increasing the radiative decay rate (kr) is effective in increasing the photoluminescence quantum yield PLQY. Because the radiative decay rate (kr) is proportional to the square of the transition dipole moment as described above, it is effective to increase the transition dipole moment.

The transition dipole moment in an Ir complex is proportional to the degree of charge transfer (CT) between the highest occupied molecular orbital (HOMO) and the lowest unoccupied molecular orbital (LUMO) (see J. Phys. Chem. 94, 239-243 (1990)). In an Ir complex, the HOMO is distributed in an aromatic ring σ-bonded to the Ir metal, and the LUMO is distributed in a heterocycle coordinately bonded to the Ir metal. For example, in a typical Ir complex Ir(ppy)₃, it is known that the HOMO is distributed in a benzene ring, and the LUMO is distributed in a pyridine ring.

The present inventors found that, in an Ir complex having a ligand with the dibenzo[f,h]quinoline skeleton, bridging atoms in the dibenzo[f,h]quinoline skeleton to form a ring structure can improve the CT properties between an aromatic ring moiety and a heterocyclic moiety. More specifically, it was found that the CT properties can be improved by a bridged structure at a position corresponding to the para position with respect to the Ir metal in an aromatic ring or a heterocycle composed of six atoms including an atom bonded to the Ir metal in the dibenzo[f,h]quinoline skeleton. More specifically, it was found that the CT properties can be improved by a bridged structure at the 9- or 4-position of the dibenzo[f,h]quinoline skeleton. Consequently, the transition dipole moment can be increased, and the photoluminescence quantum yield PLQY can be increased.

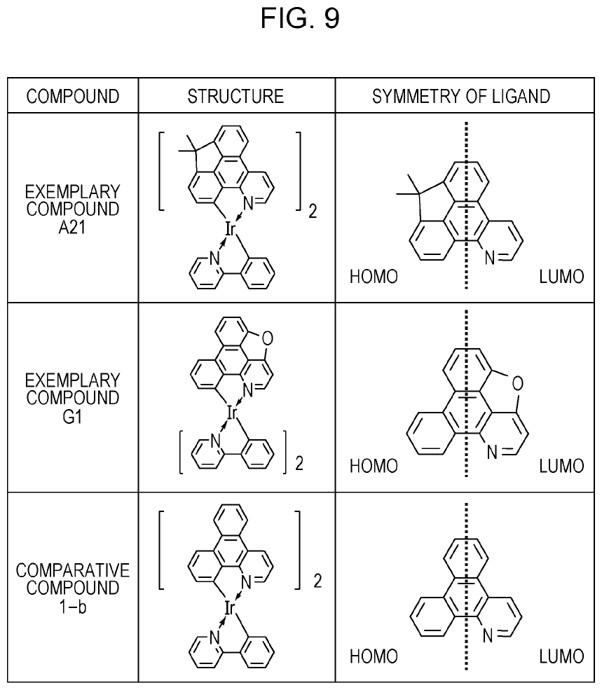

As illustrated in FIG. 9, in the exemplary compound A21, an aromatic ring composed of six atoms including a carbon atom σ-bonded to the Ir metal has the bridged structure via a methylene chain (the dimethylmethylene group) at a position corresponding to the para position with respect to the Ir metal. An electron-donating alkyl group in an aromatic ring in which the HOMO is distributed enhances the electron-donating ability, causes charge polarization, and improves the CT properties.

In an exemplary compound G1, a heterocycle composed of six atoms including a nitrogen atom coordinately bonded to the Ir metal has a bridged structure via an oxygen atom at a position corresponding to the para position with respect to the Ir metal. An electronegative oxygen atom in a heterocycle in which the LUMO is distributed enhances the electron-withdrawing ability, causes charge polarization, and improves the CT properties.

Although a structure with an aromatic ring bridged by an electron-donating substituent and a structure with a heterocycle bridged by an electron-withdrawing substituent are exemplified, the present disclosure is not limited to these structures. More specifically, regardless of whether a substituent constituting a bridged structure is electron-donating or electron-withdrawing, the partial structure IrL represented by the general formula [A-1] or [A-2] can break symmetry and cause charge polarization in an aromatic ring or a heterocycle. This enhances the CT properties, increases transition dipole moment, and increases the radiative decay rate (kr). This probably results in improved photoluminescence quantum yield (PLQY).

On the other hand, a ligand with the dibenzo[f,h]quinoline skeleton in the comparative compound 1-b has higher symmetry and less charge polarization than the organic compound according to the present embodiment. This probably results in poor CT properties, low transition dipole moment, and a low quantum yield.

The above formula also shows that decreasing the non-radiative decay rate (knr) is also effective in increasing the photoluminescence quantum yield (PLQY).

Non-radiative transition (non-radiative deactivation) is a deactivation process caused by converting the excited-state energy of a molecule into the vibration mode of the molecule. Non-radiative transition can be reduced by reducing molecular vibration. The molecular vibration can be effectively reduced by improving molecular rigidity. This is because in a molecule with high rigidity a bond forming the molecule has less stretching vibration, rotational vibration, and bending vibration.

In the organic compound according to the present embodiment, the ligand has a structure having bridged atoms in the dibenzo[f,h]quinoline skeleton. Thus, the ligand has less vibration than a simple dibenzo[f,h]quinoline ligand without the bridged structure and has improved rigidity. Thus, it is thought that the ligand has a smaller non-radiative decay rate (knr) and a higher photoluminescence quantum yield (PLQY) than a ligand without the bridged structure.

(2) Lower symmetry of the ligand and high sublimability because the ligand has a ring structure with the dibenzo[f,h]quinoline skeleton bridged by Z₁ or Z₂.

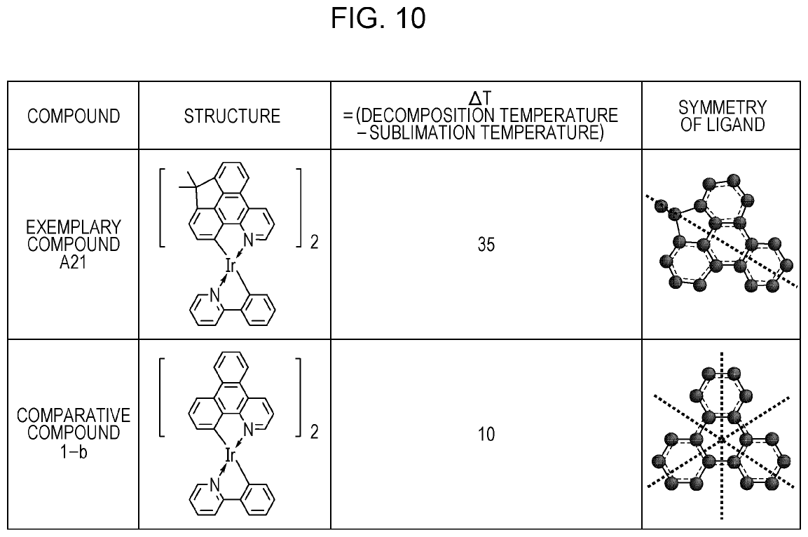

The present inventors have focused on the structural symmetry of a ligand in the development of an organic compound according to the present disclosure. To simply discuss the structural symmetry of a ligand, the molecular structures of ligands are compared assuming a nitrogen atom to be a carbon atom, as illustrated in FIG. 10. The ligand of the comparative compound 1-b has high symmetry due to one three-fold axis perpendicular to the molecular plane and three two-fold axes parallel to the molecular plane (indicated by the dotted lines in FIG. 10). By contrast, the exemplary compound A21 has lower symmetry than the comparative compound 1-b due to the bridged structure and only one two-fold axis parallel to the molecular plane (indicated by the dotted line in FIG. 10).

The symmetry of the ligand can be reduced to decrease the sublimation temperature. This is because with a ligand of lower symmetry an organic compound is less likely to aggregate. By contrast, with a ligand of higher symmetry, an organic compound is likely to aggregate, thus resulting in a high sublimation temperature. A low sublimation temperature can result in a large difference between the sublimation temperature and the thermal decomposition temperature, less thermal decomposition during sublimation, and higher sublimability.

FIG. 10 shows the comparison results of the sublimability of the exemplary compound A21, which is an organic compound according to the present embodiment, and the comparative compound 1-b. For the evaluation of sublimability, the difference between the sublimation temperature and the decomposition temperature is compared. A higher temperature difference indicates higher sublimability. The decomposition temperature is a temperature at which the weight loss reaches 5% in TG/DTA measurement. The sublimation temperature is a temperature at which a sufficient sublimation rate is achieved while the temperature is slowly increased in a vacuum of 1×10⁻¹ Pa in an Ar flow to perform sublimation purification.

FIG. 10 shows that the exemplary compound A21, which is an organic compound according to the present embodiment, is a material that has a large difference between the sublimation temperature and the decomposition temperature and high sublimability. Furthermore, due to high sublimability, sublimation purification can be stably performed without decomposition. This also indicates high vapor deposition stability in the production of an organic light-emitting element. More specifically, a high-purity evaporated film can be formed without decomposition during vapor deposition, and a long-life organic light-emitting element can be provided.

Low symmetry also provides the following advantages. The comparative compound 1-b has a ligand having a dibenzo[f,h]quinoline structure with an extended π-conjugated system. Thus, an organic compound easily aggregates by π-π interaction, which facilitates concentration quenching in an organic light-emitting element. On the other hand, the organic compound according to the present embodiment has lower symmetry due to the bridged structure, though the ligand has the dibenzo[f,h]quinoline skeleton. This can reduce the π-π interaction compared with compounds without the bridged structure and reduce the aggregation of the organic compound. Thus, a high-efficiency organic light-emitting element with less concentration quenching can be provided.

The evaluation of the characteristics (1) and (2) of the organic compound according to the present embodiment is described in more detail in the exemplary embodiments described later.

Next, other characteristics of organic compounds with the partial structure IrL represented by any one of the general formulae [A-11] to [A-14] and [A-21] to [A-24] are described. These organic compounds have the following characteristics and therefore can be suitably used for an organic light-emitting element.

(3) When Z₁ or Z₂ in the general formula [A-1] or [A-2] is any one of SiR₁R₂, CR₁R₂, and GeR₁R₂, a substituent group extending in a direction perpendicular to the in-plane direction of the dibenzo[f,h]quinoline structure further enhances sublimability.

(4) When Z₁ or Z₂ in the general formula [A-1] or [A-2] is an oxygen atom or a sulfur atom, a lone pair of the oxygen atom or the sulfur atom enhances the CT properties and further increases the quantum yield.

(5) When the partial structure IrL is represented by the general formula [A-14] or [A-24], the ligand has higher chemical stability because the carbon atoms constituting the basic skeleton of the ligand are composed only of sp2 carbon atoms.

These characteristics are described below.

(3) When Z₁ or Z₂ in the general formula [A-1] or [A-2] is any one of SiR₁R₂, CR₁R₂, and GeR₁R₂, a substituent group extending in a direction perpendicular to the in-plane direction of the dibenzo[f,h]quinoline structure further enhances sublimability.

As represented by the general formula [A-1] or [A-2], the organic compound according to the present embodiment has a highly planar ligand having the dibenzo[f,h]quinoline skeleton as the basic skeleton and having an extended π-conjugated system. The bridged structure lowers the symmetry of the ligand and suppresses the stacking of the ligands. When Z₁ or Z₂ is any one of SiR₁R₂, CR₁R₂, and GeR₁ R₂, the substituents R₁ and R₂ can reduce the planarity of the ligand and further suppress the stacking of the ligands.

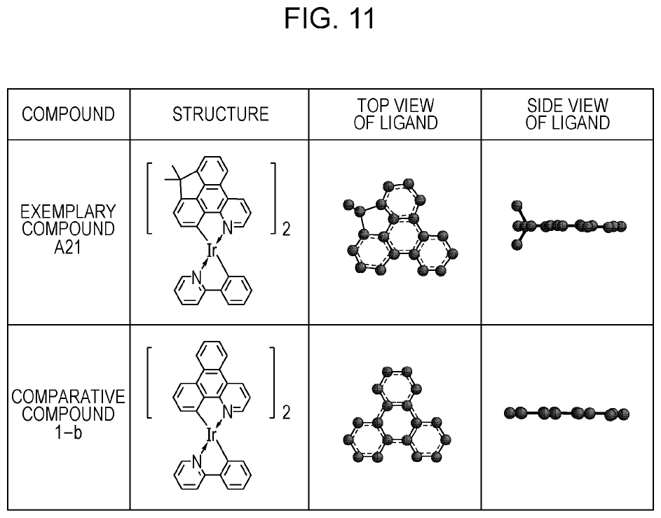

The planarity is compared between the ligands of the exemplary compound A21 and the comparative compound 1-b. As illustrated in FIG. 11, in the exemplary compound A21, the ligand has the bridged structure via the dimethylmethylene group, and the substituents bonded to the methylene chain (the methyl groups) extend in a direction perpendicular to the plane of the ligand. Thus, the steric hindrance effect of the substituents makes it difficult for the ligands to aggregate and can further reduce the aggregation of the organic compound. This can further lower the sublimation temperature and further enhance sublimability. Thus, the organic compound can have higher resistance to concentration quenching.

In particular, Z₁ or Z₂ in the general formula [A-1] or [A-2] can be CR₁R₂. In other words, the partial structure IrL can be represented by the general formula [A-11] or [A-21].

(4) When Z₁ or Z₂ in the general formula [A-1] or [A-2] is an oxygen atom or a sulfur atom, a lone pair of the oxygen atom or the sulfur atom enhances the CT properties and further increases the quantum yield.

When Z₁ or Z₂ in the general formula [A-1] or [A-2] is an oxygen atom or a sulfur atom, the organic compound according to the present embodiment has a structure having carbon atoms bridged by an oxygen atom or a sulfur atom in the dibenzo[f,h]quinoline skeleton. The oxygen atom has high electronegativity and abundant lone pairs, and the sulfur atom has abundant lone pairs. Thus, the oxygen atom or the sulfur atom of Z₁ or Z₂ enhances polarization in the ligand, increases the amount of change in electron density, and can therefore further enhance the CT properties. Consequently, as shown in Table 2, the organic compound has a higher quantum yield. The quantum yield is measured as described above and is expressed by a value relative to the quantum yield of the exemplary compound A21, which is set to 1.0.

TABLE 2

| Compound | Structure | Quantum yield |
|---|---|---|
| Exemplary compound A21 | 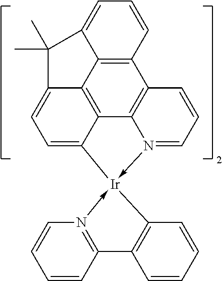 | 1.0 |
| Exemplary compound G21 | 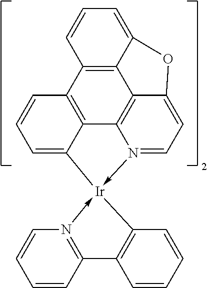 | 1.1 |

Thus, from the perspective of quantum yield, Z₁ or Z₂ in the general formula [A-1] or [A-2] can be an oxygen atom or a sulfur atom. In other words, the partial structure IrL can be represented by any one of the general formulae [A-12], [A-13], [A-22], and [A-23].

(5) When the partial structure IrL is represented by the general formula [A-14] or [A-24], the ligand has higher chemical stability because the carbon atoms constituting the basic skeleton of the ligand are composed only of sp2 carbon atoms.

When the partial structure IrL is represented by the general formula [A-14] or [A-24], the organic compound according to the present embodiment has a structure having carbon atoms bridged by an ethylene chain in the dibenzo[f,h]quinoline skeleton. Such a structure can improve the chemical stability of the ligand of the organic compound. This is because the carbon atoms constituting the basic skeleton of the ligand are composed only of sp2 carbon atoms. In other words, the carbon atoms constituting the basic skeleton of the ligand L can be composed only of sp2 carbon atoms. The basic skeleton of the ligand in the present specification refers to a structure in which all of Y₁ to Y₁₆ in the general formula [A-1] or [A-2] are carbon atoms having hydrogen atoms.

In an organic light-emitting element, oxidation-reduction occurs repeatedly while the element is driven, and the element has excited high-energy molecules. Thus, molecules constituting the element can be stable against oxidation-reduction and can have a structure composed only of bonds with high bond energy that are not cleaved even in a high energy state.

When the partial structure IrL is represented by the general formula [A-14] or [A-24],the carbon atoms constituting the basic skeleton of the ligand are composed only of sp2 carbon atoms. Thus, when the organic compound is used for an organic light-emitting element, the organic light-emitting element can have particularly high drive durability. When at least one of X₂₅ to X₃₄ in the general formula [A-14] and X₅₉ to X₆₈ in the general formula [A-24] is a nitrogen atom, like carbon atoms, the bond constituting the basic skeleton of the ligand is composed only of an sp2 hybrid orbital. Thus, the ligand has a basic skeleton composed of bonds with sufficiently high bond energy and therefore has high chemical stability.

Examples of Organic Compounds According to the Present Embodiment

Specific examples of the organic compound according to the present embodiment are described below. However, the present disclosure is not limited to these examples.

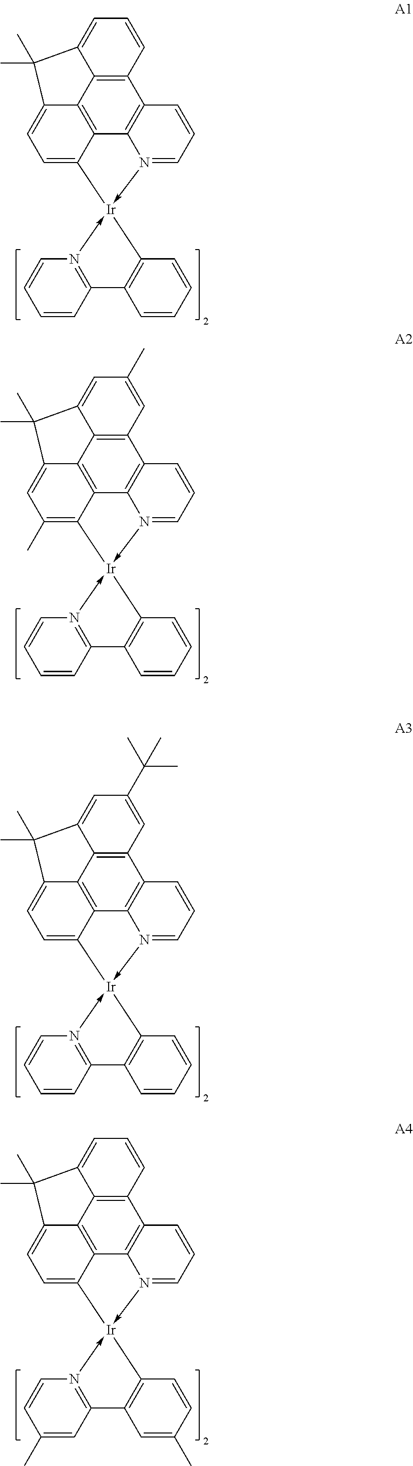

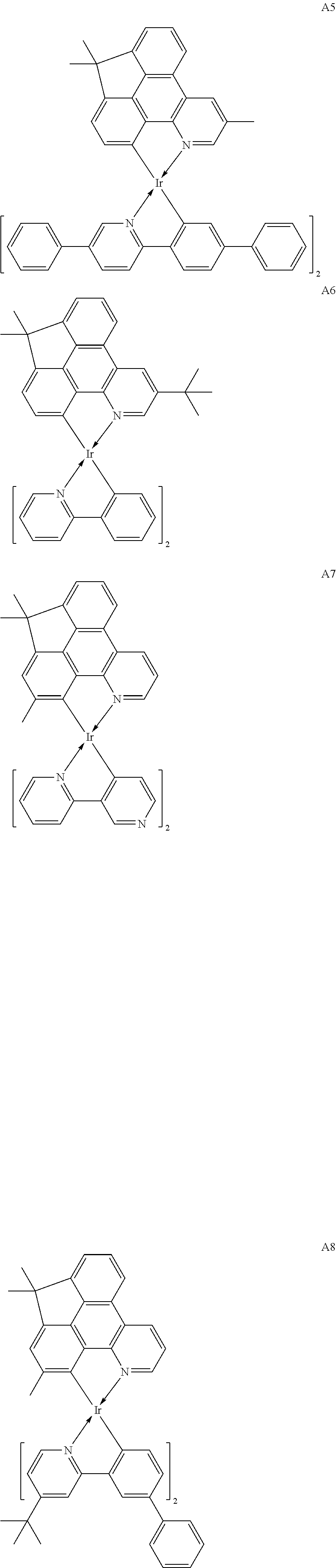

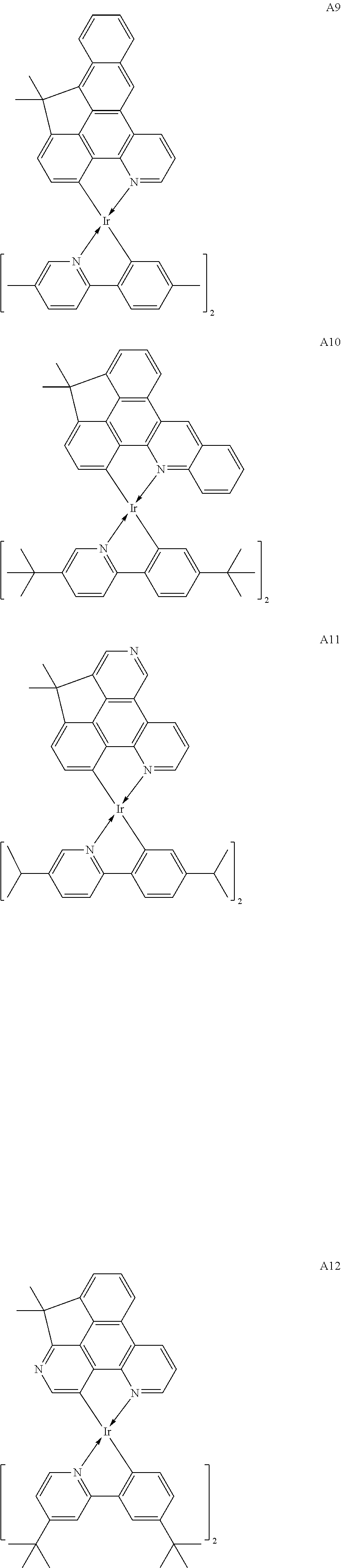

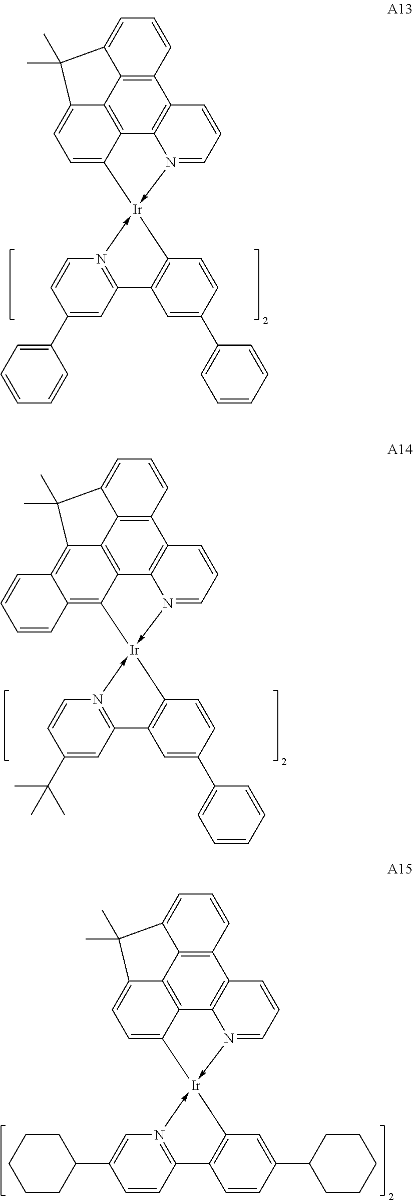

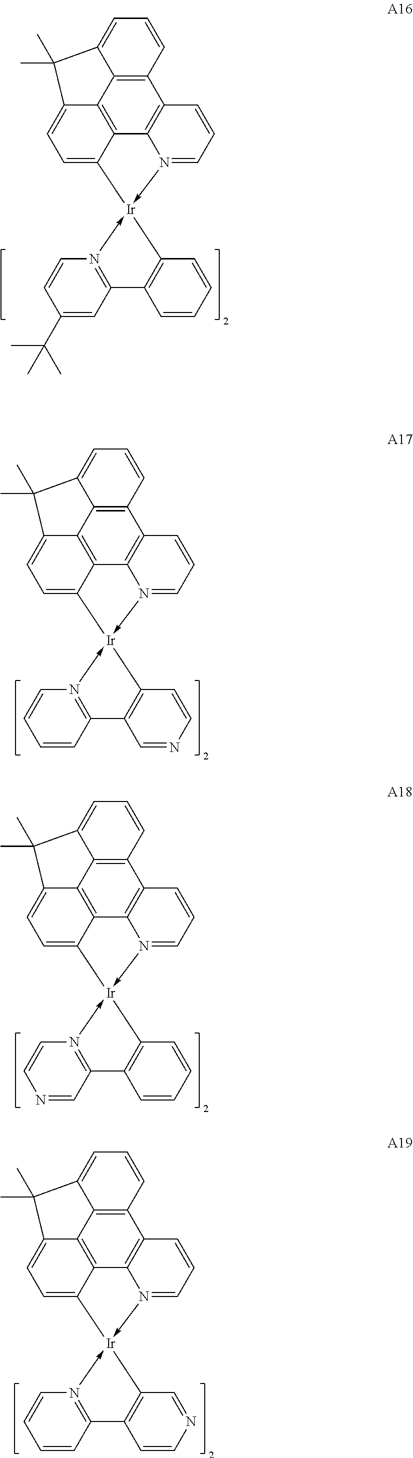

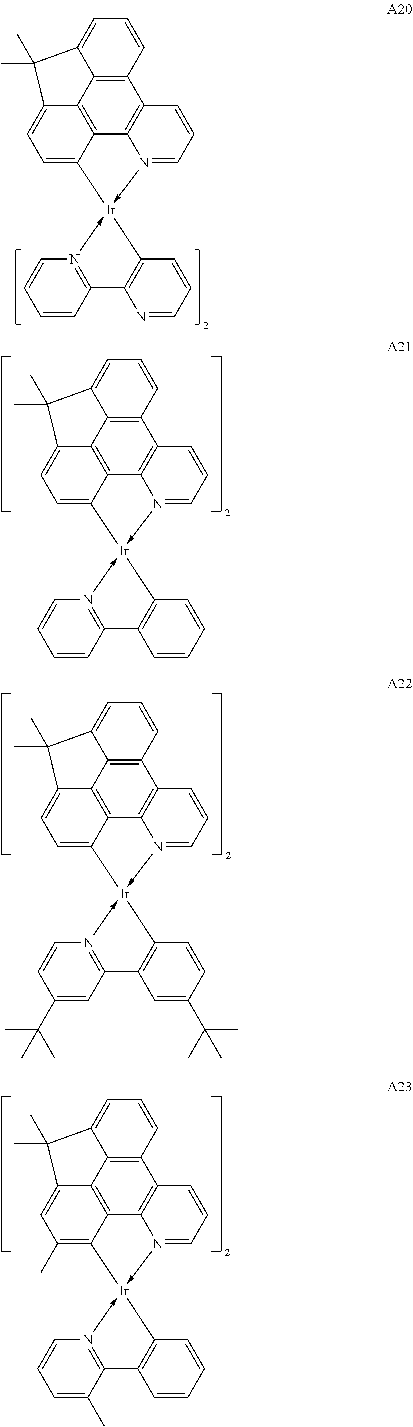

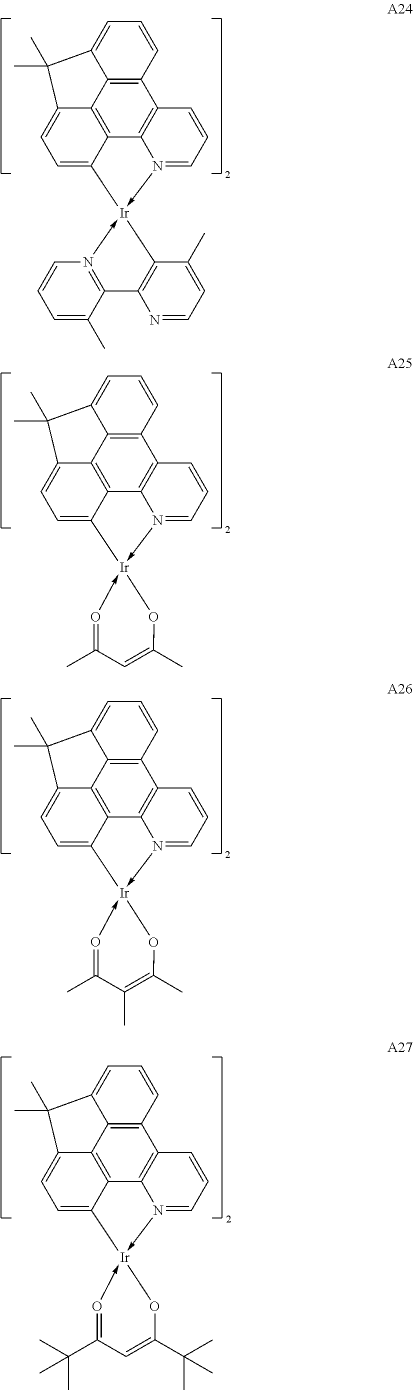

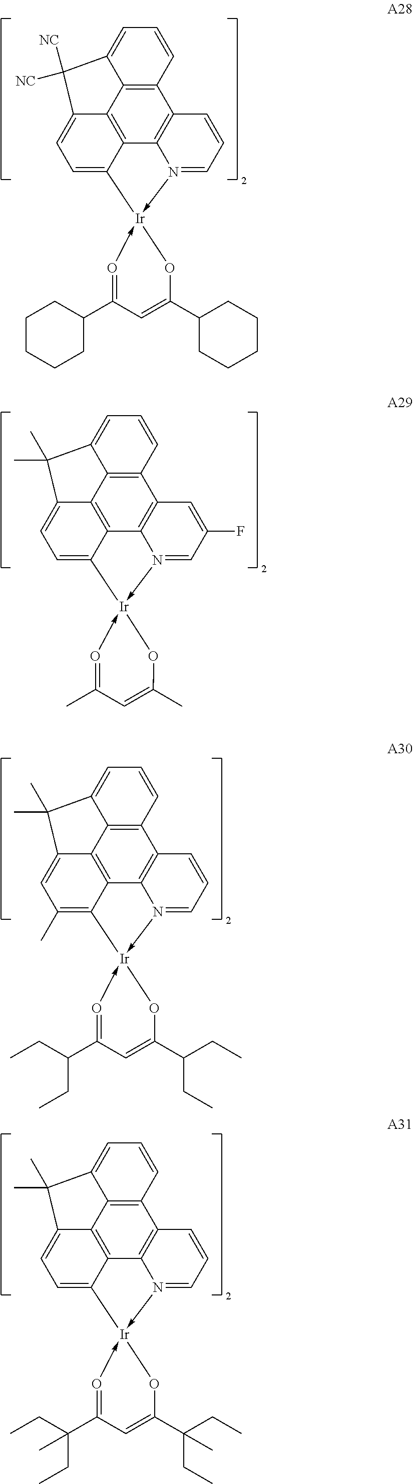

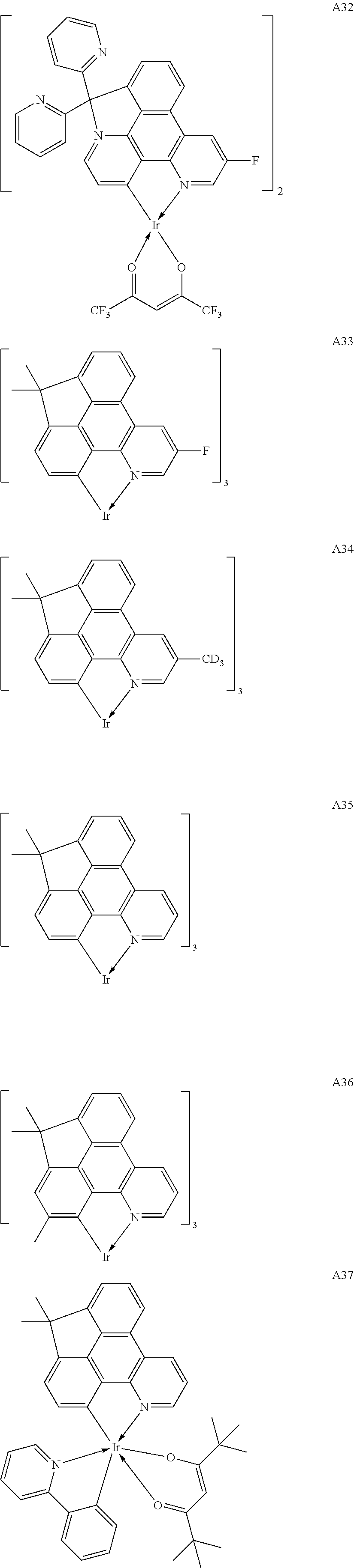

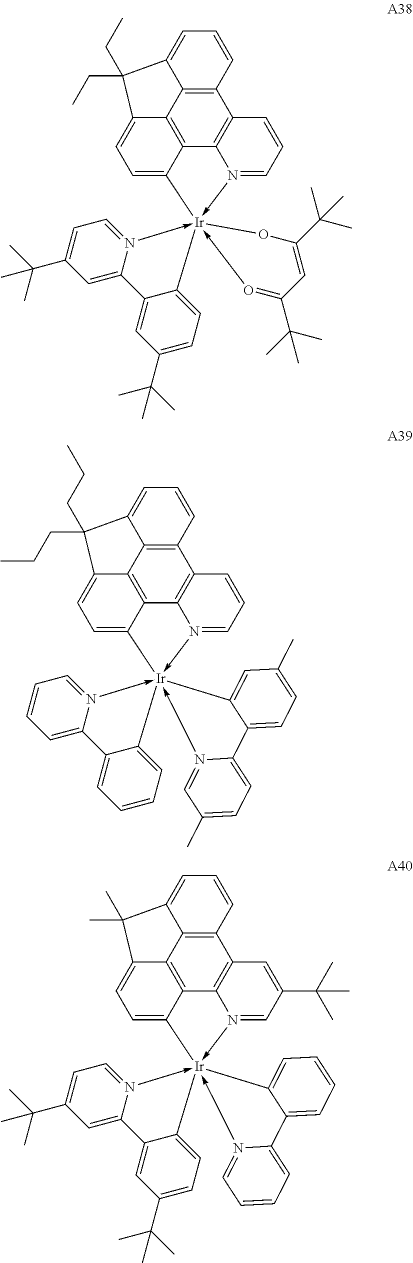

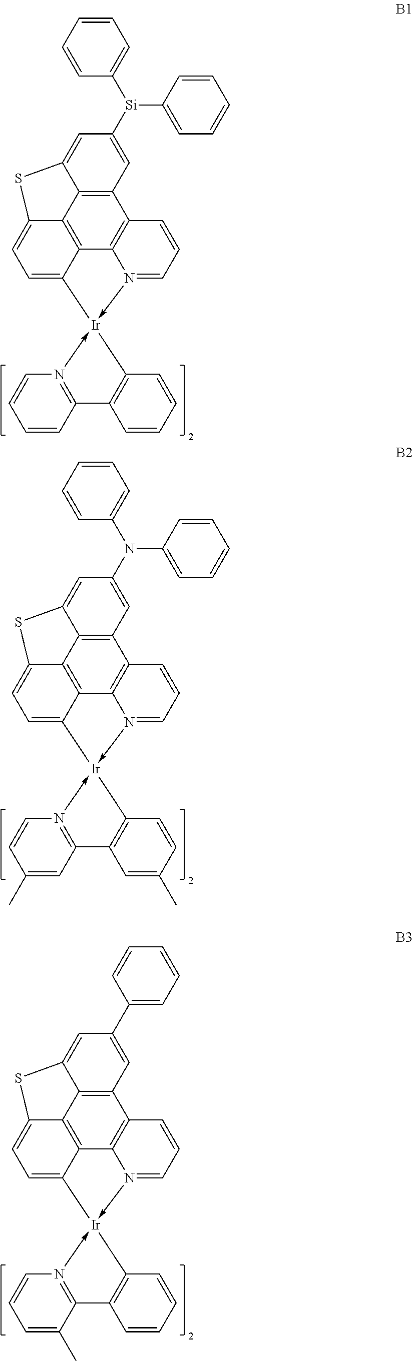

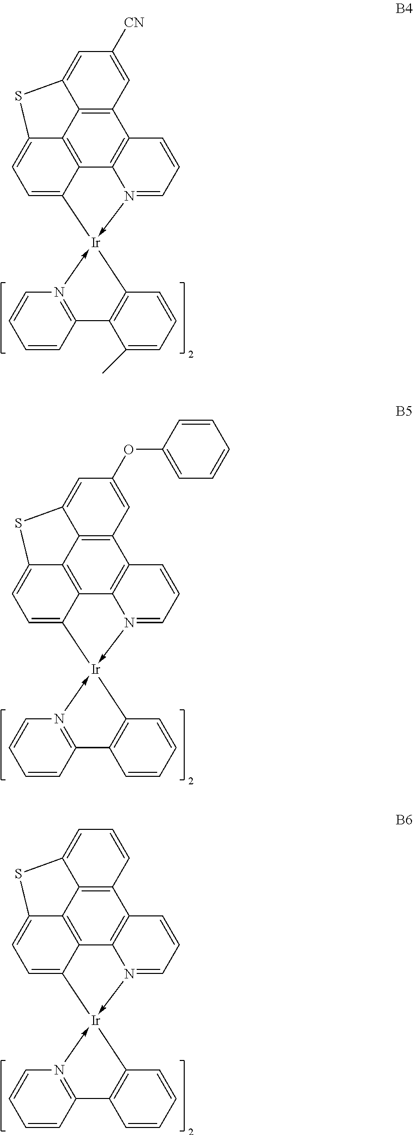

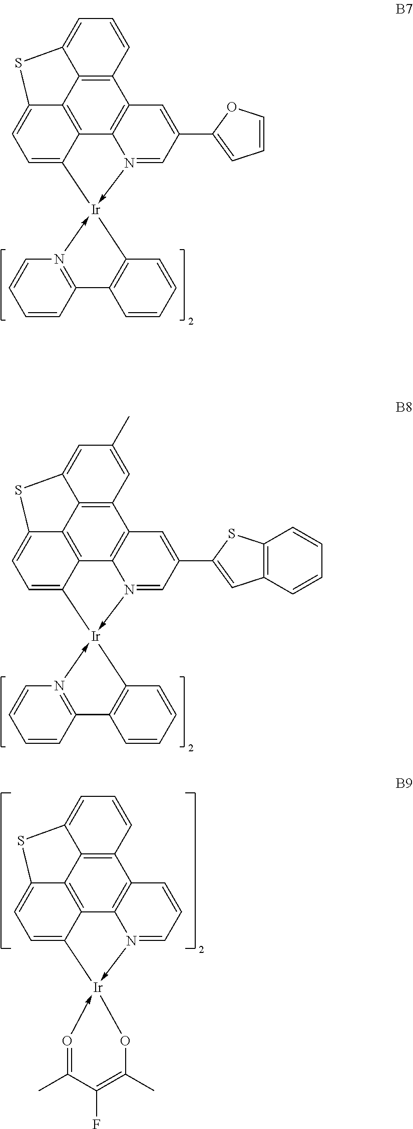

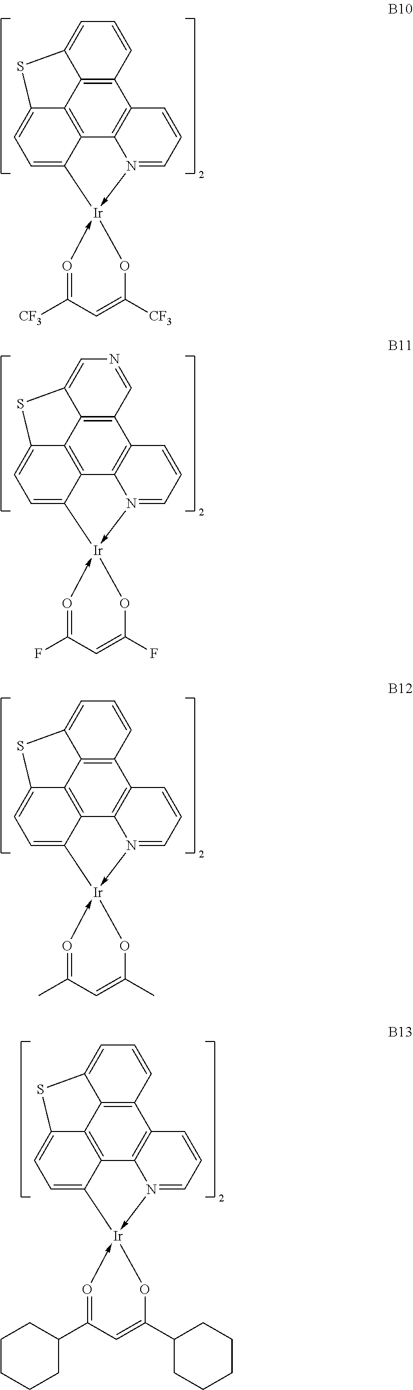

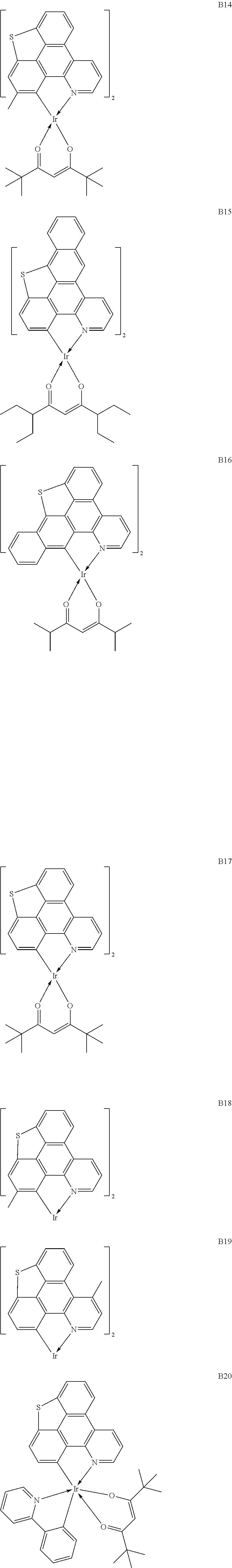

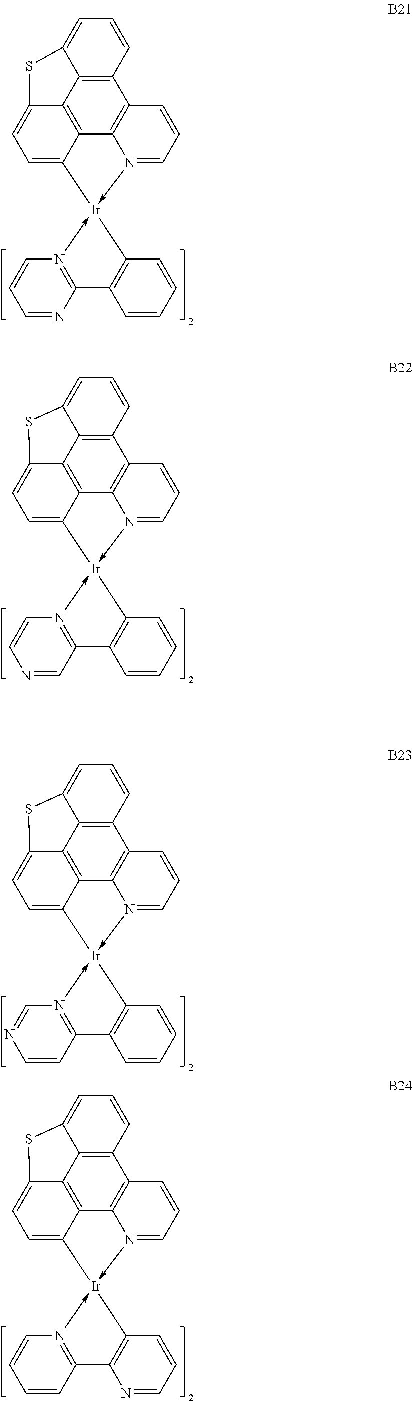

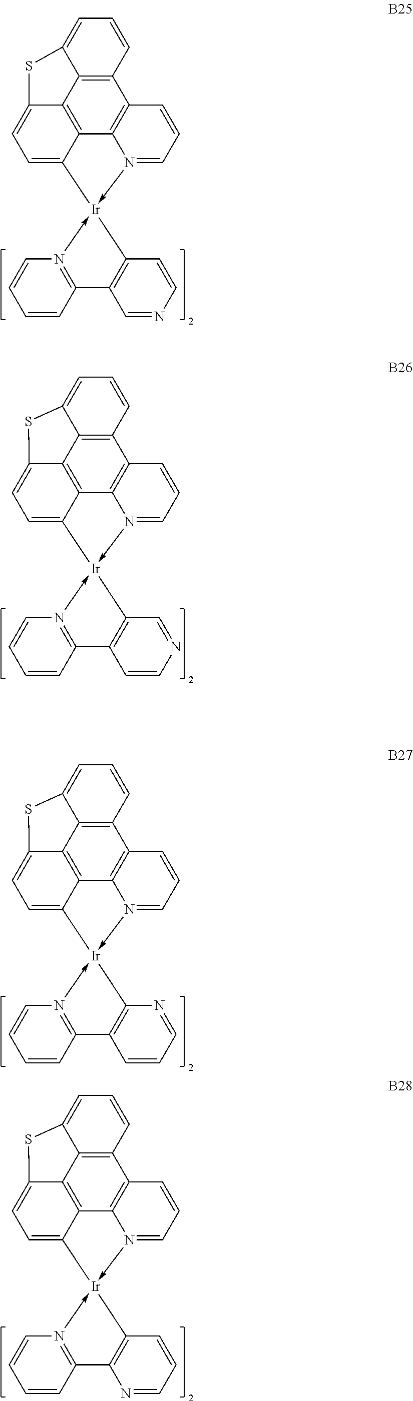

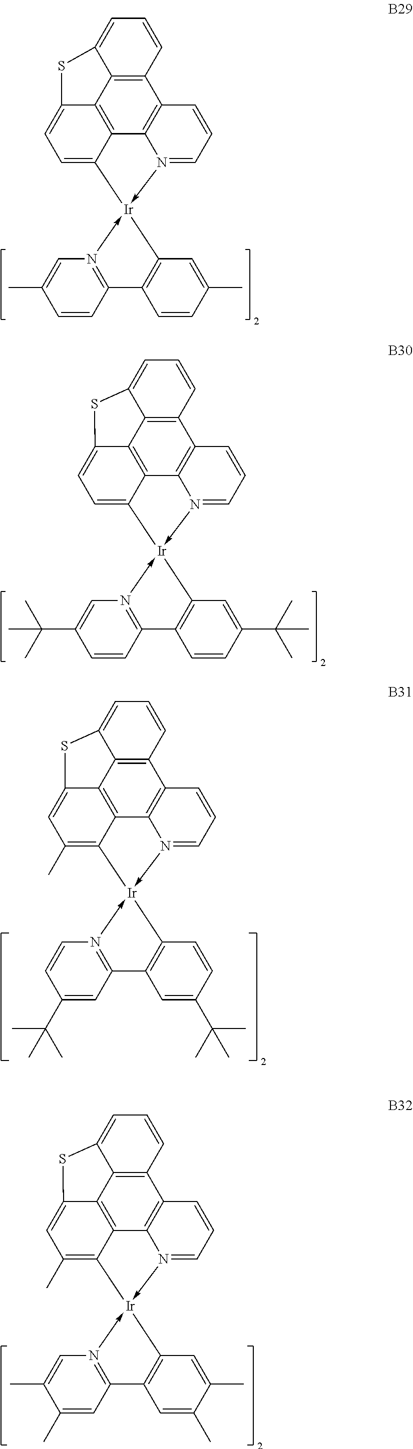

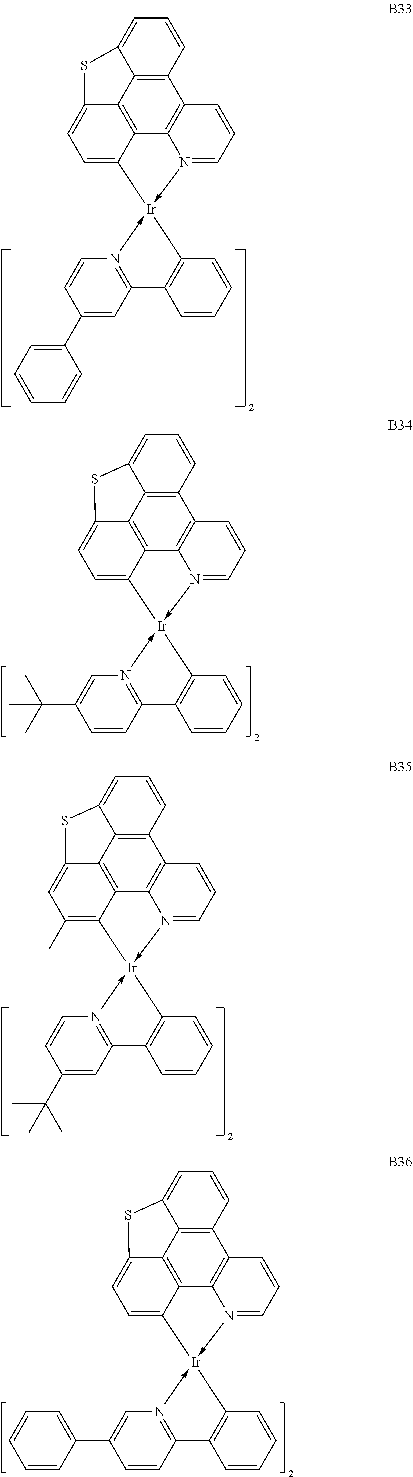

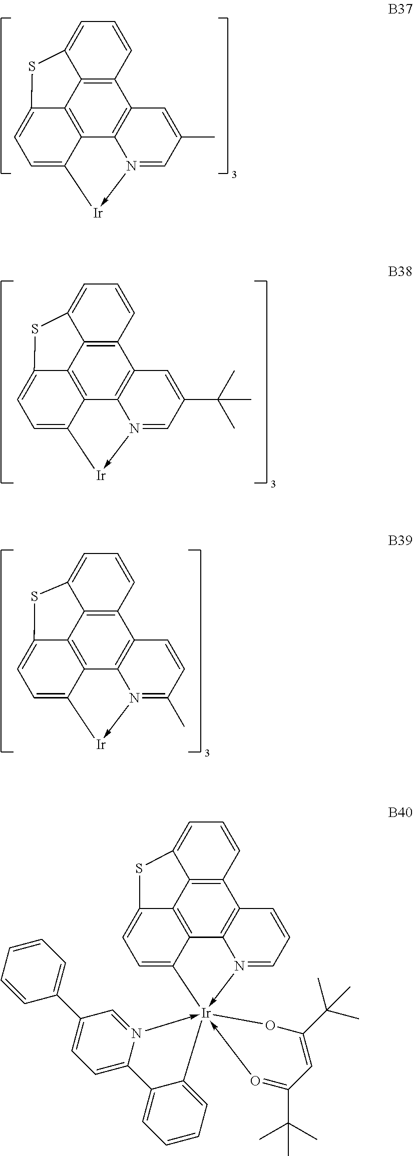

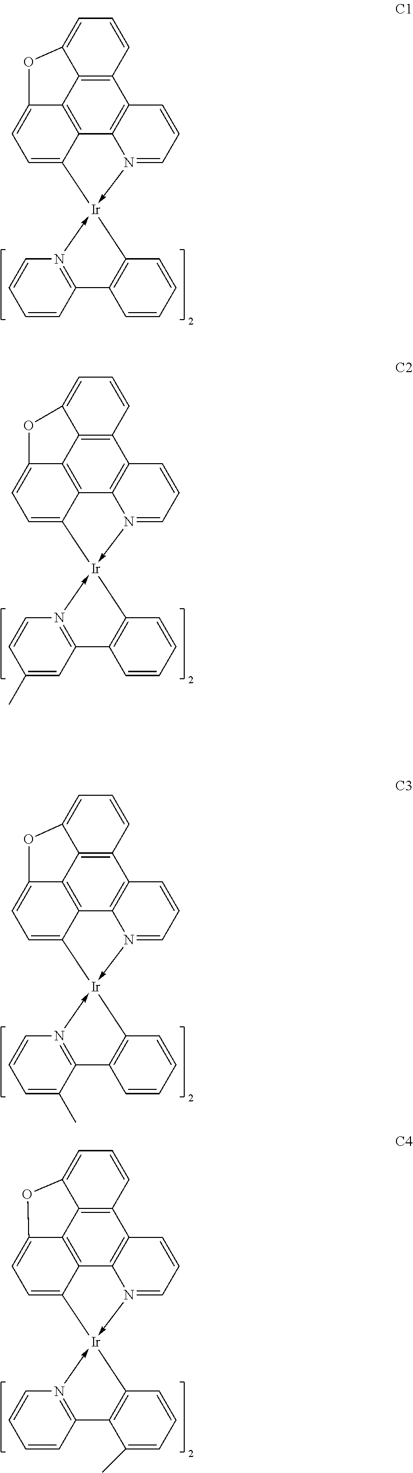

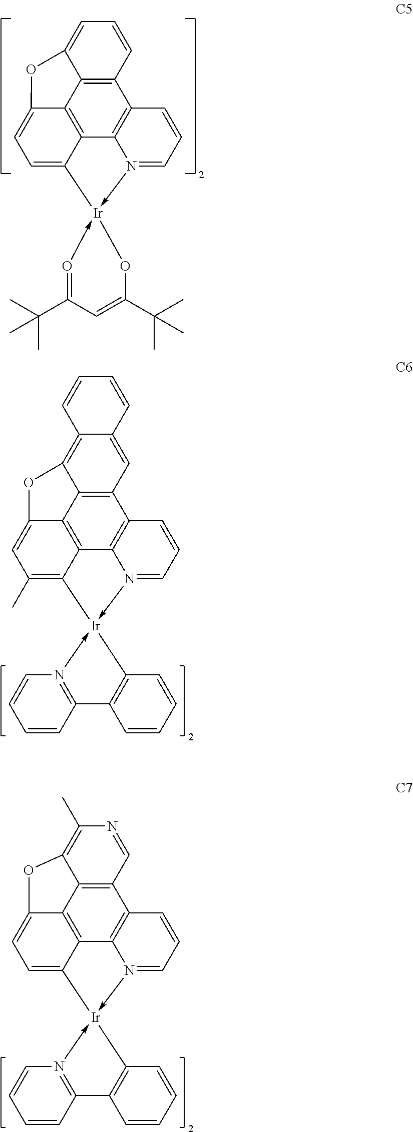

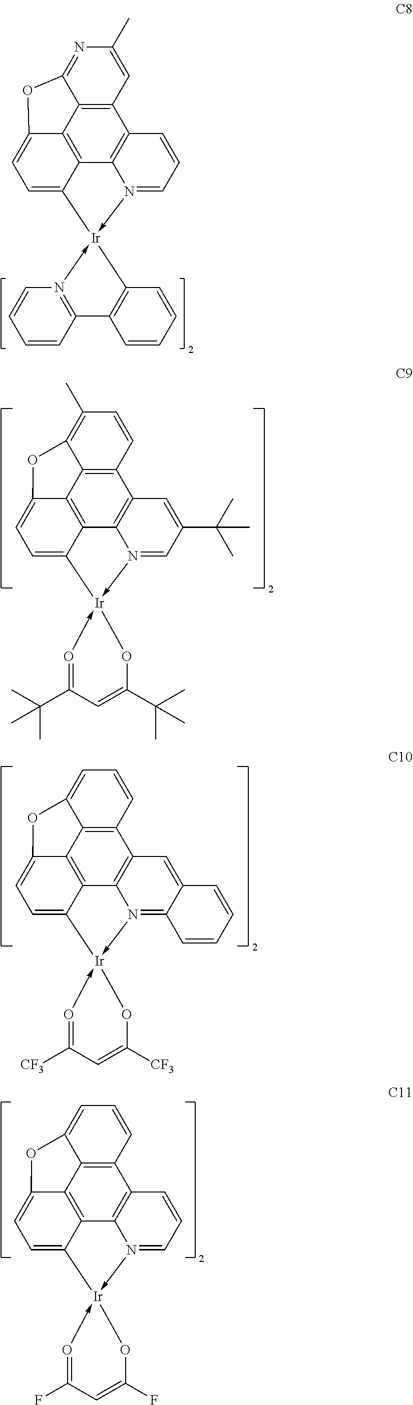

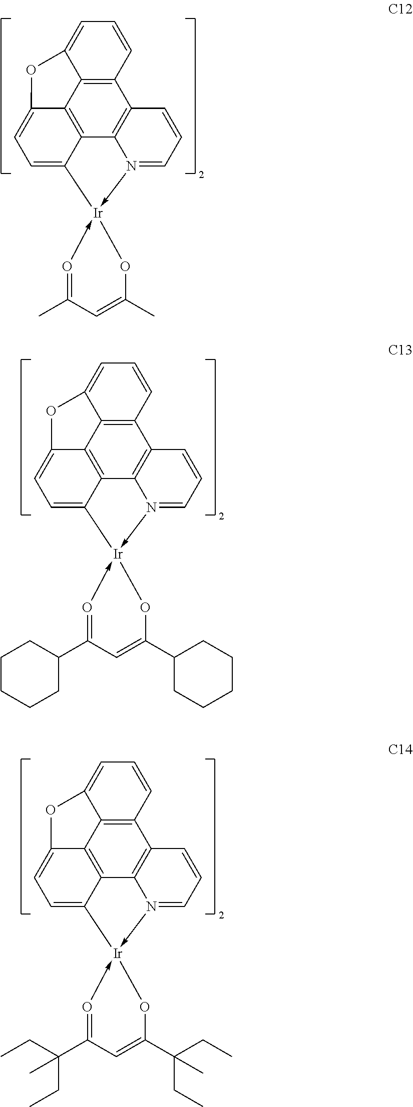

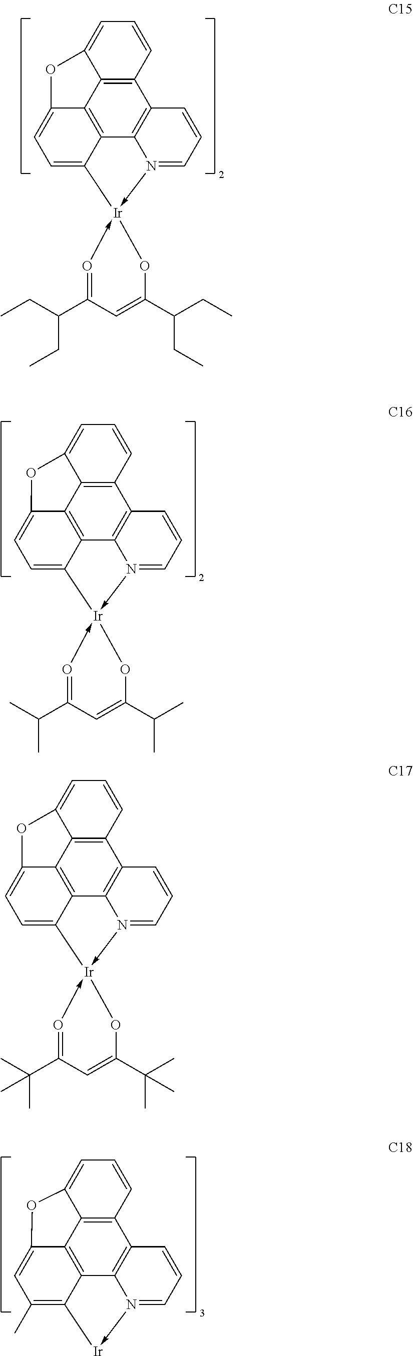

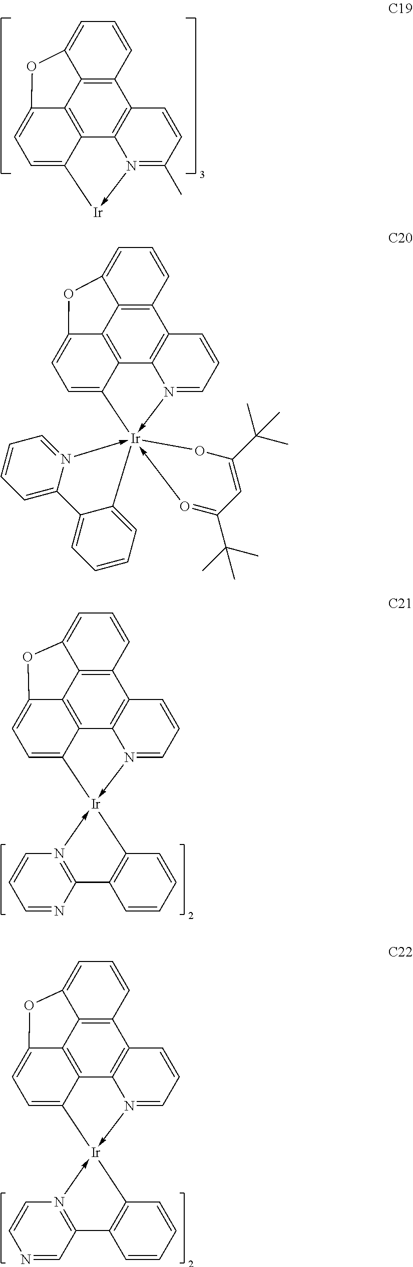

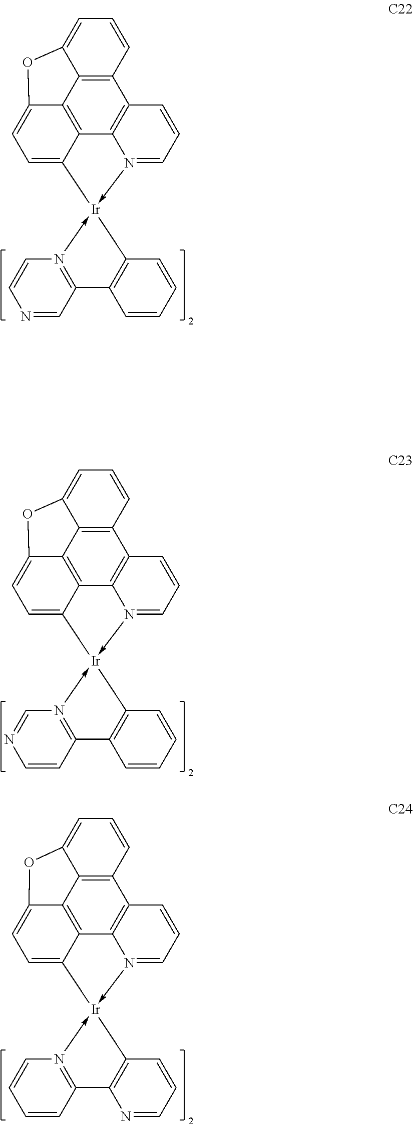

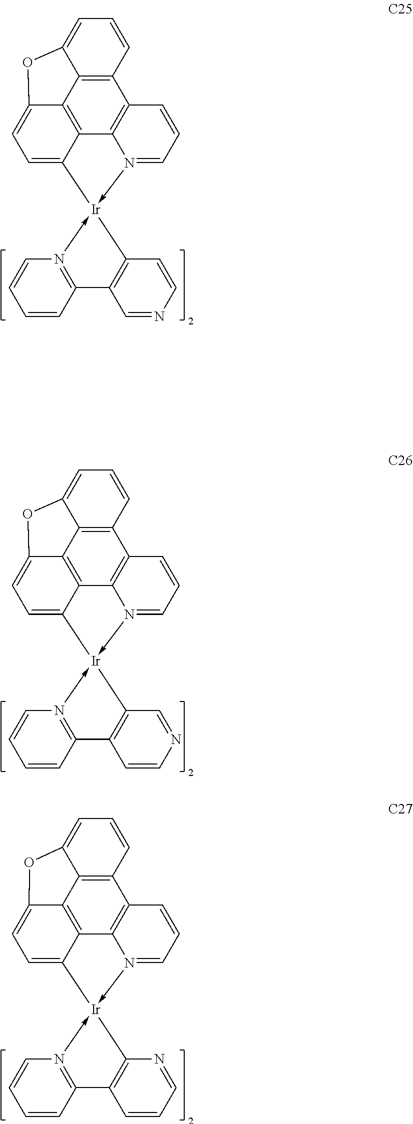

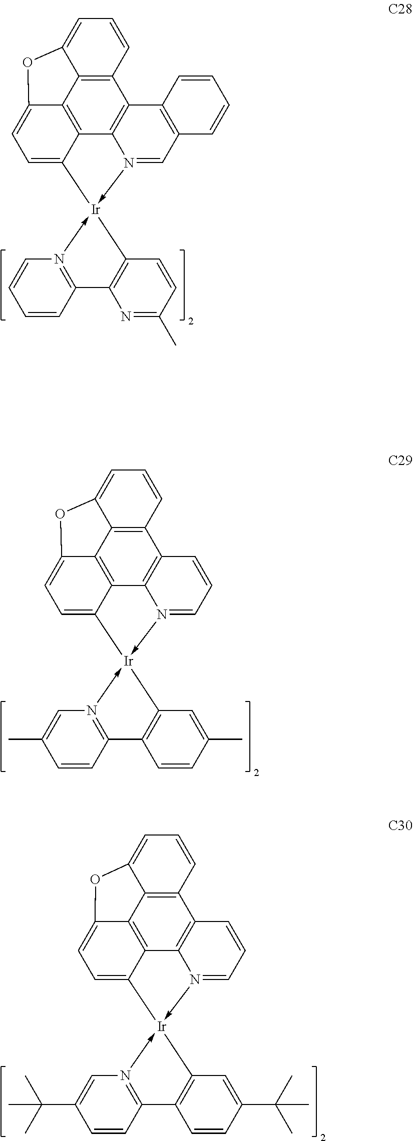

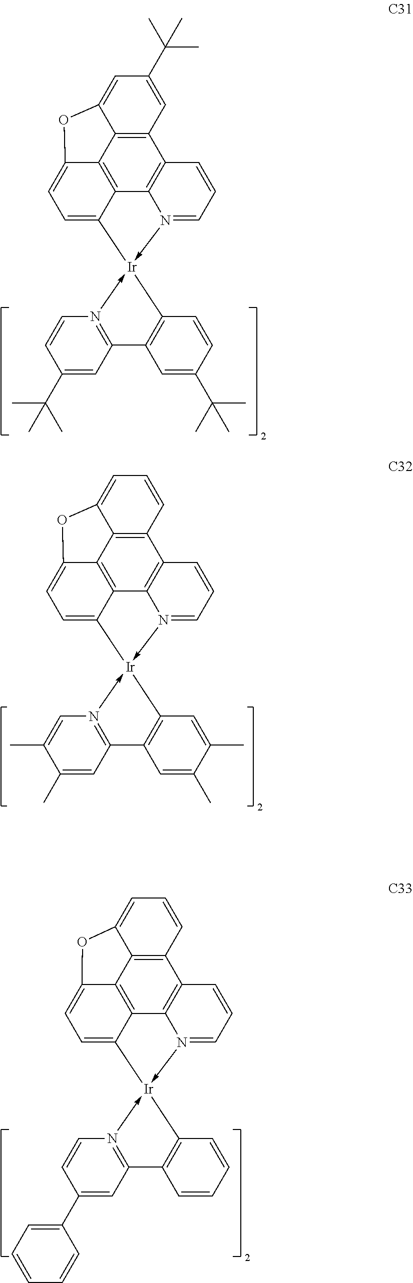

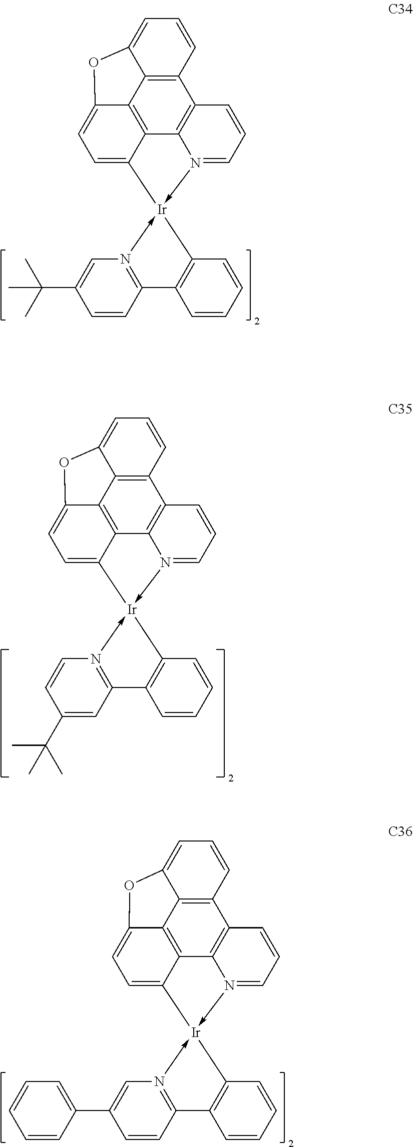

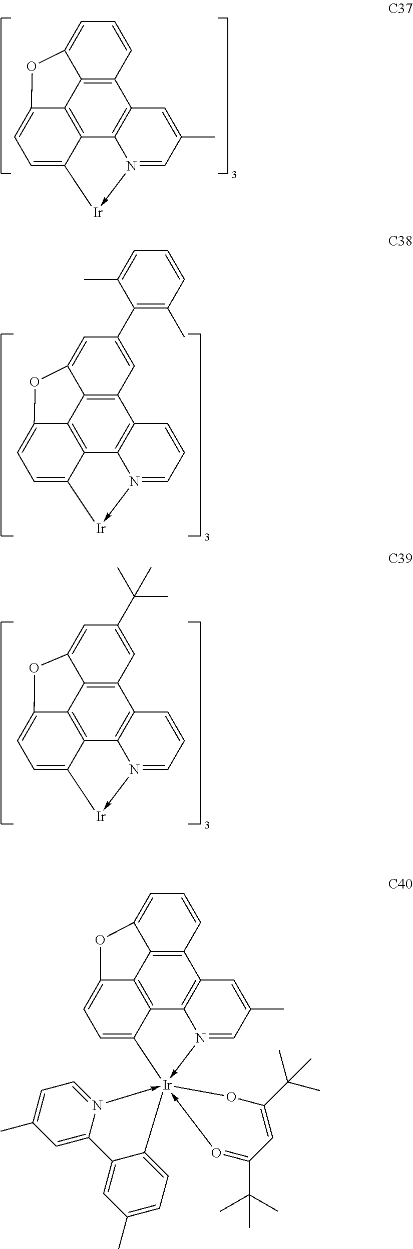

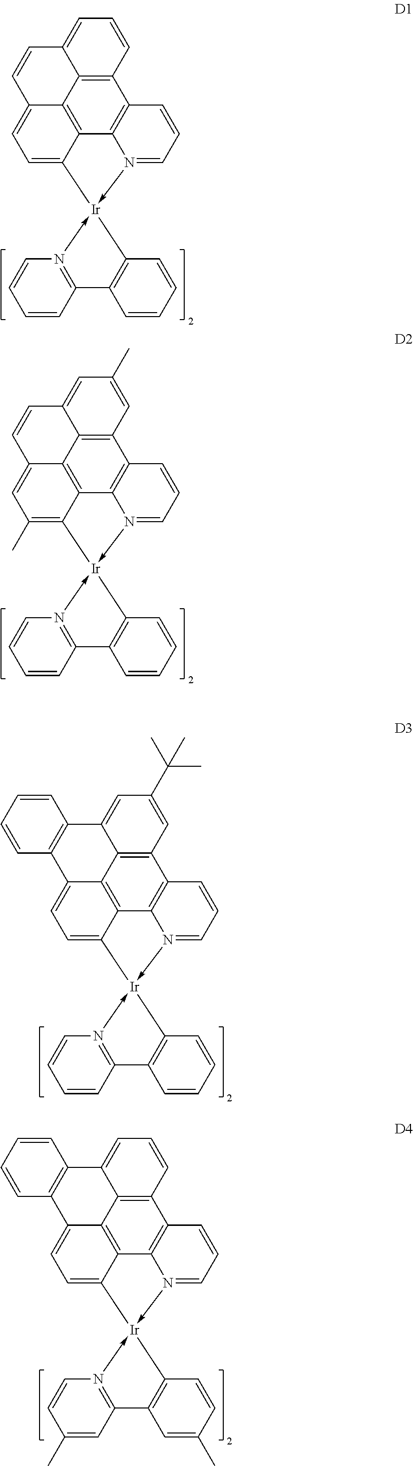

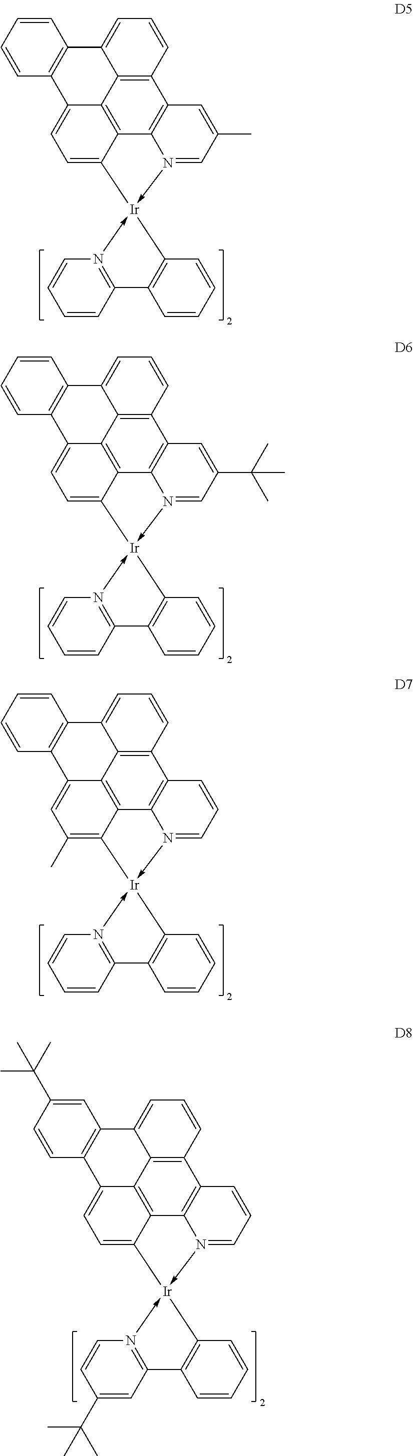

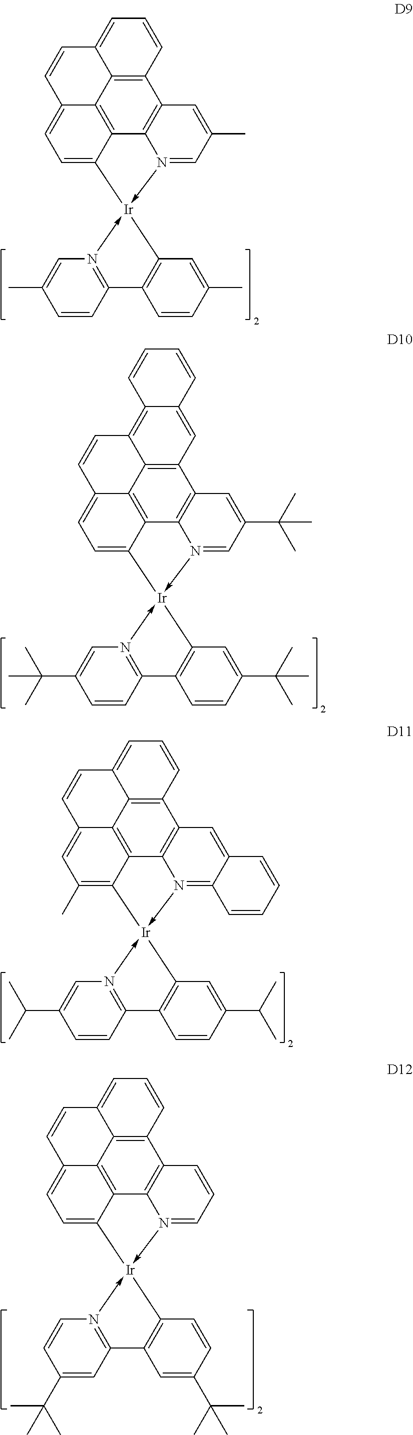

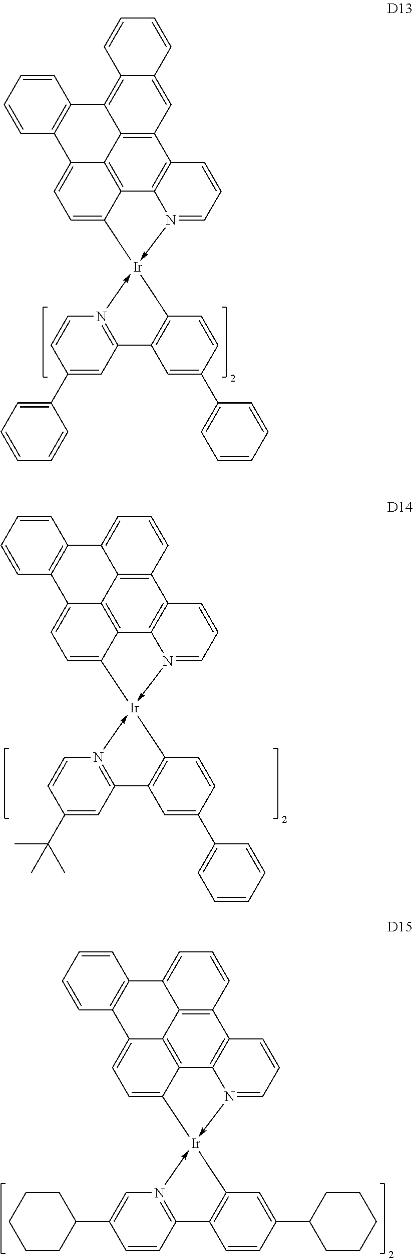

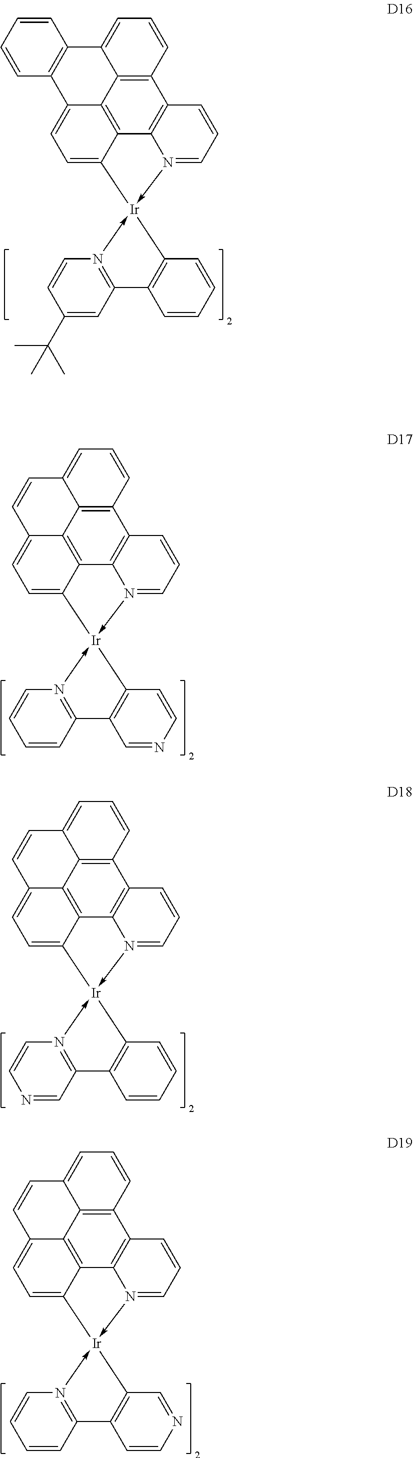

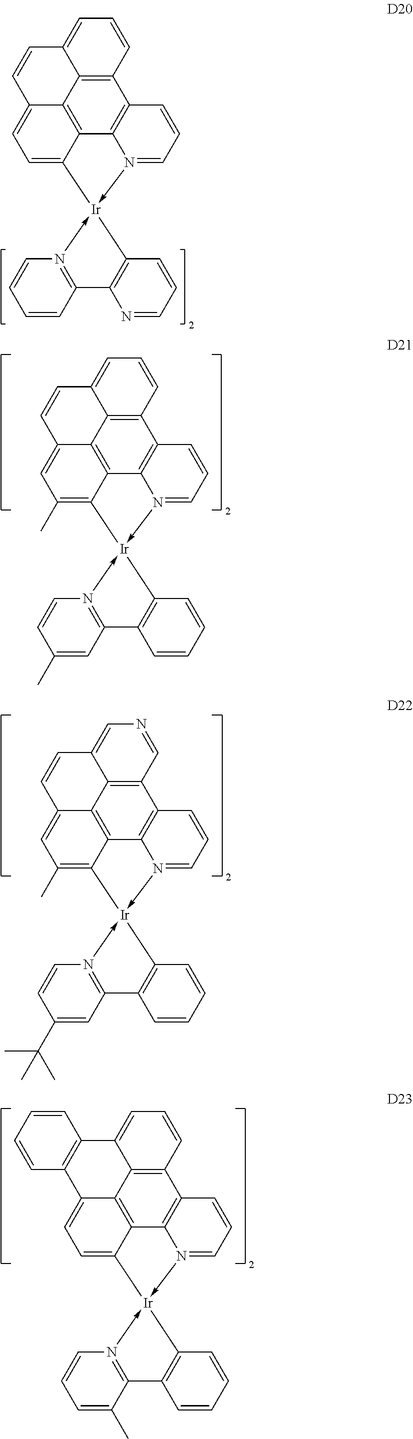

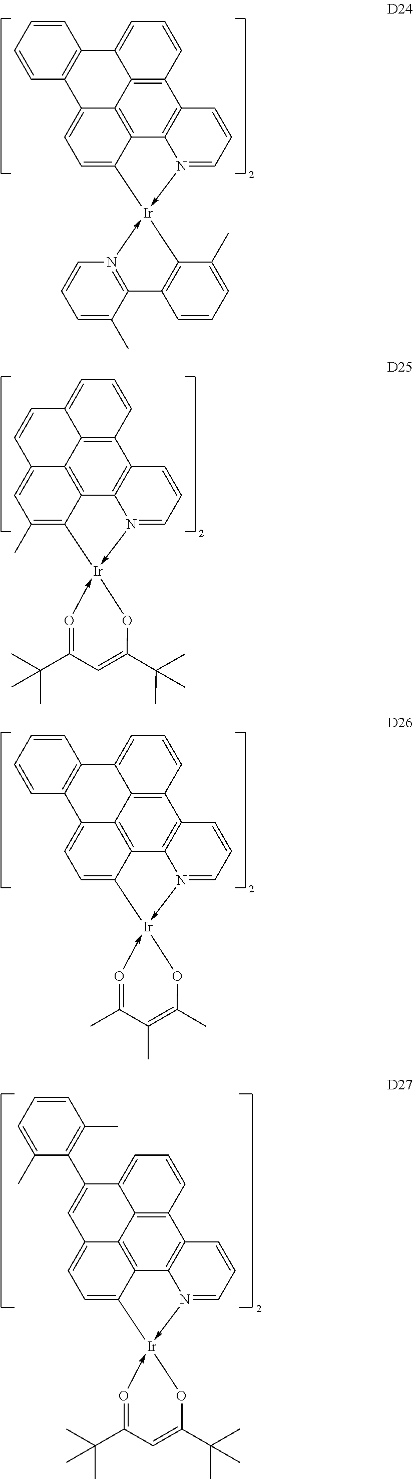

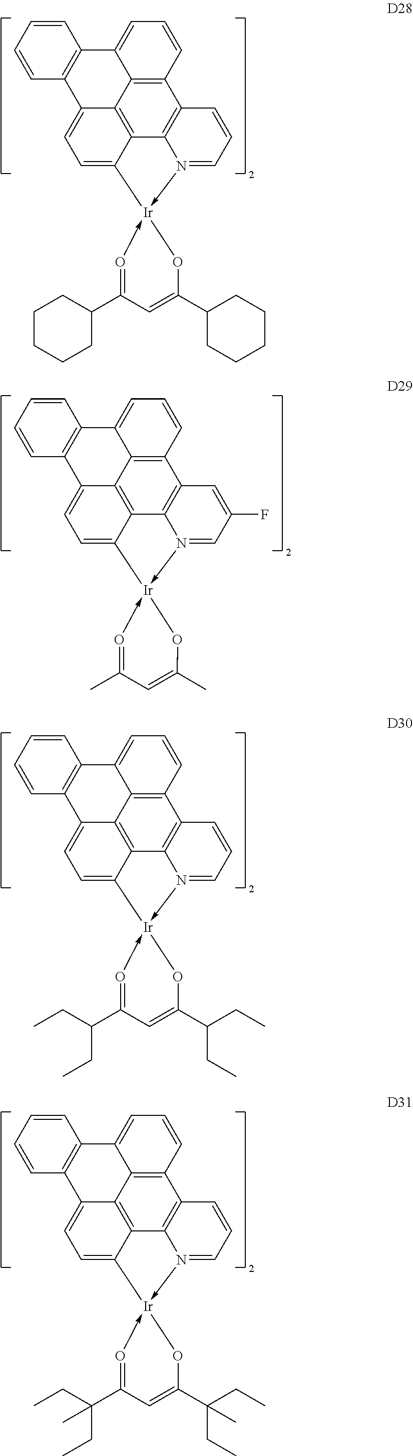

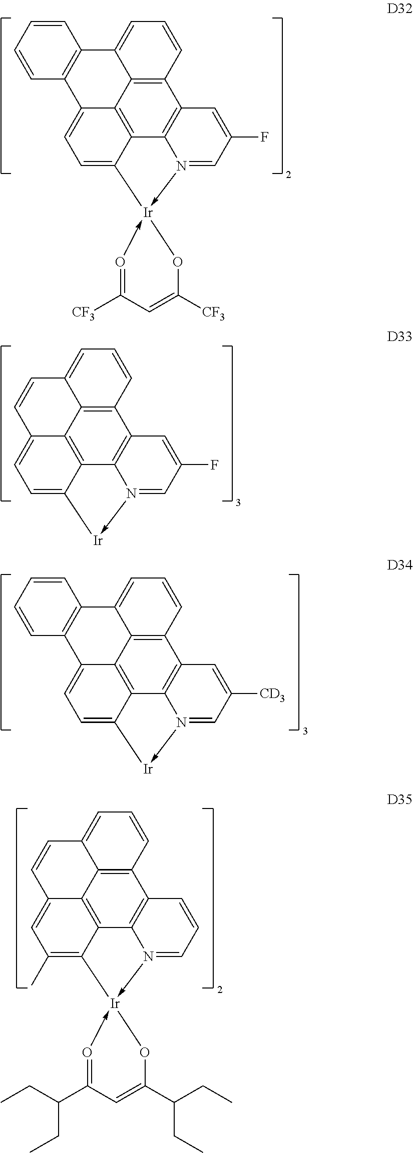

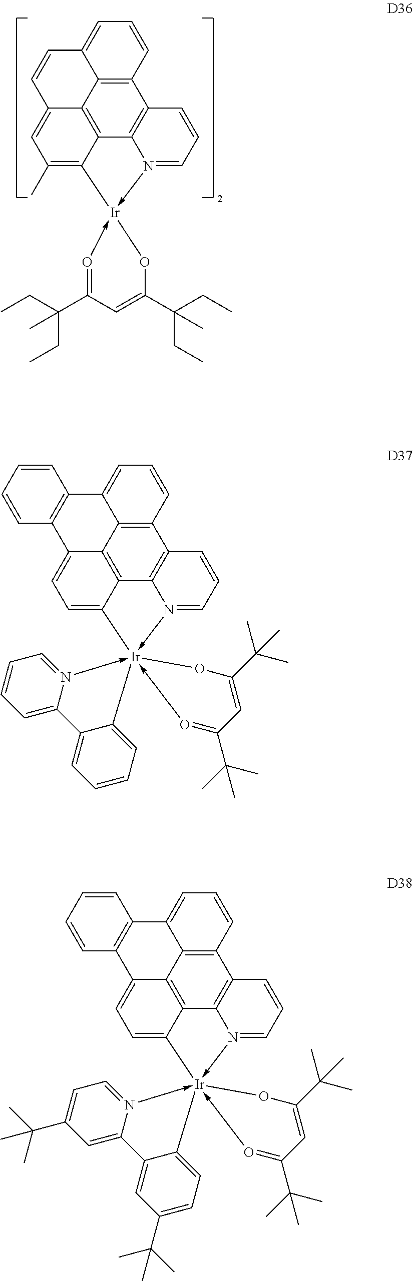

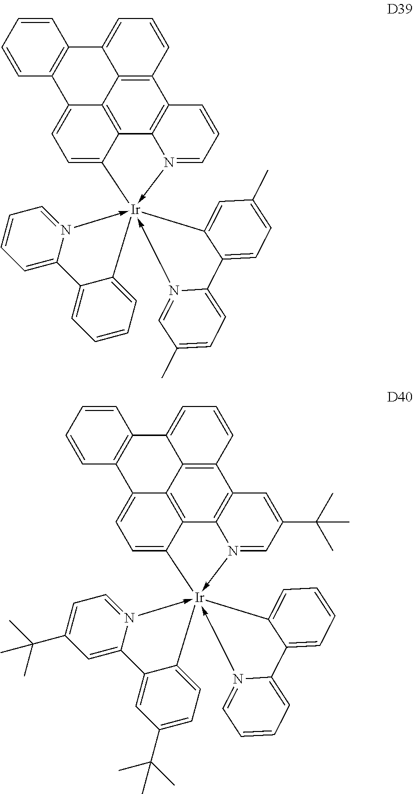

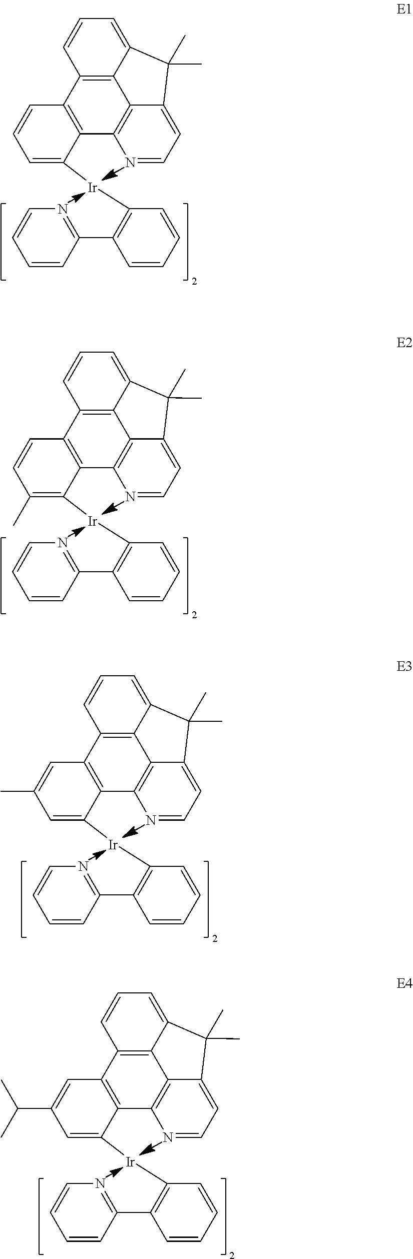

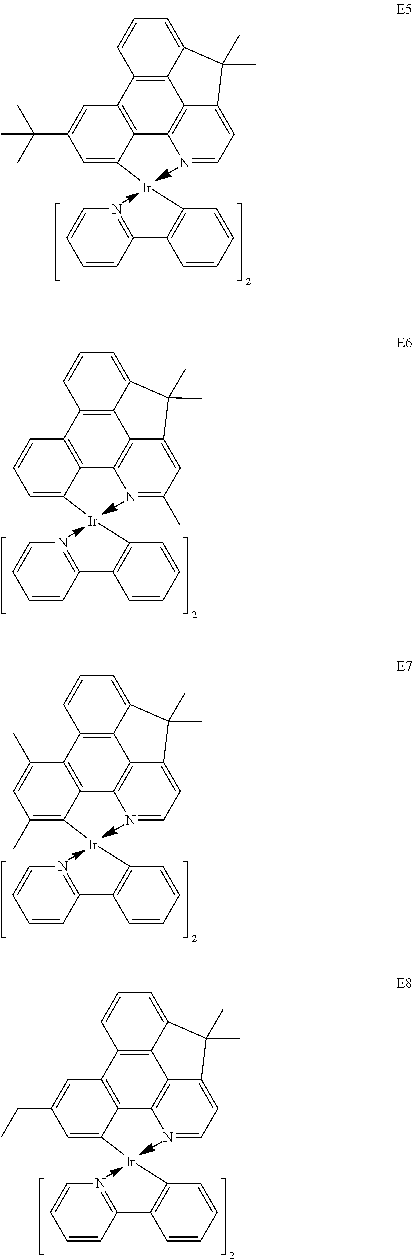

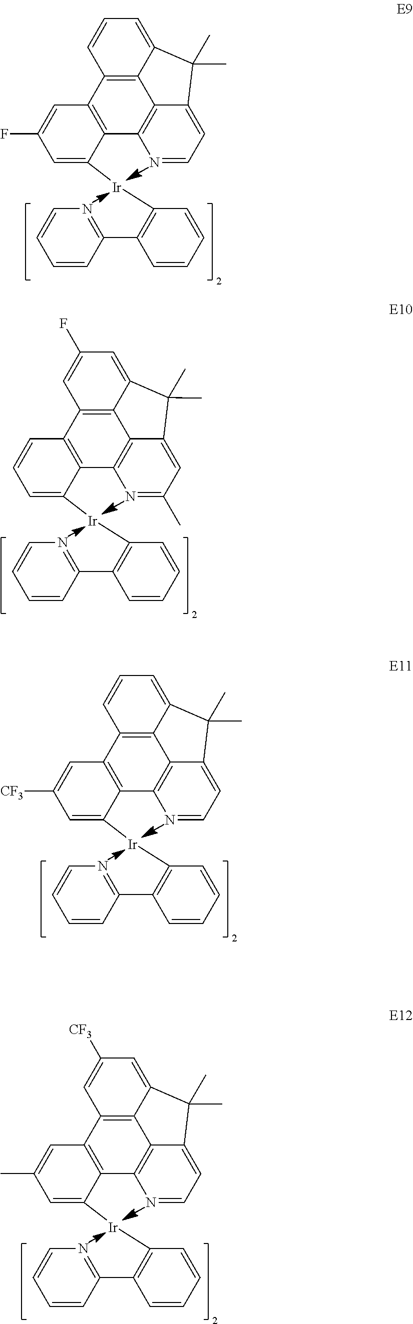

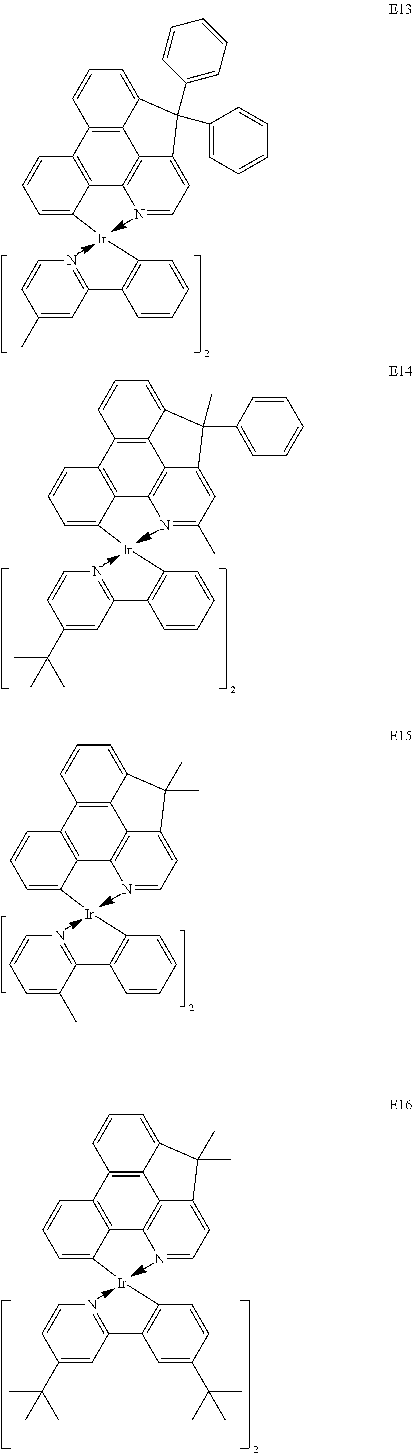

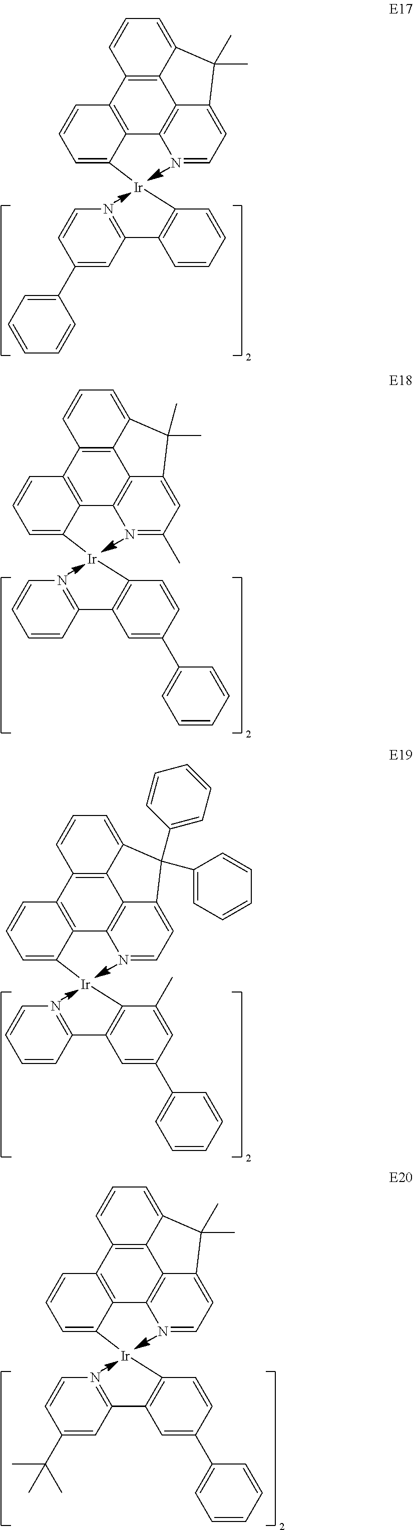

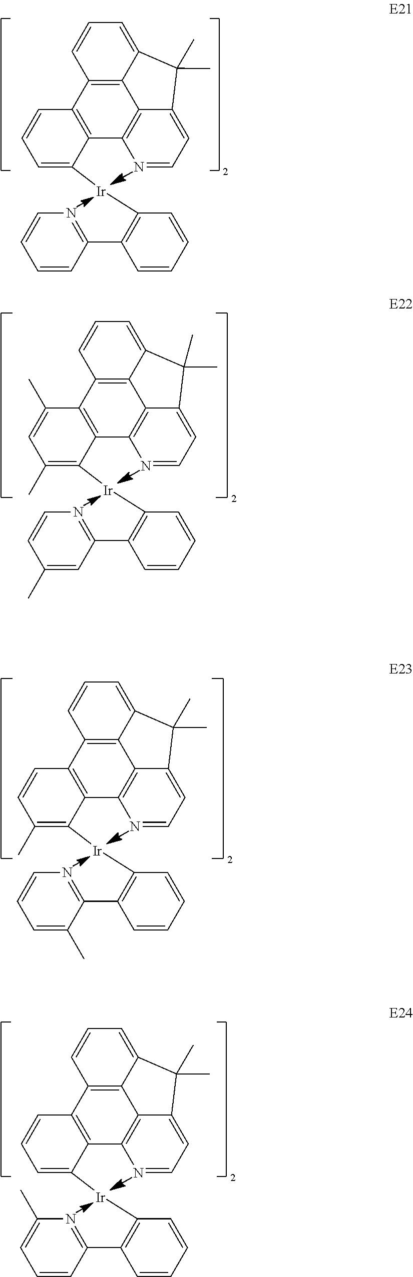

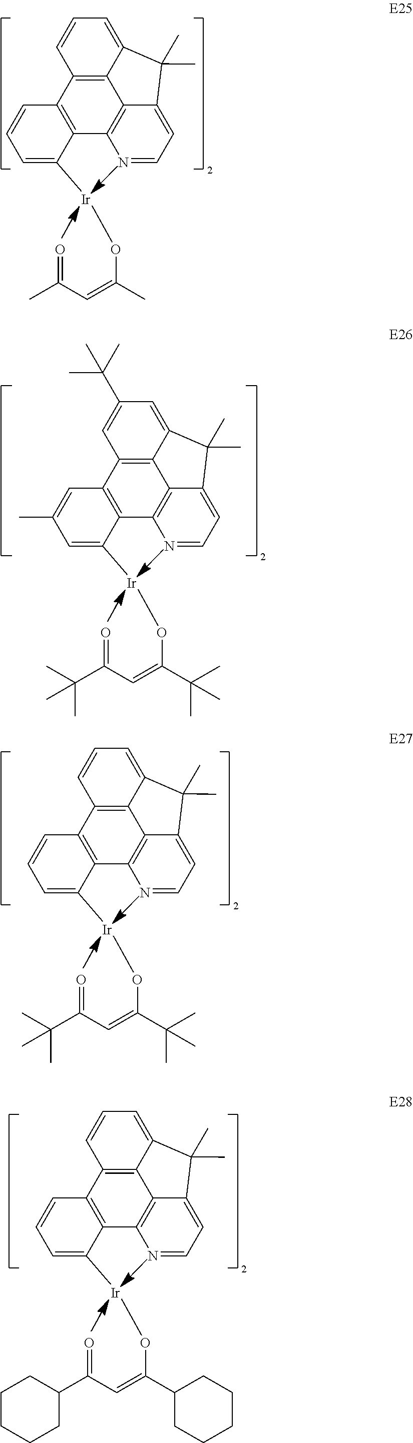

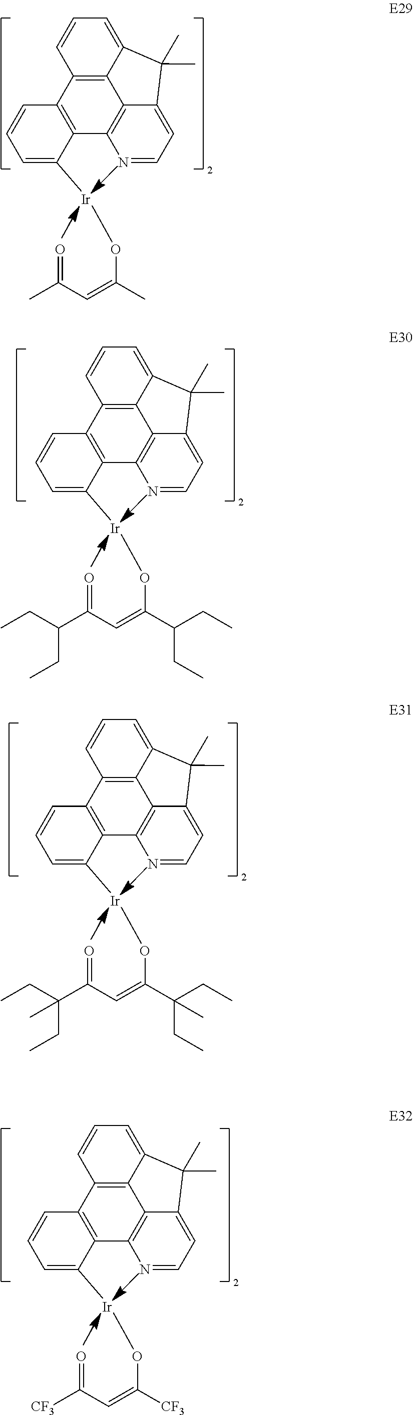

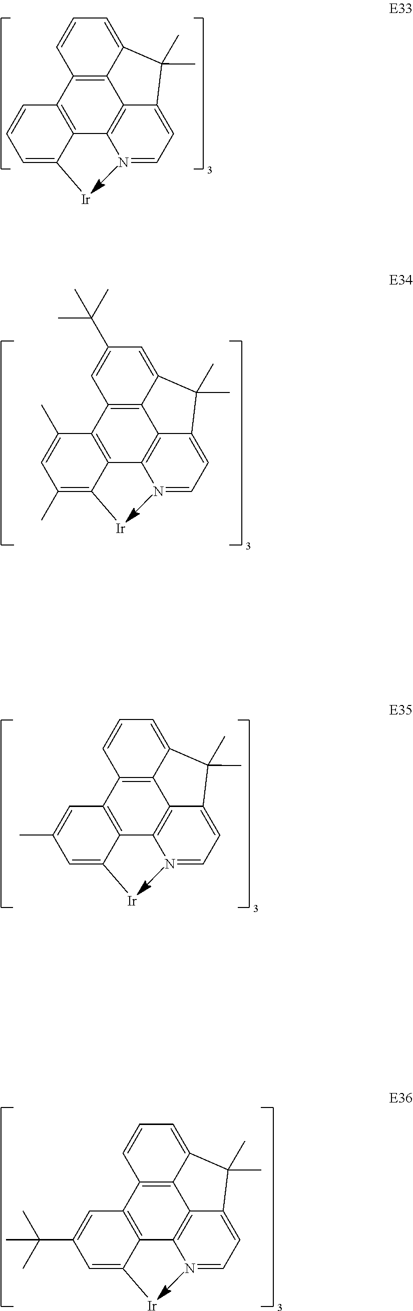

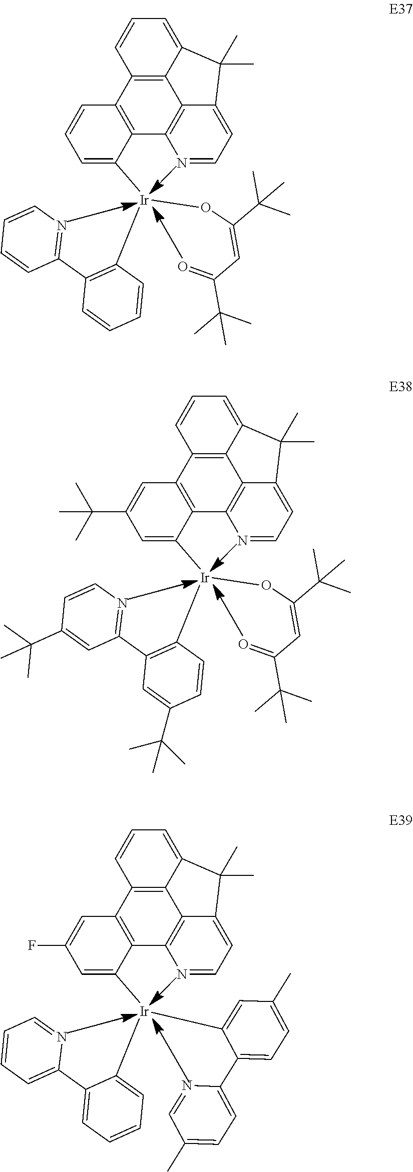

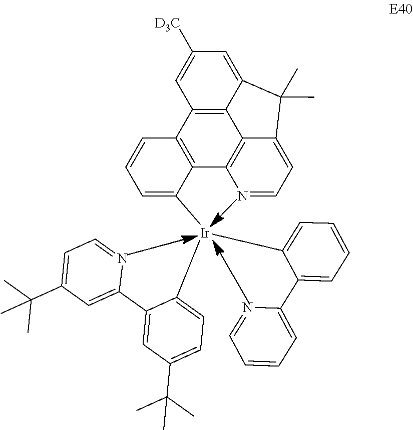

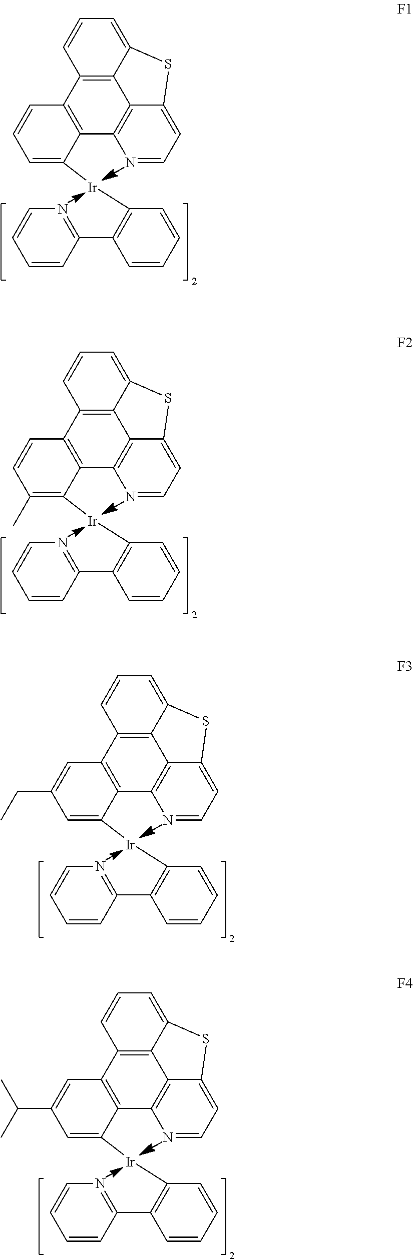

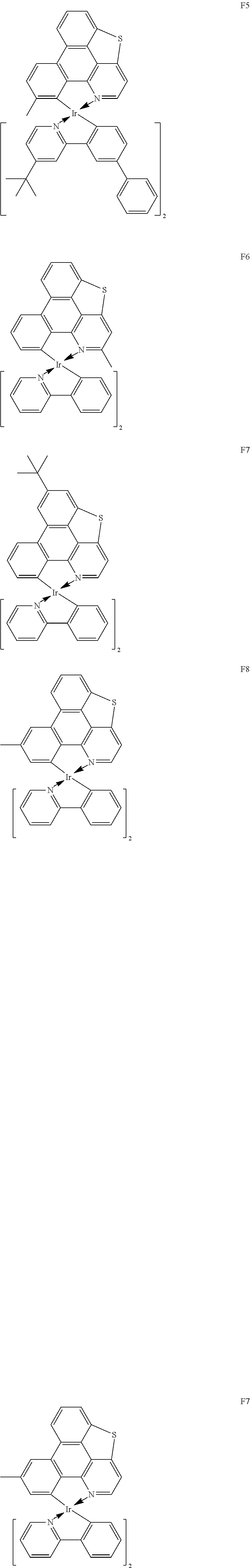

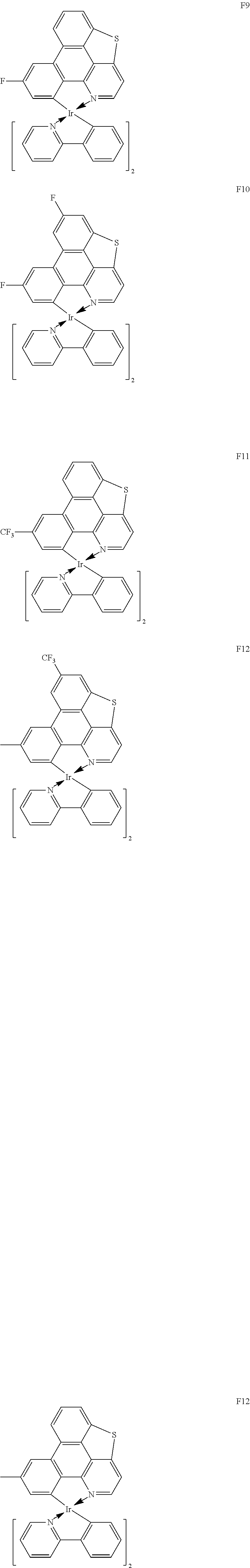

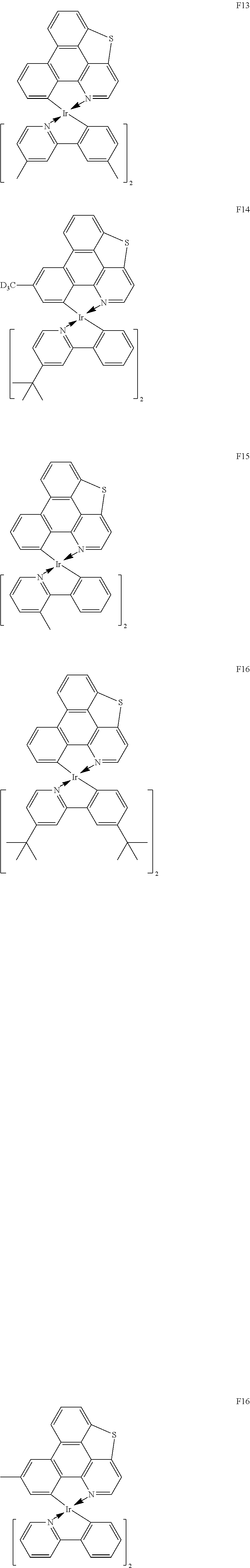

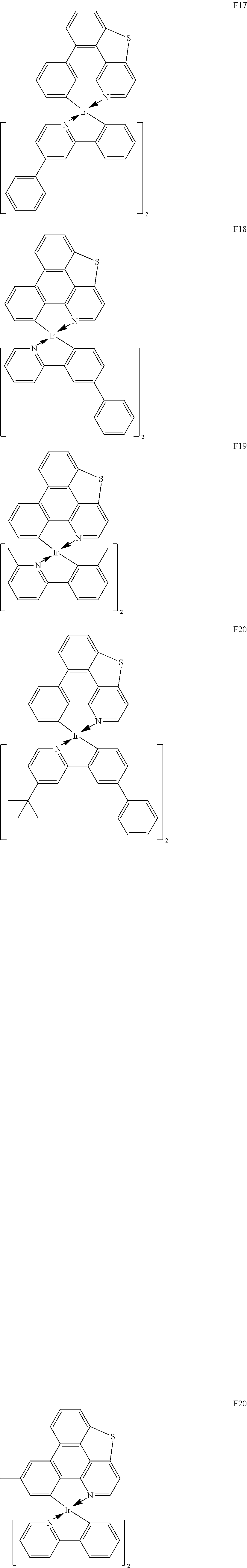

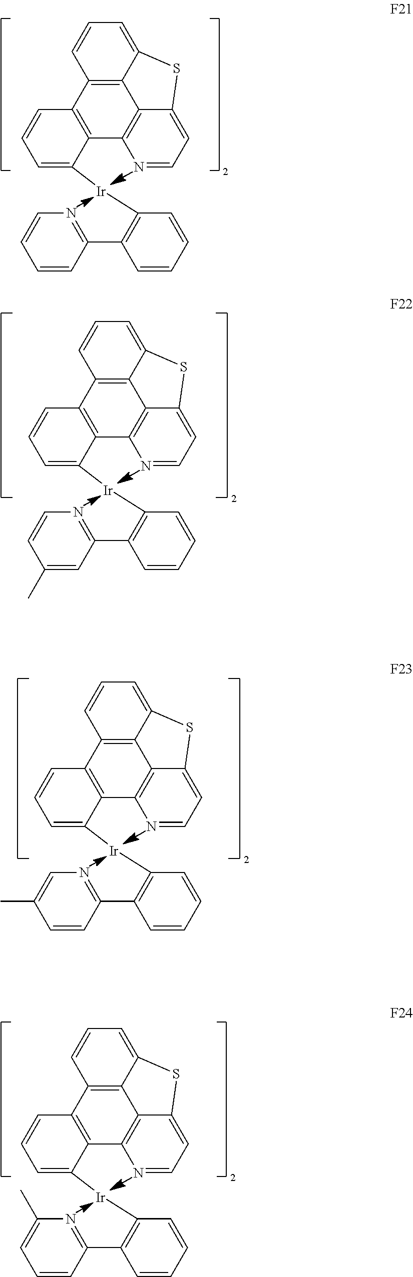

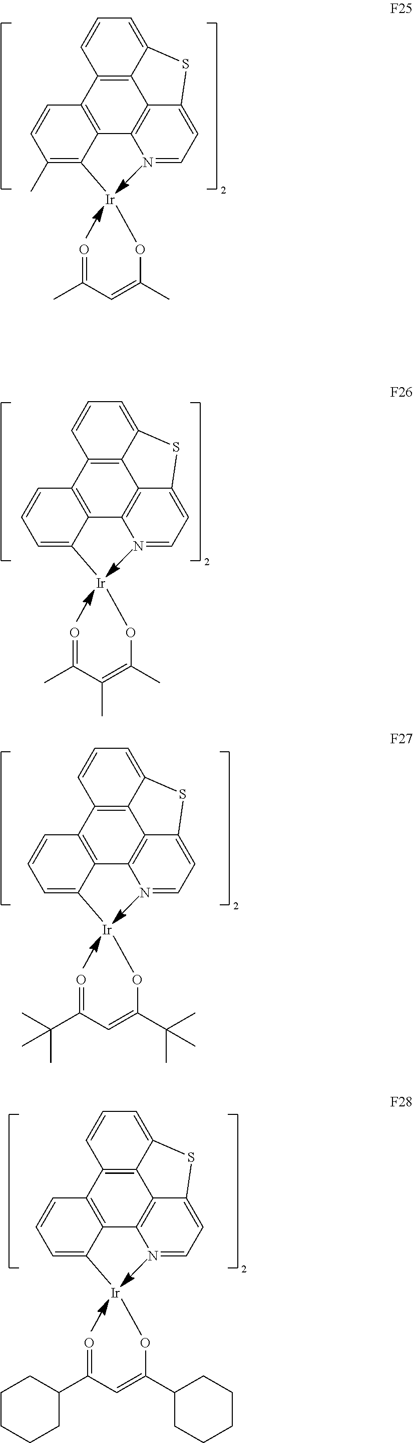

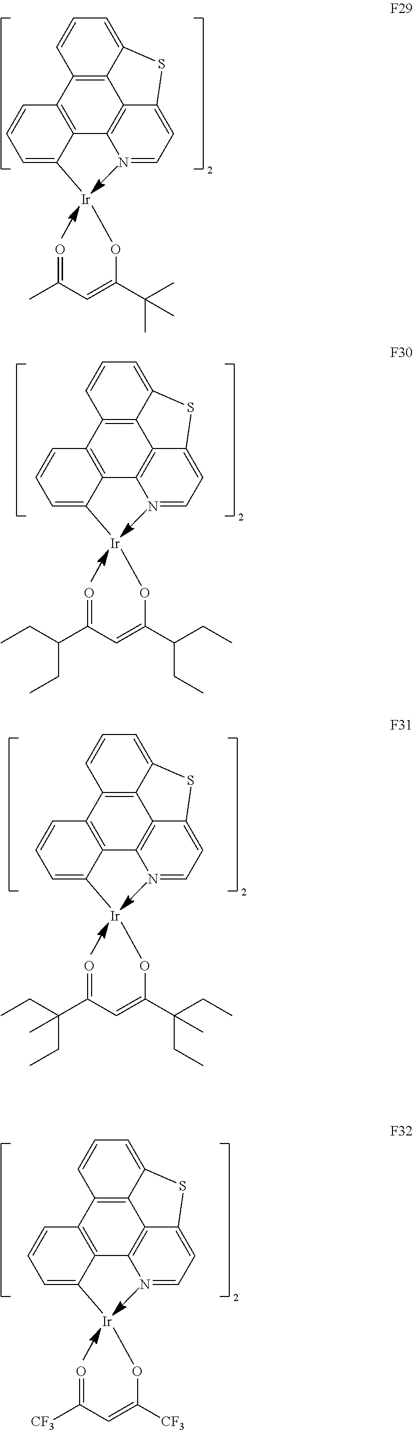

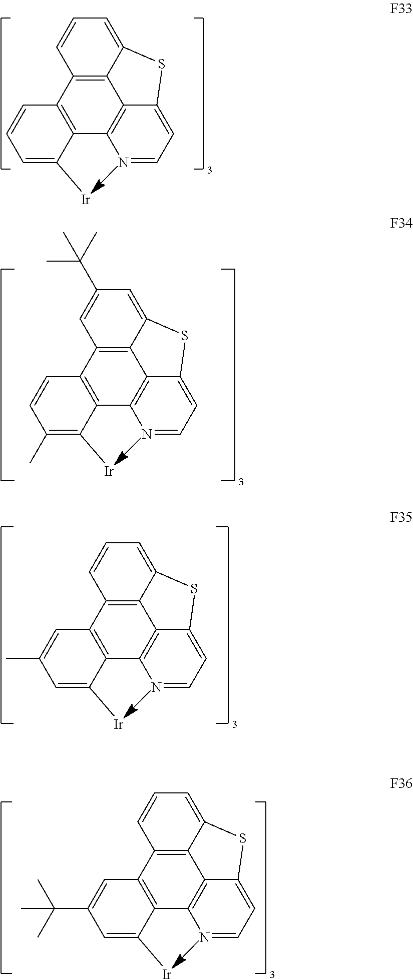

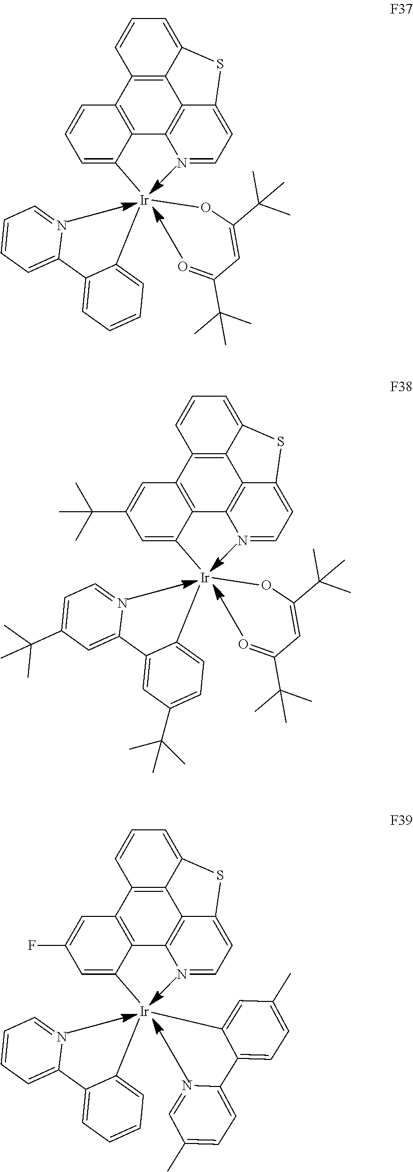

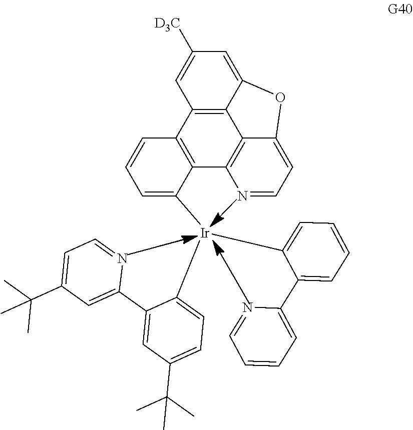

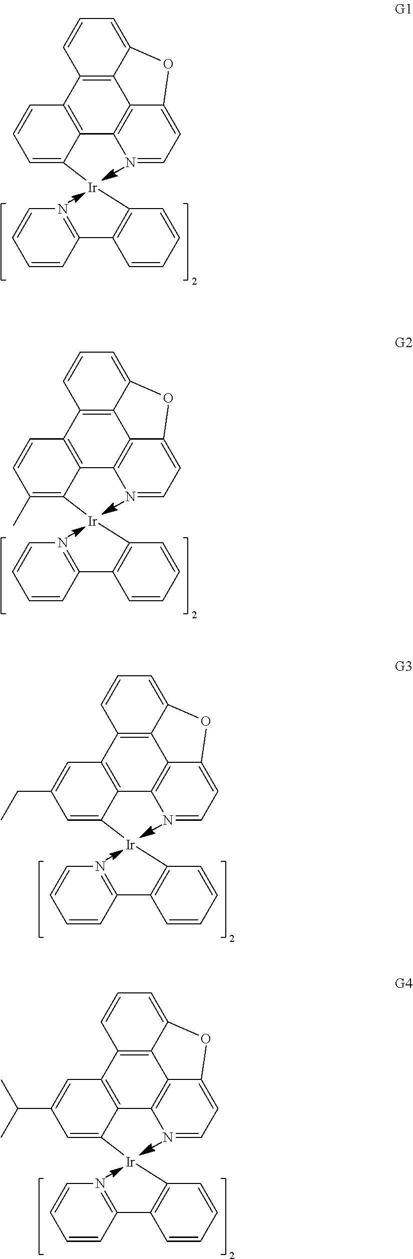

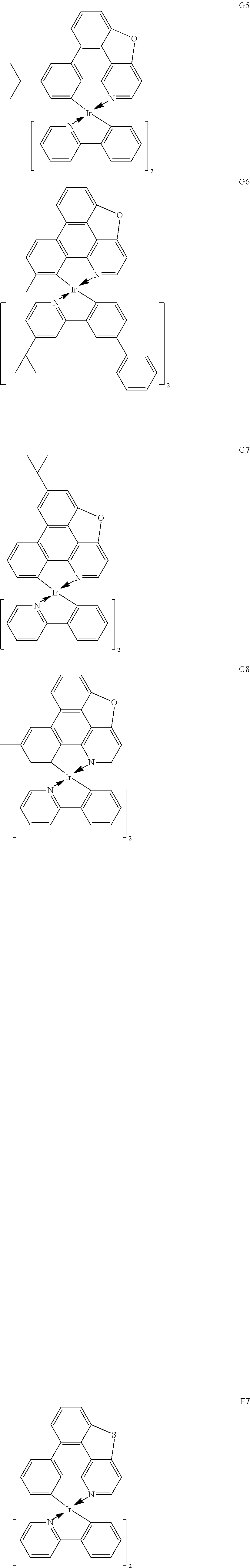

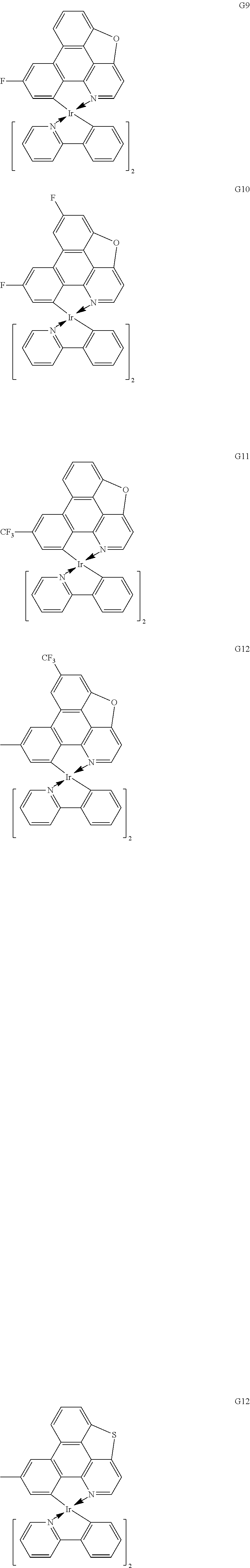

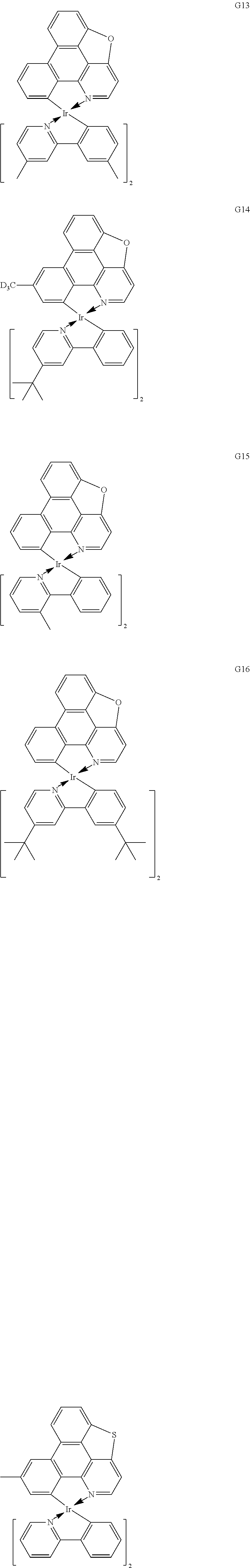

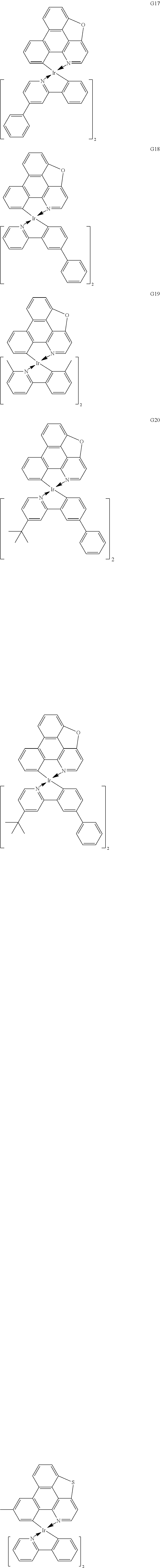

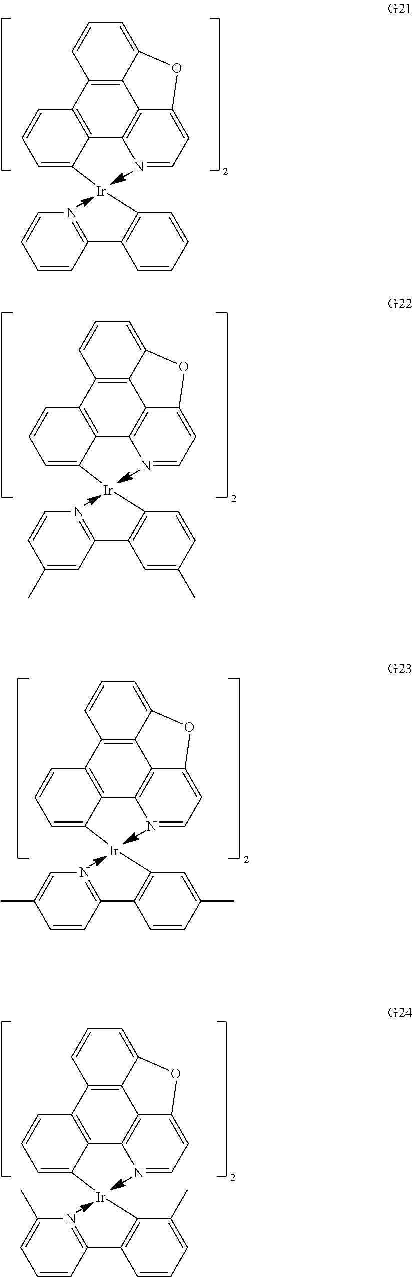

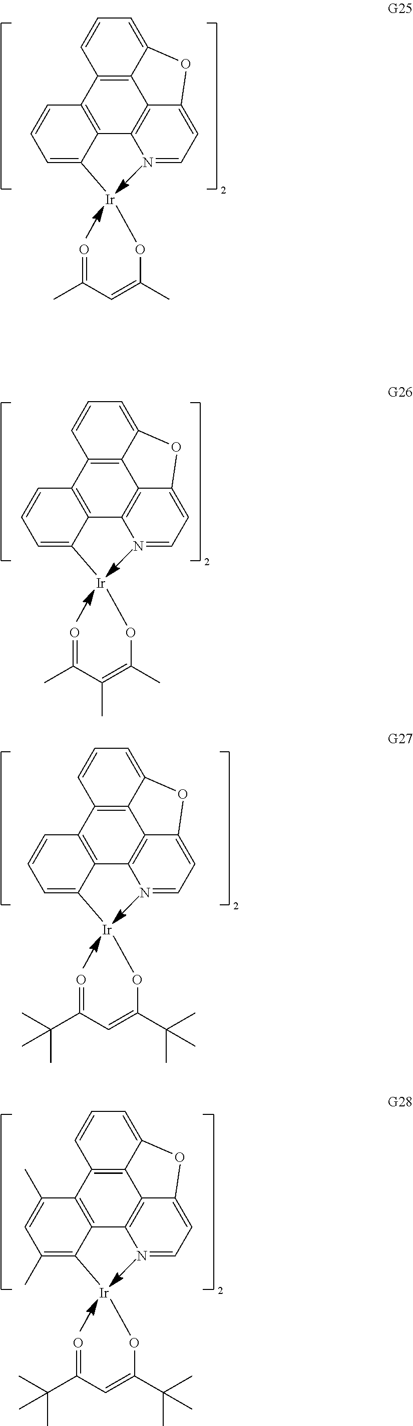

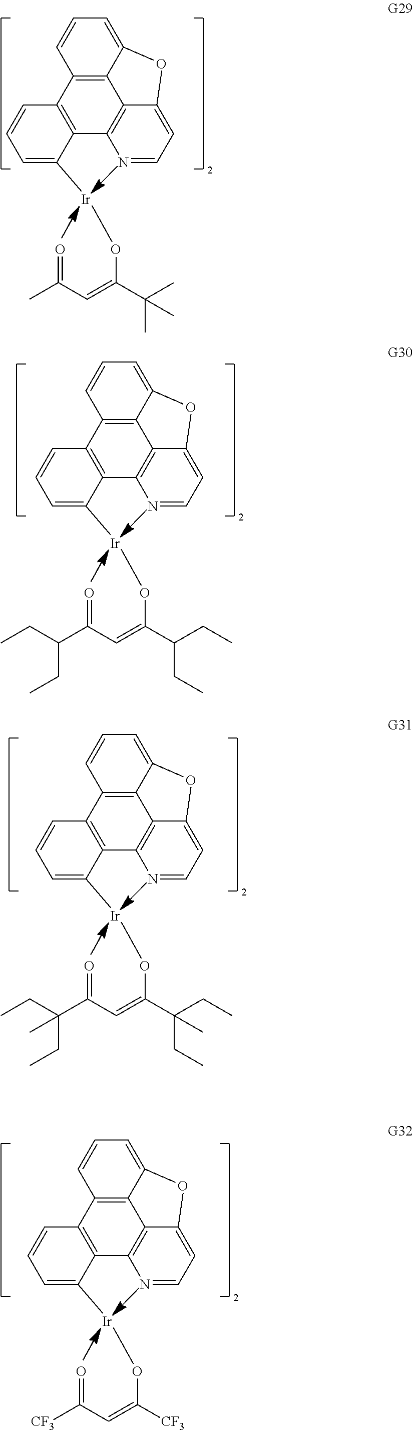

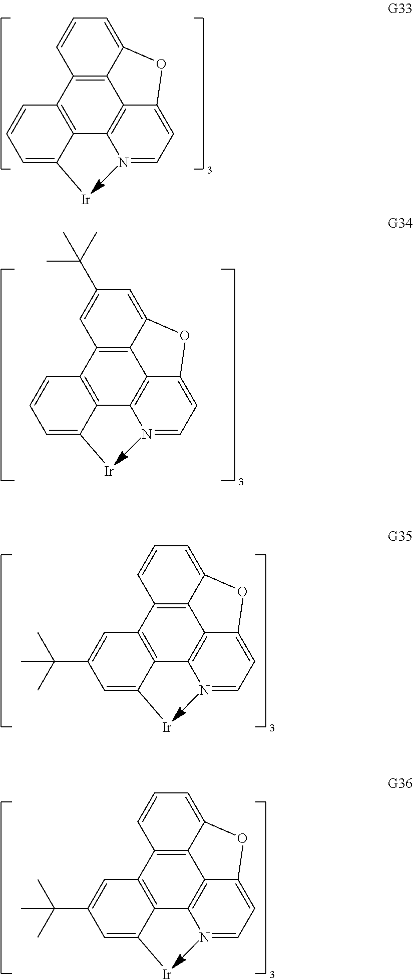

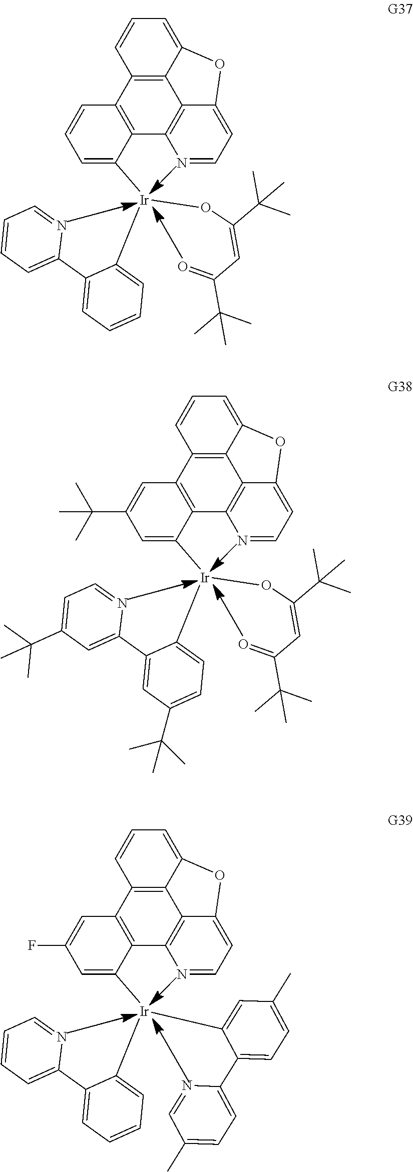

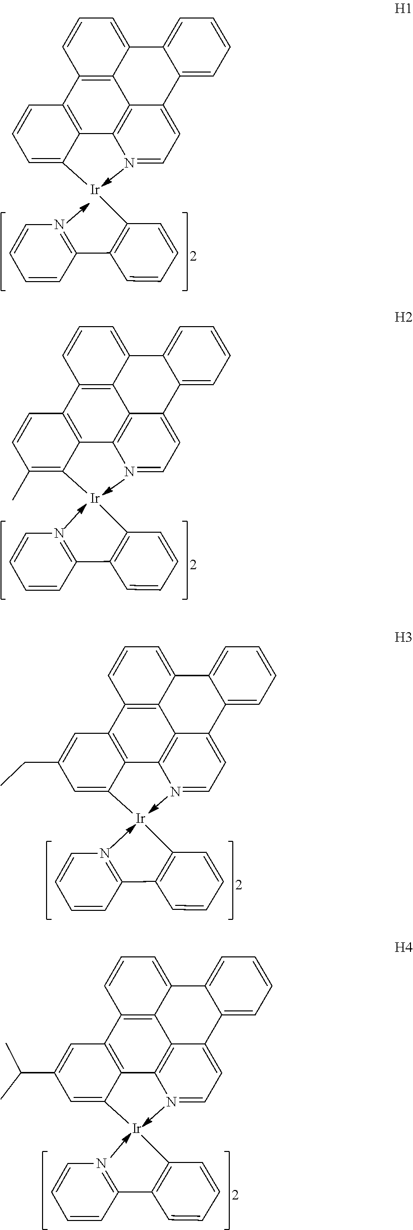

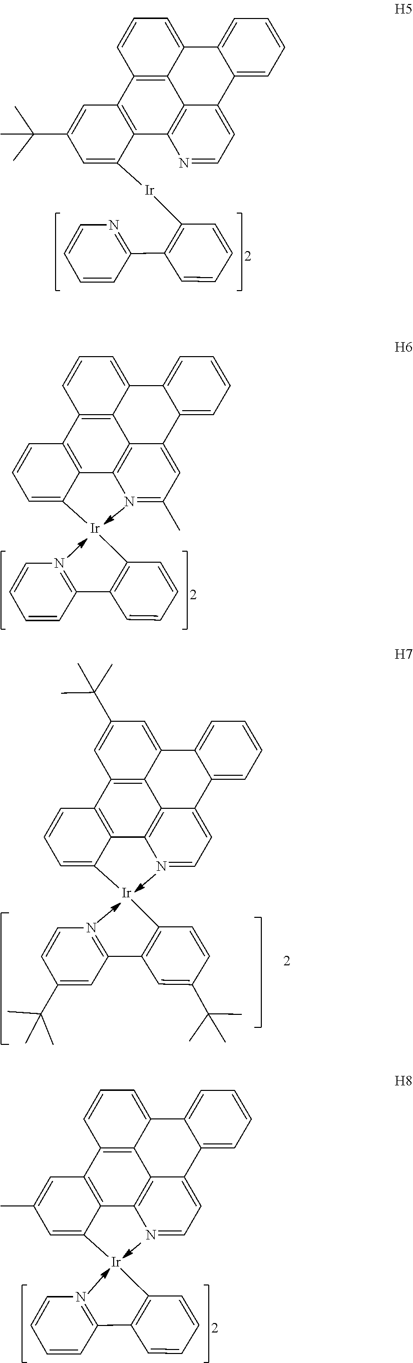

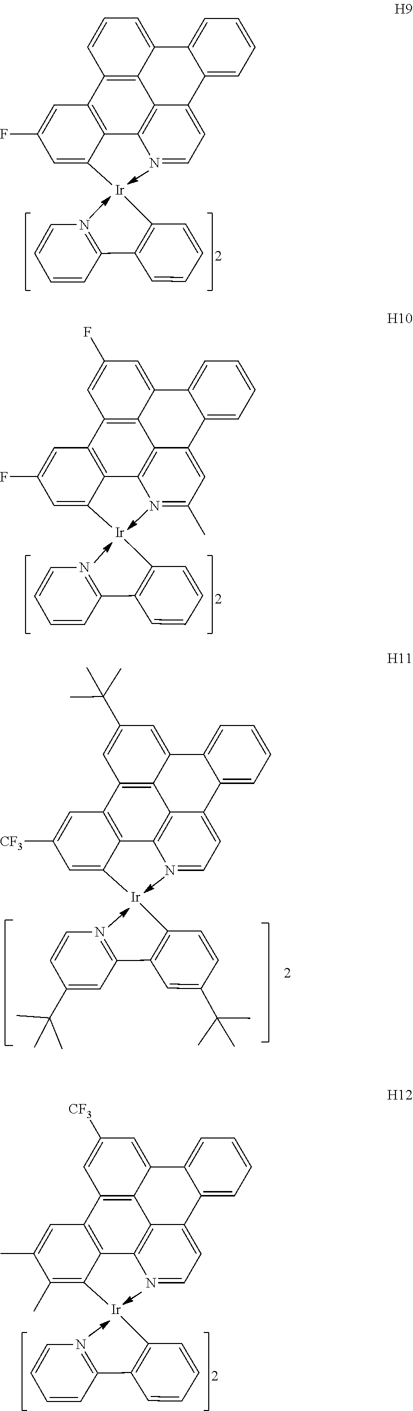

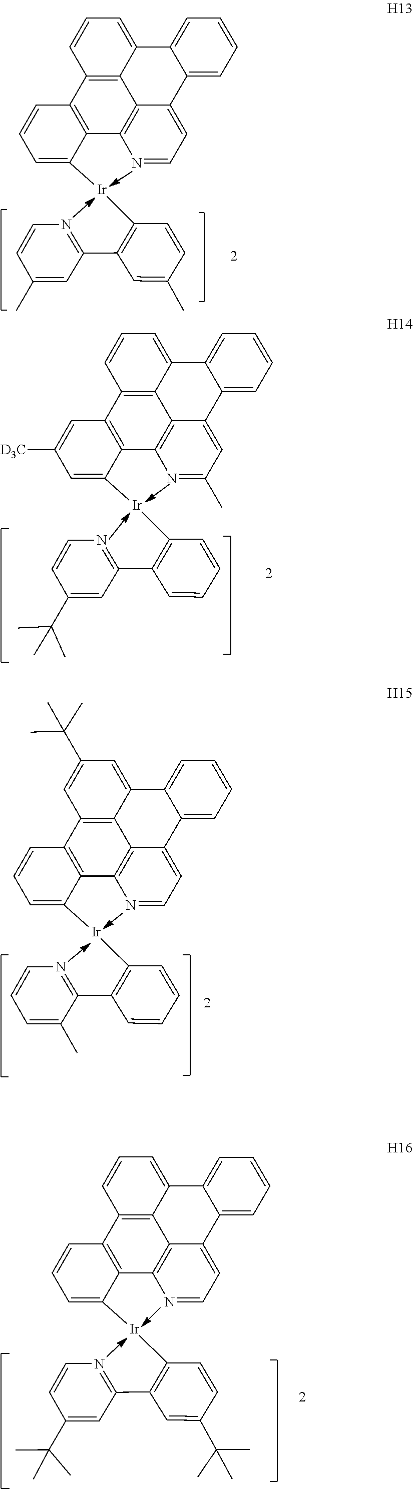

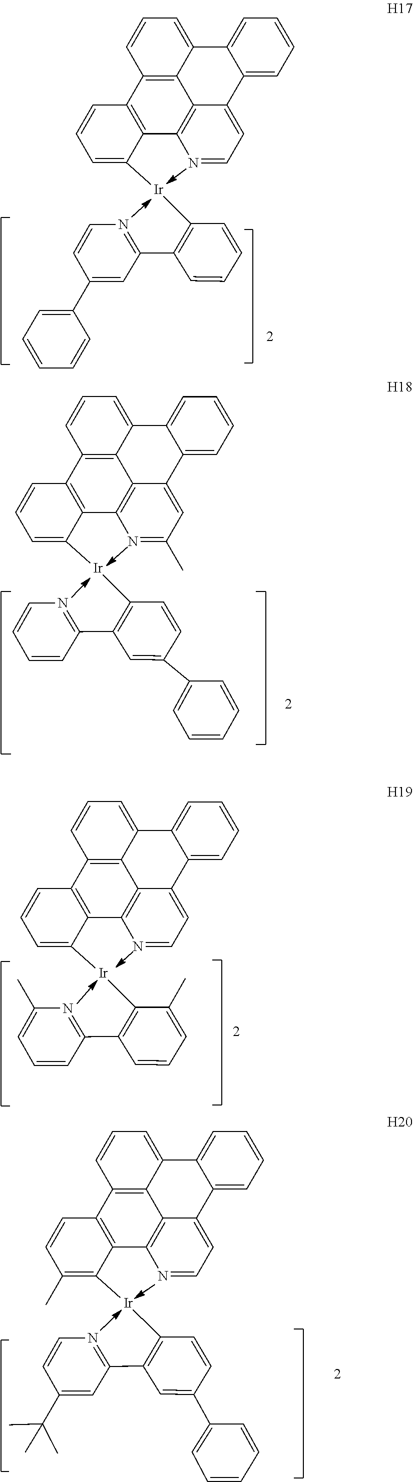

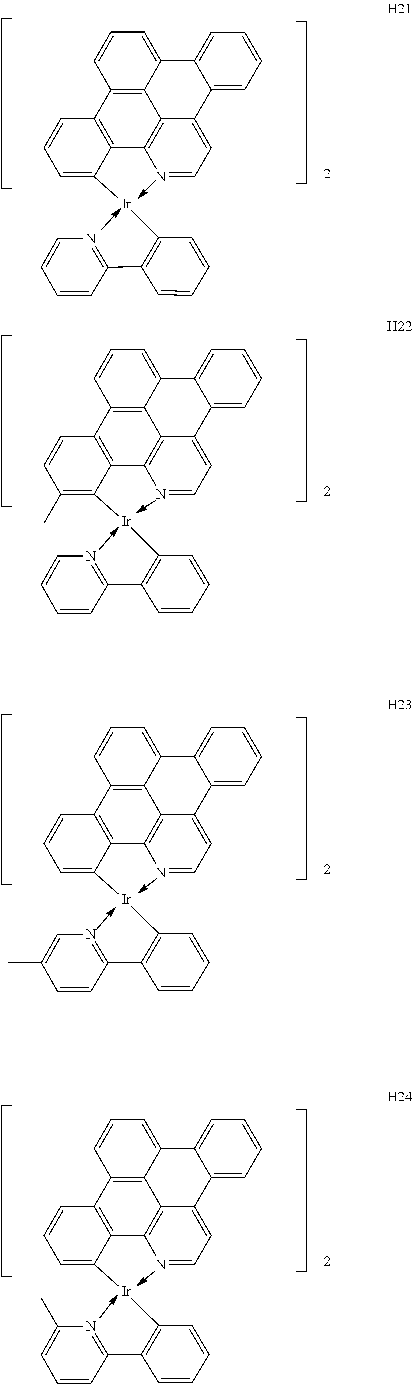

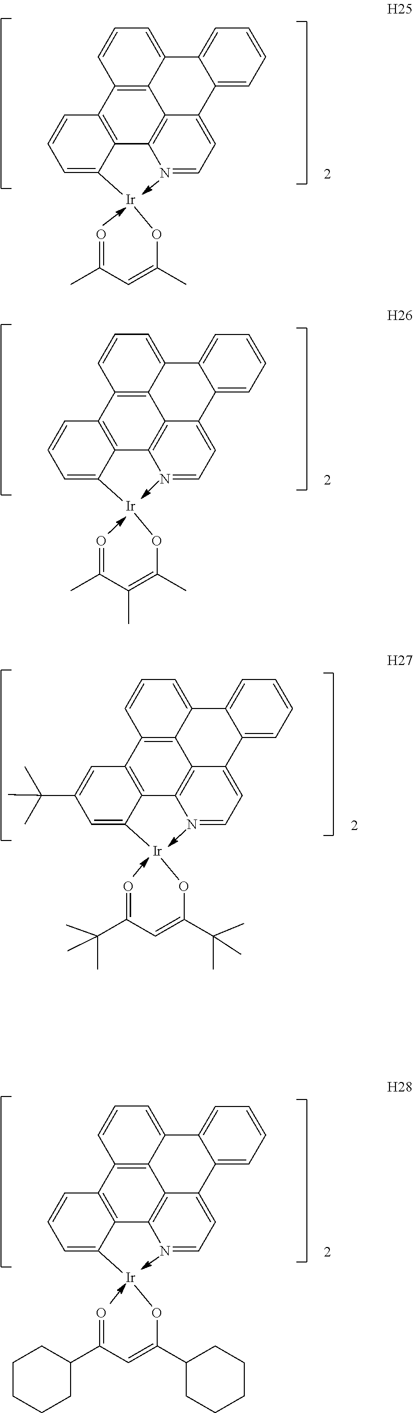

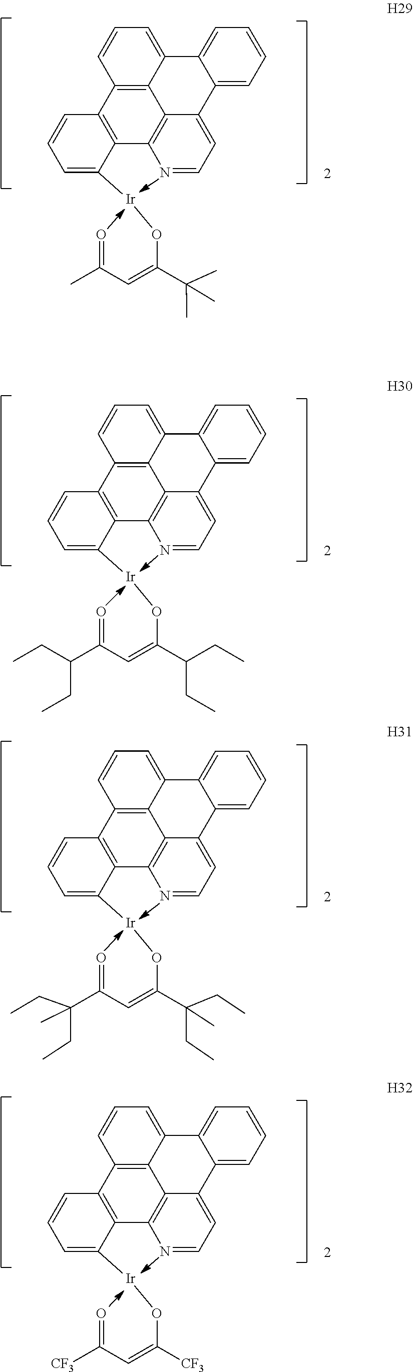

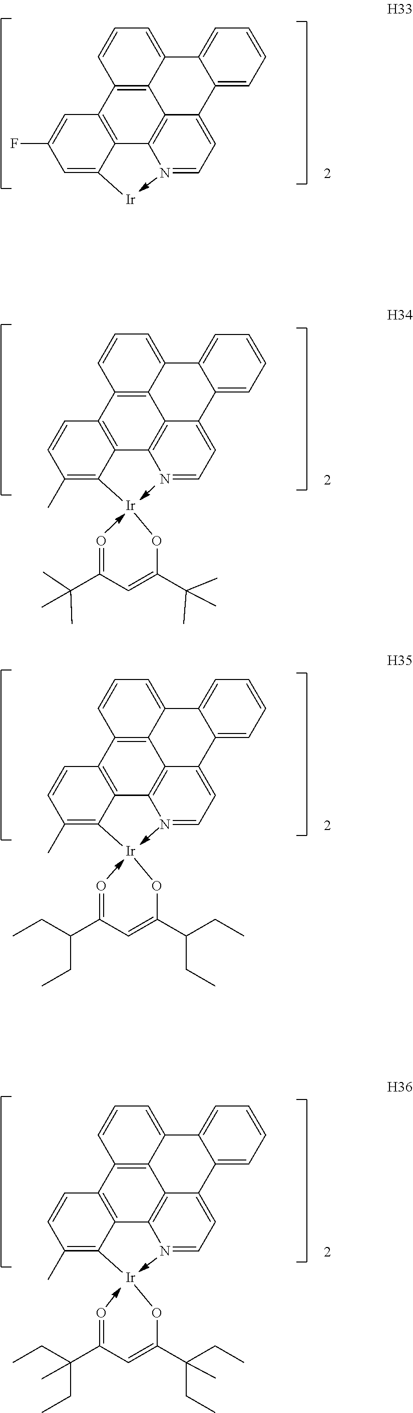

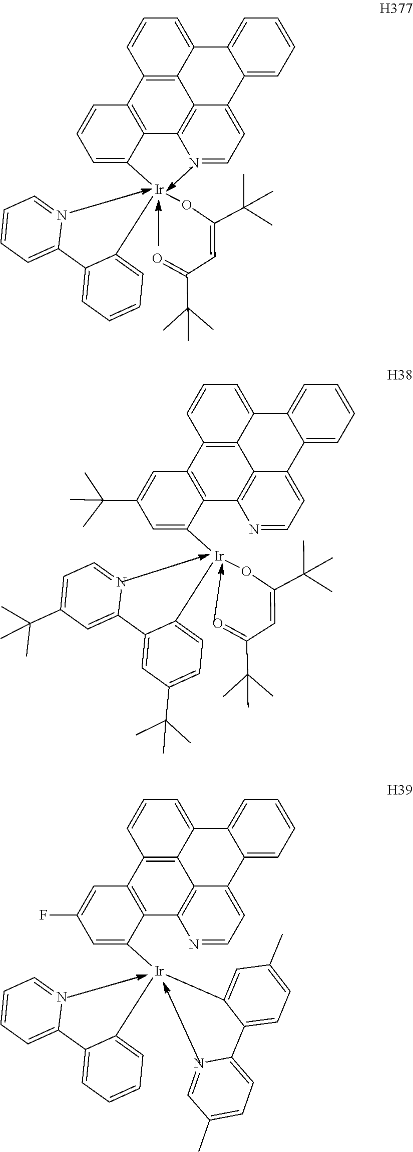

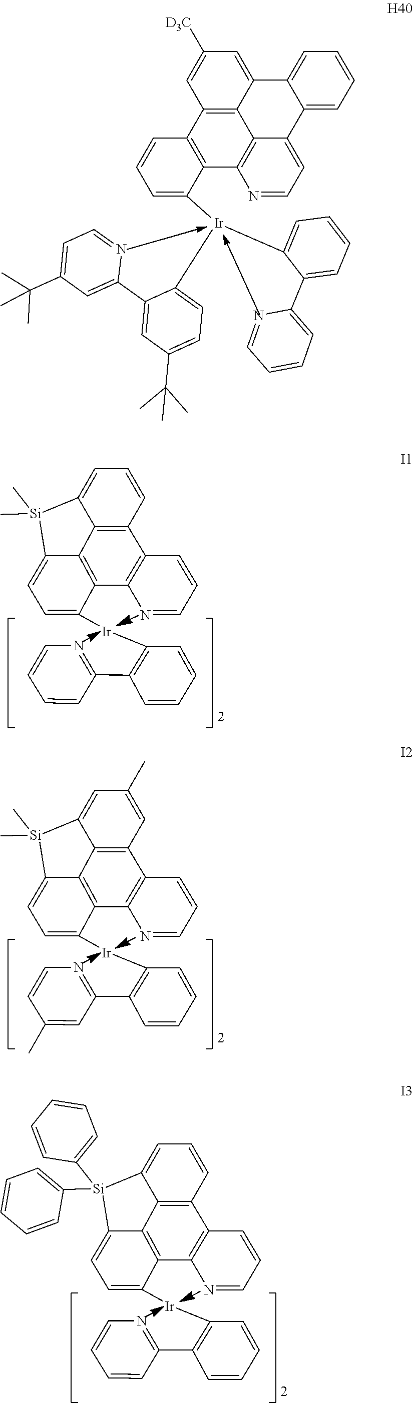

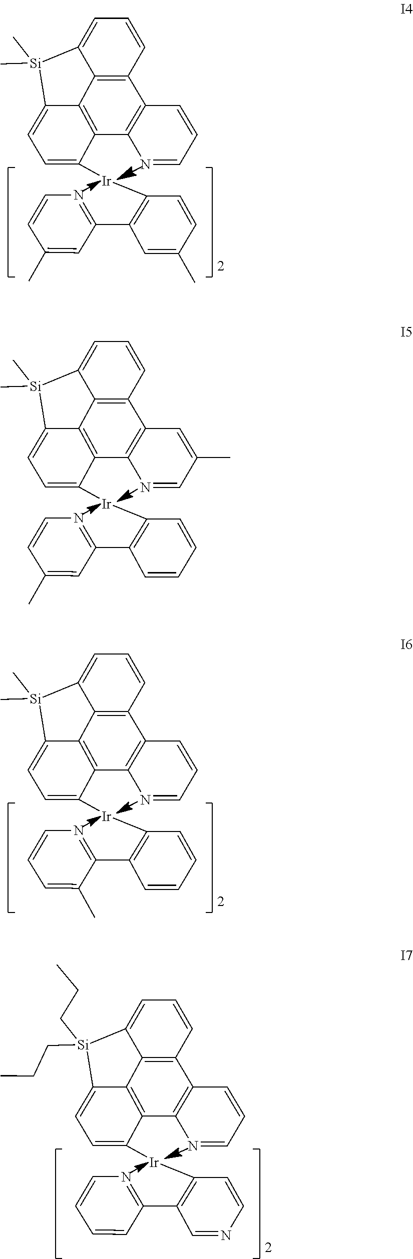

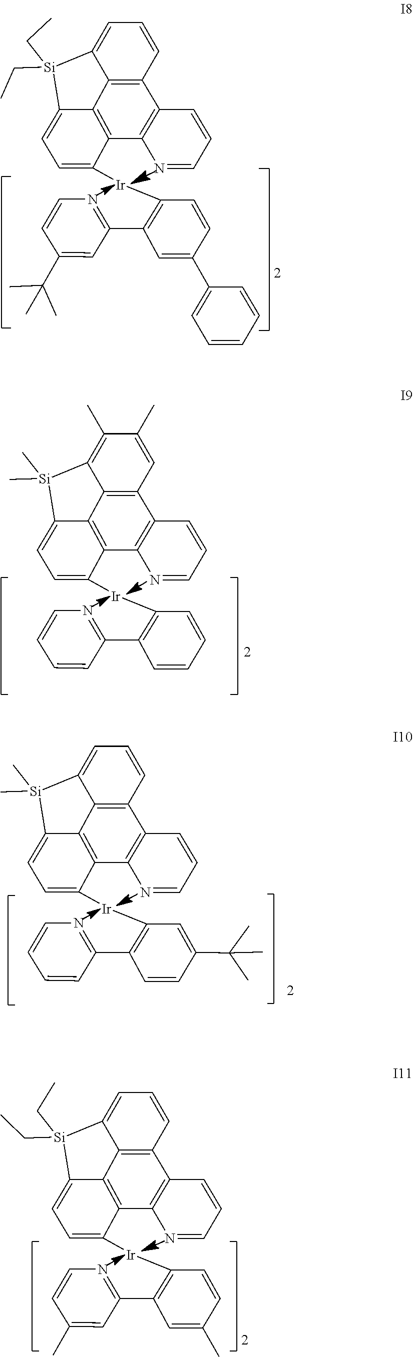

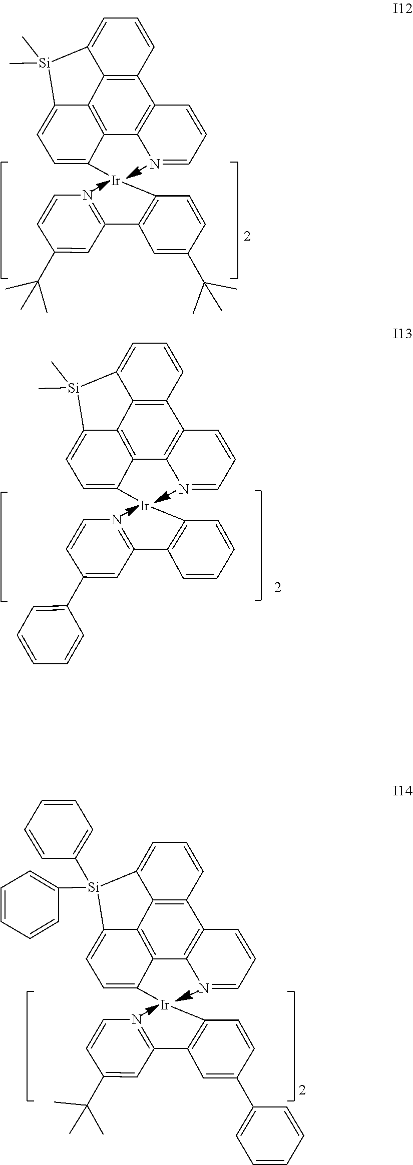

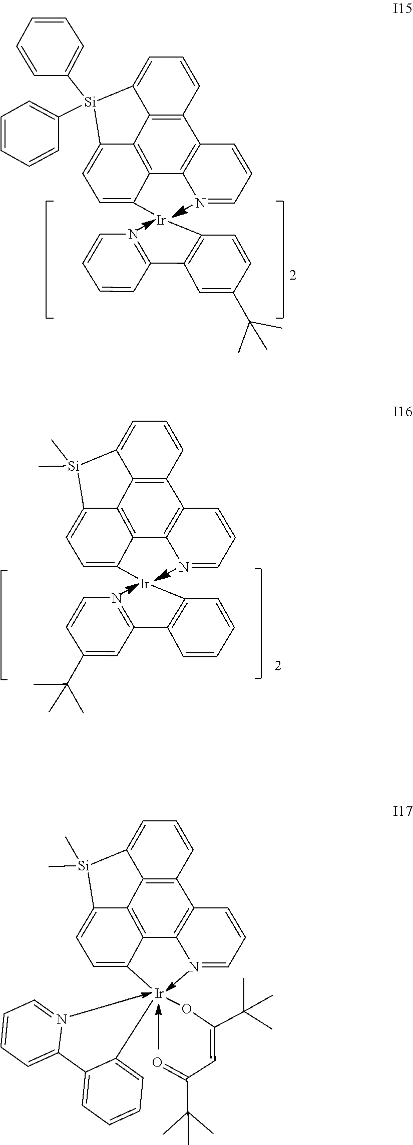

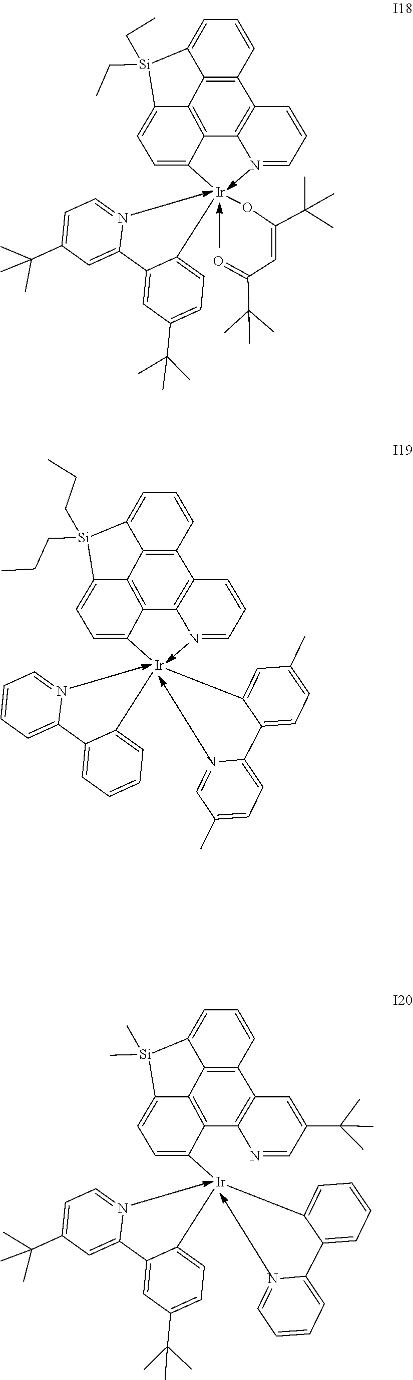

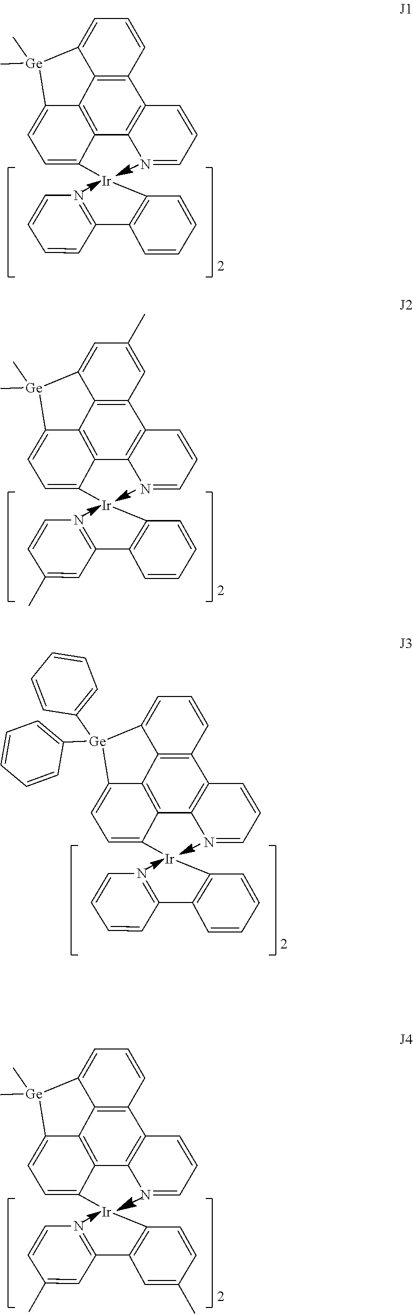

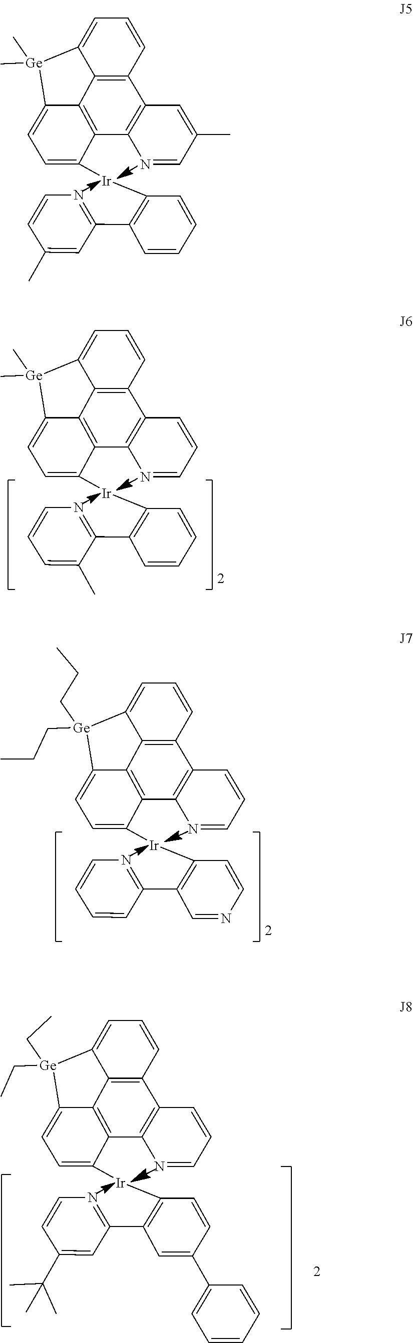

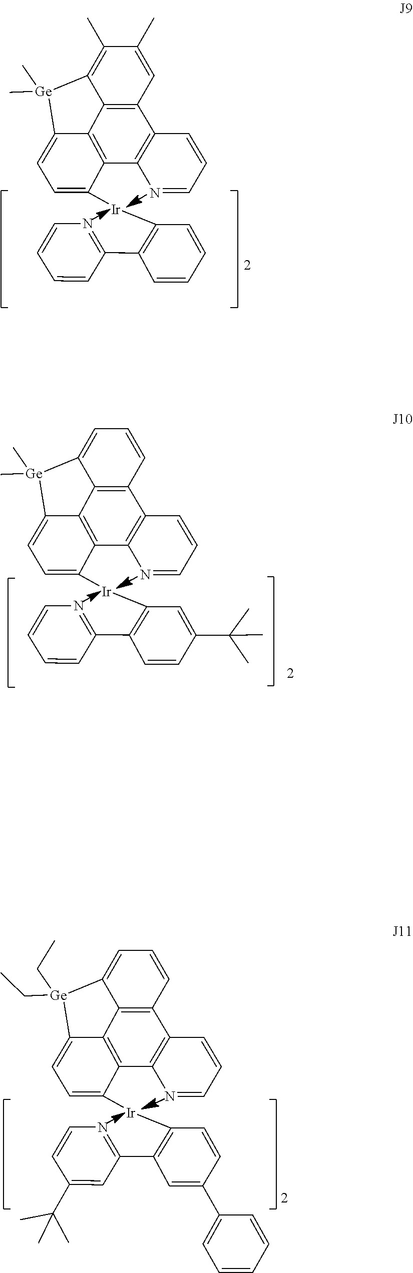

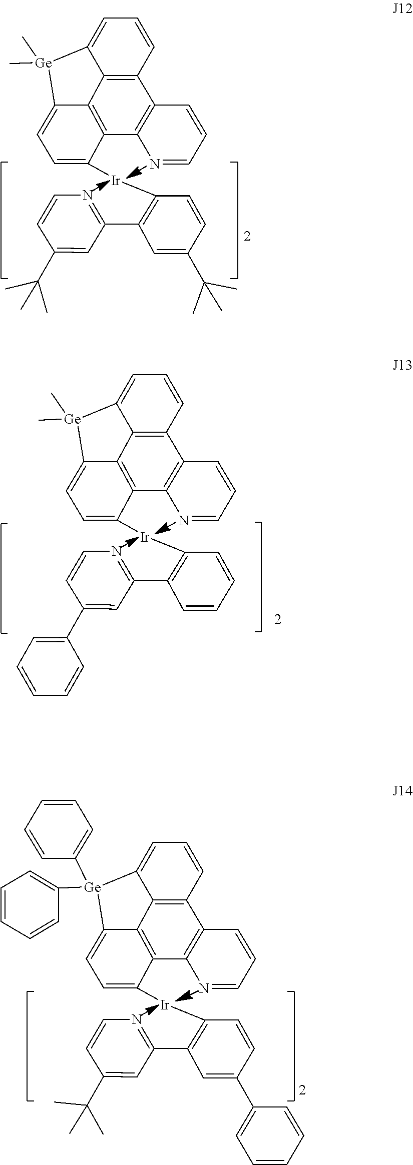

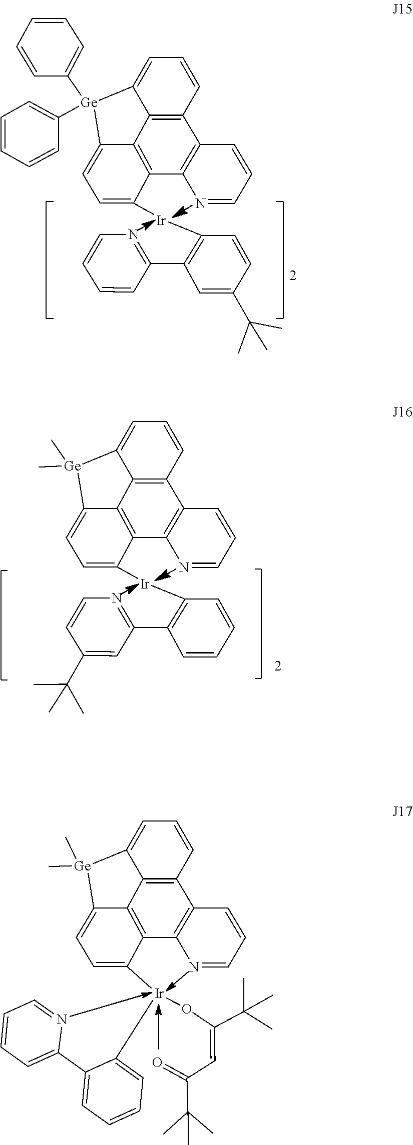

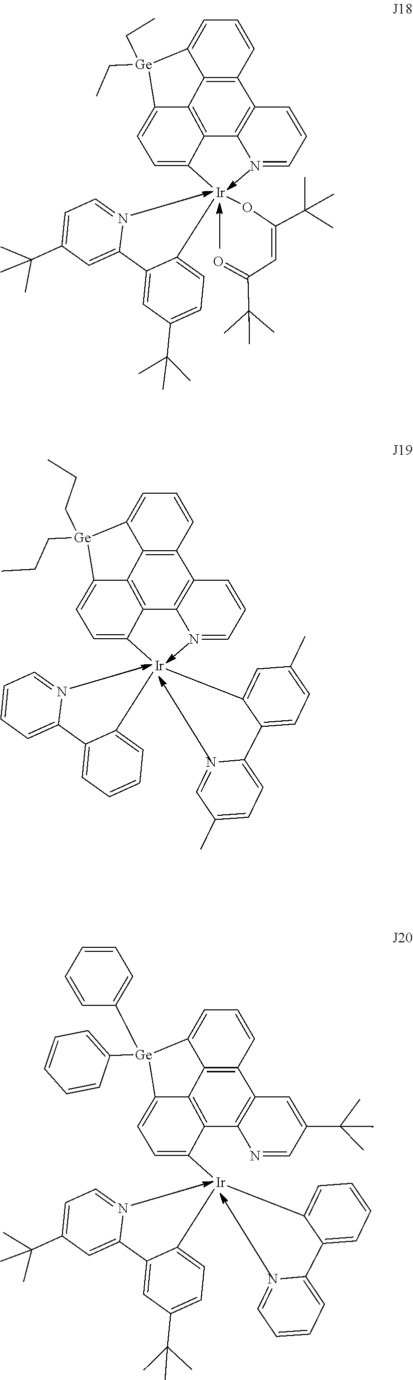

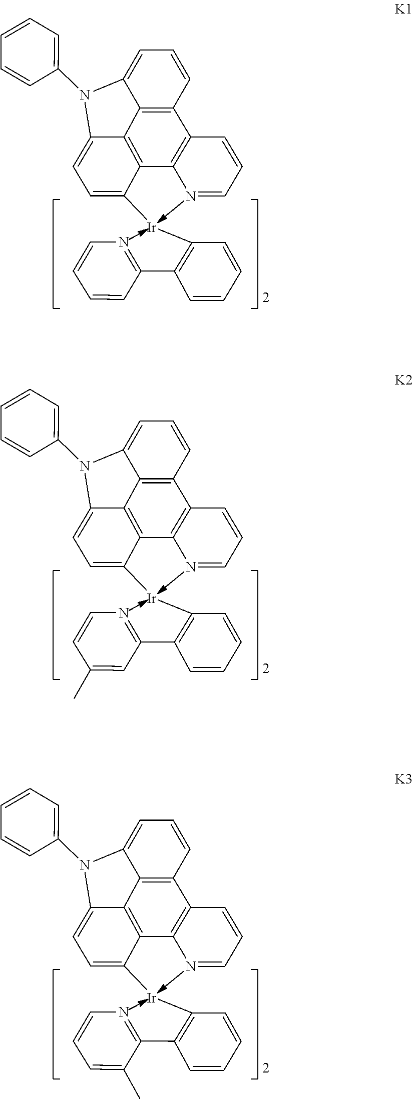

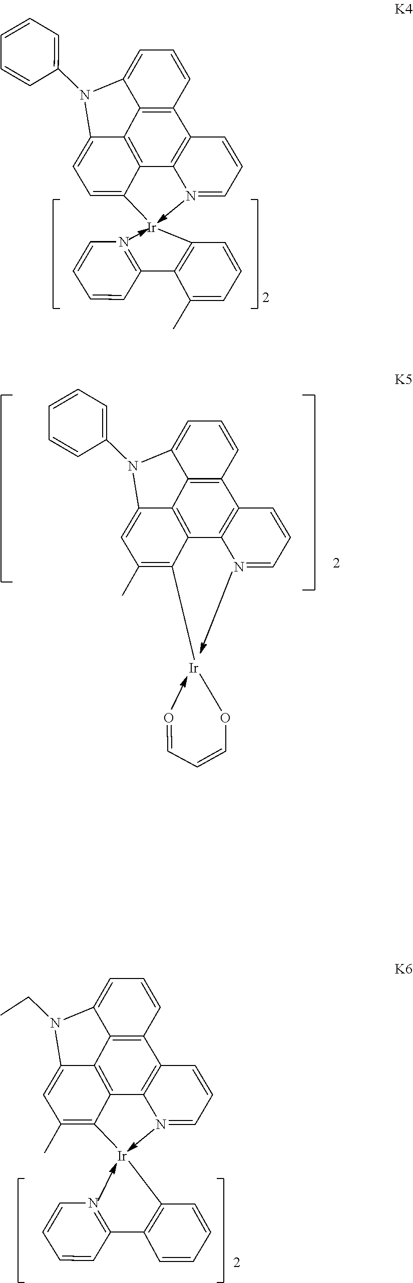

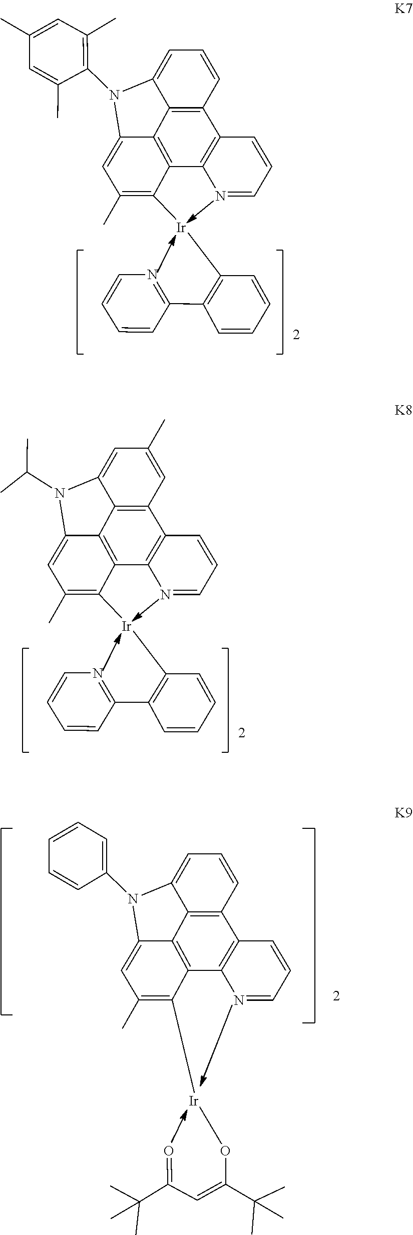

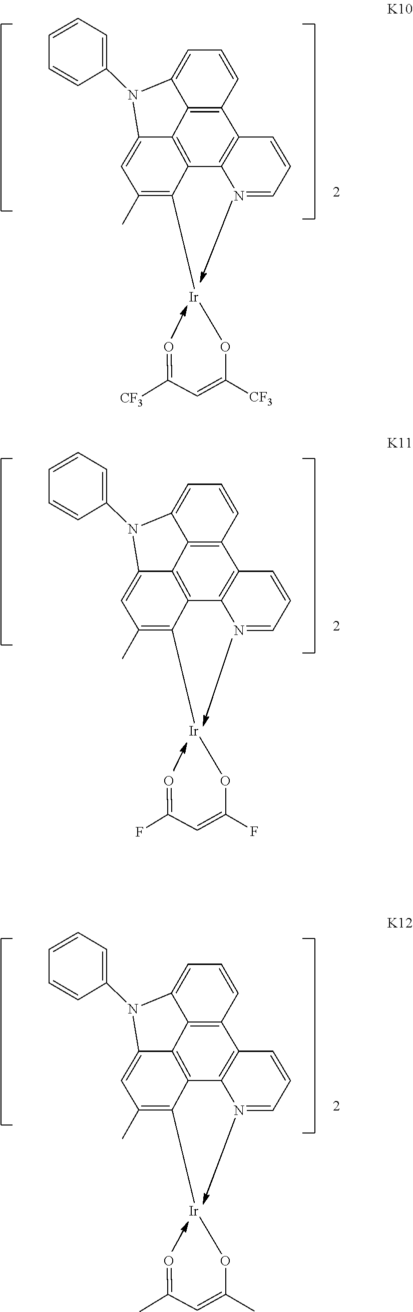

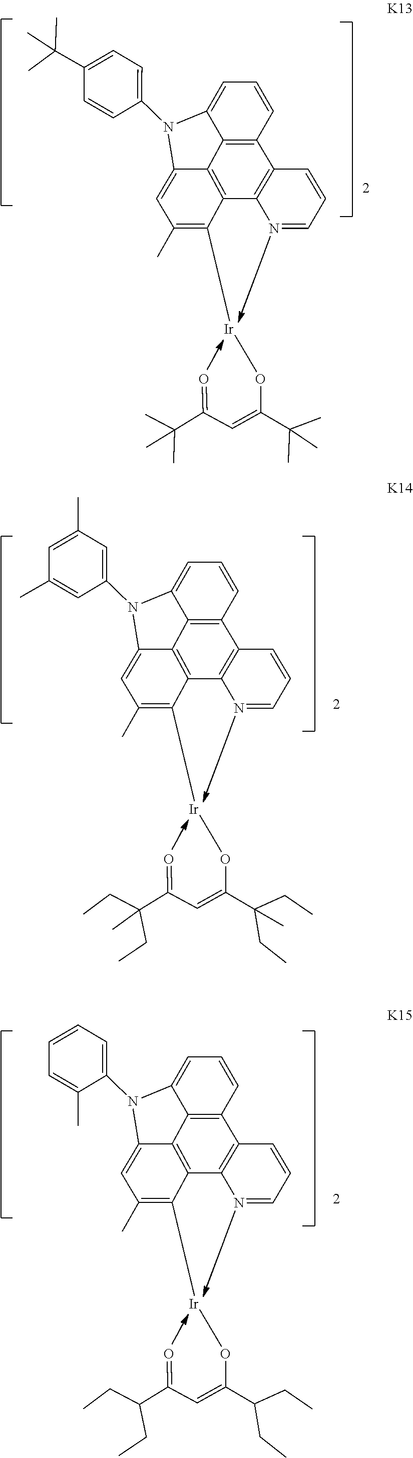

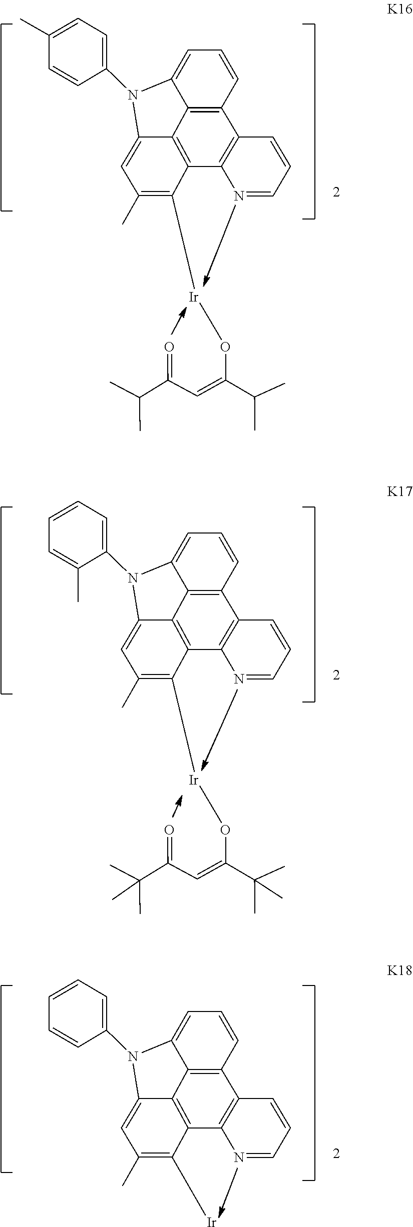

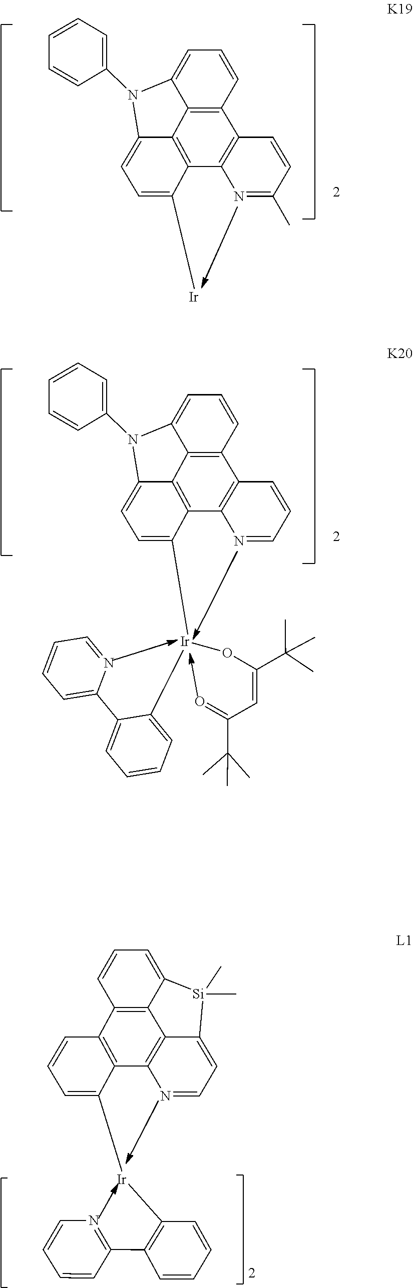

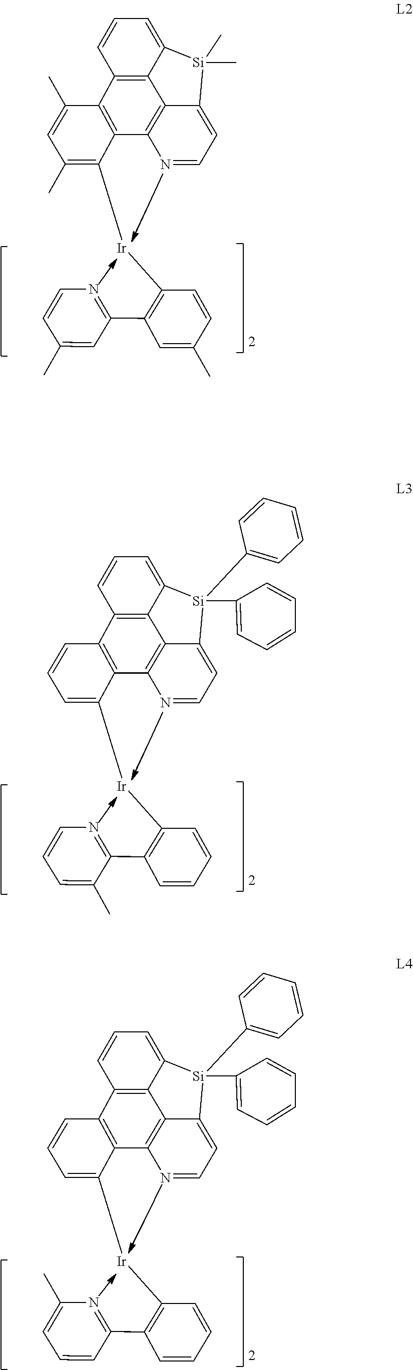

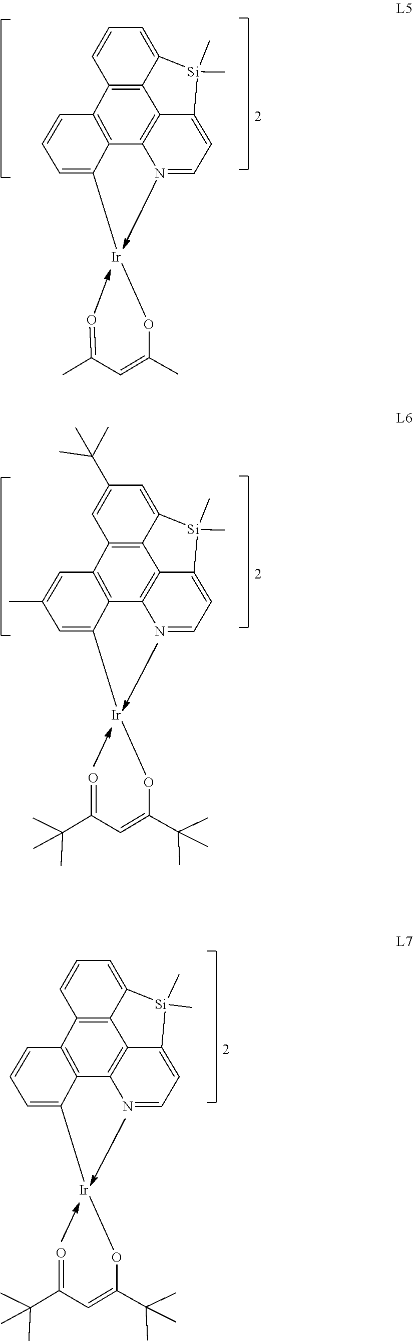

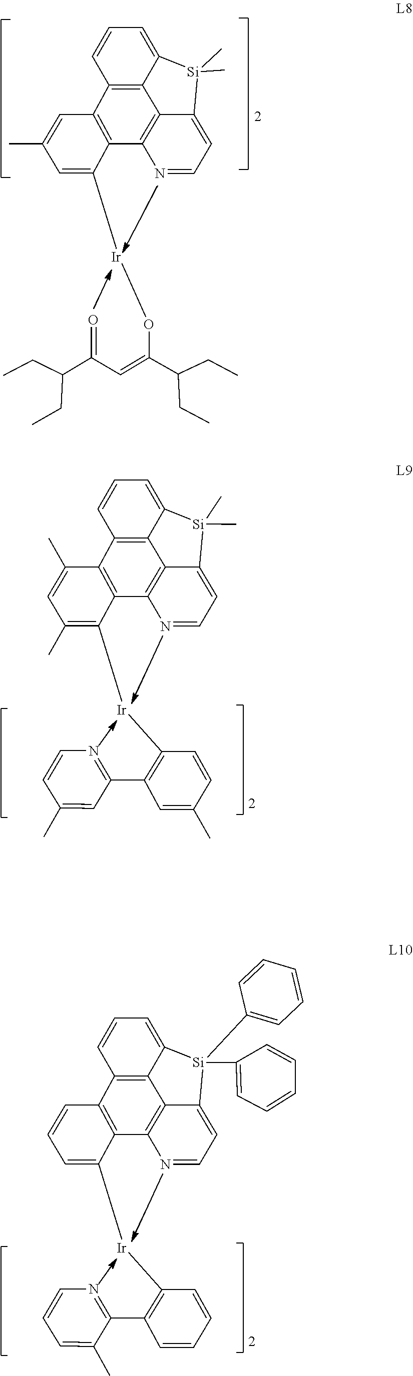

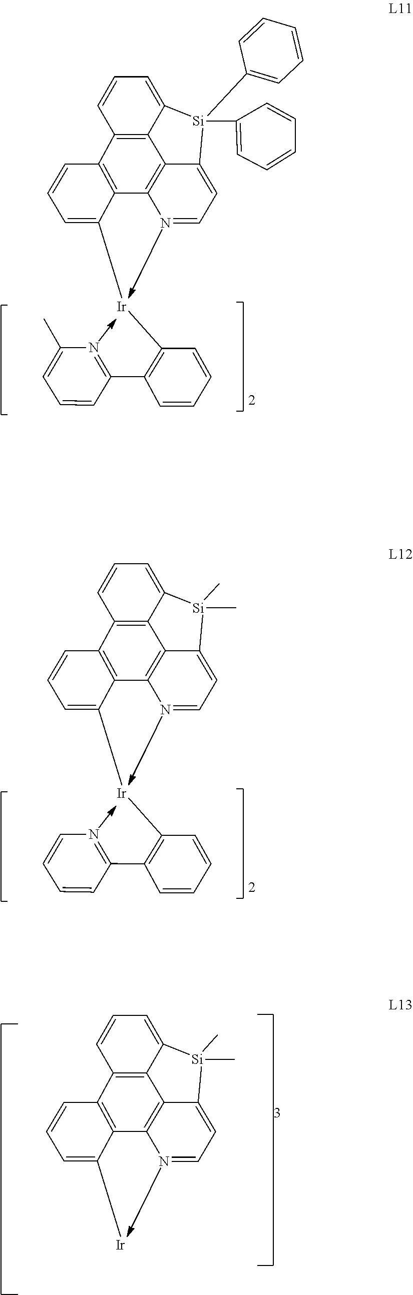

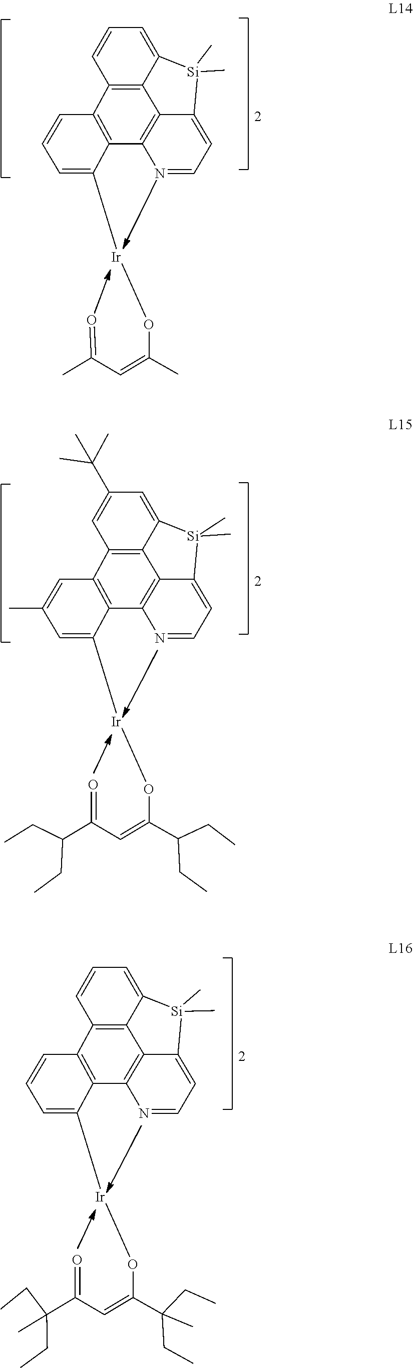

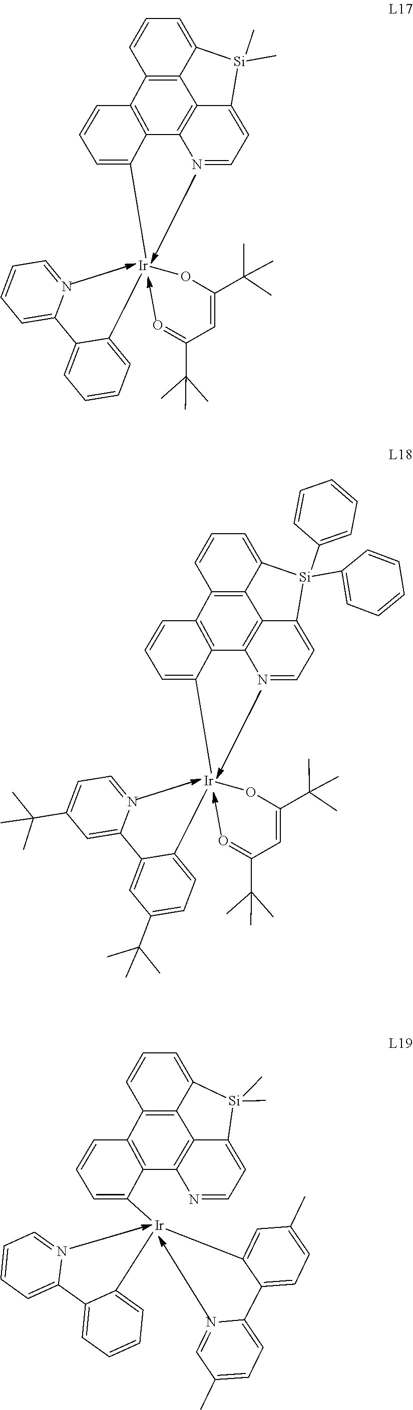

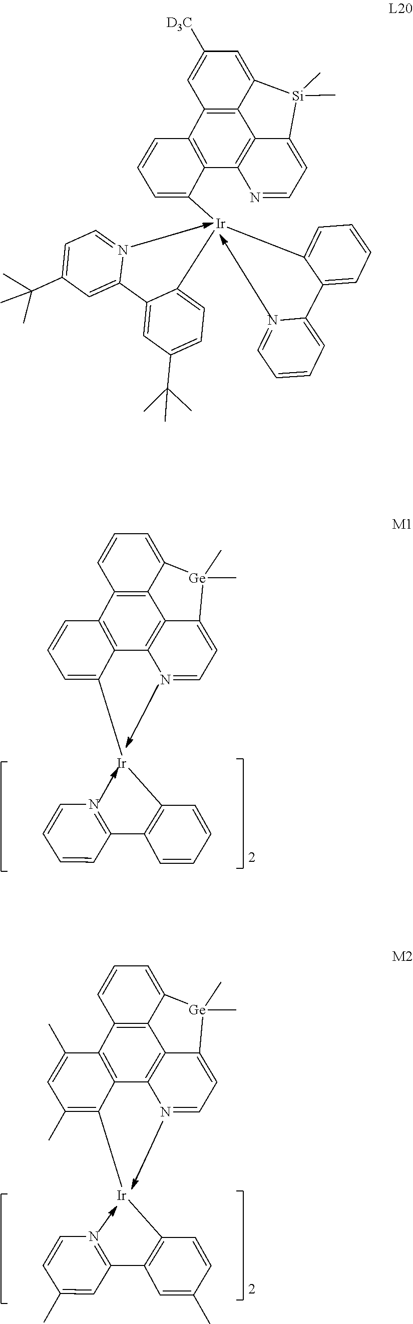

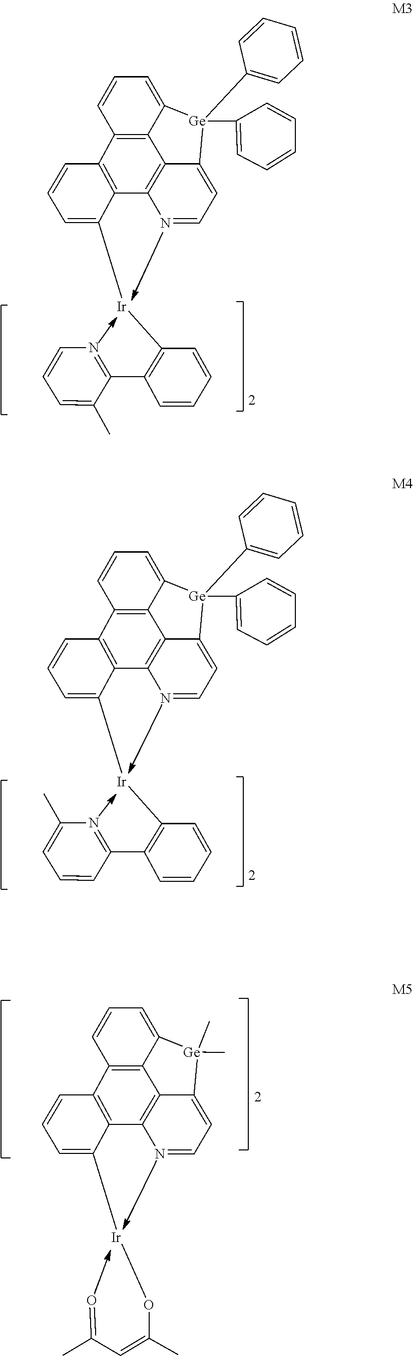

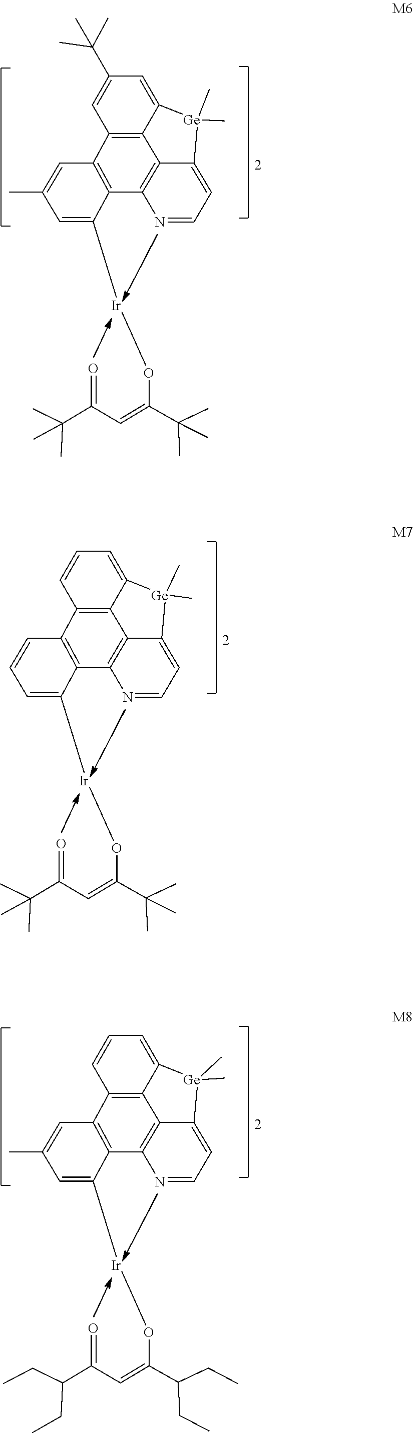

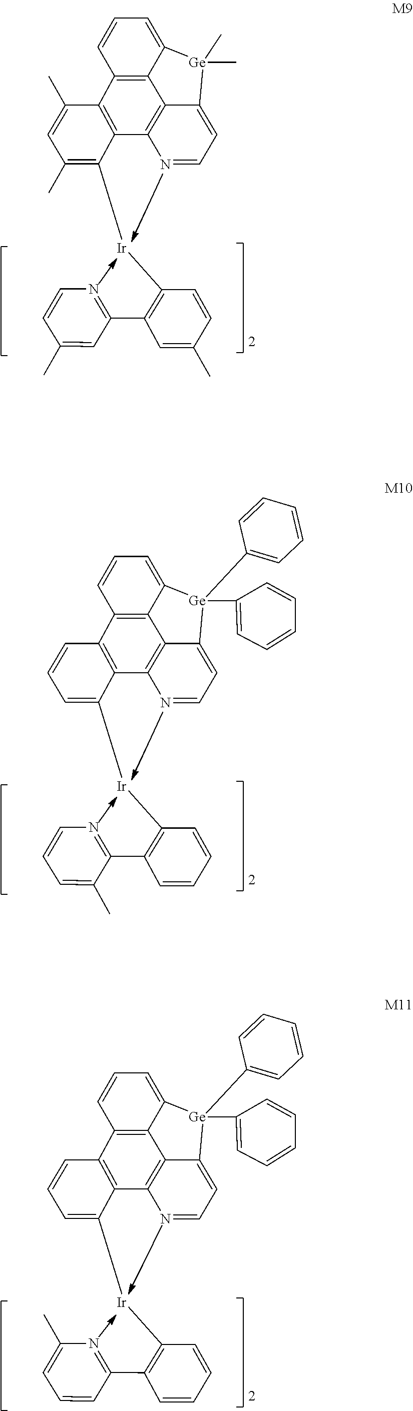

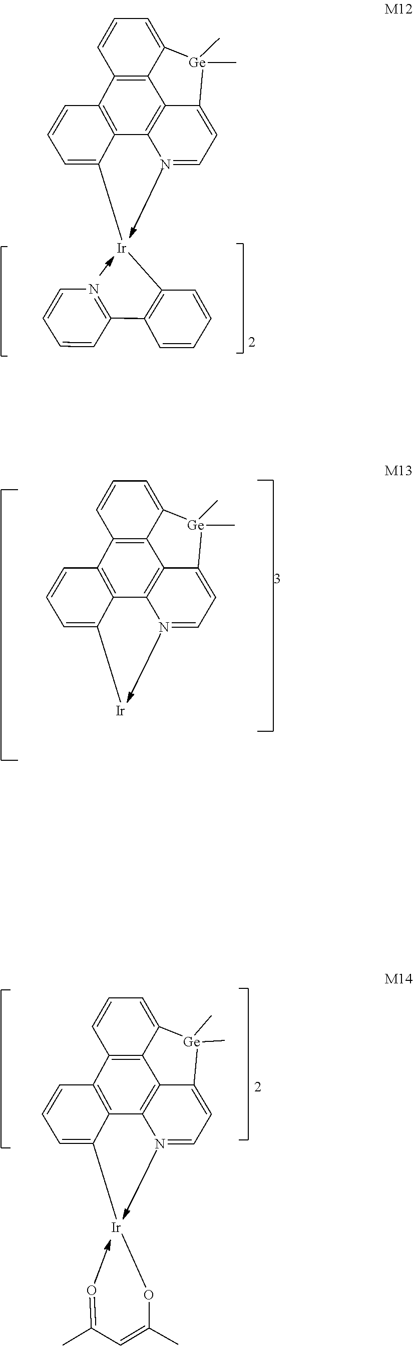

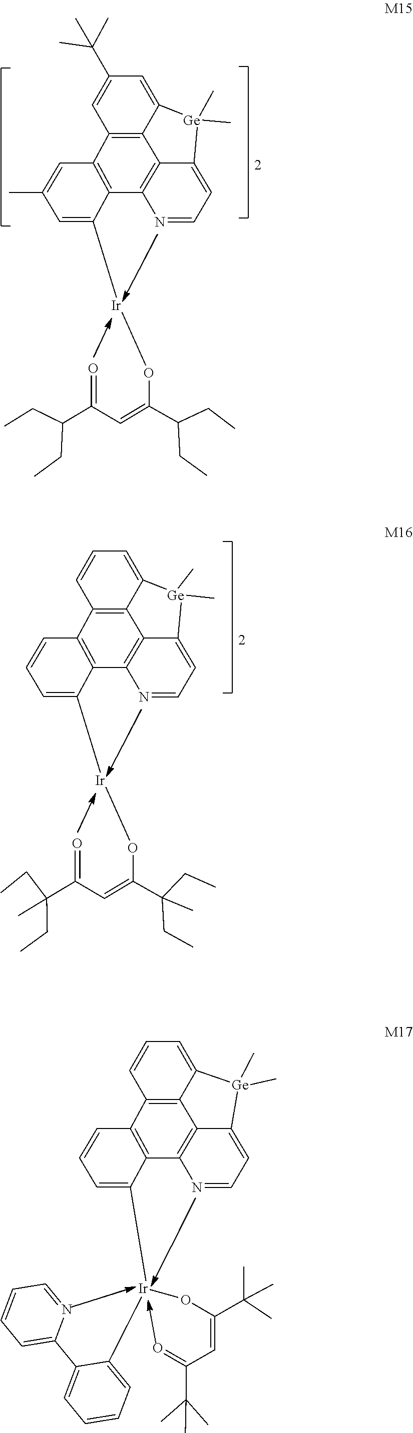

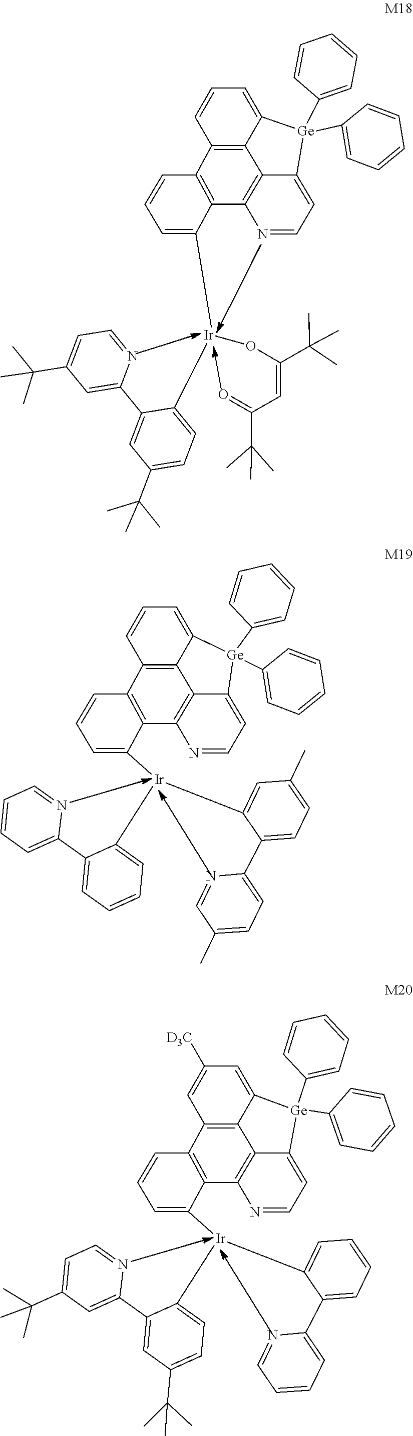

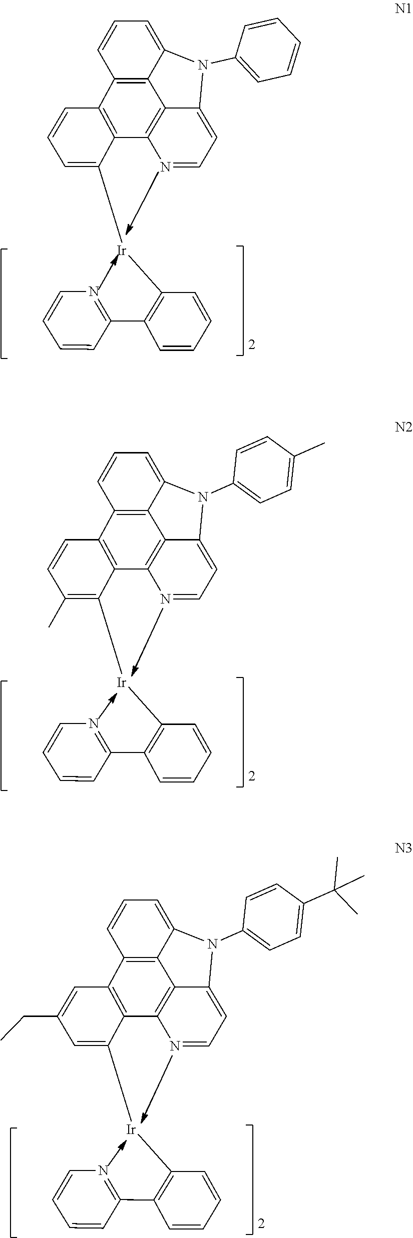

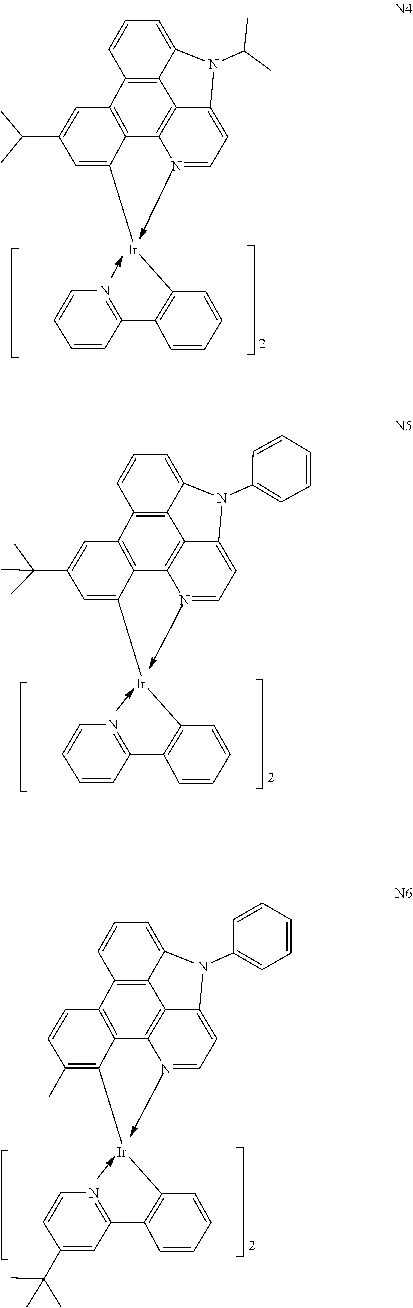

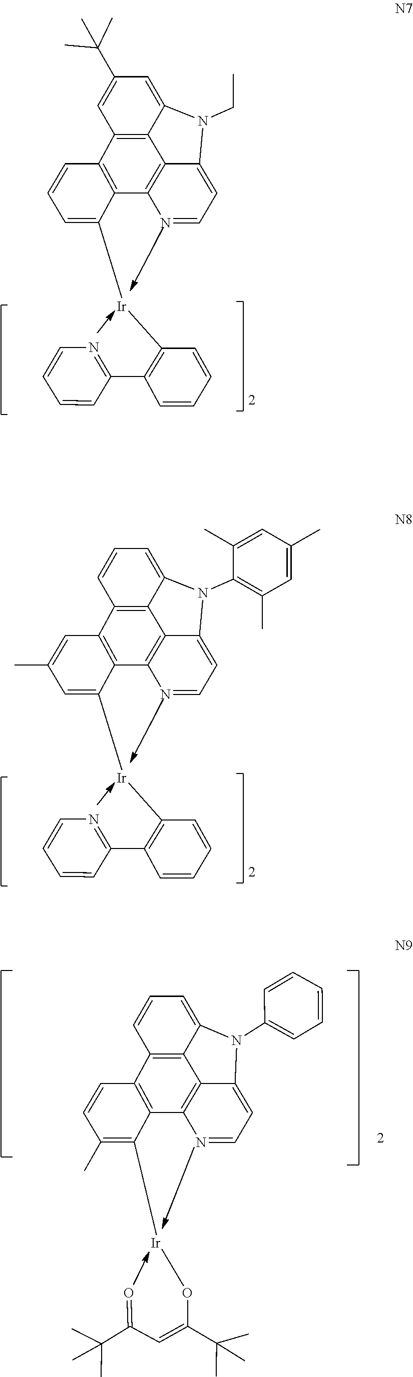

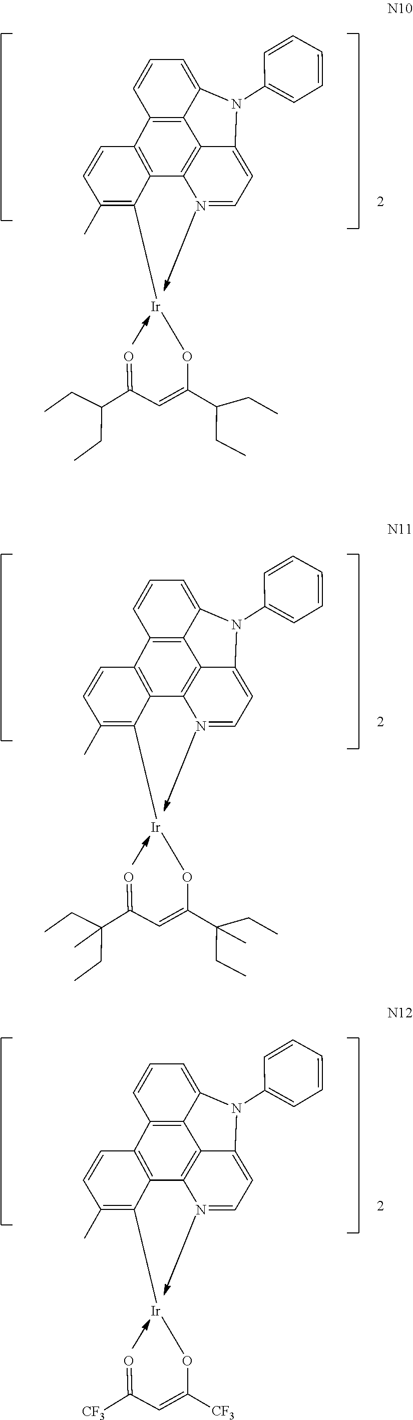

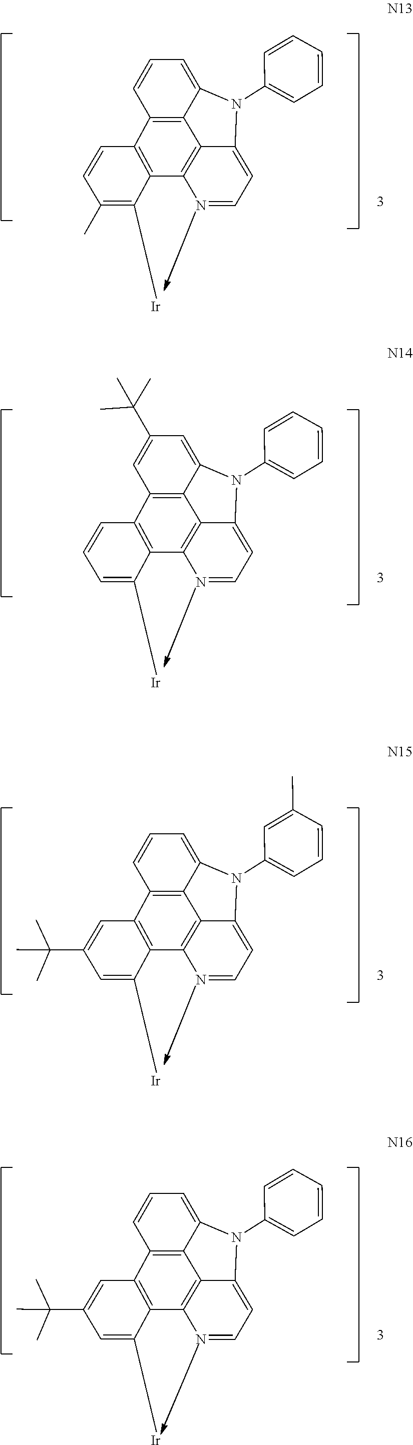

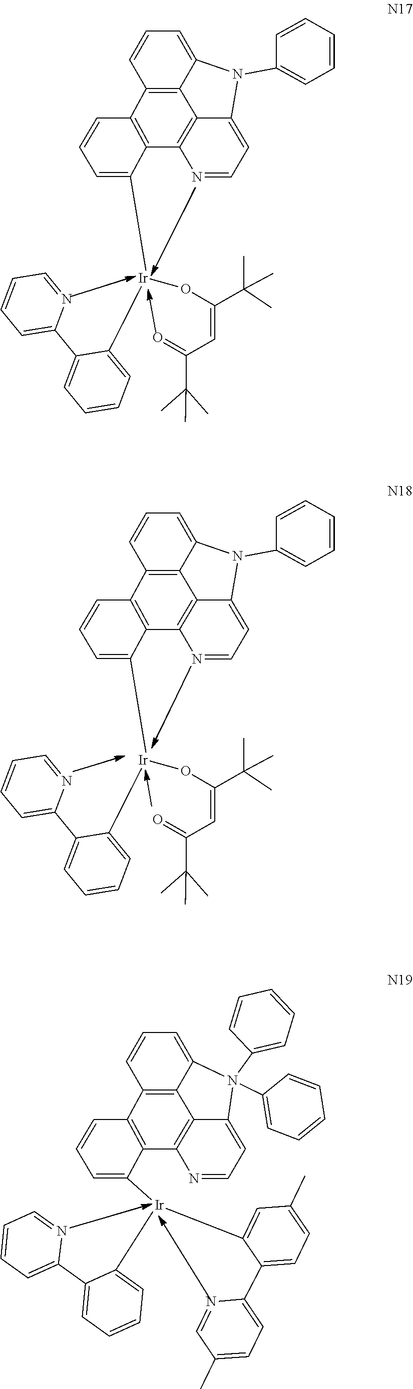

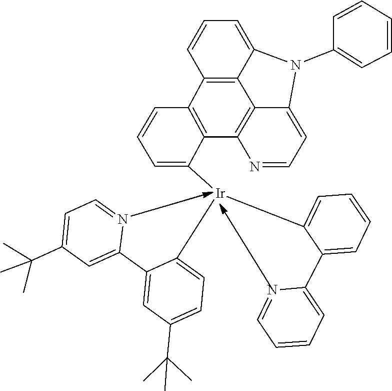

Among the exemplary compounds, the exemplary compounds belonging to the group A (A1 to A40) are organic compounds represented by the general formula [A-1] in which Z₁ denotes CR₁R₂. In other words, the partial structure IrL of the organic compounds is represented by the general formula [A-11]. These compounds have the characteristics (1), (2), and (3) and are highly sublimable among the compounds described above.

Among the exemplary compounds, the exemplary compounds belonging to the group B (B1 to B40) are organic compounds represented by the general formula [A-1] in which Z₁ denotes a sulfur atom. In other words, the partial structure IrL of the organic compounds is represented by the general formula [A-12]. These compounds have the characteristics (1), (2), and (4) and have better emission properties among the exemplary compounds described above.

Among the exemplary compounds, the exemplary compounds belonging to the group C (C1 to C40) are organic compounds represented by the general formula [A-1] in which Z₁ denotes an oxygen atom. In other words, the partial structure IrL of the organic compounds is represented by the general formula [A-13]. These compounds have the characteristics (1), (2), and (4) and have better emission properties among the exemplary compounds described above.

Among the exemplary compounds, the exemplary compounds belonging to the group D (D1 to D40) are organic compounds represented by the general formula [A-1] in which Z₁ denotes CR₁═CR₂. In other words, the partial structure IrL of the organic compounds is represented by the general formula [A-14]. These compounds have the characteristics (1), (2), and (5) and have higher chemical stability among the exemplary compounds described above.

Among the exemplary compounds, the exemplary compounds belonging to the group E (E1 to E40) are organic compounds represented by the general formula [A-2] in which Z₁ denotes CR₁R₂. In other words, the partial structure IrL of the organic compounds is represented by the general formula [A-21]. These compounds have the characteristics (1), (2), and (3) and are highly sublimable among the compounds described above.

Among the exemplary compounds, the exemplary compounds belonging to the group F (F1 to F40) are organic compounds represented by the general formula [A-2] in which Z₁ denotes a sulfur atom. In other words, the partial structure IrL of the organic compounds is represented by the general formula [A-22]. These compounds have the characteristics (1), (2), and (4) and have better emission properties among the exemplary compounds described above.

Among the exemplary compounds, the exemplary compounds belonging to the group G (G1 to G40) are organic compounds represented by the general formula [A-2] in which Z₁ denotes an oxygen atom. In other words, the partial structure IrL of the organic compounds is represented by the general formula [A-23]. These compounds have the characteristics (1), (2), and (4) and have better emission properties among the exemplary compounds described above.

Among the exemplary compounds, the exemplary compounds belonging to the group H (H1 to H40) are organic compounds represented by the general formula [A-2] in which Z₁ denotes CR₁═CR₂. In other words, the partial structure IrL of the organic compounds is represented by the general formula [A-24]. These compounds have the characteristics (1), (2), and (5) and have higher chemical stability among the exemplary compounds described above.

Among the exemplary compounds, the exemplary compounds belonging to the group I (I1 to I20) are organic compounds represented by the general formula [A-1] in which Z₁ denotes SiR₁R₂. The exemplary compounds belonging to the group J (J1 to J20) are organic compounds represented by the general formula [A-1] in which Z₁ denotes GeR₁R₂. These compounds have the characteristics (1), (2), and (3) and are highly sublimable among the compounds described above.

Among the exemplary compounds, the exemplary compounds belonging to the group K (K1 to K20) are organic compounds represented by the general formula [A-1] in which Z₂ denotes NR₁. These compounds have a structure having carbon atoms bridged by a nitrogen atom in the dibenzo[f,h]quinoline skeleton. Like the oxygen atom and the sulfur atom, the nitrogen atom has a lone pair and has the characteristic (4), thus resulting in a compound with good CT properties and a high quantum yield. Furthermore, when the substituent (R₁) of the nitrogen atom is a bulky substituent, such as a benzene ring, the substituent can more effectively reduce the aggregation of the ligand due to the steric hindrance effect, and therefore the compound has higher sublimability.

Among the exemplary compounds, the exemplary compounds belonging to the group L (L1 to L20) are organic compounds represented by the general formula [A-2] in which Z₂ denotes SiR₁R₂. The exemplary compounds belonging to the group M (M1 to M20) are organic compounds represented by the general formula [A-2] in which Z₂ denotes GeR₁R₂. These compounds have the characteristics (1), (2), and (3) and are highly sublimable among the compounds described above.

Among the exemplary compounds, the exemplary compounds belonging to the group N (N1 to N20) are organic compounds represented by the general formula [A-2] in which Z₂ denotes NR₁. These compounds have a structure having carbon atoms bridged by a nitrogen atom in the dibenzo[f,h]quinoline skeleton. Like the oxygen atom and the sulfur atom, the nitrogen atom has a lone pair and has the characteristic (4), thus resulting in a compound with good CT properties and a high quantum yield. Furthermore, when the substituent (R₁) of the nitrogen atom is a bulky substituent, such as a benzene ring, the substituent can more effectively reduce the aggregation of the ligand due to the steric hindrance effect, and therefore the compound has higher sublimability.

In the general formula [1], m is preferably 1 or 2, more preferably 2. In other words, it can be represented by Ir(L)(L′)₂. In the present embodiment, the partial structure IrL is represented by the general formula [A-1] or [A-2], and the ligand L has a high molecular weight and a highly planar structure. Thus, the organic compound with the ligand L associates easily due to the interaction therebetween and tends to have a higher molecular weight. However, at m=1, the organic compound can have a lower molecular weight as a whole, have smaller interaction therebetween, and therefore have a lower sublimation temperature. Consequently, sublimation purification can be performed at a lower temperature, and an element can be produced by vacuum deposition at a lower temperature.

In the general formula [A-1] or [A-2], in the aromatic ring σ-bonded to the Ir metal, a carbon atom adjacent to the carbon atom σ-bonded to the Ir metal can have a methyl group. This improves the balance between the metal to ligand charge transfer (MLCT) properties, which are interactions between the ligand and the Ir metal, and the π-π* properties of the ligand. The same applies to the general formulae [A-11] to [A-14] and [A-21] to [A-24].

Thus, in the general formula [1], the partial structure IrL can be a partial structure represented by the following general formula [C-1] or [C-2].

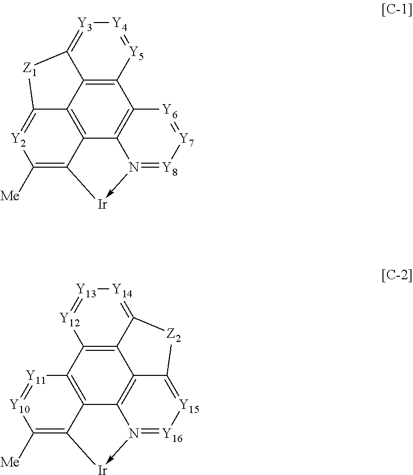

Furthermore, in the general formula [1], the partial structure IrL can be a partial structure represented by any one of the following general formulae [C-11] to [C-14] and [C-21] to [C-24].

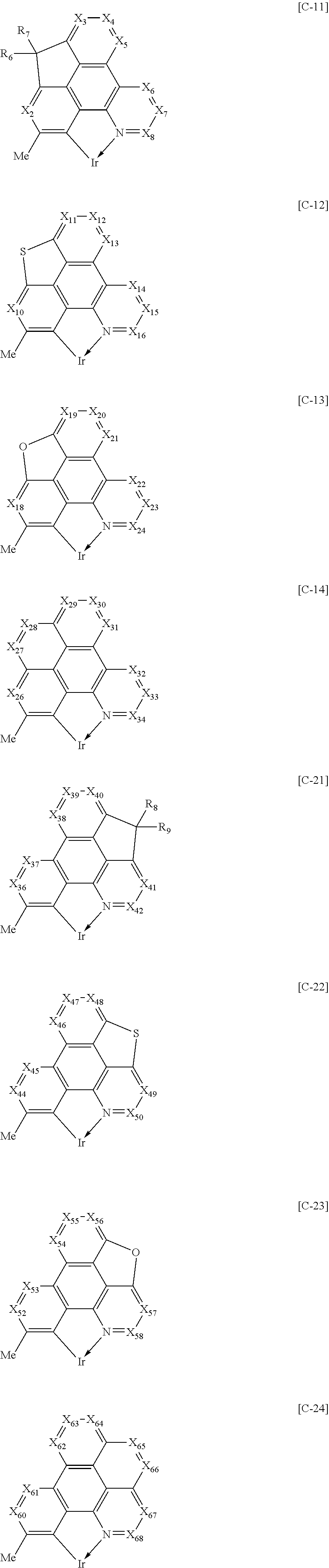

Y₂ to Y₁₆ in the general formula [C-1] and [C-2] are the same as Y₂ to Y₁₆ in the general formula [A-1] and [A-2]. Furthermore, X₂ to X₆₈ in the general formulae [C-11] to [C-14] and [C-21] to [C-24] are the same as X₂ to X₆₈ in the general formulae [A-11] to [A-14] and [A-21] to [A-24].

Furthermore, in the general formula [1], all three ligands can have different structures. When all three ligands have different structures, the Ir complex can have lower symmetry as a whole, have improved sublimability, and have higher resistance to concentration quenching. In other words, it can be an organic compound represented by the following general formula [2].

Ir L L′L″ [2]

In the general formula [2], Ir denotes iridium. L, L′, and L″ denote different bidentate ligands. The partial structure IrL denotes a partial structure represented by the general formula [A-1] or [A-2], and the partial structure IrL′ denotes a partial structure represented by the general formula [B-1] or [B-2]. The partial structure IrL″ is a partial structure represented by any one of the general formulae [A-1], [A-2], [B-1], and [B-2]. The partial structure IrL″ can be a partial structure represented by the general formula [B-1] or [B-2].

### Organic Light-Emitting Element

Next, an organic light-emitting element according to the present embodiment is described.

A specific element structure of the organic light-emitting element according to the present embodiment may be a multilayer element structure including an electrode layer and an organic compound layer shown in the following (1) to (6) sequentially stacked on a substrate. More specifically, the organic light-emitting element according to the present embodiment includes at least a pair of electrodes, a first electrode and a second electrode, and an organic compound layer between the electrodes. The first electrode may be a positive electrode, and the second electrode may be a negative electrode. In any of the element structures, the organic compound layer always includes a light-emitting layer containing a light-emitting material.

(1) Positive electrode/light-emitting layer/negative electrode
(2) Positive electrode/hole-transport layer/light-emitting layer/electron-transport layer/negative electrode
(3) Positive electrode/hole-transport layer/light-emitting layer/electron-transport layer/electron-injection layer/negative electrode
(4) Positive electrode/hole-injection layer/hole-transport layer/light-emitting layer/electron-transport layer/negative electrode
(5) Positive electrode/hole-injection layer/hole-transport layer/light-emitting layer/electron-transport layer/electron-injection layer/negative electrode
(6) Positive electrode/hole-transport layer/electron-blocking layer/light-emitting layer/hole-blocking layer/electron-transport layer/negative electrode

These element structure examples are only basic element structures, and the element structure of an organic light-emitting element of the present disclosure is not limited to these element structures. For example, an insulating layer, an adhesive layer, or an interference layer may be provided at an interface between an electrode and an organic compound layer. An electron-transport layer or a hole-transport layer may have a multilayered structure having two layers with different ionization potentials. A light-emitting layer may have a multilayered structure having two layers each containing different light-emitting materials. Thus, a first light-emitting layer for emitting first light and a second light-emitting layer for emitting second light may be provided between a positive electrode and a negative electrode. An organic light-emitting element for emitting white light can be produced in which the white light is composed of first light and second light of different colors. In addition to such structures, various other layer structures can be employed.

In the present embodiment, the mode (element form) of extracting light from a light-emitting layer may be a bottom emission mode of extracting light from an electrode on the substrate side or a top emission mode of extracting light from the side opposite to the substrate side. The mode may also be a double-sided extraction mode of extracting light from the substrate side and from the side opposite to the substrate side.

Among the element structures shown in (1) to (6), the structure (6) has both an electron-blocking layer (electron-stopping layer) and a hole-blocking layer (hole-stopping layer). Thus, the electron-blocking layer and the hole-blocking layer in (6) can securely confine both carriers of holes and electrons in the light-emitting layer. Thus, the organic light-emitting element has no carrier leakage and high luminescence efficiency.

The organic light-emitting element according to the present embodiment contains an organic compound represented by the general formula [1] in an organic compound layer. The organic light-emitting element according to the present embodiment can contain an organic compound represented by the general formula [1] in a light-emitting layer. However, the present disclosure is not limited thereto, and it can be used as a constituent material of an organic compound layer other than the light-emitting layer constituting the organic light-emitting element according to the present embodiment. More specifically, it may be used as a constituent material of an electron-transport layer, an electron-injection layer, an electron-blocking layer, a hole-transport layer, a hole-injection layer, a hole-blocking layer, or the like.

In the organic light-emitting element according to the present embodiment, when the light-emitting layer contains an organic compound represented by the general formula [1], the light-emitting layer may be a layer composed only of the organic compound represented by the general formula [1]. Alternatively, the light-emitting layer may be a layer composed of an organic compound represented by the general formula [1] and another compound. When an organic compound represented by the general formula [1] is used as a guest (hereinafter also referred to as a guest material), the light-emitting layer may contain a first compound. The light-emitting layer may further contain a second compound. The first compound may be a host (hereinafter also referred to as a host material). The second compound may be an assist (hereinafter also referred to as an assist material). For a light-emitting layer composed of an organic compound represented by the general formula [1] and another compound, the organic compound according to the present embodiment may be used as a host or a guest of the light-emitting layer. The organic compound may also be used as an assist material that may be contained in the light-emitting layer.

The host is a compound with the highest mass ratio among the compounds constituting the light-emitting layer. The guest is a compound that has a lower mass ratio than the host among the compounds constituting the light-emitting layer and that is a principal light-emitting compound. The assist material is a compound that has a lower mass ratio than the host among the compounds constituting the light-emitting layer and that assists the guest in emitting light. The assist material is also referred to as a second host.

The host can be a material with a higher LUMO than the guest (a material with a LUMO closer to the vacuum level). This allows electrons supplied to the host of the light-emitting layer to be efficiently delivered to the guest and improves luminescence efficiency. Furthermore, when an assist material is used in addition to the host and the guest, the host can be a material with a higher LUMO than the assist material (a material with a LUMO closer to the vacuum level). This allows electrons supplied to the host of the light-emitting layer to be efficiently delivered to the assist material, and the assist material can play a role in exciton recombination. This enables efficient energy transfer to the guest.

The energy (singlet energy) of the excited singlet state (S₁) of the host is denoted by Sₕ₁, the energy (triplet energy) of the excited triplet state (T₁) is denoted by Tₕ₁, the energy of S₁ of the guest is denoted by S_{g1}, and the energy of T₁ of the guest is denoted by T_{g1}. Then, Sₕ₁>S_{g1} can be satisfied. Tₕ₁>T_{g1} can also be satisfied. Furthermore, the energy Sₐ₁ of S₁ and the energy Tₐ₁ of T₁ of the assist material can satisfy Sₐ₁>S_{g1} and Tₐ₁>T_{g1}. Furthermore, Sₕ₁>Sₐ₁>S_{g1} can be satisfied, and Tₕ₁>Tₐ₁>T_{g1} can also be satisfied.

The present inventors conducted various studies and found that an organic light-emitting element with high luminescence efficiency and durability can be produced when an organic compound represented by the general formula [1] is used as a host or a guest in a light-emitting layer, particularly as a guest in the light-emitting layer.

When the organic light-emitting element according to the present embodiment contains an organic compound represented by the general formula [1] in the light-emitting layer, the following conditions are satisfied with respect to a compound contained in the light-emitting layer. Two or more of the following conditions may be simultaneously satisfied. As described above, an organic compound represented by the general formula [1] can be used as a guest in the light-emitting layer, and a second organic compound can be a host of the light-emitting layer under the following conditions.

(7) The light-emitting layer contains an organic compound represented by the general formula [1] at a concentration in the range of 1% to 30% by mass of the entire light-emitting layer.
(8) The light-emitting layer contains an organic compound represented by the general formula [1] and a second organic compound with at least one structure selected from the group consisting of a triphenylene structure, a phenanthrene structure, a chrysene structure, and a fluoranthene structure.
(9) The light-emitting layer contains an organic compound represented by the general formula [1] and a second organic compound with a carbazole structure.
(10) The light-emitting layer contains an organic compound represented by the general formula [1] and a second organic compound with at least one of a dibenzothiophene structure and a dibenzofuran structure.
(11) The light-emitting layer contains an organic compound represented by the general formula [1] and a second organic compound without sp3 carbon.

Each of the conditions is described below.

(7) The light-emitting layer contains an organic compound represented by the general formula [1] at a concentration in the range of 1% to 30% by mass of the entire light-emitting layer.

When an organic compound represented by the general formula [1] is used for a light-emitting layer, the amount of the organic compound preferably ranges from 1% to 30% by mass of the entire light-emitting layer. Furthermore, the amount of the organic compound more preferably ranges from 5% to 15% by mass of the entire light-emitting layer. When an organic compound represented by the general formula [1] is used for a light-emitting layer, a lower concentration can result in better characteristics. A low concentration can result in a light-emitting element with high efficiency and color purity.

This results from the structural characteristics of an organic compound represented by the general formula [1]. An organic compound represented by the general formula [1] has the ligand L, which has an extended π-conjugated system. Thus, when an organic compound represented by the general formula [1] is mixed in the light-emitting layer at an excessively high concentration, the organic compound may aggregate and cause concentration quenching, thereby reducing luminescence efficiency. On the other hand, an organic compound represented by the general formula [1] at a relatively low concentration in the range of 1% to 30% by mass of the entire light-emitting layer is less likely to aggregate and can increase luminescence efficiency.

(8) The light-emitting layer contains an organic compound represented by the general formula [1] and a second organic compound with at least one structure selected from the group consisting of a triphenylene structure, a phenanthrene structure, a chrysene structure, and a fluoranthene structure.

In an organic compound represented by the general formula [1], the ligand has the dibenzo[f,h]quinoline skeleton and a highly planar structure with an extended π-conjugated system. Thus, the second organic compound used in combination with an organic compound represented by the general formula [1] can have an aromatic ring and a highly planar structure. This is because a highly planar moiety of the second organic compound with a highly planar structure can interact with and approach to a highly planar moiety of an organic compound represented by the general formula [1]. More specifically, the ligand L of an organic compound represented by the general formula [1] approaches easily to the planar moiety of the second organic compound. Thus, the intermolecular distance between an organic compound represented by the general formula [1] and the second organic compound can be expected to be shortened.

It is known that triplet energy for phosphorescence in an organic light-emitting element is transferred by the Dexter mechanism. The Dexter mechanism includes energy transfer by intermolecular contact. More specifically, the intermolecular distance between a host and a guest is shortened for efficient energy transfer from the host to the guest.

The use of a highly planar organic compound as the second organic compound shortens the intermolecular distance between an organic compound represented by the general formula [1] and the second organic compound and causes more efficient energy transfer between the two compounds by the Dexter mechanism. More specifically, the use of the second organic compound as a host improves the efficiency of energy transfer from the second organic compound to an organic compound represented by the general formula [1]. Consequently, an organic light-emitting element that efficiently emits light can be provided.

The highly planar structure specifically refers to a triphenylene structure, a phenanthrene structure, a chrysene structure, or a fluoranthene structure. A compound with at least one of these structures used as a second organic compound in combination with an organic compound represented by the general formula [1] can provide a more efficient light-emitting element.

(9) The light-emitting layer contains an organic compound represented by the general formula [1] and a second organic compound with a carbazole structure.

As shown in Table 3 below, an organic compound represented by the general formula [1] has a HOMO site composed of an Ir metal and an aromatic ring and a LUMO site composed of an Ir metal and a heterocycle. In Table 3 below, the HOMO site and the LUMO site are surrounded by a dotted circle. In Table 3, a portion surrounded by a dotted square is a vacant orbital. Thus, an orbital exists after the HOMO site is localized near the Ir metal and the benzene ring bonded to the Ir metal, and therefore an organic compound represented by the general formula [1] tends to have lower hole transport ability due to this vacant orbital.

Thus, the present inventors have found that an organic compound represented by the general formula [1] can be used in combination with an organic compound with a carbazole structure. The carbazole structure is a heterocycle with high hole transport ability. Thus, an organic compound with the carbazole structure has high hole transport ability. Thus, the combined use of an organic compound with the carbazole structure can be expected to compensate for the hole transport ability lowered by an organic compound represented by the general formula [1] and improve the hole transport ability of the light-emitting layer.

Furthermore, an organic compound represented by the general formula [1] can be used in combination with a second organic compound with the carbazole structure and an azine ring. The azine ring, such as pyridine, pyrazine, pyrimidine, or triazine, is a heterocycle with high electron transport ability. Thus, further introducing the azine ring into an organic compound with the carbazole structure can enhance not only the hole transport ability but also the electron transport ability. Thus, a light-emitting layer with improved electron transport ability and hole transport ability can be formed.

TABLE 3

| Compound | HOMO | LUMO |
|---|---|---|
| Exemplary compound A35 | 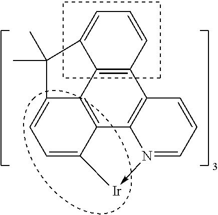 | 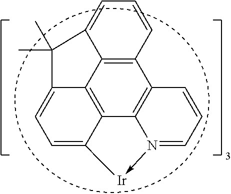 |

(10) The light-emitting layer contains an organic compound represented by the general formula [1] and a second organic compound with at least one of a dibenzothiophene structure and a dibenzofuran structure.

In general, Ir complexes are known to be hole-trapping compounds. Furthermore, as described above, an organic compound represented by the general formula [1] has a vacant orbital and therefore has particularly low hole transport ability.

To compensate for the hole transport ability, a second organic compound used in combination with an organic compound represented by the general formula [1] can be a material having a skeleton with high hole transport ability. The skeleton with high hole transport ability is a skeleton with abundant lone pairs and high electron-donating ability. More specifically, it is a skeleton with an electron-donating nitrogen atom, such as carbazole, as described above in (9), or a skeleton with a chalcogen atom having abundant lone pairs, such as a dibenzothiophene structure or a dibenzofuran structure.

Among these, the second organic compound that can be suitably used in combination with an organic compound represented by the general formula [1] can have at least one skeleton of a dibenzothiophene structure and a dibenzofuran structure. A skeleton with a dibenzothiophene structure or a dibenzofuran structure is less likely to have an extremely shallow HOMO, can therefore adjust the carrier balance between holes and electrons, and is suitable for a skeleton that assists the hole transport ability of an organic compound represented by the general formula [1]. In particular, the second organic compound can have a dibenzothiophene structure with abundant lone pairs.

(11) The light-emitting layer contains an organic compound represented by the general formula [1] and a second organic compound without sp3 carbon.

As described above in (8), shortening the intermolecular distance between an organic compound represented by the general formula [1] and the second organic compound can improve the emission properties of the organic light-emitting element. The use of an organic compound without sp3 carbon as the second organic compound can further shorten the intermolecular distance from an organic compound represented by the general formula [1].

In the presence of sp3 carbon, the hydrophobic interaction and steric hindrance of the alkyl group increase the intermolecular distance between an organic compound represented by the general formula [1] and the second organic compound. By contrast, without sp3 carbon, and consequently without the hydrophobic interaction and steric hindrance of the alkyl group, the effect of increasing the intermolecular distance does not occur, and the intermolecular distance from an organic compound represented by the general formula [1] can be shortened. This can improve the emission properties of the organic light-emitting element.

The following are specific examples of the first compound according to the present embodiment, more specifically, specific examples of compounds suitable for host materials. However, the present disclosure is not limited to these examples.

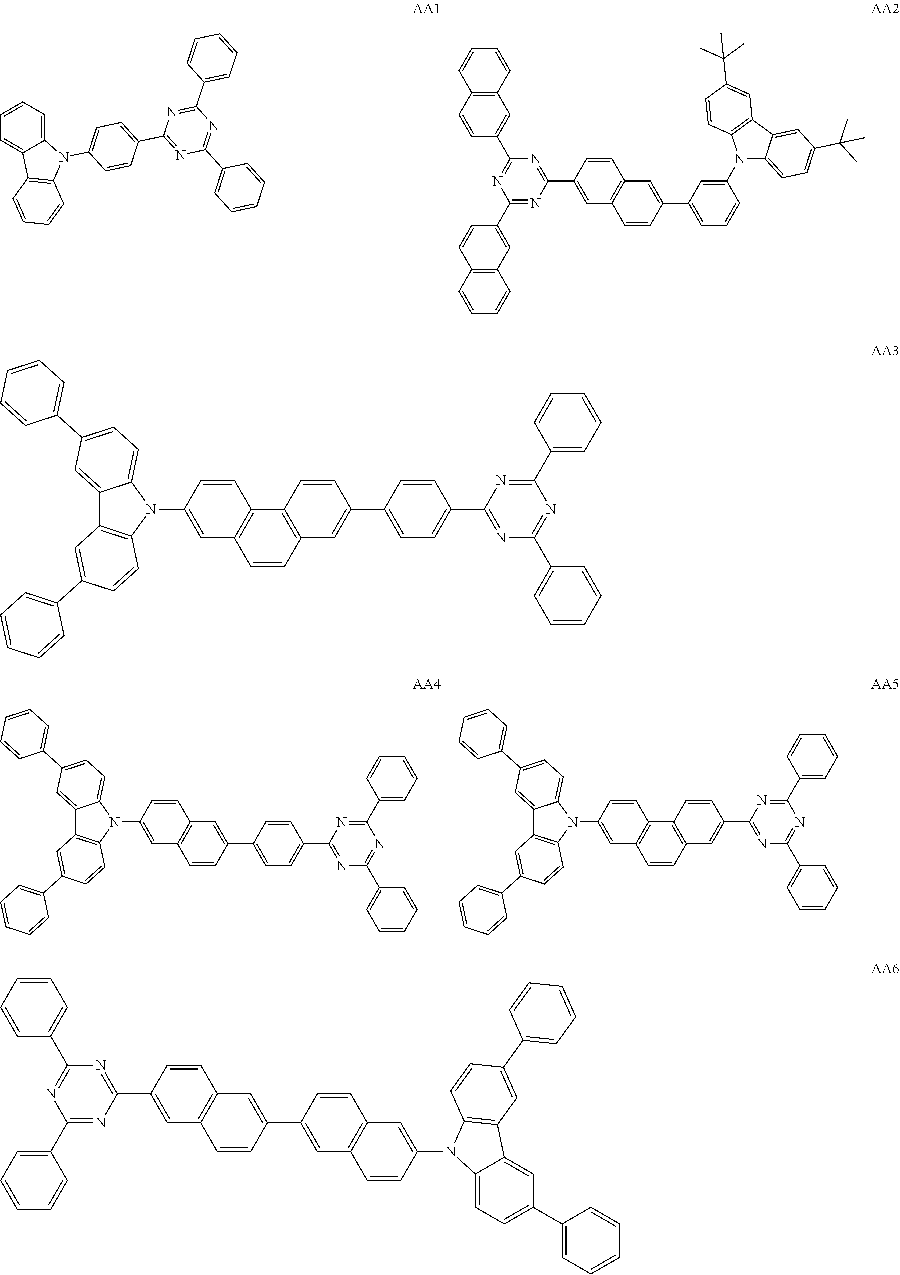

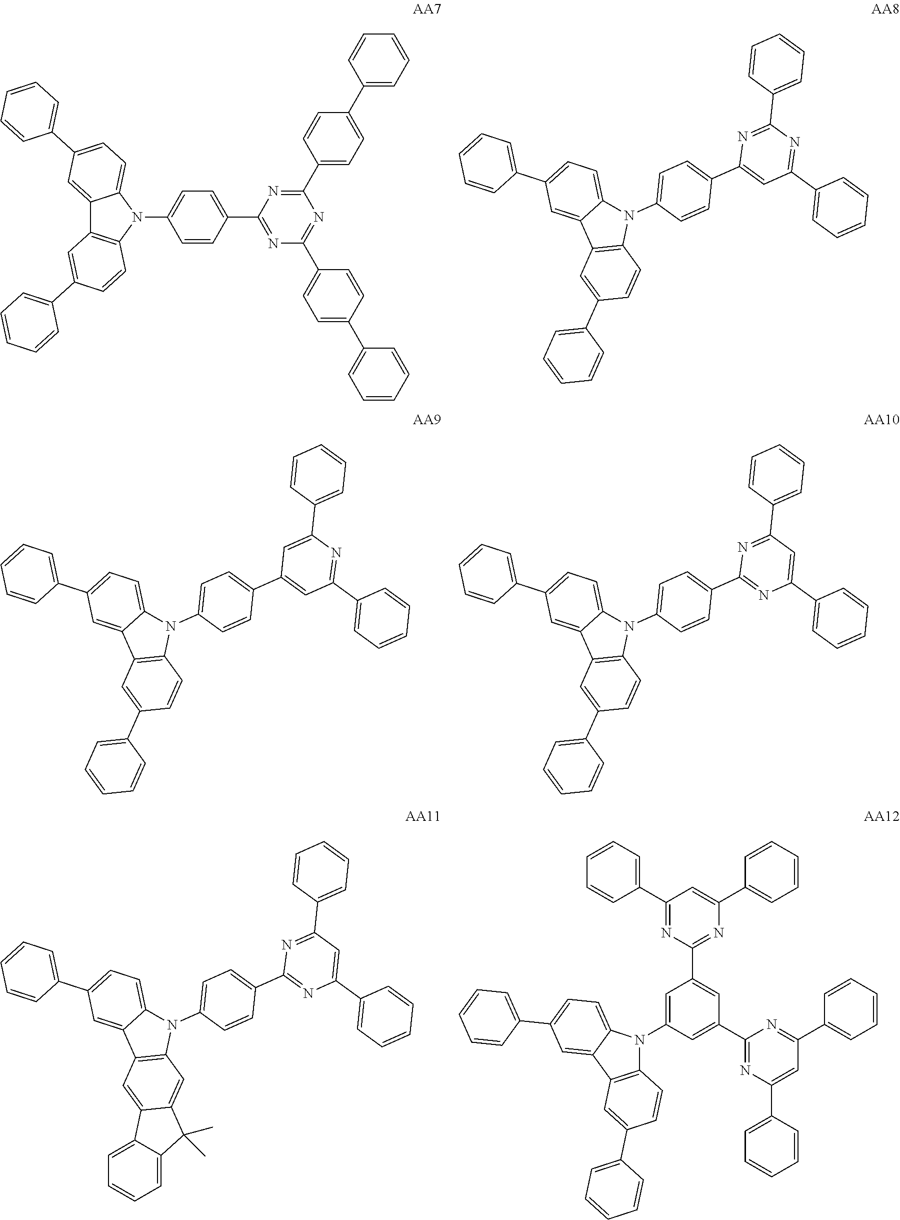

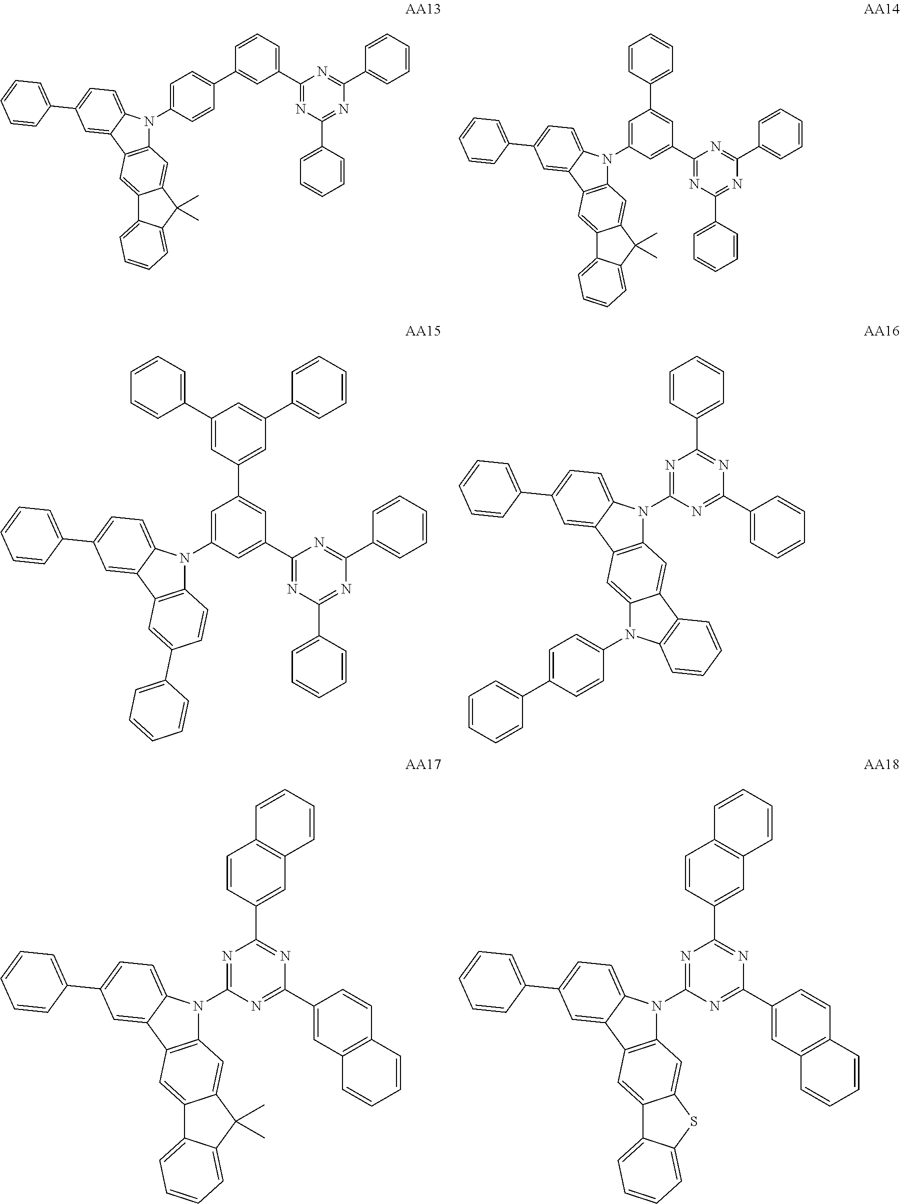

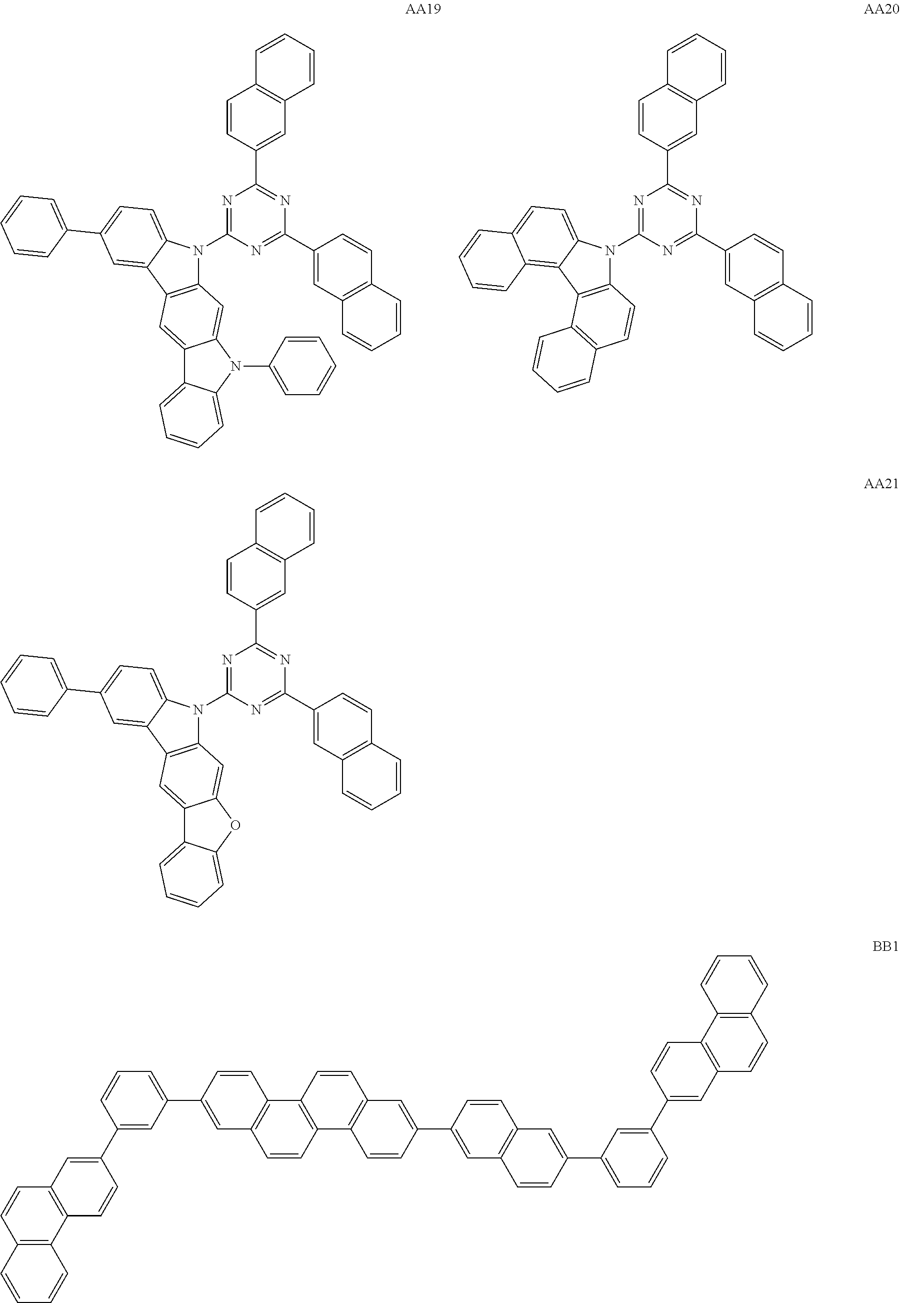

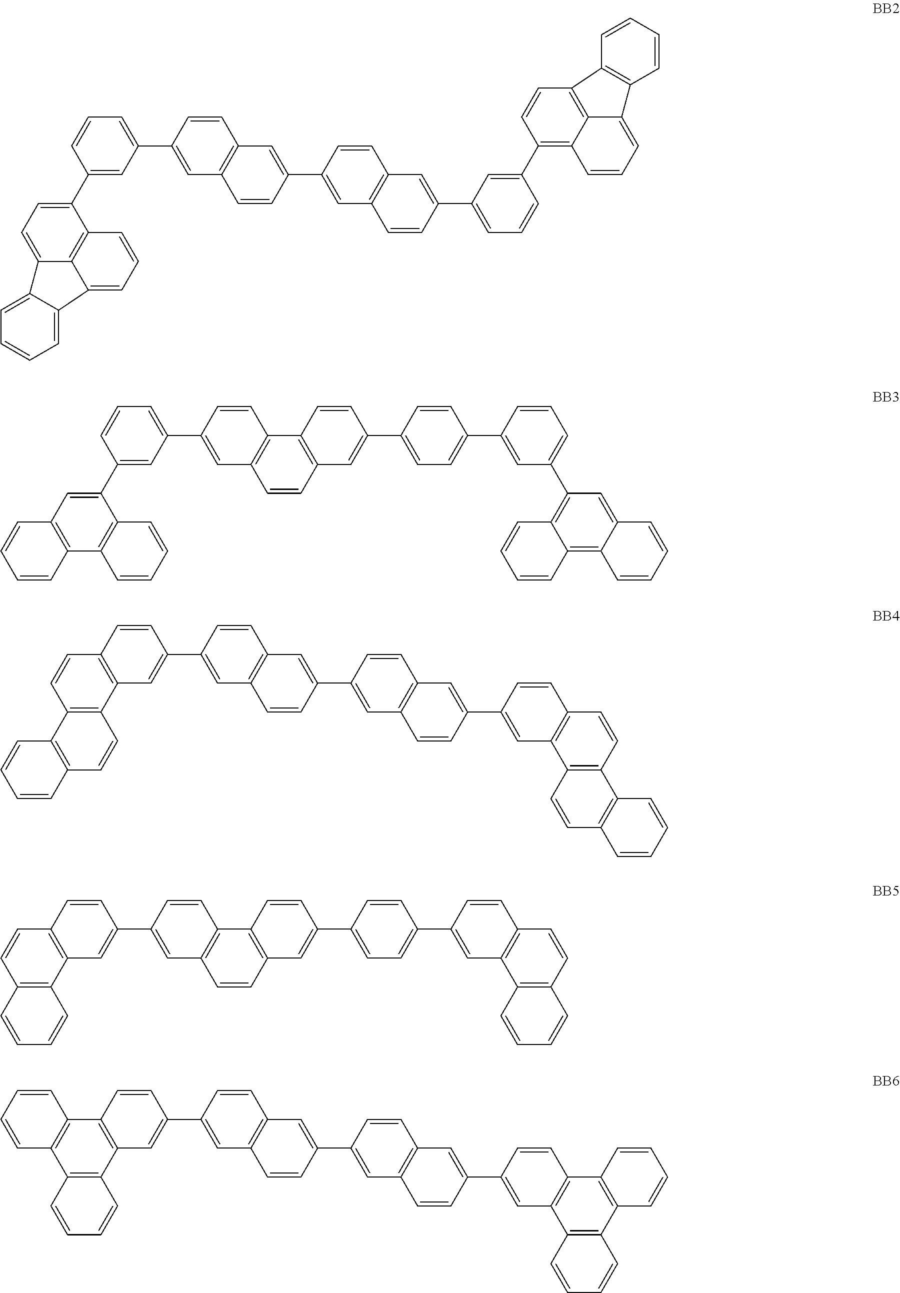

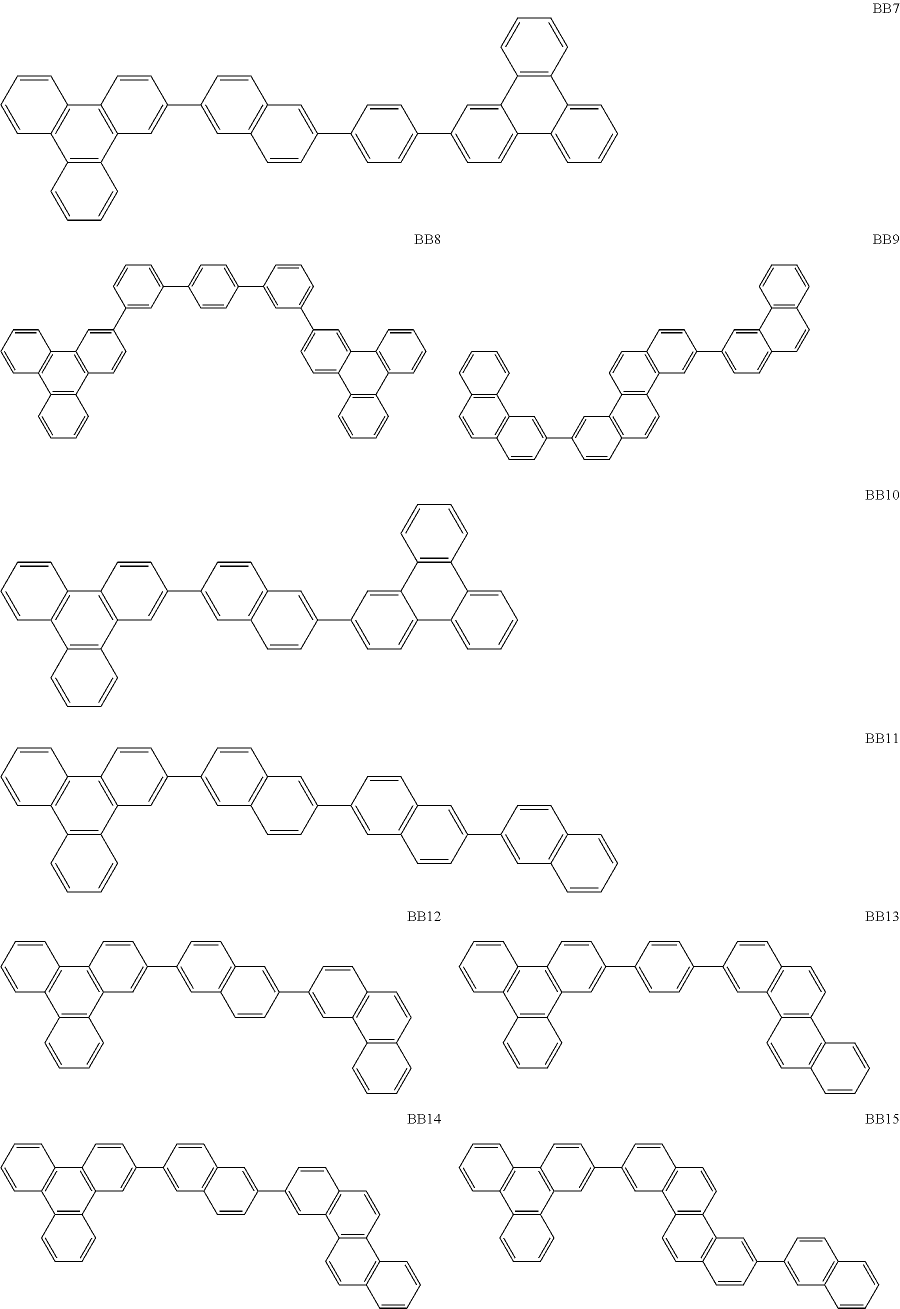

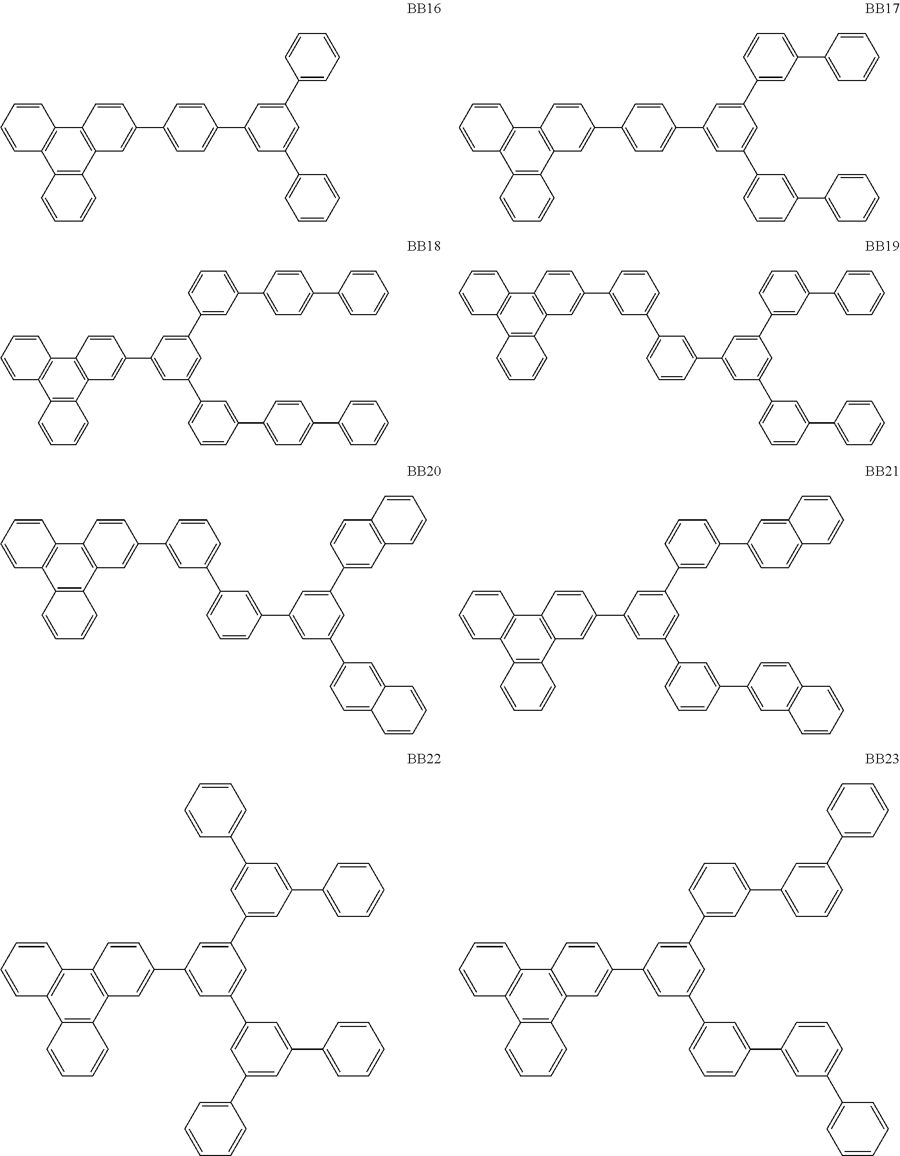

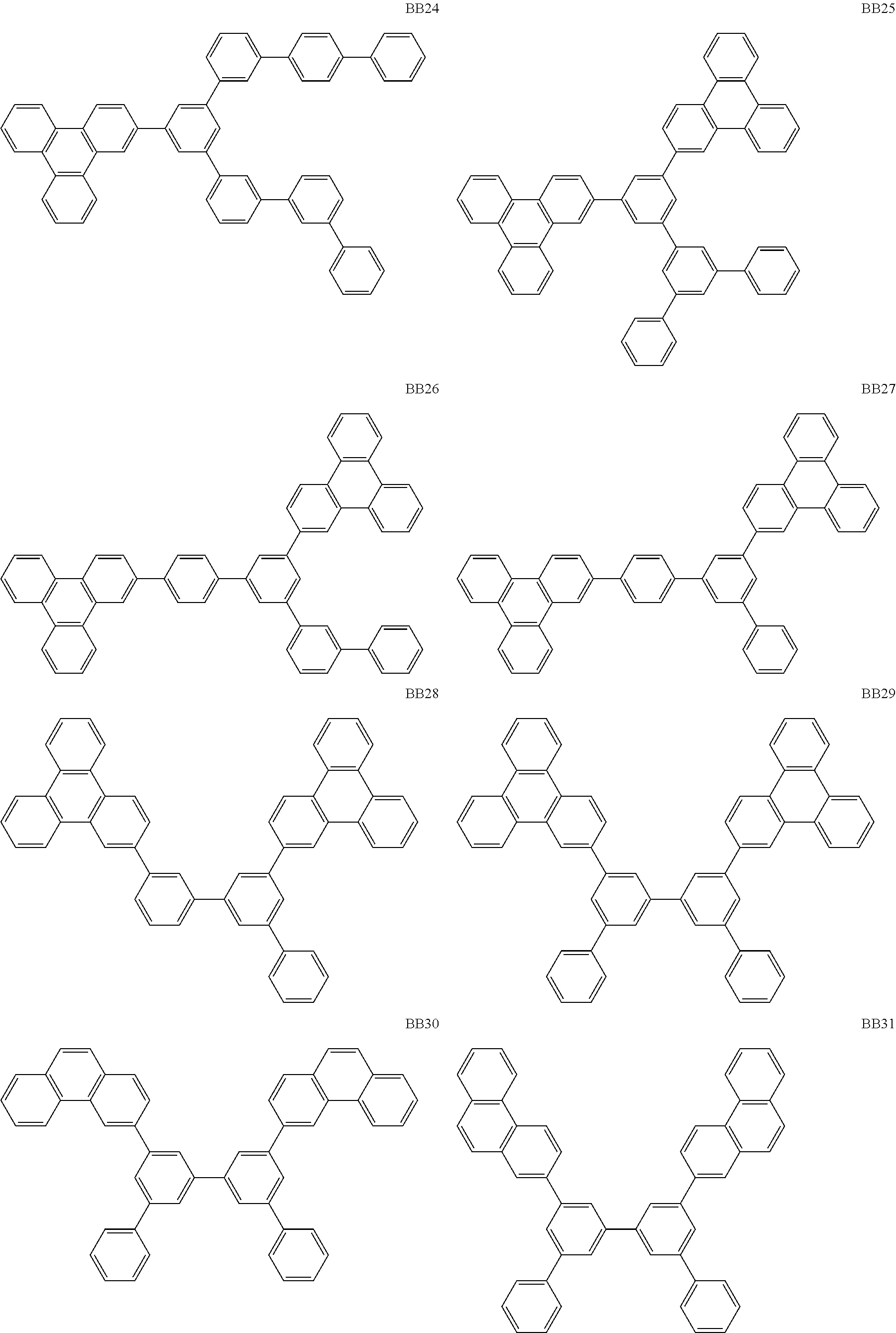

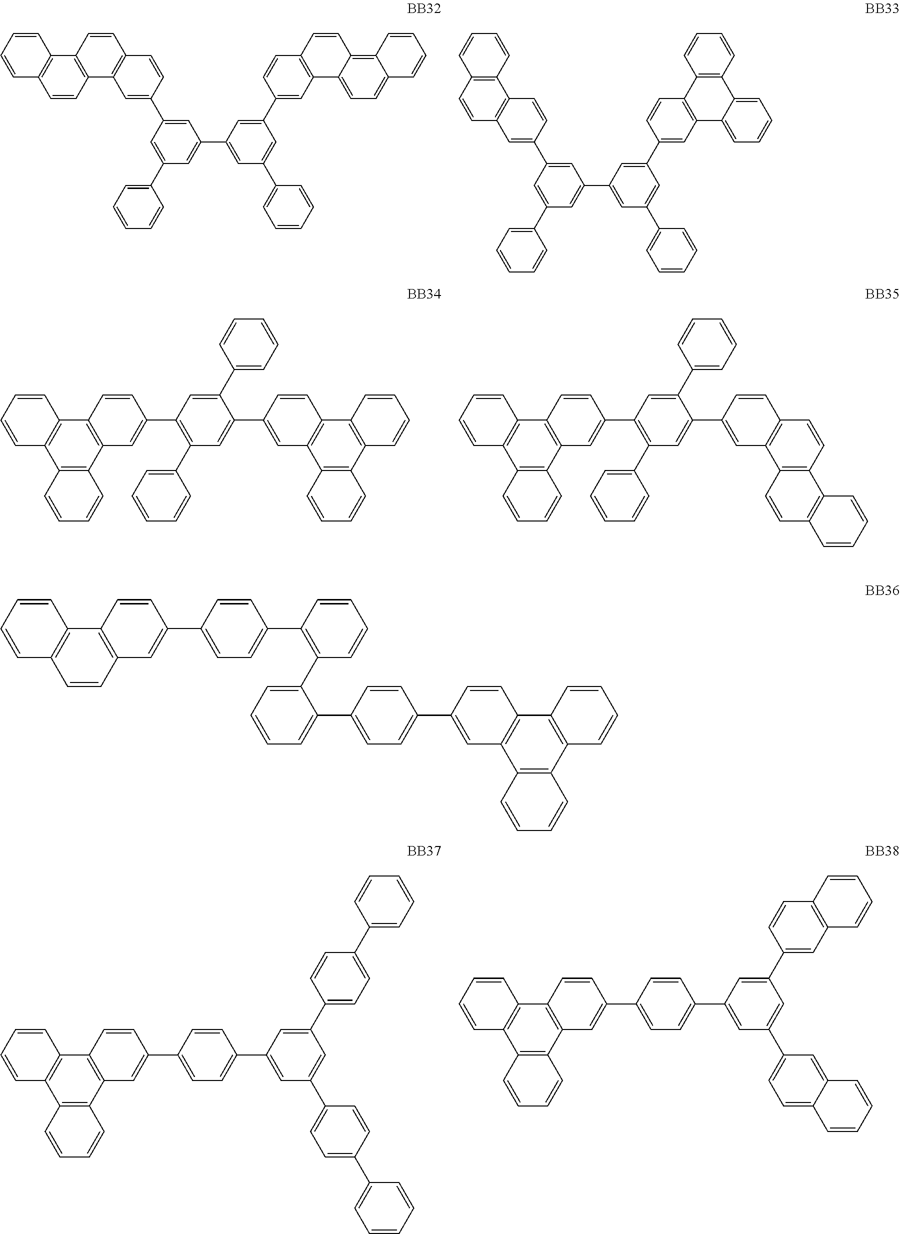

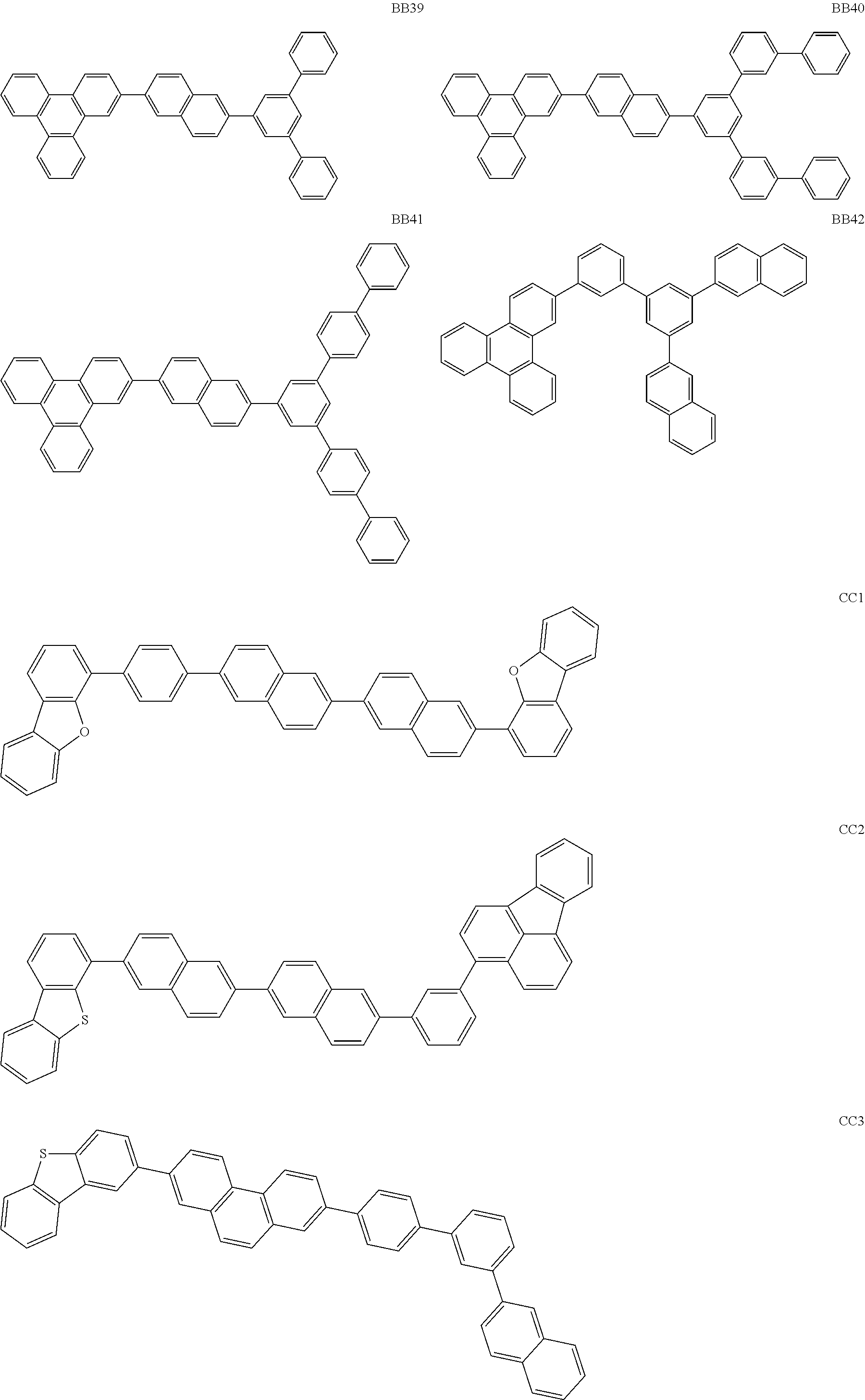

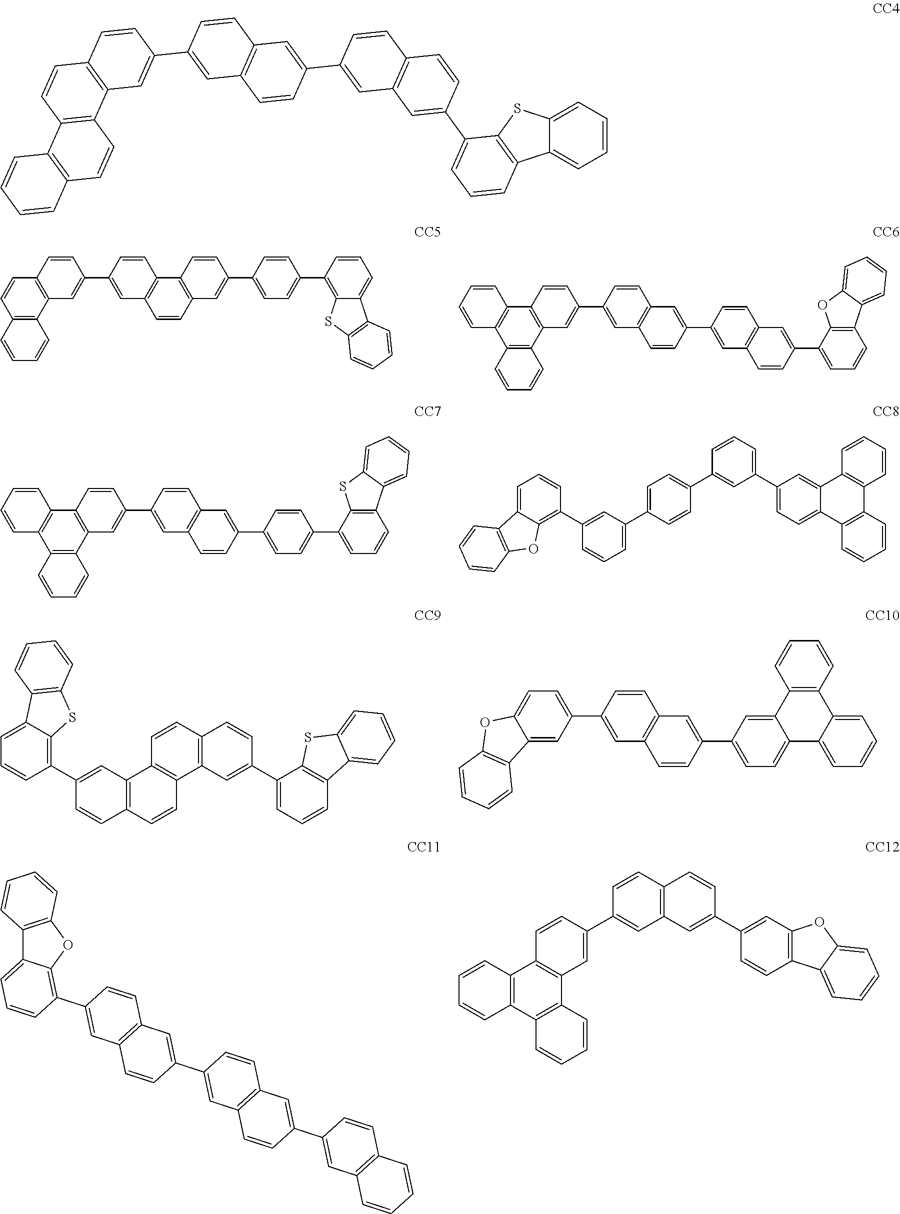

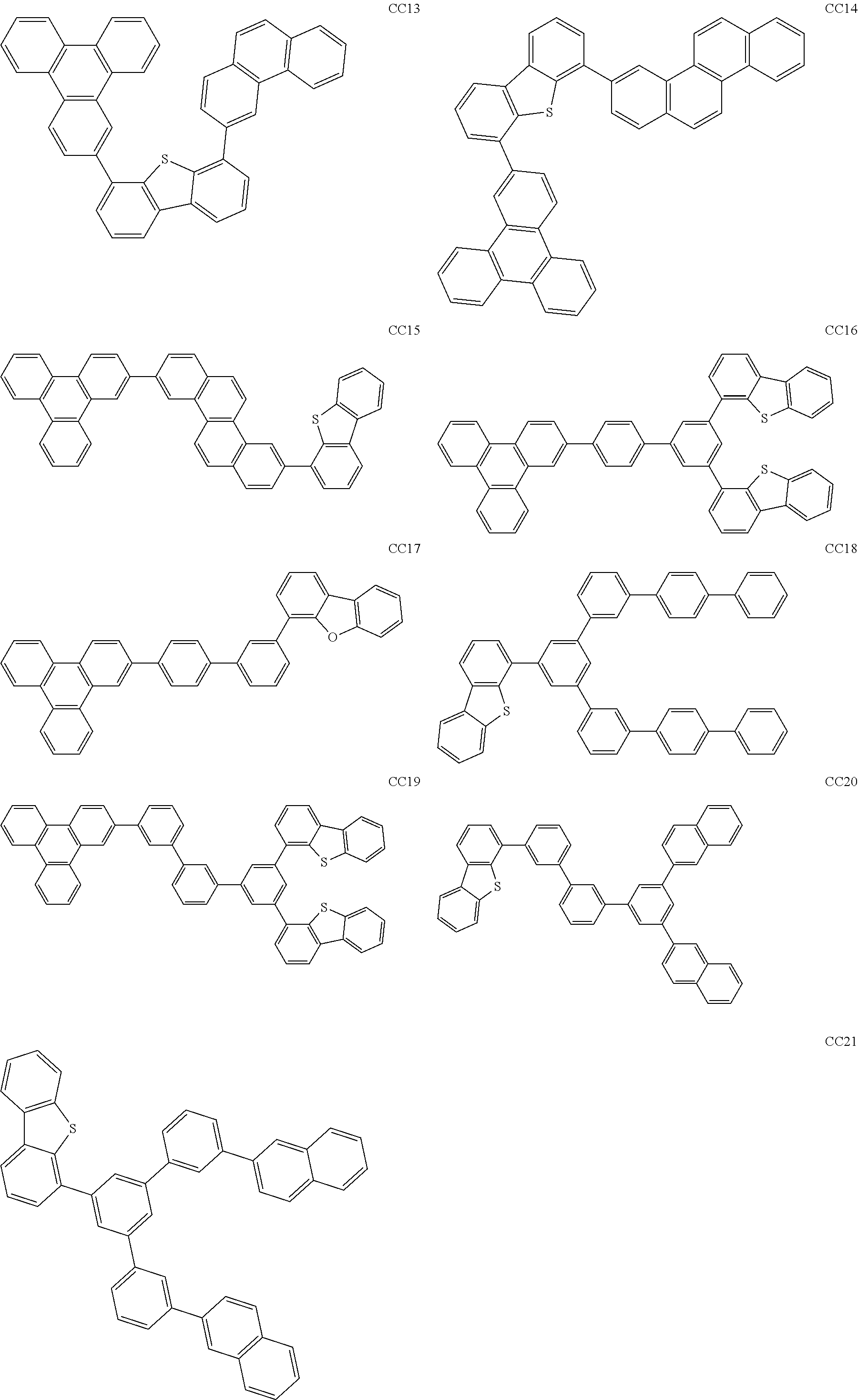

Among these compounds, exemplary compounds belonging to the group AA (AA1 to AA21) are compounds with the carbazole structure. Thus, these compounds have high hole transport ability due to the carbazole structure. This can compensate for the relatively low hole transport ability of an organic compound represented by the general formula [1]. Thus, a light-emitting layer also having high hole transport ability can be formed, and the organic light-emitting element can have high luminescence efficiency.

Among these compounds, the exemplary compounds belonging to the group BB (BB1 to BB42) are compounds having a skeleton with at least one selected from the group consisting of a triphenylene structure, a phenanthrene structure, a chrysene structure, and a fluoranthene structure in the skeleton and having no sp3 carbon. Thus, when these compounds are combined with an organic compound represented by the general formula [1] to form a layer, the intermolecular distance between them can be shortened. This allows efficient intermolecular energy transfer, more specifically, energy transfer from the second organic compound to a compound represented by the general formula [1] and can improve luminescence efficiency. Among these compounds, compounds with a triphenylene structure, more specifically, BB6 to BBB, BB10 to BB29, and BB34 to BB42 have particularly high planarity.

Among the compounds, the exemplary compounds belonging to the group CC (CC1 to CC21) are compounds with a dibenzothiophene structure or a dibenzofuran structure in the skeleton and without sp3 carbon. Thus, when these compounds are combined with an organic compound represented by the general formula [1] to form a light-emitting layer, the balance between HOMO and LUMO is improved. This results in a good carrier balance and an organic light-emitting element with high luminescence efficiency. Among these compounds, compounds with a dibenzothiophene structure, more specifically, CC2 to CCS, CC7, CC9, CC13 to CC16, and CC18 to CC21 result in a good carrier balance.

### Other Compounds

Examples of other compounds that can be used for the organic light-emitting element according to the present embodiment are described below.

The hole-injection/transport material suitably used for the hole-injection layer or the hole-transport layer can be a material with high hole mobility that can facilitate hole injection from the positive electrode and that can transport injected holes to the light-emitting layer. Furthermore, a material with a high glass transition temperature can be used to reduce degradation of film quality, such as crystallization, in the organic light-emitting element. Examples of the low-molecular-weight or high-molecular-weight material with hole-injection/transport ability include, but are not limited to, triarylamine derivatives, aryl carbazole derivatives, phenylenediamine derivatives, stilbene derivatives, phthalocyanine derivatives, porphyrin derivatives, polyvinylcarbazole, polythiophene, and other electrically conductive polymers. The hole-injection/transport material is also suitably used for an electron-blocking layer.

Specific examples of compounds that can be used as hole-transport materials include, but are not limited to, the following.

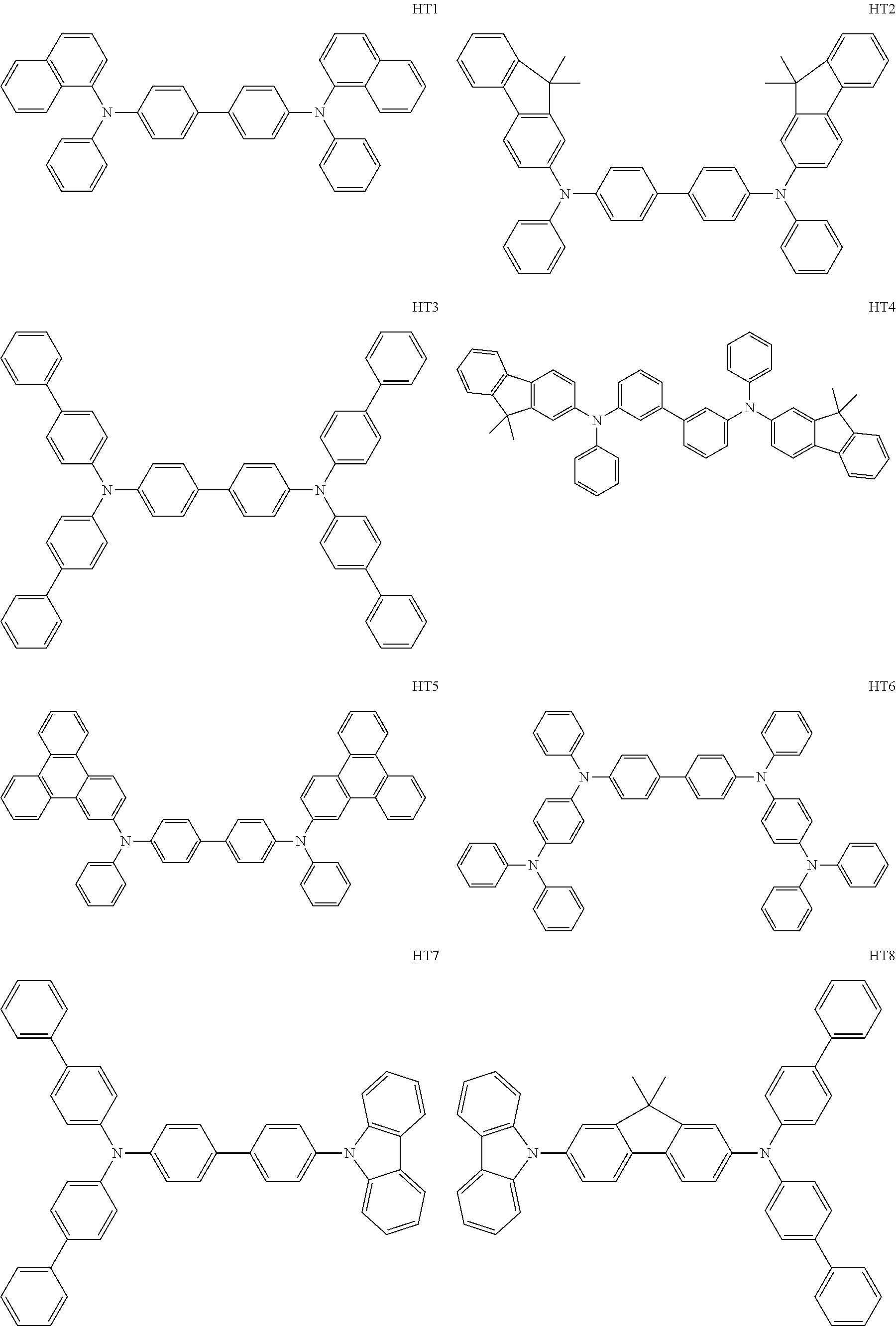

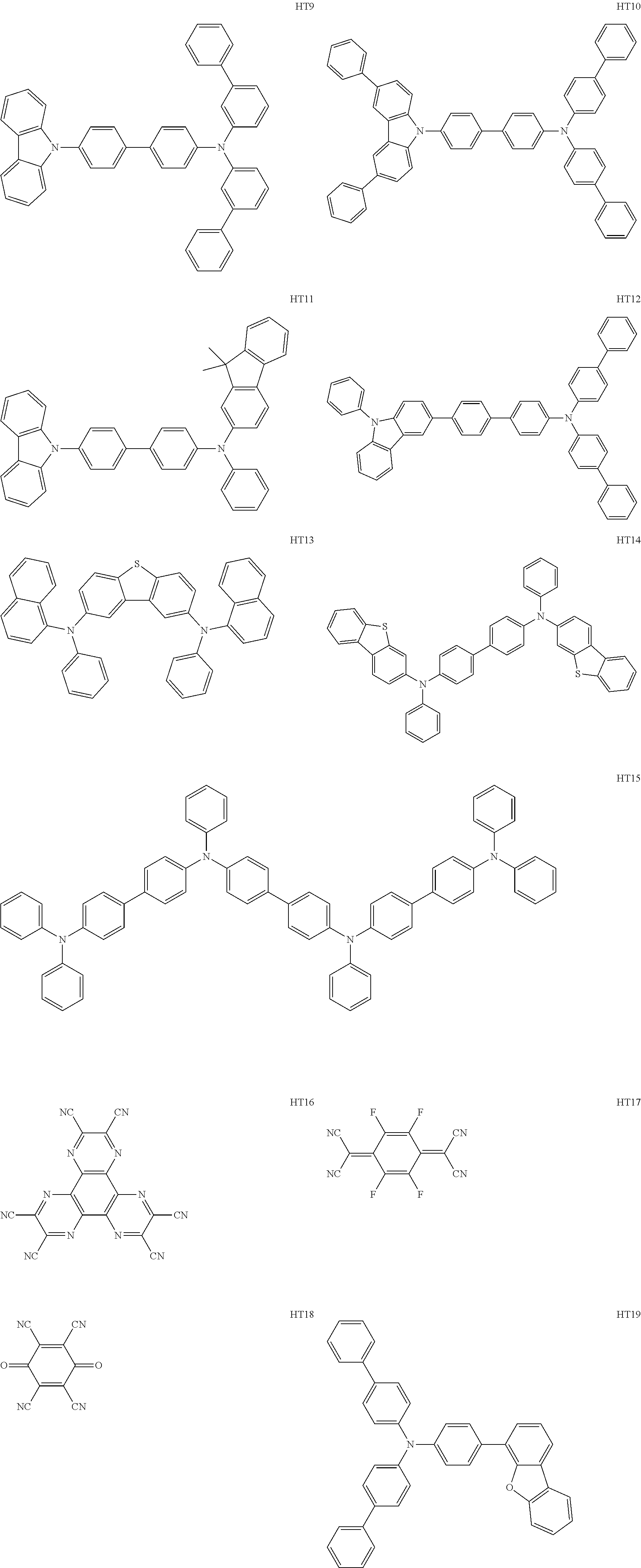

Examples of a light-emitting material mainly related to the light-emitting function include, in addition to the organic compounds represented by the general formula [1], fused-ring compounds (for example, fluorene derivatives, naphthalene derivatives, pyrene derivatives, perylene derivatives, tetracene derivatives, anthracene derivatives, rubrene, etc.), quinacridone derivatives, coumarin derivatives, stilbene derivatives, organoaluminum complexes, such as tris(8-quinolinolato)aluminum, iridium complexes, platinum complexes, rhenium complexes, copper complexes, europium complexes, ruthenium complexes, and polymer derivatives, such as poly(phenylene vinylene) derivatives, polyfluorene derivatives, and polyphenylene derivatives.

Specific examples of compounds that can be used as light-emitting materials include, but are not limited to, the following.

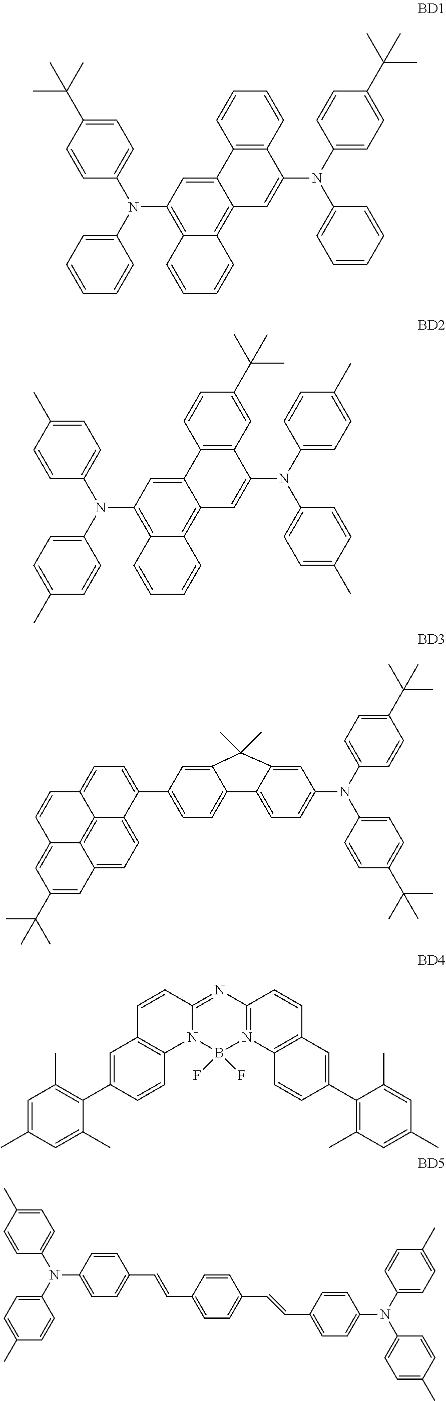

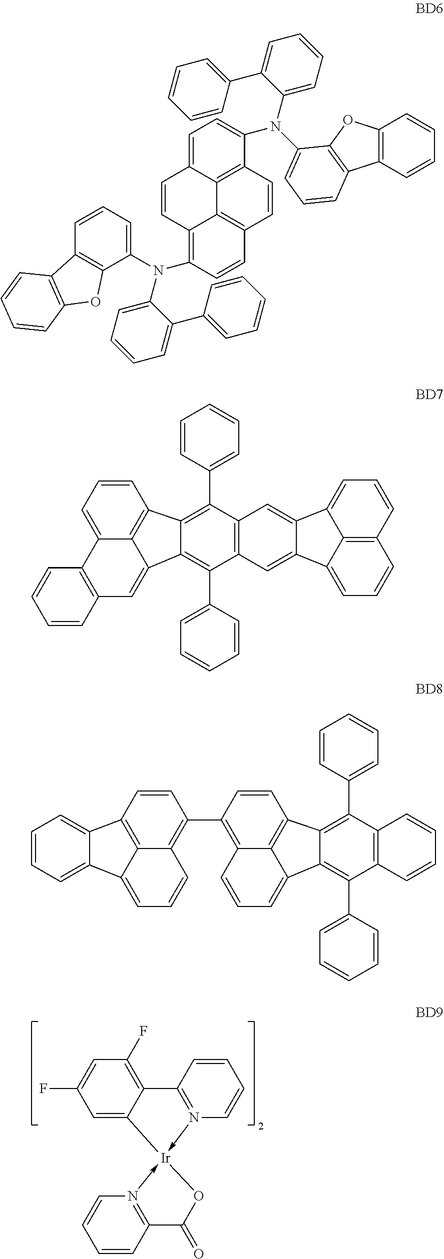

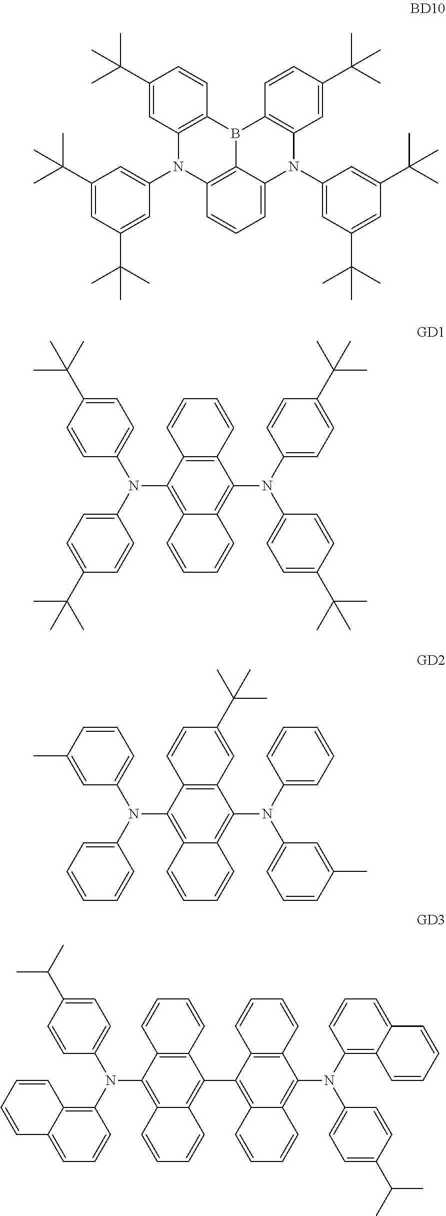

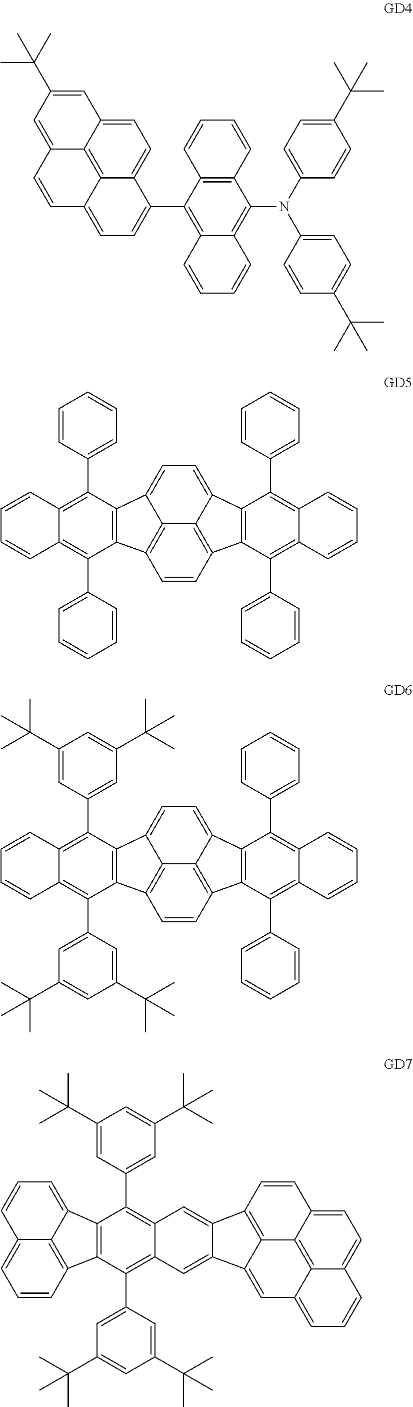

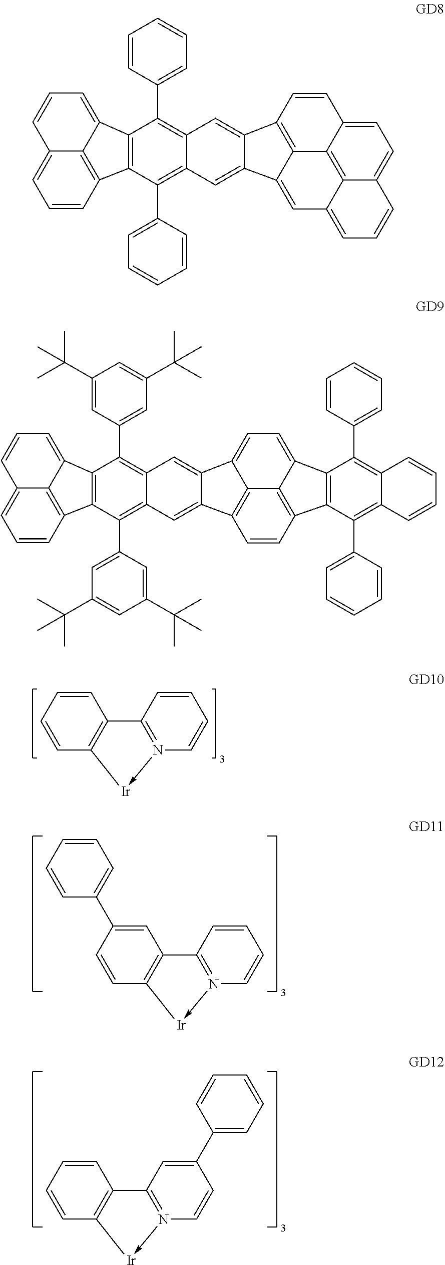

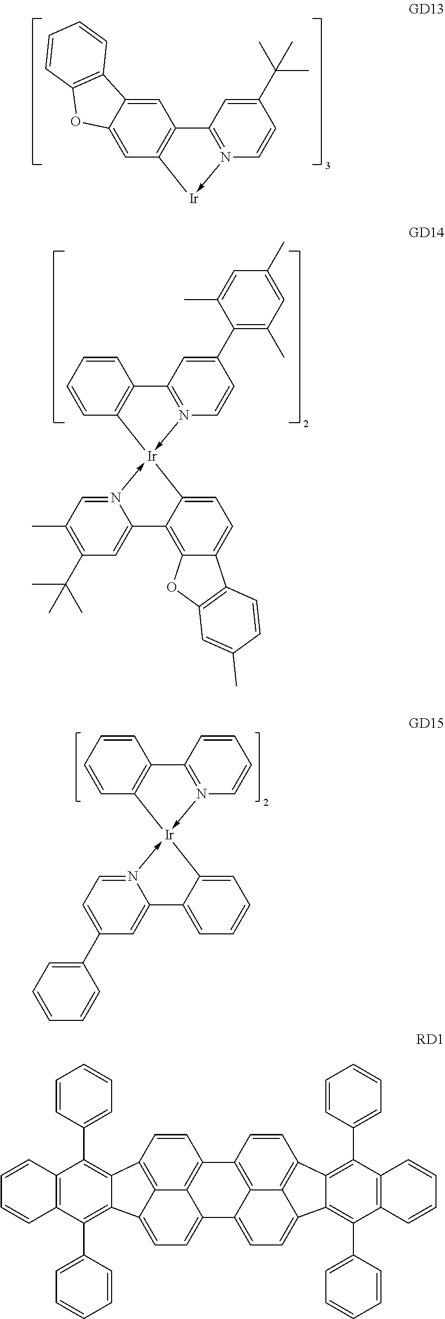

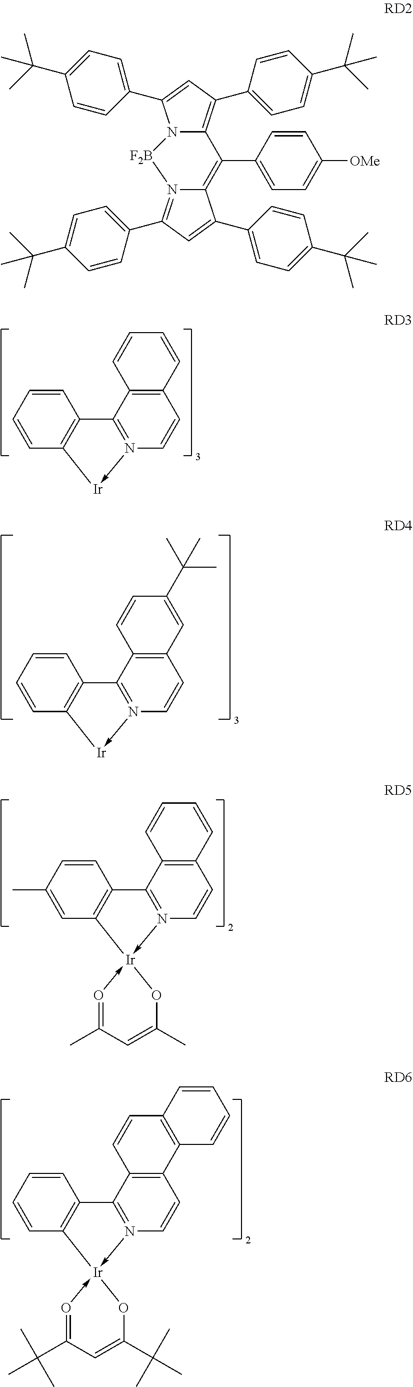

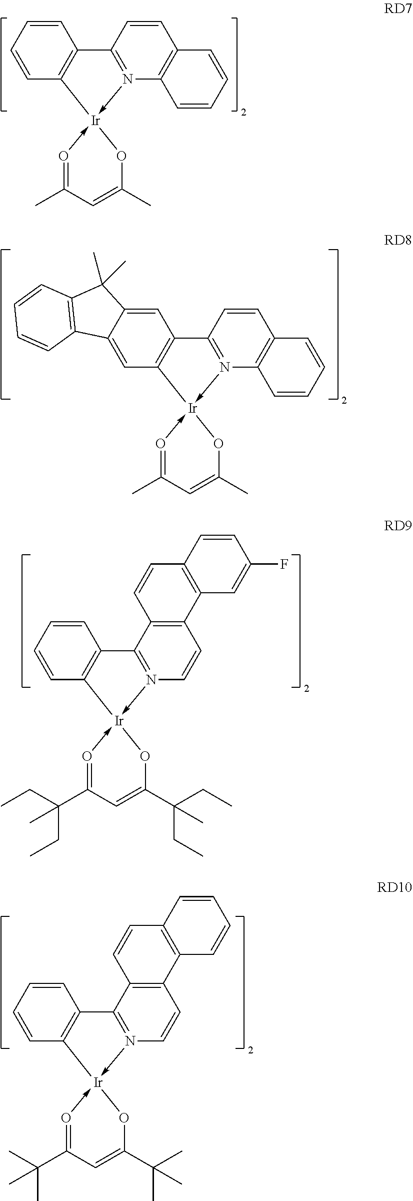

Examples of a light-emitting layer host or assist material in the light-emitting layer include, in addition to the materials of the AA, BB, and CC groups, aromatic hydrocarbon compounds and derivatives thereof, carbazole derivatives, dibenzofuran derivatives, dibenzothiophene derivatives, organoaluminum complexes, such as tris(8-quinolinolato)aluminum, and organoberyllium complexes.

The assist material can be a compound with at least one structure selected from a xanthone structure, a thioxanthone structure, and a benzophenone structure, which have a deep LUMO (far from the vacuum level) like an azine ring. More specifically, EM28 to EM31 described below can be used. The assist material can also be a compound with an azine ring.

Specific examples of a compound that can be used as a host or assist material in a light-emitting layer include, but are not limited to, the following.

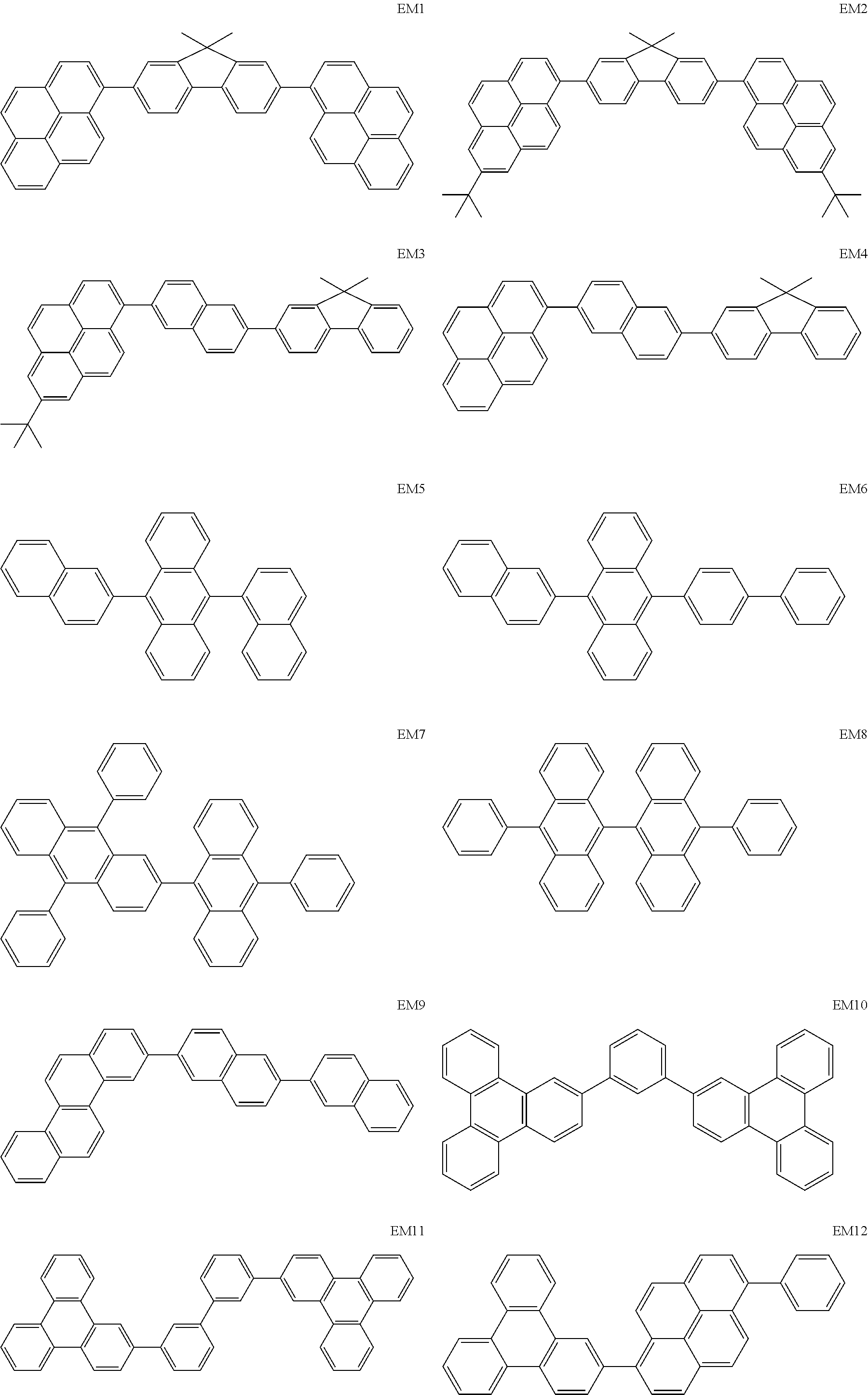

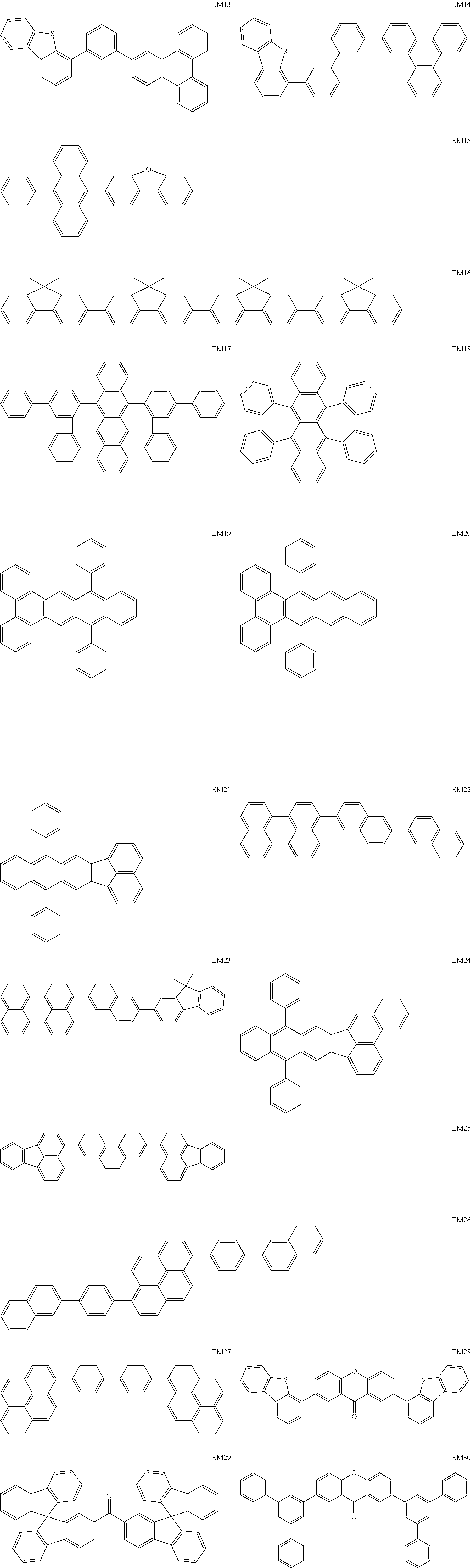

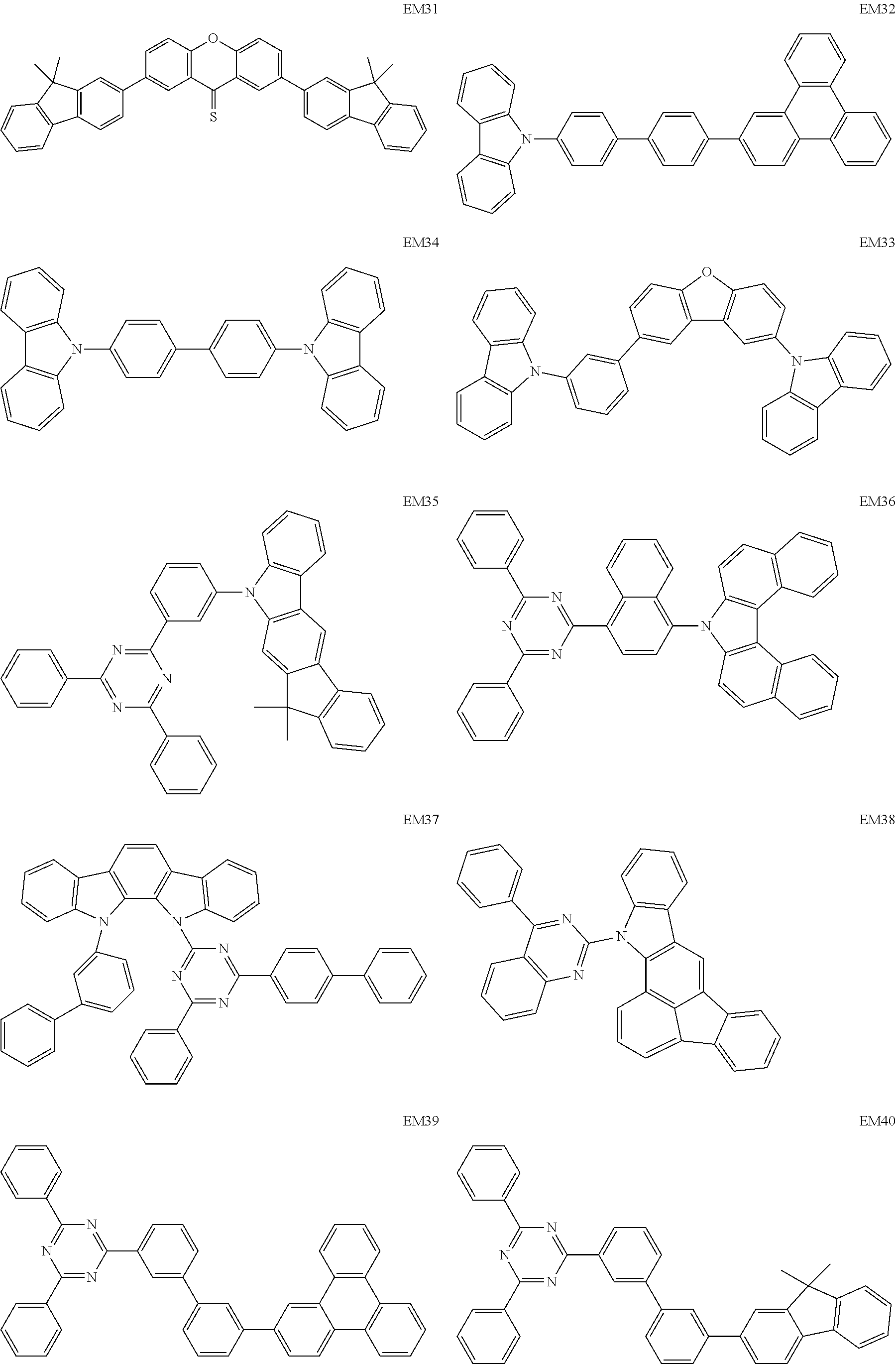

An electron-transport material can be selected from materials that can transport electrons injected from the negative electrode to the light-emitting layer and is selected in consideration of the balance with the hole mobility of a hole-transport material and the like. Examples of materials with electron-transport ability include, but are not limited to, oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused-ring compounds (for example, fluorene derivatives, naphthalene derivatives, chrysene derivatives, and anthracene derivatives). Furthermore, the electron-transport material is also suitably used for a hole-blocking layer.

Specific examples of compounds that can be used as electron-transport materials include, but are not limited to, the following.

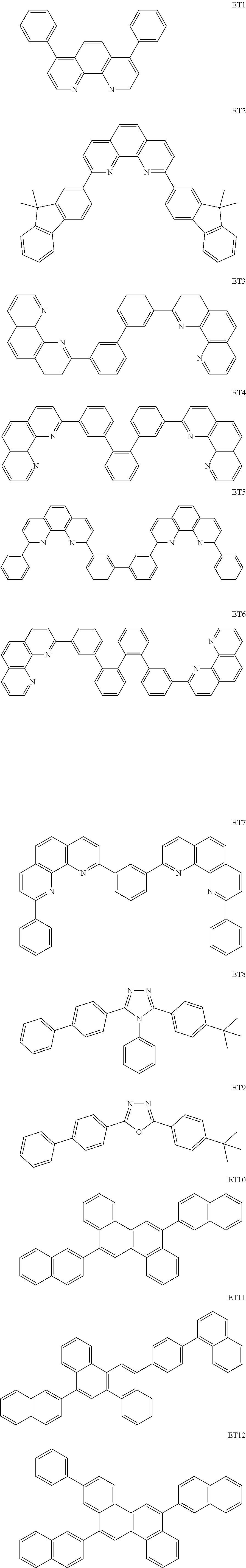

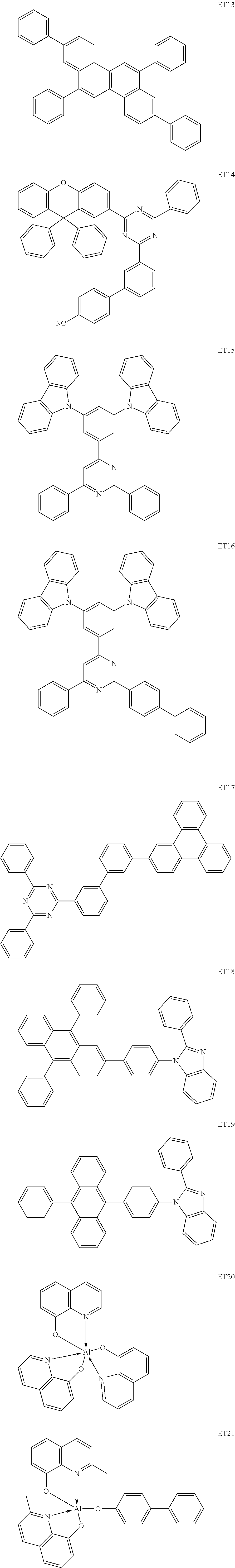

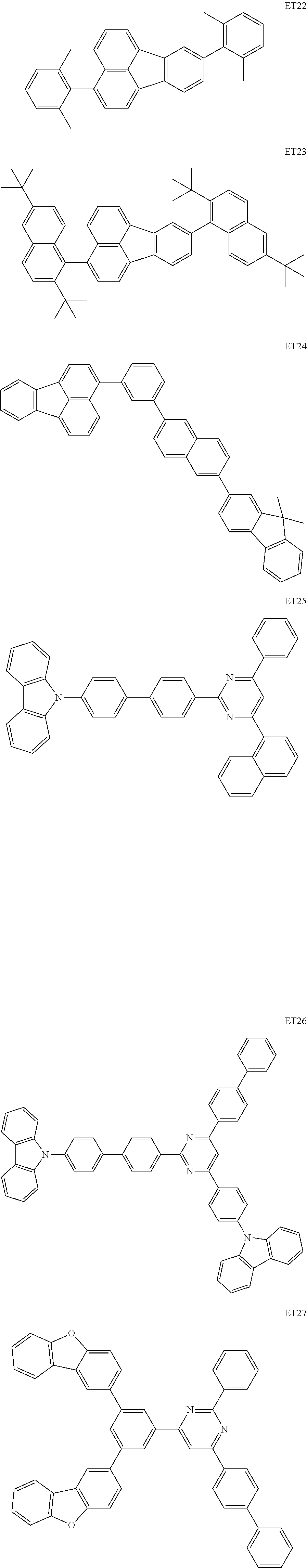

Constituents other than the organic compound layers constituting the organic light-emitting element according to the present embodiment are described below. The organic light-emitting element may include a first electrode, an organic compound layer, and a second electrode on a substrate. One of the first electrode and the second electrode is a positive electrode, and the other is a negative electrode. A protective layer, a color filter, or the like may be provided on the second electrode. When a color filter is provided, a planarization layer may be provided between the color filter and a protective layer. The planarization layer may be composed of an acrylic resin or the like.

The substrate may be formed of quartz, glass, silicon, resin, metal, or the like. The substrate may have a switching element, such as a transistor, and wiring, on which an insulating layer may be provided. The insulating layer may be formed of any material, provided that the insulating layer can have a contact hole to ensure electrical connection between the positive electrode and wiring and can be insulated from unconnected wiring. For example, the insulating layer may be formed of a resin, such as polyimide, silicon oxide, or silicon nitride.

A constituent material of the positive electrode can have as large a work function as possible. Examples of the constituent material include metal elements, such as gold, platinum, silver, copper, nickel, palladium, cobalt, selenium, vanadium, and tungsten, mixtures thereof, alloys thereof, and metal oxides, such as tin oxide, zinc oxide, indium oxide, indium tin oxide (ITO), and indium zinc oxide. Electrically conductive polymers, such as polyaniline, polypyrrole, and polythiophene, may also be used. These electrode materials may be used alone or in combination. The positive electrode may be composed of a single layer or a plurality of layers. When used as a reflective electrode, for example, chromium, aluminum, silver, titanium, tungsten, molybdenum, an alloy thereof, or a laminate thereof can be used. When used as a transparent electrode, an oxide transparent conductive layer, such as indium tin oxide (ITO) or indium zinc oxide, can be used. However, the present disclosure is not limited thereto. The positive electrode may be formed by photolithography.

A constituent material of the negative electrode can be a material with a small work function. For example, an alkali metal, such as lithium, an alkaline-earth metal, such as calcium, a metal element, such as aluminum, titanium, manganese, silver, lead, or chromium, or a mixture thereof may be used. An alloy of these metal elements may also be used. For example, magnesium-silver, aluminum-lithium, aluminum- magnesium, silver-copper, or zinc-silver may be used. A metal oxide, such as indium tin oxide (ITO), may also be used. These electrode materials may be used alone or in combination. The negative electrode may be composed of a single layer or a plurality of layers. Among them, silver can be used, and a silver alloy can be used to reduce the aggregation of silver. As long as the aggregation of silver can be reduced, the alloy may have any ratio. For example, it may be 1:1.

The negative electrode may be, but is not limited to, an oxide conductive layer, such as ITO, for a top emission element or a reflective electrode, such as aluminum (Al), for a bottom emission element. The negative electrode may be formed by any method. A direct-current or alternating-current sputtering method can achieve good film coverage and easily decrease resistance.

A protective layer may be provided after the negative electrode is formed. For example, a glass sheet with a moisture absorbent may be attached to the negative electrode to decrease the amount of water or the like entering the organic compound layer and reduce the occurrence of display defects. In another embodiment, a passivation film, such as silicon nitride, may be provided on the negative electrode to decrease the amount of water or the like entering the organic compound layer. For example, after the negative electrode is formed, the negative electrode is transferred to another chamber without breaking the vacuum, and a silicon nitride film with a thickness of 2 μm may be formed as a protective layer by a chemical vapor deposition (CVD) method. The protective layer may be formed by the CVD method followed by an atomic layer deposition (ALD) method.

Furthermore, each pixel may be provided with a color filter. For example, a color filter that matches the size of the pixel may be provided on another substrate and may be bonded to the substrate of the organic light-emitting element, or a color filter may be patterned by photolithography on the protective layer formed of silicon oxide or the like.

An organic compound layer (a hole-injection layer, a hole-transport layer, an electron-blocking layer, a light-emitting layer, a hole-blocking layer, an electron-transport layer, an electron-injection layer, etc.) constituting the organic light-emitting element according to the present embodiment is formed by the following method. That is, an organic compound layer may be formed by a dry process, such as a vacuum deposit method, an ionized deposition method, sputtering, or plasma. Instead of the dry process, a wet process may also be employed in which a layer is formed by a known coating method (for example, spin coating, dipping, a casting method, an LB method, an ink jet method, etc.) using an appropriate solvent. A layer formed by a vacuum deposit method, a solution coating method, or the like undergoes little crystallization or the like and has high temporal stability. When a film is formed by a coating method, the film may also be formed in combination with an appropriate binder resin. Examples of the binder resin include, but are not limited to, polyvinylcarbazole resins, polycarbonate resins, polyester resins, ABS resins, acrylic resins, polyimide resins, phenolic resins, epoxy resins, silicone resins, and urea resins. The binder resins may be used alone as a homopolymer or a copolymer or may be used in combination. If necessary, an additive agent, such as a known plasticizer, oxidation inhibitor, and/or ultraviolet absorbent, may also be used.

### Apparatus Including Organic Light-Emitting Element

The organic light-emitting element according to the present embodiment can be used as a constituent of a display apparatus or a lighting apparatus. Other applications include an exposure light source of an electrophotographic image-forming apparatus, a backlight of a liquid crystal display, and a light-emitting apparatus with a color filter in a white light source.

The display apparatus may be an image-information-processing apparatus that includes an image input unit for inputting image information from an area CCD, a linear CCD, a memory card, or the like, includes an information processing unit for processing the input information, and displays an input image on a display unit. The display apparatus may have a plurality of pixels, and at least one of the pixels may include the organic light-emitting element according to the present embodiment and a transistor coupled to the organic light-emitting element. The substrate may be a semiconductor substrate formed of silicon or the like, and the transistor may be a MOSFET formed on the substrate.

A display unit of an imaging apparatus or an ink jet printer may have a touch panel function. A driving system of the touch panel function may be, but is not limited to, an infrared radiation system, an electrostatic capacitance system, a resistive film system, or an electromagnetic induction system. The display apparatus may be used for a display unit of a multifunction printer.

Next, the display apparatus according to the present embodiment is described with reference to the accompanying drawings.

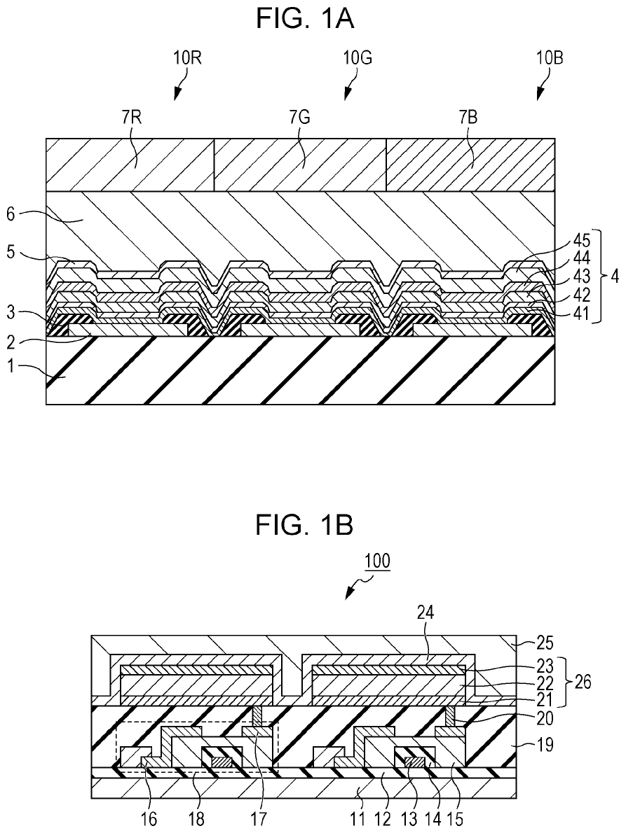

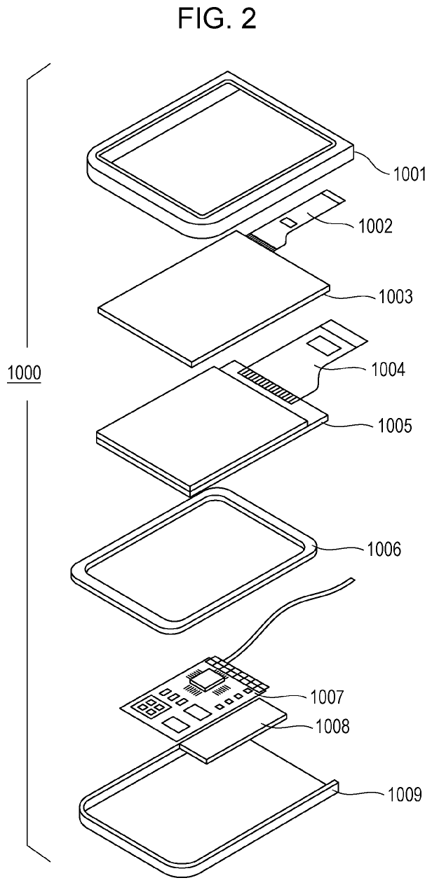

FIGS. 1A and 1B are schematic cross-sectional views of an example of a display apparatus that includes an organic light-emitting element and a transistor coupled to the organic light-emitting element. The transistor is an example of an active element. The transistor may be a thin-film transistor (TFT).

FIG. 1A illustrates an example of a pixel serving as a constituent of the display apparatus according to the present embodiment. The pixel has subpixels 10. The subpixels are 10R, 10G, and 10B with different emission colors. The emission colors may be distinguished by the wavelength of light emitted from the light-emitting layer, or light emitted from each subpixel may be selectively transmitted or color-converted with a color filter or the like. Each subpixel has, on an interlayer insulating layer 1, a reflective electrode 2 as a first electrode, an insulating layer 3 covering the ends of the reflective electrode 2, organic compound layers 4 covering the first electrode and the insulating layer, a transparent electrode 5, a protective layer 6, and a color filter 7.

A transistor and/or a capacitor element may be provided under or inside the interlayer insulating layer 1. The transistor may be electrically connected to the first electrode via a contact hole (not shown) or the like.

The insulating layer 3 is also referred to as a bank or a pixel separation film. The insulating layer 3 covers the ends of the first electrode and surrounds the first electrode. A portion of the first electrode not covered with the insulating layer is in contact with the organic compound layers 4 and serves as a light-emitting region.

The organic compound layers 4 include a hole-injection layer 41, a hole-transport layer 42, a first light-emitting layer 43, a second light-emitting layer 44, and an electron-transport layer 45.

The second electrode 5 may be a transparent electrode, a reflective electrode, or a semitransparent electrode.

The protective layer 6 reduces the penetration of moisture into the organic compound layers. The protective layer is illustrated as a single layer but may be a plurality of layers. The protective layer may include an inorganic compound layer and an organic compound layer.

The color filter 7 is divided into 7R, 7G, and 7B according to the color. The color filter may be formed on a planarizing film (not shown). Furthermore, a resin protective layer (not shown) may be provided on the color filter. The color filter may be formed on the protective layer 6. Alternatively, the color filter may be bonded after being provided on an opposite substrate, such as a glass substrate.

A display apparatus 100 illustrated in FIG. 1B includes an organic light-emitting element 26 and a TFT 18, which is an example of a transistor. The display apparatus 100 includes a substrate 11 made of glass, silicon, or the like and an insulating layer 12 on the substrate 11. An active element, such as the TFT 18, and a gate electrode 13, a gate-insulating film 14, and a semiconductor layer 15 of the active element are provided on the insulating layer 12.

The TFT 18 includes the semiconductor layer 15, a drain electrode 16, and a source electrode 17. The TFT 18 is covered with an insulating film 19. A positive electrode 21 constituting the organic light-emitting element 26 is connected to the source electrode 17 via a contact hole 20.

Electrical connection between the electrodes of the organic light-emitting element 26 (the positive electrode 21 and a negative electrode 23) and the electrodes of the TFT (the source electrode 17 and the drain electrode 16) is not limited to that illustrated in FIG. 1B. More specifically, it is only necessary to electrically connect either the positive electrode 21 or the negative electrode 23 to either the source electrode 17 or the drain electrode 16 of the TFT 18.

Although an organic compound layer 22 is a single layer in the display apparatus 100 illustrated in FIG. 1B, the organic compound layer 22 may be composed of a plurality of layers. The negative electrode 23 is covered with a first protective layer 25 and a second protective layer 24 for preventing degradation of the organic light-emitting element.

The transistor used as a switching element in the display apparatus 100 illustrated in FIG. 1B may be replaced with another switching element, such as a metal insulator metal (MIM) element.

The transistor used in the display apparatus 100 in FIG. 1B is not limited to a thin-film transistor including an active layer on an insulating surface of a substrate and may also be a transistor including a single crystal silicon wafer. The active layer may be single-crystal silicon, non-single-crystal silicon, such as amorphous silicon or microcrystalline silicon, or a non-single-crystal oxide semiconductor, such as indium zinc oxide or indium gallium zinc oxide. The thin-film transistor is also referred to as a TFT element.

The transistor in the display apparatus 100 of FIG. 1B may be formed within a substrate, such as a Si substrate. The phrase “formed within a substrate” means that the substrate, such as a Si substrate, itself is processed to form the transistor. Thus, the transistor within the substrate can be considered that the substrate and the transistor are integrally formed.

In the organic light-emitting element according to the present embodiment, the luminous brightness is controlled with the TFT, which is an example of a switching element. The organic light-emitting element can be provided in a plurality of planes to display an image at each luminous brightness. The switching element according to the present embodiment is not limited to the TFT and may be a transistor formed of low-temperature polysilicon or an active-matrix driver formed on a substrate, such as a Si substrate. “On a substrate” may also be referred to as “within a substrate”. Whether a transistor is provided within a substrate or a TFT is used depends on the size of a display unit. For example, for an approximately 0.5-inch display unit, an organic light-emitting element can be provided on a Si substrate.

FIG. 2 is a schematic view of an example of the display apparatus according to the present embodiment. A display apparatus 1000 may include a touch panel 1003, a display panel 1005, a frame 1006, a circuit substrate 1007, and a battery 1008 between an upper cover 1001 and a lower cover 1009. The touch panel 1003 and the display panel 1005 are coupled to flexible print circuits FPC 1002 and 1004, respectively. Transistors are printed on the circuit substrate 1007. The battery 1008 may not be provided when the display apparatus is not a mobile device, or may be provided at another position even when the display apparatus is a mobile device.

The display apparatus according to the present embodiment may be used for a display unit of an imaging apparatus that includes an optical unit with a plurality of lenses and an imaging element for receiving light passing through the optical unit. The imaging apparatus may include a display unit for displaying information acquired by the imaging element. The display unit may be a display unit exposed outside from the imaging apparatus or a display unit located in a finder. The imaging apparatus may be a digital camera or a digital video camera. The imaging apparatus may also be referred to as a photoelectric conversion apparatus.

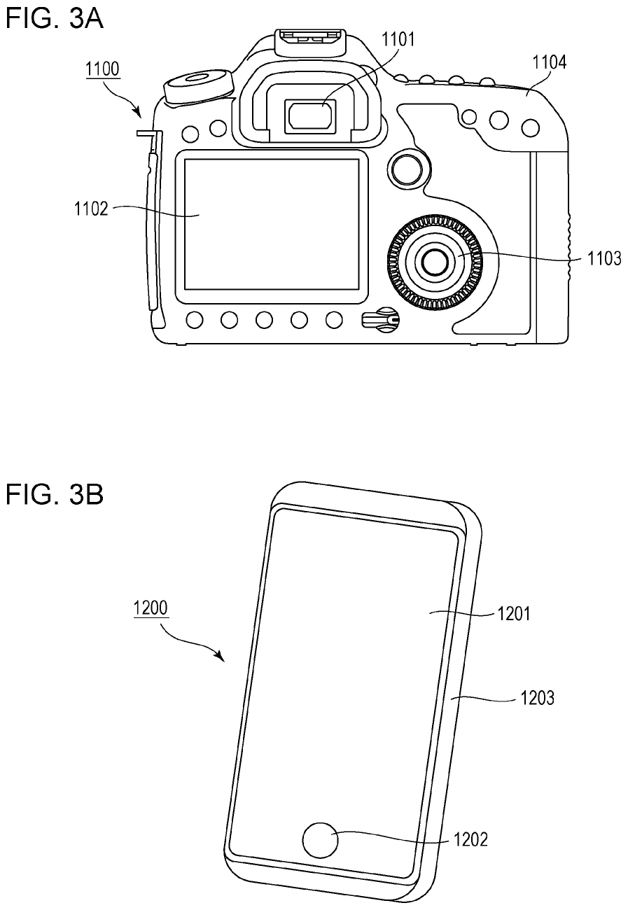

FIG. 3A is a schematic view of an example of an imaging apparatus according to the present embodiment. An imaging apparatus 1100 may include a viewfinder 1101, a rear display 1102, an operating unit 1103, and a housing 1104. The viewfinder 1101 may include the display apparatus according to the present embodiment. In such a case, the display apparatus may display environmental information, imaging instructions, and the like as well as an image to be captured. The environmental information may include the intensity of external light, the direction of external light, the travel speed of the photographic subject, the possibility that the photographic subject is shielded by a shielding material, and the like.

Because the appropriate timing for imaging is a short time, it is better to display information as soon as possible. Thus, a display apparatus including the organic light-emitting element according to the present embodiment can be used. This is because the organic light-emitting element has a high response speed. A display apparatus including the organic light-emitting element can be more suitably used than these apparatuses and liquid crystal displays that require a high display speed.

The imaging apparatus 1100 includes an optical unit (not shown). The optical unit has a plurality of lenses and focuses an image on an imaging element in the housing 1104. The focus of the lenses can be adjusted by adjusting their relative positions. This operation can also be automatically performed.

The display apparatus according to the present embodiment may include color filters of red, green, and blue colors. In the color filters, the red, green, and blue colors may be arranged in a delta arrangement.

The display apparatus according to the present embodiment may be used for a display unit of electronic equipment, such as a mobile terminal. Such a display apparatus may have both a display function and an operation function. Examples of the mobile terminal include mobile phones, such as smartphones, tablets, and head-mounted displays.

FIG. 3B is a schematic view of an example of electronic equipment according to the present embodiment. Electronic equipment 1200 includes a display unit 1201, an operating unit 1202, and a housing 1203. The housing 1203 may include a circuit, a printed circuit board including the circuit, a battery, and a communication unit. The operating unit 1202 may be a button or a touch panel response unit. The operating unit may be a biometric recognition unit that recognizes a fingerprint and releases the lock. Electronic equipment with a communication unit may also be referred to as communication equipment.

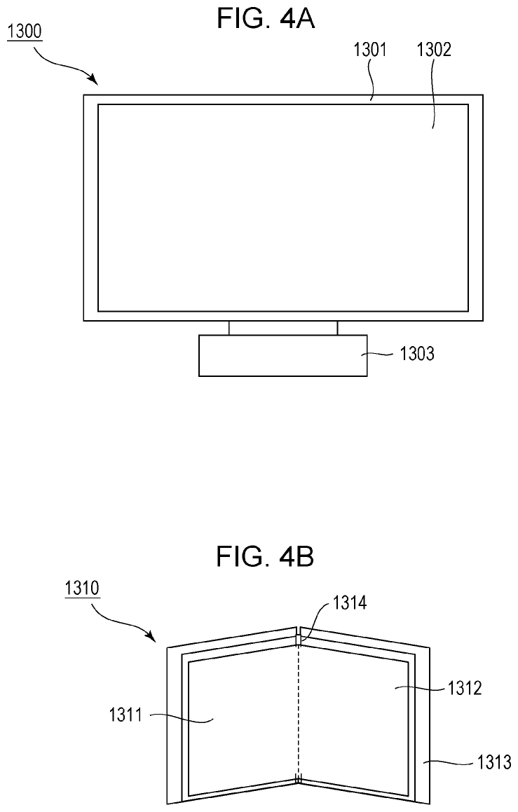

FIGS. 4A and 4B are schematic views of an example of the display apparatus according to the present embodiment. FIG. 4A illustrates a display apparatus, such as a television monitor or a PC monitor. A display apparatus 1300 includes a frame 1301 and a display unit 1302. The light-emitting apparatus according to the present embodiment may be used for the display unit 1302. The display apparatus 1300 includes a base 1303 for supporting the frame 1301 and the display unit 1302. The base 1303 is not limited to the structure illustrated in FIG. 4A. The lower side of the frame 1301 may also serve as the base. The frame 1301 and the display unit 1302 may be bent so that the display surface of the display unit 1302 is curved. The radius of curvature may range from 5000 to 6000 mm

FIG. 4B is a schematic view of another example of the display apparatus according to the present embodiment. A display apparatus 1310 in FIG. 4B is configured to be foldable and is a so-called foldable display apparatus. The display apparatus 1310 includes a first display unit 1311, a second display unit 1312, a housing 1313, and a folding point 1314. The first display unit 1311 and the second display unit 1312 may include the light-emitting apparatus according to the present embodiment. The first display unit 1311 and the second display unit 1312 may be a single display apparatus without a joint. The first display unit 1311 and the second display unit 1312 can be divided by a folding point. The first display unit 1311 and the second display unit 1312 may display different images or one image.

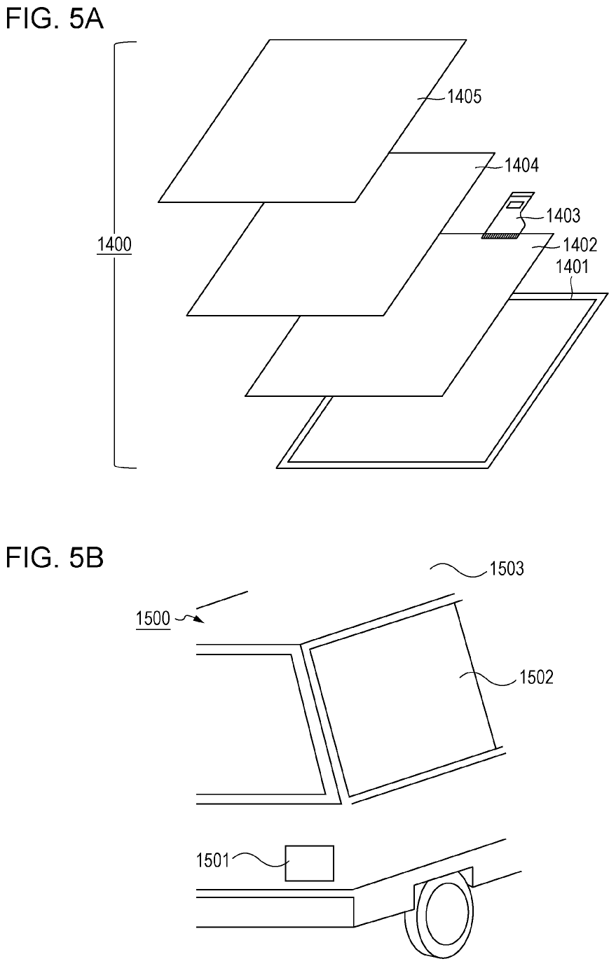

FIG. 5A is a schematic view of an example of a lighting apparatus according to the present embodiment. A lighting apparatus 1400 may include a housing 1401, a light source 1402, a circuit substrate 1403, an optical filter 1404 that transmits light emitted by the light source 1402, and a light-diffusing unit 1405. The light source 1402 may include the organic light-emitting element according to the present embodiment. The optical filter may be a filter for improving the color rendering properties of the light source. The light-diffusing unit can effectively diffuse light from the light source and widely spread light as in illumination. The optical filter and the light-diffusing unit may be provided on the light output side of the illumination. If necessary, a cover may be provided on the outermost side.

For example, the lighting apparatus is an interior lighting apparatus. The lighting apparatus may emit white light, neutral white light, or light of any color from blue to red. The lighting apparatus may have a light control circuit for controlling such light or a color control circuit for controlling emission color. The lighting apparatus may include the organic light-emitting element according to the present embodiment and a power supply circuit coupled thereto. The power supply circuit is a circuit that converts an AC voltage to a DC voltage. White has a color temperature of 4200 K, and neutral white has a color temperature of 5000 K. The lighting apparatus may have a color filter.

The lighting apparatus according to the present embodiment may include a heat dissipation unit. The heat dissipation unit releases heat from the apparatus to the outside and may be a metal or liquid silicon with a high specific heat.

FIG. 5B is a schematic view of an automobile as an example of a moving body according to the present embodiment. The automobile has a taillight as an example of a lamp. An automobile 1500 may have a taillight 1501, which comes on when a brake operation or the like is performed.

The taillight 1501 may include the organic light-emitting element according to the present embodiment. The taillight 1501 may have a protective member for protecting an organic EL element. The protective member may be formed of any transparent material with moderately high strength and can be formed of polycarbonate or the like. The polycarbonate may be mixed with a furan dicarboxylic acid derivative, an acrylonitrile derivative, or the like.

The automobile 1500 may have a body 1503 and a window 1502 on the body 1503. The window 1502 may be a transparent display as long as it is not a window for checking the front and rear of the automobile. The transparent display may include the organic light-emitting element according to the present embodiment. In such a case, constituent materials, such as electrodes, of the organic light-emitting element are transparent materials.

The moving body according to the present embodiment may be a ship, an aircraft, a drone, or the like. The moving body may include a body and a lamp provided on the body. The lamp may emit light to indicate the position of the body. The lamp includes the organic light-emitting element according to the present embodiment.

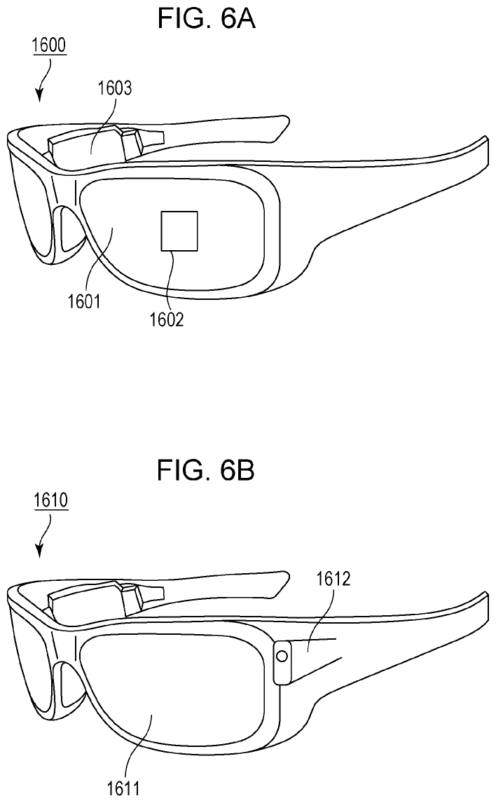

Application examples of the display apparatus according to each of the embodiments are described below with reference to FIGS. 6A and 6B. The display apparatus can be applied to a system that can be worn as a wearable device, such as smart glasses, a head-mounted display (HMD), or smart contact lenses. An imaging and displaying apparatus used in such an application includes an imaging apparatus that can photoelectrically convert visible light and a display apparatus that can emit visible light.

FIG. 6A illustrates glasses 1600 (smart glasses) according to one application example. An imaging apparatus 1602, such as a complementary metal-oxide semiconductor (CMOS) sensor or a single-photon avalanche photodiode (SPAD), is provided on the front side of a lens 1601 of the glasses 1600. The display apparatus according to one of the embodiments is provided on the back side of the lens 1601.

The glasses 1600 further include a controller 1603. The controller 1603 functions as a power supply for supplying power to the imaging apparatus 1602 and the display apparatus according to one of the embodiments. The controller 1603 controls the operation of the imaging apparatus 1602 and the display apparatus. The lens 1601 has an optical system for focusing light on the imaging apparatus 1602.

FIG. 6B illustrates glasses 1610 (smart glasses) according to one application example. The glasses 1610 have a controller 1612, which includes an imaging apparatus corresponding to the imaging apparatus 1602 and a display apparatus. A lens 1611 includes an optical system for projecting light from the imaging apparatus of the controller 1612 and the display apparatus, and an image is projected on the lens 1611. The controller 1612 functions as a power supply for supplying power to the imaging apparatus and the display apparatus and controls the operation of the imaging apparatus and the display apparatus. The controller may include a line-of-sight detection unit for detecting the line of sight of the wearer. Infrared radiation may be used to detect the line of sight. An infrared radiation unit emits infrared light to an eyeball of a user who is gazing at a display image. Reflected infrared light from the eyeball is detected by an imaging unit including a light-receiving element to capture an image of the eyeball. A reduction unit for reducing light from the infrared radiation unit to a display unit in a plan view is provided to reduce degradation in image quality.

The line of sight of the user for the display image is detected from the image of the eyeball captured by infrared imaging. Any known technique can be applied to line-of-sight detection using the image of the eyeball. For example, it is possible to use a line-of-sight detection method based on a Purkinje image obtained by reflection of irradiation light by the cornea.

More specifically, a line-of-sight detection process based on a pupil-corneal reflection method is performed. The line of sight of the user is detected by calculating a line-of-sight vector representing the direction (rotation angle) of an eyeball on the basis of an image of a pupil and a Purkinje image included in a captured image of the eyeball using the pupil-corneal reflection method.

A display apparatus according to an embodiment of the present disclosure may include an imaging apparatus including a light-receiving element and may control a display image on the basis of line-of-sight information of a user from the imaging apparatus.

More specifically, on the basis of the line-of-sight information, the display apparatus determines a first visibility region at which the user gazes and a second visibility region other than the first visibility region. The first visibility region and the second visibility region may be determined by the controller of the display apparatus or may be received from an external controller. In the display region of the display apparatus, the first visibility region may be controlled to have higher display resolution than the second visibility region. In other words, the second visibility region may have lower resolution than the first visibility region.

The display region has a first display region and a second display region different from the first display region, and the priority of the first display region and the second display region depends on the line-of-sight information. The first visibility region and the second visibility region may be determined by the controller of the display apparatus or may be received from an external controller. A region with a higher priority may be controlled to have higher resolution than another region. In other words, a region with a lower priority may have lower resolution.

The first visibility region or a region with a higher priority may be determined by artificial intelligence (AI). The AI may be a model configured to estimate the angle of the line of sight and the distance to a target ahead of the line of sight from an image of an eyeball using the image of the eyeball and the direction in which the eyeball actually viewed in the image as teaching data. The AI program may be stored in the display apparatus, the imaging apparatus, or an external device. The AI program stored in an external device is transmitted to the display apparatus via communication.

For display control based on visual recognition detection, the present disclosure can be applied to smart glasses further having an imaging apparatus for imaging the outside. Smart glasses can display captured external information in real time.

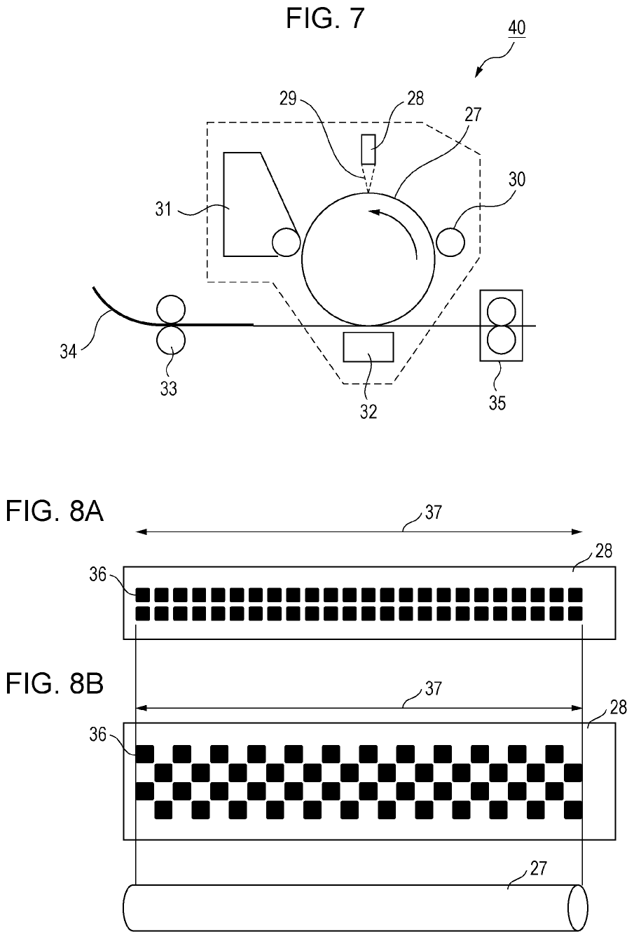

FIG. 7 is a schematic view of an example of an image-forming apparatus according to the present embodiment. An image-forming apparatus 40 is an electrophotographic image-forming apparatus and includes a photosensitive unit 27, an exposure light source 28, a charging unit 30, a developing unit 31, a transfer unit 32, a conveying roller 33, and a fixing unit 35. The exposure light source 28 emits light 29, and an electrostatic latent image is formed on the surface of the photosensitive unit 27. The exposure light source 28 includes the organic light-emitting element according to the present embodiment. The developing unit 31 contains toner and the like. The charging unit 30 electrifies the photosensitive unit 27. The transfer unit 32 transfers a developed image onto a recording medium 34. The conveying roller 33 conveys the recording medium 34. The recording medium 34 is paper, for example. The fixing unit 35 fixes an image on the recording medium 34.

FIGS. 8A and 8B are schematic views of the exposure light source 28, wherein a plurality of light-emitting portions 36 are arranged on a long substrate. An arrow 37 indicates a longitudinal direction in which the organic light-emitting elements are arranged. The longitudinal direction is the same as the direction of the rotation axis of the photosensitive unit 27. This direction can also be referred to as the major-axis direction of the photosensitive unit 27. In FIG. 8A, the light-emitting portions 36 are arranged in the major-axis direction of the photosensitive unit 27. In FIG. 8B, unlike FIG. 8A, the light-emitting portions 36 are alternately arranged in the longitudinal direction in the first and second rows. The first row and the second row are arranged at different positions in the transverse direction. In the first row, the light-emitting portions 36 are arranged at intervals. In the second row, the light-emitting portions 36 are arranged at positions corresponding to the spaces between the light-emitting portions 36 of the first row. Thus, the light-emitting portions 36 are also arranged at intervals in the transverse direction. The arrangement in FIG. 8B can also be referred to as a grid-like pattern, a staggered pattern, or a checkered pattern, for example.

As described above, an apparatus including the organic light-emitting element according to the present embodiment can be used to stably display a high-quality image for extended periods.

### EXAMPLES

The present disclosure is described below with exemplary embodiments. However, the present disclosure is not limited these exemplary embodiments.

### Exemplary Embodiment 1 (Synthesis of Exemplary Compounds A25 and A35)

Exemplary compounds A25 and A35 were synthesized by the following synthesis scheme.

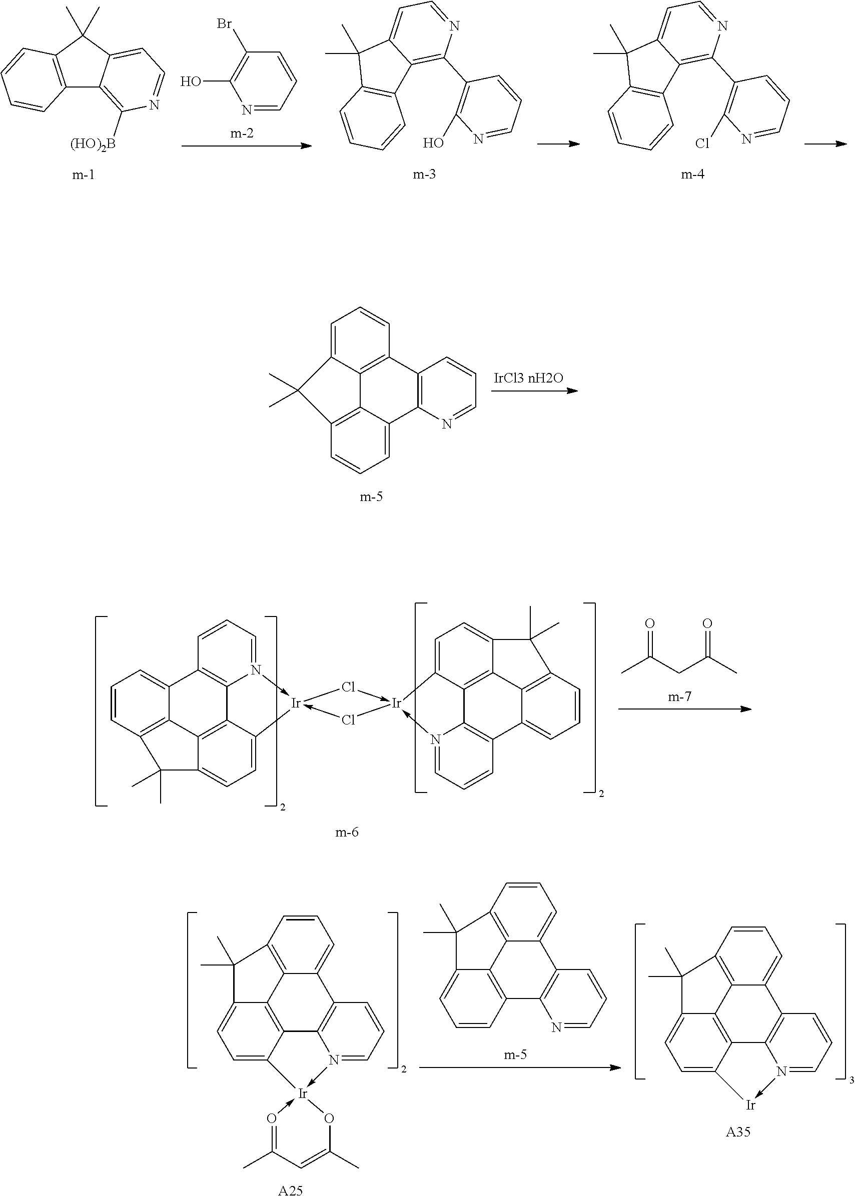

### (1) Synthesis of Compound m-3

A 200-ml recovery flask was charged with the following reagents and solvents.
Compound m-1: 4.0 g (16.8 mmol)
Compound m-2: 3.2 g (18.5 mmol)
Pd(PPh₃)₄ : 0.19 g
Toluene: 20 ml
Ethanol: 10 ml
2 M aqueous sodium carbonate: 20 ml

The reaction solution was then heated and stirred under reflux in a nitrogen stream for 6 hours. After completion of the reaction, water was added to the product, and liquid separation was performed. The resulting product was then dissolved in chloroform and was purified by column chromatography (chloroform). Recrystallization from chloroform/methanol gave 3.7 g (yield: 76%) of a compound m-3 as a pale yellow solid.

### (2) Synthesis of Compound m-4

A 200-ml recovery flask was charged with the following reagent and solvent.
Compound m-3: 3.5 g (12.2 mmol)
Phosphorus oxychloride: 105 ml

The reaction solution was then heated to 130° C. in a nitrogen stream and was stirred for 3 days. After completion of the reaction, water was added to the product, and liquid separation was performed. The resulting product was then dissolved in chloroform and was purified by column chromatography (chloroform). Recrystallization from chloroform/methanol gave 2.0 g (yield: 55%) of a compound m-4 as a pale yellow solid.

### (3) Synthesis of Compound m-5

A 200-ml recovery flask was charged with the following reagents and solvent.
Compound m-4: 2.0 g (6.5 mmol)
Pd(dba)₂: 0.23 g
P(Cy)₃-HBF₄: 0.29 g
DMAc: 20 ml
Potassium carbonate: 2.7 g (19.6 mmol)

The reaction solution was then heated to 150° C. in a nitrogen stream and was stirred for 6 hours. After completion of the reaction, water was added to the product, and liquid separation was performed. The resulting product was then dissolved in chloroform and was purified by column chromatography (chloroform). Recrystallization from chloroform/methanol gave 0.49 g (yield: 28%) of a compound m-5 as a pale yellow solid.

### (4) Synthesis of Compound m-6

A 200-ml recovery flask was charged with the following reagents and solvent.
2-ethoxyethanol: 12 ml
Iridium (III) chloride hydrate: 0.19 g
Compound m-5: 0.4 g (1.5 mmol)

The reaction solution was then heated to 120° C. and was stirred for 6 hours. After cooling, water was added to the product, and the product was filtered and washed with water. Drying the product gave 0.5 g (yield: 90%) of a compound m-6 as a yellow solid.

### (5) Synthesis of Exemplary Compound A25

A 200-ml recovery flask was charged with the following reagents and solvent.
2-ethoxyethanol: 30 ml
Compound m-6: 0.5 g (0.3 mmol)
Compound m-7: 0.13 g (1.3 mmol)
Sodium carbonate: 0.3 g (3.3 mmol)

The reaction solution was then heated to 100° C. and was stirred for 6 hours. After cooling, methanol was added to the product, and the product was filtered and washed with methanol. Drying the product gave 0.3 g (yield: 63%) of an exemplary compound A25 as a yellow solid.

The exemplary compound A25 was subjected to mass spectrometry with MALDI-TOF-MS (Autoflex LRF manufactured by Bruker).

[MALDI-TOF-MS]

Actual value: m/z=828 calculated value: C₄₅H₃₅IrN₂O₂=828

### (6) Synthesis of Exemplary Compound A35

A 50-ml recovery flask was charged with the following reagents and solvent.
Exemplary compound A25: 0.2 g (0.2 mmol)
Compound m-5: 0.7 g (2.4 mmol)
Glycerol: 15 ml

The reaction solution was then heated to 230° C. and was stirred for 3 hours. After cooling to 100° C., 2 mL of toluene was added to the product, which was then cooled to room temperature with stirring. Heptane was then added to the product, which was then filtered. The filter residue was purified by silica gel column chromatography (ethyl acetate), yielding 0.06 g (yield: 24%) of the exemplary compound A35 as a dark yellow solid.

The exemplary compound A35 was subjected to mass spectrometry with MALDI-TOF-MS (Autoflex LRF manufactured by Bruker).

[MALDI-TOF-MS]

Actual value: m/z=997 calculated value: C₆₀H₄₂IrN₃=997

### Exemplary Embodiments 2 to 7 (Synthesis of Exemplary Compounds)

As shown in Table 4, exemplary compounds of Exemplary Embodiments 2 to 7 were synthesized in the same manner as in Exemplary Embodiment 1 except that the raw materials m-1, m-2, and m-7 of Exemplary Embodiment 1 were changed. Actual values m/z measured by mass spectrometry in the same manner as in Exemplary Embodiment 1 are also shown.

TABLE 4

| Exemplary | Exemplary | Raw material | Raw material | Raw material | |
|---|---|---|---|---|---|
| embodiment | compound | m-1 | m-2 | m-7 | m/z |
| 2 | C17 | 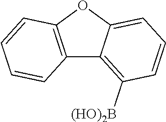 [SMILES: OB(O)c1cccc2oc3ccccc3c12] | 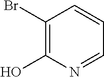 | 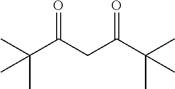 | 860 |
| 3 | B12 | 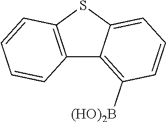 [SMILES: OB(O)c1cccc2sc3ccccc3c12] | 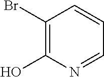 | 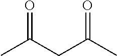 | 808 |
| 4 | C5 | 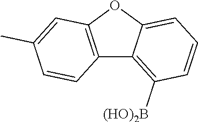 [SMILES: Cc1ccc2c(c1)oc1cccc(B(O)O)c12] | 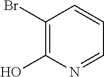 | 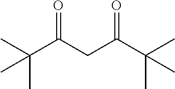 | 888 |
| 5 | K9 | 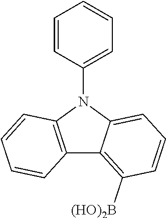 [SMILES: OB(O)c1cccc2c1c1ccccc1n2-c1ccccc1] | 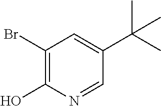 | 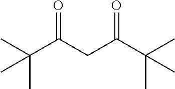 | 1122 |
| 6 | D27 | 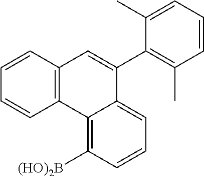 [SMILES: Cc1cccc(C)c1-c1cc2ccccc2c2c(B(O)O)cccc12] | 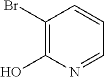 | 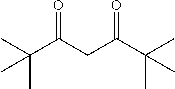 | 1088 |
| 7 | B14 | 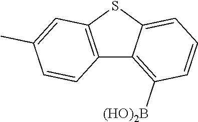 [SMILES: Cc1ccc2c(c1)sc1cccc(B(O)O)c12] | 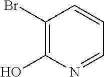 | 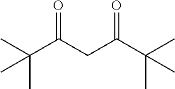 | 920 |

### Exemplary Embodiment 8 (Synthesis of Exemplary Compounds E29 and E33)

Exemplary compounds E29 and E33 were synthesized by the following synthesis scheme.

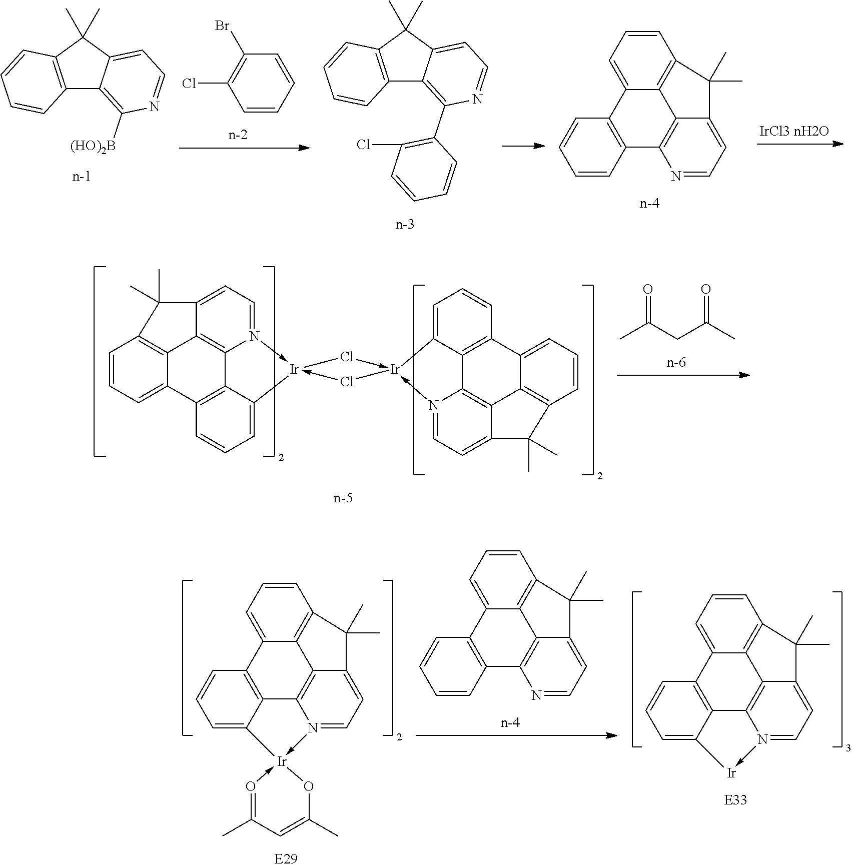

### (1) Synthesis of Compound n-3

A 200-m1 recovery flask was charged with the following reagents and solvents.
Compound n-1: 4.0 g (16.7 mmol)
Compound n-2: 3.5 g (18.4 mmol)
Pd(PPh3)4: 0.19 g
Toluene: 20 ml
Ethanol: 10 ml
2 M aqueous sodium carbonate: 20 ml

The reaction solution was then heated and stirred under reflux in a nitrogen stream for 6 hours. After completion of the reaction, water was added to the product, and liquid separation was performed. The resulting product was then dissolved in chloroform and was purified by column chromatography (chloroform). Recrystallization from chloroform/methanol gave 3.3 g (yield: 64%) of a compound n-3 as a pale yellow solid.

### (3) Synthesis of Compound m-4

A 200-ml recovery flask was charged with the following reagents and solvent.
Compound n-3: 3.0 g (9.8 mmol)
P(dba)₂: 0.34 g
P(Cy)₃-HBF4: 0.43 g
DMAc: 30 ml
Potassium carbonate: 4.1 g (29.4 mmol)

The reaction solution was then heated to 150° C. in a nitrogen stream and was stirred for 6 hours. After completion of the reaction, water was added to the product, and liquid separation was performed. The resulting product was then dissolved in chloroform and was then purified by column chromatography (chloroform). Recrystallization from chloroform/methanol gave 0.8 g (yield: 29%) of a compound n-4 as a pale yellow solid.

### (4) Synthesis of Compound n-5

A 200-ml recovery flask was charged with the following reagents and solvent.
2-ethoxyethanol: 24 ml
Iridium (III) chloride hydrate: 0.32 g
Compound n-4: 0.7 g (2.6 mmol)

The reaction solution was then heated to 120° C. and was stirred for 6 hours. After cooling, water was added to the product, and the product was filtered and washed with water. Drying the product gave 0.9 g (yield: 89%) of a compound n-5 as a yellow solid.

### (5) Synthesis of Exemplary Compound E29

A 200-ml recovery flask was charged with the following reagents and solvent.
2-ethoxyethanol: 30 ml
Compound n-5: 0.8 g (0.6 mmol)
Compound n-6: 0.2 g (2.5 mmol)
Sodium carbonate: 0.6 g (6.3 mmol)

The reaction solution was then heated to 100° C. and was stirred for 6 hours. After cooling, methanol was added to the product, and the product was filtered and washed with methanol. Drying the product gave 0.5 g (yield: 61%) of an exemplary compound E29 as a yellow solid.

The exemplary compound E29 was subjected to mass spectrometry with MALDI-TOF-MS (Autoflex LRF manufactured by Bruker).

[MALDI-TOF-MS]

Actual value: m/z=828 calculated value: C₄₅H₃₅IrN₂O₂=828

### (6) Synthesis of Exemplary Compound E33

A 50-ml recovery flask was charged with the following reagents and solvent.
Exemplary compound E29: 0.5 g (0.5 mmol)
Compound n-4: 1.6 g (6.0 mmol)
Glycerol: 15 ml

The reaction solution was then heated to 230° C. and was stirred for 3 hours. After cooling to 100° C., 2 mL of toluene was added to the product, which was then cooled to room temperature with stirring. Heptane was then added to the product, which was then filtered. The filter residue was purified by silica gel column chromatography (ethyl acetate), yielding 0.1 g (yield: 22%) of a dark yellow solid E33.

The exemplary compound E33 was subjected to mass spectrometry with MALDI-TOF-MS (Autoflex LRF manufactured by Bruker).

[MALDI-TOF-MS]

Actual value: m/z=997 calculated value: C₆₀H₄₂IrN₃=997

### Exemplary Embodiments 9 to 16 (Synthesis of Exemplary Compounds)

As shown in Table 5, exemplary compounds of Exemplary Embodiments 9 to 16 were synthesized in the same manner as in Exemplary Embodiment 8 except that the raw materials n-1, n-2, and n-6 of Exemplary Embodiment 8 were changed. Actual values m/z measured by mass spectrometry in the same manner as in Exemplary Embodiment 8 are also shown.

TABLE 5

| Exemplary | Exemplary | | | | |
|---|---|---|---|---|---|
| embodiment | compound | Raw material n-1 | Raw material n-2 | Raw material n-6 | m/z |
| 9 | G27 | 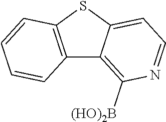 [SMILES: OB(O)c1nccc2sc3ccccc3c12] | 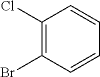 | 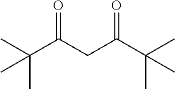 | 860 |
| 10 | G25 | 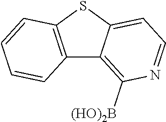 [SMILES: OB(O)c1nccc2sc3ccccc3c12] | 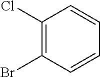 | 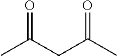 | 775 |
| 11 | G28 | 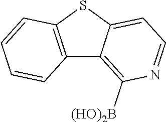 [SMILES: OB(O)c1nccc2sc3ccccc3c12] | 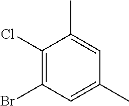 | 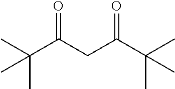 | 916 |
| 12 | F25 | 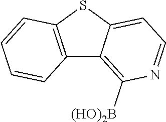 [SMILES: OB(O)c1nccc2sc3ccccc3c12] | 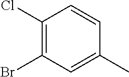 | 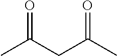 | 836 |
| 13 | H27 | 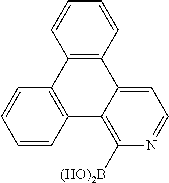 [SMILES: OB(O)c1nccc2c3ccccc3c3ccccc3c12] | 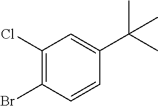 | 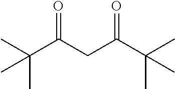 | 1092 |
| 14 | E26 | 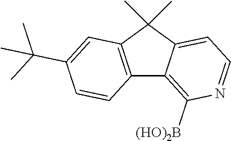 [SMILES: CC(C)(C)c1ccc2c(c1)C(C)(C)c1ccnc(B(O)O)c1-2] | 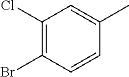 | 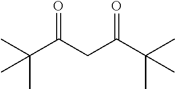 | 1052 |
| 15 | H34 | 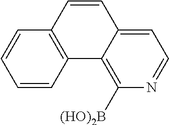 [SMILES: OB(O)c1nccc2ccc3ccccc3c12] | 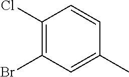 | 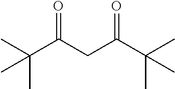 | 908 |
| 16 | H35 | 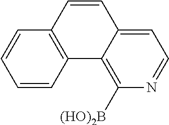 [SMILES: OB(O)c1nccc2ccc3ccccc3c12] | 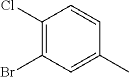 | 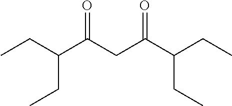 | 936 |

### Exemplary Embodiments 17 to 25 (Synthesis of Exemplary Compounds)

As shown in Table 6, exemplary compounds of Exemplary Embodiments 17 to 21 were synthesized in the same manner as in Exemplary Embodiment 1 except that the raw materials m-1, m-2, and m-5 of Exemplary Embodiment 1 were changed. As shown in Table 6, exemplary compounds of Exemplary Embodiments 22 to 25 were synthesized in the same manner as in Exemplary Embodiment 8 except that the raw materials n-1, n-2, and n-4 of Exemplary Embodiment 8 were changed. Actual values m/z measured by mass spectrometry in the same manner as in Exemplary Embodiments 1 and 8 are also shown.

TABLE 6

| Exemplary | Exemplary | Raw material | Raw material | Raw material | |
|---|---|---|---|---|---|
| embodiment | compound | m-1/n-1 | m-2/n-2 | m-7/n-4 | m/z |
| 17 | A36 | 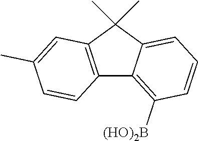 [SMILES: Cc1ccc2c(c1)C(C)(C)c1cccc(B(O)O)c1-2] | 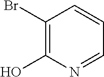 | 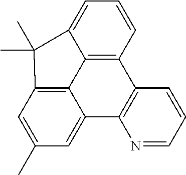 [SMILES: Cc1cc2c3c(c1)c1ncccc1c1cccc(c13)C2(C)C] | 1039 |
| 18 | C39 | 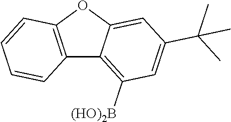 [SMILES: CC(C)(C)c1cc(B(O)O)c2c(c1)oc1ccccc12] | 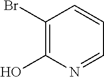 | 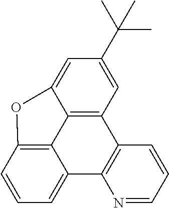 [SMILES: CC(C)(C)c1cc2oc3cccc4c5ncccc5c(c1)c2c34] | 1087 |
| 19 | B39 | 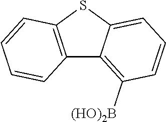 [SMILES: OB(O)c1cccc2sc3ccccc3c12] | 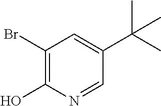 | 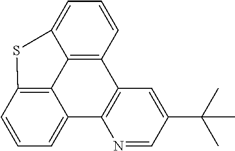 [SMILES: CC(C)(C)c1cnc2c(c1)c1cccc3sc4cccc2c4c31] | 1135 |
| 20 | D33 | 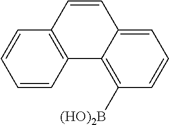 [SMILES: OB(O)c1cccc2ccc3ccccc3c12] | 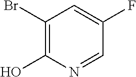 | 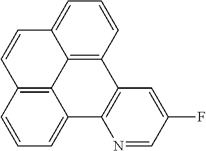 [SMILES: Fc1cnc2c(c1)c1cccc3ccc4cccc2c4c31] | 1003 |
| 21 | C38 | 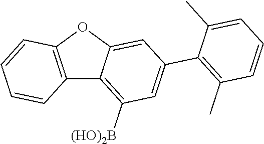 [SMILES: Cc1cccc(C)c1-c1cc(B(O)O)c2c(c1)oc1ccccc12] | 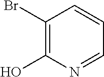 | 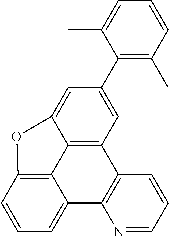 [SMILES: Cc1cccc(C)c1-c1cc2oc3cccc4c5ncccc5c(c1)c2c34] | 1231 |
| 22 | F34 | 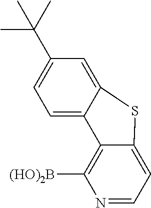 [SMILES: CC(C)(C)c1ccc2c(c1)sc1ccnc(B(O)O)c12] | 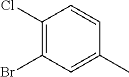 | 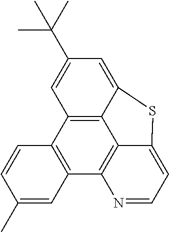 [SMILES: Cc1ccc2c(c1)c1nccc3sc4cc(C(C)(C)C)cc2c4c31] | 1176 |
| 23 | G35 | 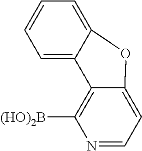 [SMILES: OB(O)c1nccc2oc3ccccc3c12] | 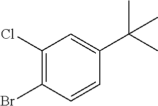 | 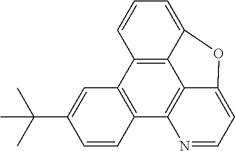 [SMILES: CC(C)(C)c1ccc2c(c1)c1cccc3oc4ccnc2c4c31] | 1087 |
| 24 | H33 | 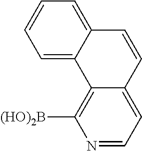 [SMILES: OB(O)c1nccc2ccc3ccccc3c12] | 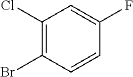 | 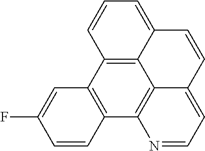 [SMILES: Fc1ccc2c(c1)c1cccc3ccc4ccnc2c4c31] | 1003 |
| 25 | E34 | 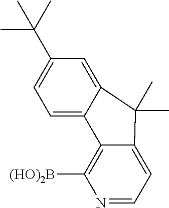 [SMILES: CC(C)(C)c1ccc2c(c1)C(C)(C)c1ccnc(B(O)O)c1-2] | 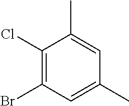 | 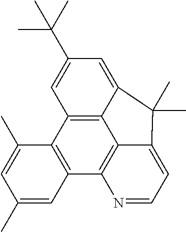 [SMILES: Cc1cc(C)c2c(c1)c1nccc3c1c1c(cc(C(C)(C)C)cc21)C3(C)C] | 1250 |

### Exemplary Embodiment 26 (Synthesis of Exemplary Compound A1)

An exemplary compound A1 was synthesized by the following synthesis scheme.

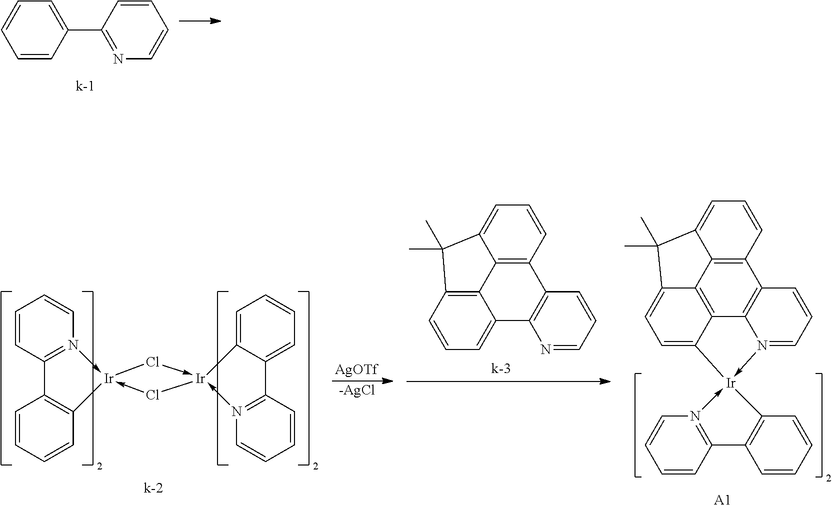

The synthesis of the compound k-2 is the same as (4) Synthesis of Compound m-6 of Exemplary Embodiment 1 and is not described here.

### (2) Synthesis of Exemplary Compound A1

A 200-ml recovery flask was charged with the following reagents and solvents.
Compound k-2: 1.0 g (0.9 mmol)
AgOTf: 0.5 g (1.9 mmol)
Dichloromethane: 50 ml
Methanol: 2 ml

The reaction solution was then stirred at room temperature for 6 hours. The solvent was then distilled off under reduced pressure, and a yellow solid was formed.

A 200-m1 recovery flask was charged with the yellow solid and the following reagent and solvent.
Ethanol: 30 ml
Compound k-3: 0.4 g (1.9 mmol)

The reaction solution was then heated to 85° C. and was stirred for 3 hours. After cooling, filtration was performed. The filter residue was purified by silica gel column chromatography (chloroform:heptane=1:1), yielding 0.7 g (yield: 52%) of a dark yellow solid A1.

The exemplary compound A1 was subjected to mass spectrometry with MALDI-TOF-MS (Autoflex LRF manufactured by Bruker).

[MALDI-TOF-MS]

Actual value: m/z=769 calculated value: C₄₂H₃₀IrN₃=769

### Exemplary Embodiments 27 to 43 (Synthesis of Exemplary Compounds)

As shown in Tables 7 and 8, exemplary compounds of Exemplary Embodiments 27 to 43 were synthesized in the same manner as in Exemplary Embodiment 26 except that the raw materials k-1 and k-3 of Exemplary Embodiment 26 were changed. Actual values m/z measured by mass spectrometry in the same manner as in Exemplary Embodiment 26 are also shown.

TABLE 7

| Exemplary | Exemplary | | | |
|---|---|---|---|---|
| embodiment | compound | Raw material k-1 | Raw material k-3 | m/z |
| 27 | C1 | 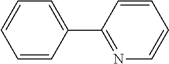 [SMILES: c1ccc(-c2ccccn2)cc1] | 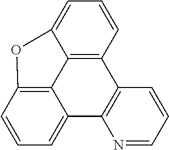 [SMILES: c1cnc2c(c1)c1cccc3oc4cccc2c4c31] | 743 |
| 28 | B30 | 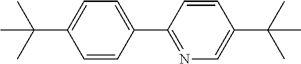 [SMILES: CC(C)(C)c1ccc(-c2ccc(C(C)(C)C)cn2)cc1] | 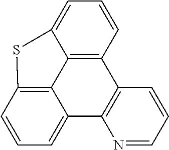 [SMILES: c1cnc2c(c1)c1cccc3sc4cccc2c4c31] | 983 |
| 29 | C31 | 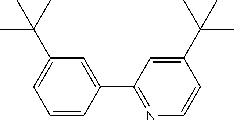 [SMILES: CC(C)(C)c1cccc(-c2cc(C(C)(C)C)ccn2)c1] | 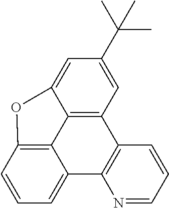 [SMILES: CC(C)(C)c1cc2oc3cccc4c5ncccc5c(c1)c2c34] | 1023 |
| 30 | A5 | 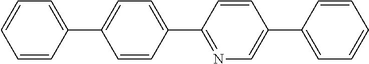 [SMILES: c1ccc(-c2ccc(-c3ccc(-c4ccccc4)cn3)cc2)cc1] | 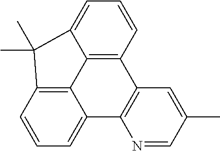 | 1087 |
| 31 | A7 | 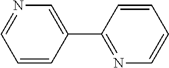 [SMILES: c1ccc(-c2cccnc2)nc1] | 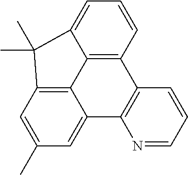 [SMILES: Cc1cc2c3c(c1)c1ncccc1c1cccc(c13)C2(C)C] | 785 |
| 32 | D8 | 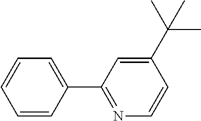 [SMILES: CC(C)(C)c1ccnc(-c2ccccc2)c1] | 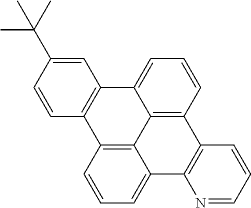 [SMILES: CC(C)(C)c1ccc2c(c1)c1cccc3c4cccnc4c4cccc2c4c13] | 971 |
| 33 | A8 | 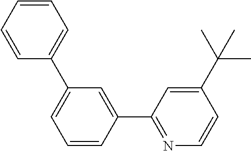 [SMILES: CC(C)(C)c1ccnc(-c2cccc(-c3ccccc3)c2)c1] | 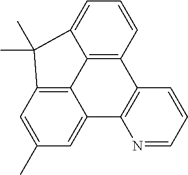 [SMILES: Cc1cc2c3c(c1)c1ncccc1c1cccc(c13)C2(C)C] | 1047 |
| 34 | A21 | 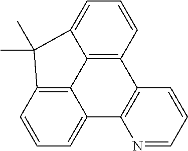 [SMILES: CC1(C)c2cccc3c4cccnc4c4cccc1c4c23] | 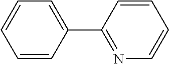 [SMILES: c1ccc(-c2ccccn2)cc1] | 883 |
| 35 | A22 | 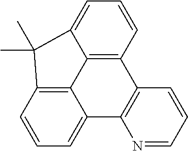 [SMILES: CC1(C)c2cccc3c4cccnc4c4cccc1c4c23] | 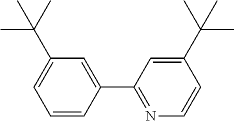 [SMILES: CC(C)(C)c1cccc(-c2cc(C(C)(C)C)ccn2)c1] | 995 |

TABLE 8

| Exemplary | Exemplary | | | |
|---|---|---|---|---|
| embodiment | compound | Raw material k-1 | Raw material k-3 | m/z |
| 36 | A23 | 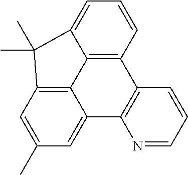 [SMILES: Cc1cc2c3c(c1)c1ncccc1c1cccc(c13)C2(C)C] | 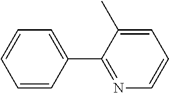 | 925 |
| 37 | E7 | 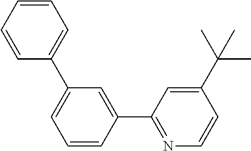 [SMILES: CC(C)(C)c1ccnc(-c2cccc(-c3ccccc3)c2)c1] | 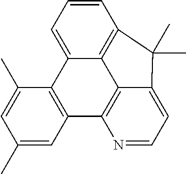 [SMILES: Cc1cc(C)c2c(c1)c1nccc3c1c1c(cccc21)C3(C)C] | 1089 |
| 38 | G6 | 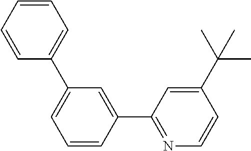 [SMILES: CC(C)(C)c1ccnc(-c2cccc(-c3ccccc3)c2)c1] | 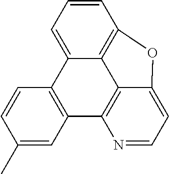 [SMILES: Cc1ccc2c(c1)c1nccc3oc4cccc2c4c31] | 1007 |
| 39 | F5 | 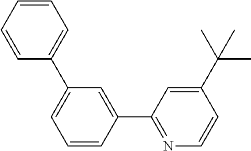 [SMILES: CC(C)(C)c1ccnc(-c2cccc(-c3ccccc3)c2)c1] | 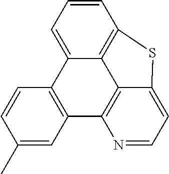 [SMILES: Cc1ccc2c(c1)c1nccc3sc4cccc2c4c31] | 1079 |
| 40 | H7 | 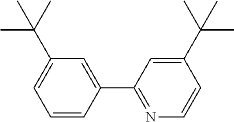 [SMILES: CC(C)(C)c1cccc(-c2cc(C(C)(C)C)ccn2)c1] | 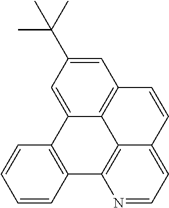 [SMILES: CC(C)(C)c1cc2ccc3ccnc4c5ccccc5c(c1)c2c34] | 1033 |
| 41 | E21 | 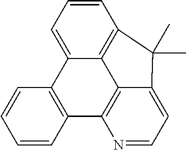 [SMILES: CC1(C)c2cccc3c4ccccc4c4nccc1c4c23] | 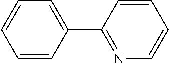 [SMILES: c1ccc(-c2ccccn2)cc1] | 883 |
| 42 | E22 | 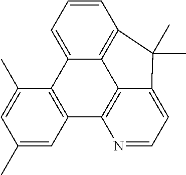 [SMILES: Cc1cc(C)c2c(c1)c1nccc3c1c1c(cccc21)C3(C)C] | 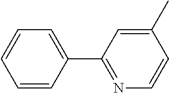 | 953 |
| 43 | E23 | 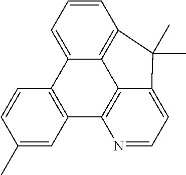 [SMILES: Cc1ccc2c(c1)c1nccc3c1c1c(cccc21)C3(C)C] | 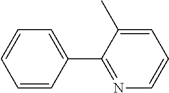 | 925 |

### Exemplary Embodiment 44

An organic light-emitting element of a bottom emission type was produced. The organic light-emitting element included a positive electrode, a hole-injection layer, a hole-transport layer, an electron-blocking layer, a light-emitting layer, a hole-blocking layer, an electron-transport layer, an electron-injection layer, and a negative electrode sequentially formed on a substrate.

First, an ITO film was formed on a glass substrate and was subjected to desired patterning to form an ITO electrode (positive electrode). The ITO electrode had a thickness of 100 nm. The substrate on which the ITO electrode was formed was used as an ITO substrate in the following process. Vacuum deposition was then performed by resistance heating in a vacuum chamber at 1.33×10⁻⁴ Pa to continuously form an organic compound layer and an electrode layer shown in Table 9 on the ITO substrate. The counter electrode (a metal electrode layer, a negative electrode) had an electrode area of 3 mm².

TABLE 9

| | | | | Ratio in light-emitting | |
|---|---|---|---|---|---|
| | | | Raw | layer | Thickness |
| | | | material | (mass %) | (nm) |
| Electrode layer | Negative electrode | | Al | — | 100 |
| Organic compound | Electron-injection layer (EIL) | | LiF | — | 1 |
| layer | Electron-transport layer (ETL) | | ET2 | — | 20 |
| | Hole-blocking layer (HBL) | | ET11 | — | 20 |
| | Light-emitting layer | Host | BB37 | 90 | 20 |
| | (EML) | Guest | A1 | 10 | |

| | | | |
|---|---|---|---|
| Electron-blocking layer (EBL) | HT19 | — | 15 |
| Hole-transport layer (HTL) | HT3 | — | 30 |
| Hole-injection layer (HIL) | HT16 | — | 5 |

The characteristics of the element were measured and evaluated. The light-emitting element had a maximum emission wavelength of 522 nm and a maximum external quantum efficiency (E.Q.E.) of 12%. A continuous operation test was performed at a current density of 100 mA/cm² to measure the time (LT95) when the luminance degradation rate reached 5%. Assuming that the time (LT95) when the luminance degradation rate of Comparative Example 1 reached 5% was 1.0, the LT95 (relative value) of the present exemplary embodiment was 1.4.

In the present exemplary embodiment, with respect to measuring apparatuses, more specifically, the current-voltage characteristics were measured with a microammeter 4140B manufactured by Hewlett-Packard Co., and the luminous brightness was measured with a BM7 manufactured by Topcon Corporation.

### [Exemplary Embodiments 45 to 68, Comparative Examples 1 and 2]

Organic light-emitting elements were produced in the same manner as in Exemplary Embodiment 44 except that the materials for forming each layer were appropriately changed to the compounds shown in Table 10. A layer not shown in Table 10 had the same structure as in Exemplary Embodiment 44. The characteristics of the elements were measured and evaluated in the same manner as in Exemplary Embodiment 44. Table 10 shows the measurement results together with the results of Exemplary Embodiment 44.

TABLE 10

| | | | | EML | | | | E.Q.E | LT95 |
|---|---|---|---|---|---|---|---|---|---|
| | HIL | HTL | EBL | Host | Guest | HBL | ETL | [%] | [—] |
| Exemplary embodiment 44 | HT16 | HT3 | HT19 | BB38 | A1 | ET11 | ET2 | 12 | 1.4 |
| Exemplary embodiment 45 | HT16 | HT3 | HT19 | BB19 | A8 | ET12 | ET15 | 13 | 1.5 |
| Exemplary embodiment 46 | HT16 | HT2 | HT15 | BB18 | A10 | ET12 | ET2 | 14 | 1.4 |
| Exemplary embodiment 47 | HT16 | HT2 | HT15 | CC19 | A16 | ET11 | ET2 | 13 | 1.4 |
| Exemplary embodiment 48 | HT16 | HT3 | HT19 | CC8 | A29 | ET12 | ET15 | 10 | 1.1 |
| Exemplary embodiment 49 | HT16 | HT3 | HT19 | AA7 | A33 | ET12 | ET15 | 10 | 1.1 |
| Exemplary embodiment 50 | HT16 | HT3 | HT19 | BB29 | B6 | ET11 | ET15 | 12 | 1.4 |
| Exemplary embodiment 51 | HT16 | HT3 | HT19 | BB19 | B31 | ET12 | ET2 | 13 | 1.5 |
| Exemplary embodiment 52 | HT16 | HT2 | HT15 | BB8 | B35 | ET12 | ET15 | 13 | 1.4 |
| Exemplary embodiment 53 | HT16 | HT3 | HT19 | BB20 | D25 | ET12 | ET15 | 12 | 1.3 |
| Exemplary embodiment 54 | HT16 | HT2 | HT15 | EM16 | C5 | ET11 | ET2 | 11 | 1.1 |
| Exemplary embodiment 55 | HT16 | HT3 | HT19 | BB19 | C31 | ET12 | ET15 | 14 | 1.3 |
| Exemplary embodiment 56 | HT16 | HT2 | HT15 | BB18 | D27 | ET12 | ET2 | 10 | 1.2 |
| Exemplary embodiment 57 | HT16 | HT2 | HT15 | CC19 | E1 | ET11 | ET2 | 14 | 1.4 |
| Exemplary embodiment 58 | HT16 | HT3 | HT19 | CC8 | E2 | ET12 | ET15 | 15 | 1.3 |
| Exemplary embodiment 59 | HT16 | HT3 | HT19 | AA7 | E29 | ET12 | ET15 | 11 | 1.2 |
| Exemplary embodiment 60 | HT16 | HT3 | HT19 | BB23 | E34 | ET11 | ET15 | 12 | 1.2 |
| Exemplary embodiment 61 | HT16 | HT3 | HT19 | AA7 | F1 | ET12 | ET15 | 12 | 1.4 |
| Exemplary embodiment 62 | HT16 | HT3 | HT19 | BB23 | F7 | ET11 | ET15 | 13 | 1.4 |
| Exemplary embodiment 63 | HT16 | HT3 | HT19 | BB19 | F27 | ET12 | ET2 | 13 | 1.2 |
| Exemplary embodiment 64 | HT16 | HT2 | HT15 | BB8 | G6 | ET12 | ET15 | 13 | 1.4 |
| Exemplary embodiment 65 | HT16 | HT3 | HT19 | BB20 | H36 | ET12 | ET15 | 12 | 1.3 |
| Exemplary embodiment 66 | HT16 | HT2 | HT15 | EM16 | G27 | ET11 | ET2 | 10 | 1.1 |
| Exemplary embodiment 67 | HT16 | HT3 | HT19 | BB19 | G36 | ET12 | ET15 | 10 | 1.2 |
| Exemplary embodiment 68 | HT16 | HT2 | HT15 | BB18 | H27 | ET12 | ET2 | 10 | 1.2 |
| Comparative example 1 | HT16 | HT3 | HT19 | BB37 | Comparative | ET11 | ET2 | 8 | 1 |
| | | | | | compound 1 | | | | |
| Comparative example 2 | HT16 | HT3 | HT19 | EM33 | Comparative | ET11 | ET2 | 9 | 0.8 |
| | | | | | compound 1 | | | | |

Table 10 shows that Comparative Examples 1 and 2 had a maximum external quantum efficiency (E.Q.E.) in the range of 8% to 9%, and Exemplary Embodiments 44 to 68 had a maximum external quantum efficiency in the range of 10% to 15%. Thus, the organic light-emitting elements of Exemplary Embodiments 44 to 68 had higher luminescence efficiency. This is probably because each organic compound contained in the organic light-emitting elements of Exemplary Embodiments 44 to 68 as a guest in the light-emitting layer has a higher quantum yield than the organic compound contained in the organic light-emitting elements of Comparative Examples 1 and 2 as a guest in the light-emitting layer (comparative compound 1). The comparative compound 1 is a compound in which an ancillary ligand of the compound 1-b described in PTL 1 is changed from acetylacetone to phenylpyridine. Each organic compound contained in the organic light-emitting elements of Exemplary Embodiments 44 to 68 as a guest in the light-emitting layer has a ring structure having bridged carbon atoms constituting the dibenzo[f,h]quinoline skeleton. This results in a high radiative decay rate due to good CT properties and transition dipole moment, and a low non-radiative decay rate due to high rigidity. This results in a high quantum yield of each organic compound. Thus, it is thought that the organic light-emitting elements of Exemplary Embodiments 44 to 68 exhibited high luminescence efficiency.

Table 10 shows that Exemplary Embodiments that contained an organic compound with the partial structure IrL represented by the general formula [C-1] or [C-2] as a guest in the light-emitting layer (Exemplary Embodiments 45, 51 to 53, 58, 60, 64, and 65) had higher maximum external quantum efficiency. This is probably because, in the aromatic ring σ-bonded to the Ir metal, a carbon atom adjacent to the carbon atom σ-bonded to the Ir metal has a methyl group, which improved the balance between the MLCT properties and the π-π* properties of the ligand.

Table 10 also shows that Exemplary Embodiments 44 to 68 had a longer LT95 and a longer life (higher durability) than the organic light-emitting elements of Comparative Examples 1 and 2. This is probably because each organic compound contained in the organic light-emitting elements of Exemplary Embodiments 44 to 68 as a guest in the light-emitting layer had a ring structure having bridged carbon atoms constituting the dibenzo[f,h]quinoline skeleton, thus resulting in the ligand of lower symmetry and high sublimability. Thus, it is thought that each organic compound had high stability during sublimation purification or vapor deposition, and a high-purity evaporated film could be produced. Thus, the organic light-emitting element had a long life.

### Exemplary Embodiment 69

An organic light-emitting element was produced in the same manner as in Exemplary Embodiment 44 except that the organic compound layer and the electrode layer shown in Table 11 were continuously formed.

TABLE 11

| | | | | Ratio in light-emitting | |
|---|---|---|---|---|---|
| | | | Raw | layer | Thickness |
| | | | material | (mass %) | (nm) |
| Electrode layer | Negative electrode | | Al | — | 100 |
| Organic compound | Electron-injection layer (EIL) | | LiF | — | 1 |
| layer | Electron-transport layer (ETL) | | ET2 | — | 20 |
| | Hole-blocking layer (HBL) | | ET11 | — | 20 |
| | Light-emitting layer | Host | BB37 | 60 | 20 |
| | (EML) | Guest | A8 | 10 | |
| | | Assist | EM30 | 30 | |

| | | | |
|---|---|---|---|
| Electron-blocking layer (EBL) | HT19 | — | 15 |
| Hole-transport layer (HTL) | HT3 | — | 30 |
| Hole-injection layer (HIL) | HT16 | — | 5 |

The characteristics of the element were measured and evaluated. The light-emitting element had a green emission color and a maximum external quantum efficiency (E.Q.E.) of 19%.

### Exemplary Embodiments 70 to 100

Organic light-emitting elements were produced in the same manner as in Exemplary Embodiment 69 except that the materials for forming each layer were appropriately changed to the compounds shown in Table 12. A layer not shown in Table 12 had the same structure as in Exemplary Embodiment 69. The characteristics of the elements were measured and evaluated in the same manner as in Exemplary Embodiment 69. Table 12 shows the measurement results together with the results of Exemplary Embodiment 69.

TABLE 12

| | | | | EML | | | | | E.Q.E |
|---|---|---|---|---|---|---|---|---|---|
| | HIL | HTL | EBL | Host | Guest | Assist | HBL | ETL | [%] |
| Exemplary embodiment 69 | HT16 | HT3 | HT19 | BB37 | A8 | EM30 | ET11 | ET2 | 19 |
| Exemplary embodiment 70 | HT16 | HT3 | HT19 | BB19 | A6 | EM29 | ET26 | ET3 | 17 |
| Exemplary embodiment 71 | HT16 | HT2 | HT15 | BB18 | A26 | EM35 | ET13 | ET2 | 15 |
| Exemplary embodiment 72 | HT16 | HT2 | HT15 | CC19 | A13 | EM37 | ET13 | ET2 | 17 |
| Exemplary embodiment 73 | HT16 | HT3 | HT19 | CC18 | A36 | AA16 | ET16 | ET15 | 16 |
| Exemplary embodiment 74 | HT16 | HT3 | HT19 | AA7 | H34 | AA5 | ET16 | ET15 | 15 |
| Exemplary embodiment 75 | HT16 | HT3 | HT19 | BB28 | B6 | A16 | ET17 | ET15 | 16 |
| Exemplary embodiment 76 | HT16 | HT3 | HT19 | BB19 | B12 | EM40 | ET13 | ET2 | 15 |
| Exemplary embodiment 77 | HT16 | HT2 | HT15 | BB29 | B31 | EM28 | ET15 | ET3 | 19 |
| Exemplary embodiment 78 | HT16 | HT3 | HT19 | BB19 | B35 | ET15 | ET15 | ET15 | 17 |
| Exemplary embodiment 79 | HT16 | HT2 | HT15 | CC17 | B17 | ET17 | ET2 | ET2 | 16 |
| Exemplary embodiment 80 | HT16 | HT3 | HT19 | BB19 | C3 | EM29 | ET26 | ET3 | 16 |
| Exemplary embodiment 81 | HT16 | HT2 | HT15 | BB18 | C4 | EM30 | ET13 | ET2 | 17 |
| Exemplary embodiment 82 | HT16 | HT2 | HT15 | CC19 | C35 | EM31 | ET13 | ET2 | 18 |
| Exemplary embodiment 83 | HT16 | HT3 | HT19 | CC18 | C34 | AA16 | ET16 | ET15 | 17 |
| Exemplary embodiment 84 | HT16 | HT3 | HT19 | AA7 | D8 | AA5 | ET16 | ET15 | 14 |
| Exemplary embodiment 85 | HT16 | HT2 | HT15 | BB29 | C39 | EM28 | ET15 | ET3 | 14 |
| Exemplary embodiment 86 | HT16 | HT3 | HT19 | BB19 | D12 | ET15 | ET15 | ET15 | 15 |
| Exemplary embodiment 87 | HT16 | HT2 | HT15 | CC17 | E5 | ET17 | ET2 | ET2 | 17 |
| Exemplary embodiment 88 | HT16 | HT3 | HT19 | BB19 | E7 | EM29 | ET26 | ET3 | 18 |
| Exemplary embodiment 89 | HT16 | HT2 | HT15 | BB18 | E36 | EM30 | ET13 | ET2 | 15 |
| Exemplary embodiment 90 | HT16 | HT3 | HT19 | BB19 | E21 | EM31 | ET26 | ET3 | 16 |
| Exemplary embodiment 91 | HT16 | HT2 | HT15 | BB18 | E31 | EM39 | ET13 | ET2 | 17 |
| Exemplary embodiment 92 | HT16 | HT2 | HT15 | CC19 | F7 | EM37 | ET13 | ET2 | 15 |
| Exemplary embodiment 93 | HT16 | HT3 | HT19 | CC18 | F27 | AA16 | ET16 | ET15 | 16 |
| Exemplary embodiment 94 | HT16 | HT3 | HT19 | AA7 | F1 | EM30 | ET16 | ET15 | 17 |
| Exemplary embodiment 95 | HT16 | HT3 | HT19 | BB31 | G25 | GD10 | ET17 | ET15 | 17 |
| Exemplary embodiment 96 | HT16 | HT3 | HT19 | BB19 | G16 | EM30 | ET26 | ET3 | 18 |
| Exemplary embodiment 97 | HT16 | HT2 | HT15 | BB18 | G27 | EM39 | ET13 | ET2 | 17 |
| Exemplary embodiment 98 | HT16 | HT2 | HT15 | CC19 | G28 | EM37 | ET13 | ET2 | 19 |
| Exemplary embodiment 99 | HT16 | HT3 | HT19 | CC18 | H27 | AA16 | ET16 | ET15 | 15 |
| Exemplary embodiment 100 | HT16 | HT3 | HT19 | CC18 | H7 | EM38 | ET16 | ET15 | 14 |

As described above, the use of an organic compound represented by the general formula [1] as a guest in the light-emitting layer can provide an organic light-emitting element with high maximum external quantum efficiency and luminescence efficiency.

The present disclosure can provide an organic compound with good emission properties.

While the present disclosure has been described with reference to exemplary embodiments, it is to be understood that the disclosure is not limited to the exemplary embodiments. The scope of the following claims is to be accorded the broadest interpretation so as to encompass all such modifications and equivalent structures and functions.

What is claimed is:

1. An organic compound represented by the following general formula [1]:
Ir Lₘ L′ₙ [1]
wherein Ir denotes iridium, L and L′ denote different bidentate ligands, m denotes an integer in the range of 1 to 3, n is 2 when m is 1, n is 1 when m is 2, and n is 0 when m is 3, the partial structure IrL denotes a partial structure represented by the following general formula [A-1] or [A-2], and the partial structure IrL′ denotes a partial structure represented by the following general formula [B-1] or [B-2], when m is 2 or more, the Ls may be the same or different, and when n is 2, the L′s may be the same or different, 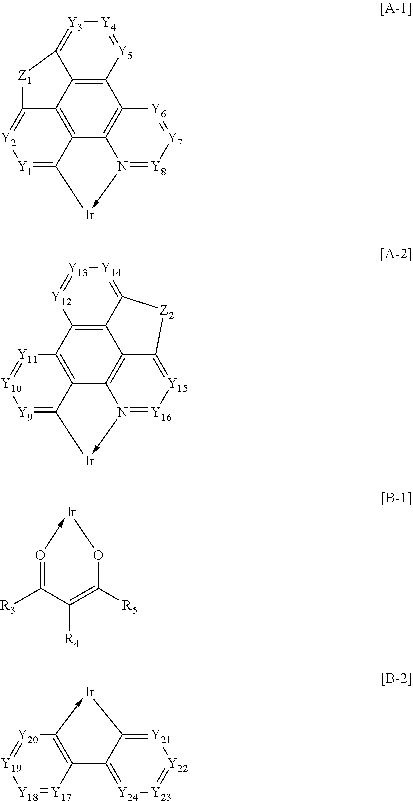
Y₁ to Y₂₄ in the general formulae [A-1], [A-2], and [B-2] are independently selected from a carbon atom and a nitrogen atom, when Y₁ to Y₂₄ denote a carbon atom, the carbon atom has a hydrogen atom, a deuterium atom, or a substituent R, and when two or more of Y₁ to Y₂₄ denote a carbon atom with the substituent R, the substituents R may have the same or different structures,
the substituent R denotes a substituent independently selected from a halogen atom, a substituted or unsubstituted alkyl group, a substituted or unsubstituted alkoxy group, a substituted or unsubstituted amino group, a substituted or unsubstituted aryloxy group, a substituted or unsubstituted silyl group, a cyano group, a substituted or unsubstituted aromatic hydrocarbon group, and a substituted or unsubstituted heterocyclic group,
when any adjacent two of Y₁ to Y₂₄ in the general formulae [A-1], [A-2], and [B-2] simultaneously denote a carbon atom and have the substituent R, the substituents R may be bonded together and form a ring, and the ring structure is a benzene ring, a naphthalene ring, an azine ring, a thiophene ring, or a furan ring,
Z₁ and Z₂ in the general formulae [A-1] and [A-2] are independently selected from an oxygen atom, a sulfur atom, S₁R₁R₂, CR₁R₂, GeR₁R₂, NR₁, and CR₁=CR₂, and R₁ and R₂ may be bonded together and form a ring, and
R₁ to R₅ in the general formulae [A-1], [A-2], and [B-1] are independently selected from a halogen atom, a substituted or unsubstituted alkyl group, a cyano group, a substituted or unsubstituted aromatic hydrocarbon group, and a substituted or unsubstituted heterocyclic group.

2. The organic compound according to claim 1, wherein the general formula [A-1] is independently selected from the following general formulae [A-11] to [A-14], and the general formula [A-2] is independently selected from the following general formulae [A-21] to [A-24], 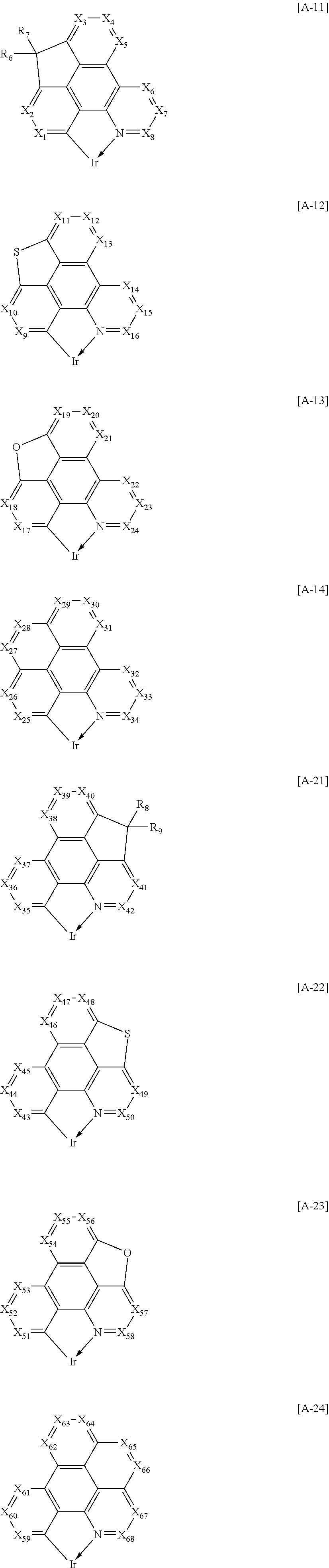
X₁ to X₆₈ in the general formulae [A-11] to [A-14] and [A-21] to [A-24] are independently selected from a carbon atom and a nitrogen atom, when X₁ to X₆₈ denote a carbon atom, the carbon atom has a hydrogen atom, a deuterium atom, or a substituent R, and when two or more of X₁ to X₆₈ denote a carbon atom with the substituent R, the substituents R may have the same or different structures,
the substituent R denotes a substituent independently selected from a halogen atom, a substituted or unsubstituted alkyl group, a substituted or unsubstituted alkoxy group, a substituted or unsubstituted amino group, a substituted or unsubstituted aryloxy group, a substituted or unsubstituted silyl group, a cyano group, a substituted or unsubstituted aromatic hydrocarbon group, and a substituted or unsubstituted heterocyclic group,
when any adjacent two of X₁ to X₆₈ in the general formulae [A-11] to [A-14] and [A-21] to [A-24] simultaneously denote a carbon atom and have the substituent R, the substituents R may be bonded together and form a ring, and the ring structure is a benzene ring, a naphthalene ring, an azine ring, a thiophene ring, or a furan ring, and
R₆ to R₉ in the general formulae [A-11] and [A-21] are independently selected from a halogen atom, a substituted or unsubstituted alkyl group, a cyano group, a substituted or unsubstituted aromatic hydrocarbon group, and a substituted or unsubstituted heterocyclic group.

3. The organic compound according to claim 2, wherein X₁ to X₆₈ denote a carbon atom.

4. An organic light-emitting element comprising:
a first electrode;
a second electrode; and
an organic compound layer located between the first electrode and the second electrode and having at least a light-emitting layer, wherein
the organic compound layer contains the organic compound according to claim 1.

5. The organic light-emitting element according to claim 4, wherein the light-emitting layer contains the organic compound.

6. The organic light-emitting element according to claim 5, wherein the light-emitting layer contains a first compound different from the organic compound.

7. The organic light-emitting element according to claim 6, wherein the amount of the organic compound in the light-emitting layer ranges from 1% to 30% by mass.

8. The organic light-emitting element according to claim 6, wherein the first compound has a carbazole structure.

9. The organic light-emitting element according to claim 8, wherein the first compound further has an azine ring.

10. The organic light-emitting element according to claim 6, wherein the first compound has at least one structure selected from a triphenylene structure, a phenanthrene structure, a chrysene structure, and a fluoranthene structure.

11. The organic light-emitting element according to claim 6, wherein the first compound has at least one structure selected from a dibenzothiophene structure and a dibenzofuran structure.

12. The organic light-emitting element according to claim 6, wherein the first compound has no sp3 carbon.

13. The organic light-emitting element according to claim 6, wherein the light-emitting layer further contains a second compound, which is different from the organic compound and the first compound.

14. The organic light-emitting element according to claim 13, wherein the second compound has an azine ring.

15. The organic light-emitting element according to claim 13, wherein the second compound has at least one structure selected from a xanthone structure, a thioxanthone structure, and a benzophenone structure.

16. The organic light-emitting element according to claim 4, wherein the light-emitting layer is a first light-emitting layer,
the organic light-emitting element further has a second light-emitting layer different from the first light-emitting layer disposed between the first light-emitting layer and the first electrode or between the first light-emitting layer and the second electrode, and
the second light-emitting layer emits light of a different color from light emitted by the first light-emitting layer.

17. A display apparatus comprising:
a plurality of pixels,
wherein at least one of the plurality of pixels includes the organic light-emitting element according to claim 4 and an active element coupled to the organic light-emitting element.

18. A photoelectric conversion apparatus comprising:
an optical unit with a plurality of lenses;
an imaging element configured to receive light passing through the optical unit; and
a display unit configured to display an image taken by the imaging element, wherein the display unit includes the organic light-emitting element according to claim 4.

19. Electronic equipment comprising:
a housing;
a communication unit configured to communicate with the outside; and
a display unit,
wherein the display unit includes the organic light-emitting element according to claim 4.

20. A lighting apparatus comprising:
a light source; and
a light-diffusing unit or an optical filter,
wherein the light source includes the organic light-emitting element according to claim 4.

21. A moving body comprising:
a body; and
a lamp provided on the body,
wherein the lamp includes the organic light-emitting element according to claim 4.

22. An exposure light source of an electrophotographic image-forming apparatus, compromising the organic light-emitting element according to claim 4.
